SEQUENCE LISTING (80 sequences)

SEQ ID NO: 1 - DNA, 1000 bp, synthetic construct
  misc_feature 1..1000: note = PD1 left homology
  source 1..1000: mol_type = other DNA
ccaagccctgaccctggcaggcatatgtttcaggaggtccttgtcttgggagcccagggtcgggggccccgtgtctgtccacatccgagtcaatggcccatctcgtctctgaagcatctttgctgtgagctctagtccccactgtcttgctggaaaatgtggaggccccactgcccactgcccagggcagcaatgcccataccacgtggtcccagctccgagcttgtcctgaaaagggggcaaagactggaccctgagcctgccaaggggccacactcctcccagggctggggtctccatgggcagccccccacccacccagaccagttacactcccctgtgccagagcagtgcagacaggaccaggccaggatgcccaagggtcaggggctggggatgggtagcccccaaacagccctttctgggggaactggcctcaacggggaagggggtgaaggctcttagtaggaaatcagggagacccaagtcagagccaggtgctgtgcagaagctgcagcctcacgtagaaggaagaggctctgcagtggaggccagtgcccatccccgggtggcagaggccccagcagagacttctcaatgacattccagctggggtggcccttccagagcccttgctgcccgagggatgtgagcaggtggccggggaggctttgtggggccacccagccccttcctcacctctctccatctctcagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacc

SEQ ID NO: 2 - DNA, 1000 bp, synthetic construct
  misc_feature 1..1000: note = PD1 right homology
  source 1..1000: mol_type = other DNA
gcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatctggggccccagcccacctgcggtctccgggggtgcccggcccatgtgtgtgcctgcctgcggtctccaggggtgcctggcccacgcgtgtgcccgcctgcggtctctgggggtgcccggcccacatatgtgcc

SEQ ID NO: 3 - DNA, 2781 bp, synthetic construct
  misc_feature 1..2781: note = PD1_T3C-L2
  source 1..2781: mol_type = other DNA
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtggtggctatcgcttccaagctggggggaaagcaggccctggagaccgtccaggcccttctcccagtgctttgccaggctcacggactgacccctgaacaggtggtggcaattgcctcacacgacgggggcaagcaggcactggagactgtccagcggctgctgcctgtcctctgccaggcccacggactcactcctgagcaggtcgtggccattgccagccacgatgggggcaaacaggctctggagaccgtgcagcgcctcctcccagtgctgtgccaggctcatgggctgaccccacagcaggtcgtcgccattgccagtaacggcggggggaagcaggccctcgaaacagtgcagaggctgctgcccgtcttgtgccaagcacacggcctgacacccgagcaggtggtggccatcgcctctcatgacggcggcaagcaggcccttgagacagtgcagagactgttgcccgtgttgtgtcaggcccacgggttgacaccccagcaggtggtcgccatcgccagcaatggcgggggaaagcaggcccttgagaccgtgcagcggttgcttccagtgttgtgccaggcacacggactgacccctcaacaggtggtcgcaatcgccagctacaagggcggaaagcaggctctggagacagtgcagcgcctcctgcccgtgctgtgtcaggctcacggactgacaccacagcaggtggtcgccatcgccagtaacgggggcggcaagcaggctttggagaccgtccagagactcctccccgtcctttgccaggcccacgggttgacacctcagcaggtcgtcgccattgcctccaacaacgggggcaagcaggccctcgaaactgtgcagaggctgctgcctgtgctgtgccaggctcatgggctgacaccccagcaggtggtggccattgcctctaacaacggcggcaaacaggcactggagaccgtgcaaaggctgctgcccgtcctctgccaagcccacgggctcactccacagcaggtcgtggccatcgcctcaaacaatggcgggaagcaggccctggagactgtgcaaaggctgctccctgtgctctgccaggcacacggactgacccctcagcaggtggtggcaatcgcttccaacaacgggggaaagcaggccctcgaaaccgtgcagcgcctcctcccagtgctgtgccaggcacatggcctcacacccgagcaagtggtggctatcgccagccacgacggagggaagcaggctctggagaccgtgcagaggctgctgcctgtcctgtgccaggcccacgggcttactccagagcaggtcgtcgccatcgccagtcatgatggggggaagcaggcccttgagacagtccagcggctgctgccagtcctttgccaggctcacggcttgactcccgagcaggtcgtggccattgcctcaaacattgggggcaaacaggccctggagacagtgcaggccctgctgcccgtgttgtgtcaggcccacggcttgacaccccagcaggtggtcgccattgcctctaatggcggcgggagacccgccttggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 4 - DNA, 2778 bp, synthetic construct
  misc_feature 1..2778: note = PD1T3R
  source 1..2778: mol_type = other DNA
atgggcgatcctaaaaagaaacgtaaggtcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtcgtcgcaatcgccagccatgatggagggaagcaagccctcgaaaccgtgcagcggttgcttcctgtgctctgccaggcccacggccttacccctcagcaggtggtggccatcgcaagtaacggaggaggaaagcaagccttggagacagtgcagcgcctgttgcccgtgctgtgccaggcacacggcctcacaccagagcaggtcgtggccattgcctcccatgacggggggaaacaggctctggagaccgtccagaggctgctgcccgtcctctgtcaagctcacggcctgactccccaacaagtggtcgccatcgcctctaatggcggcgggaagcaggcactggaaacagtgcagagactgctccctgtgctttgccaagctcatgggttgaccccccaacaggtcgtcgctattgcctcaaacggggggggcaagcaggcccttgagactgtgcagaggctgttgccagtgctgtgtcaggctcacgggctcactccacaacaggtggtcgcaattgccagcaacggcggcggaaagcaagctcttgaaaccgtgcaacgcctcctgcccgtgctctgtcaggctcatggcctgacaccacaacaagtcgtggccatcgccagtaataatggcgggaaacaggctcttgagaccgtccagaggctgctcccagtgctctgccaggcacacgggctgacccccgagcaggtggtggctatcgccagcaatattgggggcaagcaggccctggaaacagtccaggccctgctgccagtgctttgccaggctcacgggctcactccccagcaggtcgtggcaatcgcctccaacggcggagggaagcaggctctggagaccgtgcagagactgctgcccgtcttgtgccaggcccacggactcacacctgaacaggtcgtcgccattgcctctcacgatgggggcaaacaagccctggagacagtgcagcggctgttgcctgtgttgtgccaagcccacggcttgactcctcaacaagtggtcgccatcgcctcaaatggcggcggaaaacaagctctggagacagtgcagaggttgctgcccgtcctctgccaagcccacggcctgactccccaacaggtcgtcgccattgccagcaacaacggaggaaagcaggctctcgaaactgtgcagcggctgcttcctgtgctgtgtcaggctcatgggctgacccccgagcaagtggtggctattgcctctaatggaggcaagcaagcccttgagacagtccagaggctgttgccagtgctgtgccaggcccacgggctcacaccccagcaggtggtcgccatcgccagtaacaacgggggcaaacaggcattggaaaccgtccagcgcctgcttccagtgctctgccaggcacacggactgacacccgaacaggtggtggccattgcatcccatgatgggggcaagcaggccctggagaccgtgcagagactcctgccagtgttgtgccaagctcacggcctcacccctcagcaagtcgtggccatcgcctcaaacggggggggccggcctgcactggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 5 - DNA, 49 bp, synthetic construct
  misc_feature 1..49: note = PD1-T3
  source 1..49: mol_type = other DNA
tacctctgtggggccatctccctggcccccaaggcgcagatcaaagaga

SEQ ID NO: 6 - DNA, 60 bp, synthetic construct
  misc_feature 1..60: note = 2A-element
  source 1..60: mol_type = other DNA
tccggtgagggcagaggaagtcttctaacatgcggtgacgtggaggagaatccgggcccc

SEQ ID NO: 7 - DNA, 1989 bp, synthetic construct
  misc_feature 1..1989: note = apoptosis CAR
  source 1..1989: mol_type = other DNA
gctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattgcagatctcaaaaaagccaatctttgtactcttgcagagaaaattcagactatcatcctcaaggacattactagtgactcagaaaattcaaacttcagaaatgaaatccagagcttggtcgaa

SEQ ID NO: 8 - DNA, 276 bp, synthetic construct
  misc_feature 1..276: note = BGH polyA
  source 1..276: mol_type = other DNA
tctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattc

SEQ ID NO: 9 - DNA, 1000 bp, synthetic construct
  misc_feature 1..1000: note = Lck left homology
  source 1..1000: mol_type = other DNA
gggatagggggtgcctctgtgtgtgtgtgtgagagtgtgtgtgtgtagggtgtgtatatgtatagggtgtgtgtgagtgtgtgtgtgtgagagagtgtgtgtgtggcagaatagactgcggaggtggatttcatcttgatatgaaaggtctggaatgcatggtacattaaactttgaggacagcgctttccaagcactctgaggagcagccctagagaaggaggagctgcagggactccgggggcttcaaagtgagggccccactctgcttcaggcaaaacaggcacacatttatcactttatctatggagttctgcttgatttcatcagacaaaaaatttccactgctaaaacaggcaaataaacaaaaaaaaagttatggccaacagagtcactggagggttttctgctggggagaagcaagcccgtgtttgaaggaaccctgtgagatgactgtgggctgtgtgaggggaacagcgggggcttgatggtggacttcgggagcagaagcctctttctcagcctcctcagctagacaggggaattataataggaggtgtggcgtgcacacctctccagtaggggagggtctgataagtcaggtctctcccaggcttgggaaagtgtgtgtcatctctaggaggtggtcctcccaacacagggtactggcagagggagagggagggggcagaggcaggaagtgggtaactagactaacaaaggtgcctgtggcggtttgcccatcccaggtgggagggtggggctagggctcaggggccgtgtgtgaatttacttgtagcctgagggctcagagggagcaccggtttggagctgggaccccctattttagcttttctgtggctggtgaatggggatcccaggatctcacaatctcaggtacttttggaactttccagggcaaggccccattatatctgatgttgggggagcagatcttgggggagccccttcagccccctcttccattccctcagggacc

SEQ ID NO: 10 - protein, 219 aa, synthetic construct
  REGION 1..219: note = Interleukin-12 subunit alpha
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 11 - protein, 328 aa, synthetic construct
  REGION 1..328: note = Interleukin-12 subunit beta
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 12 - DNA, 1000 bp, synthetic construct
  misc_feature 1..1000: note = lck right homology
  source 1..1000: mol_type = other DNA
ggctgtggctgcagctcacacccggaagatgactggatggaaaacatcgatgtgtgtgagaactgccattatcccatagtcccactggatggcaagggcacggtaagaggcgagacaggggccttggtgagggagttgggtagagaatgcaacccaggagaaagaaatgaccagcactacaggcccttgaaagaatagagtggccctctcccctgaaatacagaaaggaaaagaggcccagagaggggaagggaatctcctaagatcacacagaaagtagttggtaaactcagggataacatctaaccaggctggagaggctgagagcagagcaggggggaagggggccagggtctgacccaatcttctgctttctgaccccaccctcatcccccactccacagctgctcatccgaaatggctctgaggtgcgggacccactggttacctacgaaggctccaatccgccggcttccccactgcaaggtgaccccaggcagcagggcctgaaagacaaggcctgcggatccctggctgttggcttccacctctcccccacctactttctccccggtcttgccttccttgtcccccaccctgtaactccaggcttcctgccgatcccagctcggttctccctgatgccccttgtctttacagacaacctggttatcgctctgcacagctatgagccctctcacgacggagatctgggctttgagaagggggaacagctccgcatcctggagcagtgagtccctctccaccttgctctggcggagtccgtgagggagcggcgatctccgcgacccgcagccctcctgcggcccttgaccagctcggggtggccgcccttgggacaaaattcgaggctcagtattgctgagccagggttgggggaggctggcttaaggggtggaggggtctttgagggagggtctcaggtcgacggctgagcgagccacactgacccacctccgtggcgcaggagcggcgagtg

SEQ ID NO: 13 - DNA, 1992 bp, synthetic construct
  misc_feature 1..1992: note = apoptosis CAR
  source 1..1992: mol_type = other DNA
atggctttgcctgtcactgccttgctgcttccacttgctctgttgttgcacgccgcaagacccgaggtcaagctccaggaaagcggaccagggctggtggcccctagtcagtcattgagcgtcacttgcaccgtcagcggcgtgtctctgcccgattacggcgtgagctggatcagacagcccccaaggaagggactggagtggctgggcgtcatctgggggagcgagactacctactacaacagcgccctgaagagcaggctgaccatcattaaggacaactccaagtcccaggtctttctgaaaatgaacagcctgcagactgatgacactgccatctactactgcgccaagcattactactacgggggcagctacgctatggactactgggggcaggggacctctgtcacagtgtcaagtggcggaggaggcagtggcggagggggaagtgggggcggcggcagcgacatccagatgacccagacaacatccagcctctccgcctctctgggcgacagagtgacaatcagctgccgggccagtcaggacatcagcaagtatctcaattggtaccagcagaaaccagacgggacagtgaaattgctgatctaccacacatccaggctgcactcaggagtccccagcaggttttccggctccggctccgggacagattacagtctgaccatttccaacctggagcaggaggatattgccacatacttttgccagcaaggcaacactctgccctataccttcggcggaggcacaaaactggagattactcggtcggatcccgagcccaaatctcctgacaaaactcacacatgcccaccgtgcccagcacctcccgtggccggcccgtcagtgttcctcttccccccaaaacccaaggacaccctcatgatcgcccggacccctgaggtcacatgcgtggtggtggacgtgagccacgaggaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtgttctcatgctccgtgatgcatgaggccctgcacaatcactatacccagaaatctctgagtctgagcccaggcaagaaggatattttggggtggctttgccttcttcttttgccaattccactaattgtttgggtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattgcagatctcaaaaaagccaatctttgtactcttgcagagaaaattcagactatcatcctcaaggacattactagtgactcagaaaattcaaacttcagaaatgaaatccagagcttggtcgaa

SEQ ID NO: 14 - DNA, 1000 bp, synthetic construct
  misc_feature 1..1000: note = Lck left homology
  source 1..1000: mol_type = other DNA
ctcataacaattctatgaggtaggaacagttatttactctattttccaaataaggaaactgggctcgcccaaggttccacaactaacatgtgtgtattattgagcatttaatttacaccagggaagcaggttgtggtggtgtgcacctgttgtccagctatttaggaggctgaggtgaaaggatcacttgaacggaggagttcaaatttgcaatgtgctatgattgtgcctgtgaacagctgctgcactccagcctgggcaacatagtgagatcccttatctaaaacattttttttaagtaaataatcaggtgggcacggtggctcacgcctgtaatccagcactttgggaggctgaggcgggcggatcacctgaggtcaggagttcaagaccagcctgaccaacatggagaaacccgtctctactaaaaatacaaaattagcttggcgtggtggtgcatgcctgtaatcccagctactcgagaagctgaggcaggagaattgtttgaacctgggaggtggaggttgcggtgagccgagatcgcaccattgcactccagcctgggcaacaagagtgaaattgcatctcaaaaaaaaagaaaaggaaataatctataccaggcactccaagtggtgtgactgatattcaacaagtacctctagtgtgaccttaccattgatgaagaccaagattcttttggattggtgctcacactgtgccagttaaatattccgaacattacccttgcctgtgggcttccagtgcctgaccttgatgtcctttcacccatcaacccgtagggatgaccaacccggaggtgattcagaacctggagcgaggctaccgcatggtgcgccctgacaactgtccagaggagctgtaccaactcatgaggctgtgctggaaggagcgcccagaggaccggcccacctttgactacctgcgcagtgtgctggaggacttcttcacggccacagagggccagtaccagcctcagcct

SEQ ID NO: 15 - DNA, 1000 bp, synthetic construct
  misc_feature 1..1000: note = lck right homology
  source 1..1000: mol_type = other DNA
gaggccttgagaggccctggggttctccccctttctctccagcctgacttggggagatggagttcttgtgccatagtcacatggcctatgcacatatggactctgcacatgaatcccacccacatgtgacacatatgcaccttgtgtctgtacacgtgtcctgtagttgcgtggactctgcacatgtcttgtacatgtgtagcctgtgcatgtatgtcttggacactgtacaaggtacccctttctggctctcccatttcctgagaccacagagagaggggagaagcctgggattgacagaagcttctgcccacctacttttctttcctcagatcatccagaagttcctcaagggccaggactttatctaatacctctgtgtgctcctccttggtgcctggcctggcacacatcaggagttcaataaatgtctgttgatgactgttgtacatctctttgctgtccactctttgtgggtgggcagtgggggttaagaaaatggtaattaggtcaccctgagttggggtgaaagatgggatgagtggatgtctggaggctctgcagaccccttcaaatgggacagtgctcctcacccctccccaaaggattcagggtgactcctacctggaatcccttagggaatgggtgcgtcaaaggaccttcctccccattataaaagggcaacagcattttttactgattcaagggctatatttgacctcagattttgtttttttaaggctagtcaaatgaagcggcgggaatggaggaggaacaaataaatctgtaactatcctcagatttttttttttttttgagactgggtctcactttttcatccaggctggagtgcagtcgcatgatcacggctcactgtagcctcaacctctccagctcaaatgctcctcctgtctcagcctcccgagtacctgggactactttcttgaggccaggaattcaagaacagagtaagatcctggtctccaaaaaaagttttaaa

SEQ ID NO: 16 - protein, 936 aa, synthetic construct
  REGION 1..936: note = TALEN TRAC
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 17 - protein, 942 aa, synthetic construct
  REGION 1..942: note = TALEN TRAC
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 18 - protein, 913 aa, synthetic construct
  REGION 1..913: note = TALEN CD25
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 19 - protein, 913 aa, synthetic construct
  REGION 1..913: note = TALEN CD25
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 20 - protein, 936 aa, synthetic construct
  REGION 1..936: note = TALEN PD1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASKLGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASYKGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 21 - protein, 941 aa, synthetic construct
  REGION 1..941: note = TALEN PD1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 22 - DNA, 2814 bp, synthetic construct
  misc_feature 1..2814: note = TALEN TRAC pCLS11370
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 23 - DNA, 2832 bp, synthetic construct
  misc_feature 1..2832: note = TALEN TRAC pCLS11369
  source 1..2832: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 24 - DNA, 2745 bp, synthetic construct
  misc_feature 1..2745: note = TALEN CD25 pCLS30480
  source 1..2745: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccgagtggcagcggaagtggcggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 25 - DNA, 2745 bp, synthetic construct
  misc_feature 1..2745: note = TALEN CD25 pCLS30479
  source 1..2745: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccgagtggcagcggaagtggcggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 26 - DNA, 2814 bp, synthetic construct
  misc_feature 1..2814: note = TALEN PD1 pCLS28959
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtggtggctatcgcttccaagctggggggaaagcaggccctggagaccgtccaggcccttctcccagtgctttgccaggctcacggactgacccctgaacaggtggtggcaattgcctcacacgacgggggcaagcaggcactggagactgtccagcggctgctgcctgtcctctgccaggcccacggactcactcctgagcaggtcgtggccattgccagccacgatgggggcaaacaggctctggagaccgtgcagcgcctcctcccagtgctgtgccaggctcatgggctgaccccacagcaggtcgtcgccattgccagtaacggcggggggaagcaggccctcgaaacagtgcagaggctgctgcccgtcttgtgccaagcacacggcctgacacccgagcaggtggtggccatcgcctctcatgacggcggcaagcaggcccttgagacagtgcagagactgttgcccgtgttgtgtcaggcccacgggttgacaccccagcaggtggtcgccatcgccagcaatggcgggggaaagcaggcccttgagaccgtgcagcggttgcttccagtgttgtgccaggcacacggactgacccctcaacaggtggtcgcaatcgccagctacaagggcggaaagcaggctctggagacagtgcagcgcctcctgcccgtgctgtgtcaggctcacggactgacaccacagcaggtggtcgccatcgccagtaacgggggcggcaagcaggctttggagaccgtccagagactcctccccgtcctttgccaggcccacgggttgacacctcagcaggtcgtcgccattgcctccaacaacgggggcaagcaggccctcgaaactgtgcagaggctgctgcctgtgctgtgccaggctcatgggctgacaccccagcaggtggtggccattgcctctaacaacggcggcaaacaggcactggagaccgtgcaaaggctgctgcccgtcctctgccaagcccacgggctcactccacagcaggtcgtggccatcgcctcaaacaatggcgggaagcaggccctggagactgtgcaaaggctgctccctgtgctctgccaggcacacggactgacccctcagcaggtggtggcaatcgcttccaacaacgggggaaagcaggccctcgaaaccgtgcagcgcctcctcccagtgctgtgccaggcacatggcctcacacccgagcaagtggtggctatcgccagccacgacggagggaagcaggctctggagaccgtgcagaggctgctgcctgtcctgtgccaggcccacgggcttactccagagcaggtcgtcgccatcgccagtcatgatggggggaagcaggcccttgagacagtccagcggctgctgccagtcctttgccaggctcacggcttgactcccgagcaggtcgtggccattgcctcaaacattgggggcaaacaggccctggagacagtgcaggccctgctgcccgtgttgtgtcaggcccacggcttgacaccccagcaggtggtcgccattgcctctaatggcggcgggagacccgccttggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 27 - DNA, 2829 bp, synthetic construct
  misc_feature 1..2829: note = TALEN PD1 pCLS18792
  source 1..2829: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtcgtcgcaatcgccagccatgatggagggaagcaagccctcgaaaccgtgcagcggttgcttcctgtgctctgccaggcccacggccttacccctcagcaggtggtggccatcgcaagtaacggaggaggaaagcaagccttggagacagtgcagcgcctgttgcccgtgctgtgccaggcacacggcctcacaccagagcaggtcgtggccattgcctcccatgacggggggaaacaggctctggagaccgtccagaggctgctgcccgtcctctgtcaagctcacggcctgactccccaacaagtggtcgccatcgcctctaatggcggcgggaagcaggcactggaaacagtgcagagactgctccctgtgctttgccaagctcatgggttgaccccccaacaggtcgtcgctattgcctcaaacggggggggcaagcaggcccttgagactgtgcagaggctgttgccagtgctgtgtcaggctcacgggctcactccacaacaggtggtcgcaattgccagcaacggcggcggaaagcaagctcttgaaaccgtgcaacgcctcctgcccgtgctctgtcaggctcatggcctgacaccacaacaagtcgtggccatcgccagtaataatggcgggaaacaggctcttgagaccgtccagaggctgctcccagtgctctgccaggcacacgggctgacccccgagcaggtggtggctatcgccagcaatattgggggcaagcaggccctggaaacagtccaggccctgctgccagtgctttgccaggctcacgggctcactccccagcaggtcgtggcaatcgcctccaacggcggagggaagcaggctctggagaccgtgcagagactgctgcccgtcttgtgccaggcccacggactcacacctgaacaggtcgtcgccattgcctctcacgatgggggcaaacaagccctggagacagtgcagcggctgttgcctgtgttgtgccaagcccacggcttgactcctcaacaagtggtcgccatcgcctcaaatggcggcggaaaacaagctctggagacagtgcagaggttgctgcccgtcctctgccaagcccacggcctgactccccaacaggtcgtcgccattgccagcaacaacggaggaaagcaggctctcgaaactgtgcagcggctgcttcctgtgctgtgtcaggctcatgggctgacccccgagcaagtggtggctattgcctctaatggaggcaagcaagcccttgagacagtccagaggctgttgccagtgctgtgccaggcccacgggctcacaccccagcaggtggtcgccatcgccagtaacaacgggggcaaacaggcattggaaaccgtccagcgcctgcttccagtgctctgccaggcacacggactgacacccgaacaggtggtggccattgcatcccatgatgggggcaagcaggccctggagaccgtgcagagactcctgccagtgttgtgccaagctcacggcctcacccctcagcaagtcgtggccatcgcctcaaacggggggggccggcctgcactggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 28 - DNA, 49 bp, synthetic construct
  misc_feature 1..49: note = TALEN target TRAC
  source 1..49: mol_type = other DNA
ttgtcccacagatatccagaaccctgaccctgccgtgtaccagctgaga

SEQ ID NO: 29 - DNA, 45 bp, synthetic construct
  misc_feature 1..45: note = TALEN target CD25
  source 1..45: mol_type = other DNA
tacaggaggaagagtagaagaacaatctagaaaaccaaaagaaca

SEQ ID NO: 30 - DNA, 49 bp, synthetic construct; identical to SEQ ID NO: 5
  misc_feature 1..49: note = TALEN target PD1
  source 1..49: mol_type = other DNA

SEQ ID NO: 31 - DNA, 2897 bp, synthetic construct
  misc_feature 1..2897: note = Matrice TRAC locus_CubiCAR CD22 pCLS30056
  source 1..2897: mol_type = other DNA
ttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagtacccctacgacgtgcccgactacgcctccggtgagggcagaggaagtcttctaacatgcggtgacgtggaggagaatccgggccccggatccgctctgcccgtcaccgctctgctgctgccactggcactgctgctgcacgctgctaggcccggagggggaggcagctgcccctacagcaaccccagcctgtgcagcggaggcggcggcagcggcggagggggtagccaggtgcagctgcagcagagcggccctggcctggtgaagccaagccagacactgtccctgacctgcgccatcagcggcgattccgtgagctccaactccgccgcctggaattggatcaggcagtccccttctcggggcctggagtggctgggaaggacatactatcggtctaagtggtacaacgattatgccgtgtctgtgaagagcagaatcacaatcaaccctgacacctccaagaatcagttctctctgcagctgaatagcgtgacaccagaggacaccgccgtgtactattgcgccagggaggtgaccggcgacctggaggatgcctttgacatctggggccagggcacaatggtgaccgtgagctccggaggcggcggatctggcggaggaggaagtgggggcggcgggagtgatatccagatgacacagtccccatcctctctgagcgcctccgtgggcgacagagtgacaatcacctgtagggcctcccagaccatctggtcttacctgaactggtatcagcagaggcccggcaaggcccctaatctgctgatctacgcagcaagctccctgcagagcggagtgccatccagattctctggcaggggctccggcacagacttcaccctgaccatctctagcctgcaggccgaggacttcgccacctactattgccagcagtcttatagcatcccccagacatttggccagggcaccaagctggagatcaagtcggatcccggaagcggagggggaggcagctgcccctacagcaaccccagcctgtgcagcggaggcggcggcagcgagctgcccacccagggcaccttctccaacgtgtccaccaacgtgagcccagccaagcccaccaccaccgcctgtccttattccaatccttccctgtgtgctcccaccacaacccccgctccaaggccccctacccccgcaccaactattgcctcccagccactctcactgcggcctgaggcctgtcggcccgctgctggaggcgcagtgcatacaaggggcctcgatttcgcctgcgatatttacatctgggcacccctcgccggcacctgcggggtgcttctcctctccctggtgattaccctgtattgcagacggggccggaagaagctcctctacatttttaagcagcctttcatgcggccagtgcagacaacccaagaggaggatgggtgttcctgcagattccctgaggaagaggaaggcgggtgcgagctgagagtgaagttctccaggagcgcagatgcccccgcctatcaacagggccagaaccagctctacaacgagcttaacctcgggaggcgcgaagaatacgacgtgttggataagagaagggggcgggaccccgagatgggaggaaagccccggaggaagaaccctcaggagggcctgtacaacgagctgcagaaggataagatggccgaggcctactcagagatcgggatgaagggggagcggcgccgcgggaaggggcacgatgggctctaccaggggctgagcacagccacaaaggacacatacgacgccttgcacatgcaggcccttccaccccgggaatagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaa

SEQ ID NO: 32 - DNA, 2688 bp, synthetic construct
  misc_feature 1..2688: note = Matrice CD25 locus_IL15_2A_sIL15Ra pCLS30519
  source 1..2688: mol_type = other DNA
gtttattattcctgttccacagctattgtctgccatataaaaacttaggccaggcacagtggctcacacctgtaatcccagcactttggaaggccgaggcaggcagatcacaaggtcaggagttcgagaccagcctggccaacatagcaaaaccccatctctactaaaaatacaaaaattagccaggcatggtggcgtgtgcactggtttagagtgaggaccacatttttttggtgccgtgttacacatatgaccgtgactttgttacaccactacaggaggaagagtagaagaacaatcggttctggcgtgaaacagactttgaattttgaccttctcaagttggcgggagacgtggagtccaacccagggcccggtaccgggtccgccaccatggactggacctggattctgttcctcgtggctgctgctacaagagtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggaagcggagctactaacttcagcctgctgaagcaggctggagacgtggaggagaaccctggacctgggaccggctctgcaaccatggattggacgtggatcctgtttctcgtggcagctgccacaagagttcacagtatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcat

SEQ ID NO: 33 - DNA, 2964 bp, synthetic construct
  misc_feature 1..2964: note = Matrice PD1 locus_IL15_2A_sIL15Ra pCLS30513
  source 1..2964: mol_type = other DNA
gactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccggttctggcgtgaaacagactttgaattttgaccttctcaagttggcgggagacgtggagtccaacccagggcccggtaccgggtccgccaccatggactggacctggattctgttcctcgtggctgctgctacaagagtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggaagcggagctactaacttcagcctgctgaagcaggctggagacgtggaggagaaccctggacctgggaccggctctgcaaccatggattggacgtggatcctgtttctcgtggcagctgccacaagagttcacagtatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttg

SEQ ID NO: 34 - DNA, 3363 bp, synthetic construct
  misc_feature 1..3363: note = Matrice CD25 locus_IL12a_2A_IL12b pCLS30520
  source 1..3363: mol_type = other DNA
gtttattattcctgttccacagctattgtctgccatataaaaacttaggccaggcacagtggctcacacctgtaatcccagcactttggaaggccgaggcaggcagatcacaaggtcaggagttcgagaccagcctggccaacatagcaaaaccccatctctactaaaaatacaaaaattagccaggcatggtggcgtgtgcactggtttagagtgaggaccacatttttttggtgccgtgttacacatatgaccgtgactttgttacaccactacaggaggaagagtagaagaacaatcggttctggcgtgaaacagactttgaattttgaccttctcaagttggcgggagacgtggagtccaacccagggcccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggaagcggagctactaacttcagcctgctgaagcaggctggagacgtggaggagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcat

SEQ ID NO: 35 - DNA, 3639 bp, synthetic construct
  misc_feature 1..3639: note = Matrice PD1 locus_IL12a_2A_IL12b pCLS30511
  source 1..3639: mol_type = other DNA
gactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccggttctggcgtgaaacagactttgaattttgaccttctcaagttggcgggagacgtggagtccaacccagggcccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggaagcggagctactaacttcagcctgctgaagcaggctggagacgtggaggagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttg

SEQ ID NO: 36 - DNA, 3017 bp, synthetic construct
  misc_feature 1..3017: note = Inserted matrice TRAC locus_CubiCAR CD22 (60 nucleotides upstream and downstream)
  source 1..3017: mol_type = other DNA
atgagatcatgtcctaaccctgatcctcttgtcccacagatatccagaaccctgaccctgttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagtacccctacgacgtgcccgactacgcctccggtgagggcagaggaagtcttctaacatgcggtgacgtggaggagaatccgggccccggatccgctctgcccgtcaccgctctgctgctgccactggcactgctgctgcacgctgctaggcccggagggggaggcagctgcccctacagcaaccccagcctgtgcagcggaggcggcggcagcggcggagggggtagccaggtgcagctgcagcagagcggccctggcctggtgaagccaagccagacactgtccctgacctgcgccatcagcggcgattccgtgagctccaactccgccgcctggaattggatcaggcagtccccttctcggggcctggagtggctgggaaggacatactatcggtctaagtggtacaacgattatgccgtgtctgtgaagagcagaatcacaatcaaccctgacacctccaagaatcagttctctctgcagctgaatagcgtgacaccagaggacaccgccgtgtactattgcgccagggaggtgaccggcgacctggaggatgcctttgacatctggggccagggcacaatggtgaccgtgagctccggaggcggcggatctggcggaggaggaagtgggggcggcgggagtgatatccagatgacacagtccccatcctctctgagcgcctccgtgggcgacagagtgacaatcacctgtagggcctcccagaccatctggtcttacctgaactggtatcagcagaggcccggcaaggcccctaatctgctgatctacgcagcaagctccctgcagagcggagtgccatccagattctctggcaggggctccggcacagacttcaccctgaccatctctagcctgcaggccgaggacttcgccacctactattgccagcagtcttatagcatcccccagacatttggccagggcaccaagctggagatcaagtcggatcccggaagcggagggggaggcagctgcccctacagcaaccccagcctgtgcagcggaggcggcggcagcgagctgcccacccagggcaccttctccaacgtgtccaccaacgtgagcccagccaagcccaccaccaccgcctgtccttattccaatccttccctgtgtgctcccaccacaacccccgctccaaggccccctacccccgcaccaactattgcctcccagccactctcactgcggcctgaggcctgtcggcccgctgctggaggcgcagtgcatacaaggggcctcgatttcgcctgcgatatttacatctgggcacccctcgccggcacctgcggggtgcttctcctctccctggtgattaccctgtattgcagacggggccggaagaagctcctctacatttttaagcagcctttcatgcggccagtgcagacaacccaagaggaggatgggtgttcctgcagattccctgaggaagaggaaggcgggtgcgagctgagagtgaagttctccaggagcgcagatgcccccgcctatcaacagggccagaaccagctctacaacgagcttaacctcgggaggcgcgaagaatacgacgtgttggataagagaagggggcgggaccccgagatgggaggaaagccccggaggaagaaccctcaggagggcctgtacaacgagctgcagaaggataagatggccgaggcctactcagagatcgggatgaagggggagcggcgccgcgggaaggggcacgatgggctctaccaggggctgagcacagccacaaaggacacatacgacgccttgcacatgcaggcccttccaccccgggaatagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaaga

SEQ ID NO: 37 - DNA, 2808 bp, synthetic construct
  misc_feature 1..2808: note = Inserted matrice CD25 locus_IL15_2A_sIL15Ra (60 nucleotides upstream and downstream)
  source 1..2808: mol_type = other DNA
agtgctggctagaaaccaagtgctttactgcatgcacatcatttagcacagttagttgctgtttattattcctgttccacagctattgtctgccatataaaaacttaggccaggcacagtggctcacacctgtaatcccagcactttggaaggccgaggcaggcagatcacaaggtcaggagttcgagaccagcctggccaacatagcaaaaccccatctctactaaaaatacaaaaattagccaggcatggtggcgtgtgcactggtttagagtgaggaccacatttttttggtgccgtgttacacatatgaccgtgactttgttacaccactacaggaggaagagtagaagaacaatcggttctggcgtgaaacagactttgaattttgaccttctcaagttggcgggagacgtggagtccaacccagggcccggtaccgggtccgccaccatggactggacctggattctgttcctcgtggctgctgctacaagagtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggaagcggagctactaacttcagcctgctgaagcaggctggagacgtggaggagaaccctggacctgggaccggctctgcaaccatggattggacgtggatcctgtttctcgtggcagctgccacaagagttcacagtatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtt

SEQ ID NO: 38 - DNA, 3084 bp, synthetic construct
  misc_feature 1..3084: note = Inserted matrice PD1 locus_IL15_2A_sIL15Ra (60 nucleotides upstream and downstream)
  source 1..3084: mol_type = other DNA
ggtggccggggaggctttgtggggccacccagccccttcctcacctctctccatctctcagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccggttctggcgtgaaacagactttgaattttgaccttctcaagttggcgggagacgtggagtccaacccagggcccggtaccgggtccgccaccatggactggacctggattctgttcctcgtggctgctgctacaagagtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggaagcggagctactaacttcagcctgctgaagcaggctggagacgtggaggagaaccctggacctgggaccggctctgcaaccatggattggacgtggatcctgtttctcgtggcagctgccacaagagttcacagtatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgcccacagcccaccccagcccctcacccaggcc

SEQ ID NO: 39 - DNA, 3475 bp, synthetic construct
  misc_feature 1..3475: note = Inserted matrice CD25 locus_IL12a_2A_IL12b (60 nucleotides upstream and downstream)
  source 1..3475: mol_type = other DNA
agtgctggctagaaaccaagtgctttactgcatgcacatcatttagcacagttagttgctgtttattattcctgttccacagctattgtctgccatataaaaacttaggccaggcacagtggctcacacctgtaatcccagcactttggaaggccgaggcaggcagatcacaaggtcaggagttcgagaccagcctggccaacatagcaaaaccccatctctactaaaaatacaaaaattagccaggcatggtggcgtgtgcactggtttagagtgaggaccacatttttttggtgccgtgttacacatatgaccgtgactttgttacaccactacaggaggaagagtagaagaacaatcggttctggcgtgaaacagactttgaattttgaccttctcaagttggcgggagacgtggagtccaacccagggcccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggaagcggagctactaacttcagcctgctgaagcaggctggagacgtggaggagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcatgaatggtatggaactctctccaccctatatgtagtataaagaaaagtaggtt

SEQ ID NO: 40 - DNA, 3759 bp, synthetic construct
  misc_feature 1..3759: note = Inserted matrice PD1 locus_IL12a_2A_IL12b (60 nucleotides upstream and downstream)
  source 1..3759: mol_type = other DNA
ggtggccggggaggctttgtggggccacccagccccttcctcacctctctccatctctcagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccggttctggcgtgaaacagactttgaattttgaccttctcaagttggcgggagacgtggagtccaacccagggcccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggaagcggagctactaacttcagcctgctgaagcaggctggagacgtggaggagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgcccacagcccaccccagcccctcacccaggcc

SEQ ID NO: 41 - DNA, 60 bp, synthetic construct
  misc_feature 1..60: note = upstream TRAC locus polynucleotide sequence
  source 1..60: mol_type = other DNA
atgagatcatgtcctaaccctgatcctcttgtcccacagatatccagaaccctgaccctg

SEQ ID NO: 42 - DNA, 60 bp, synthetic construct
  misc_feature 1..60: note = downstream TRAC locus polynucleotide sequence
  source 1..60: mol_type = other DNA
gaaacagtgagccttgttctggcagtccagagaatgacacgggaaaaaagcagatgaaga

SEQ ID NO: 43 - DNA, 60 bp, synthetic construct
  misc_feature 1..60: note = upstream CD25 locus polynucleotide sequence
  source 1..60: mol_type = other DNA
agtgctggctagaaaccaagtgctttactgcatgcacatcatttagcacagttagttgct

SEQ ID NO: 44 - DNA, 52 bp, synthetic construct
  misc_feature 1..52: note = downstream CD25 locus polynucleotide sequence
  source 1..52: mol_type = other DNA
gaatggtatggaactctctccaccctatatgtagtataaagaaaagtaggtt

SEQ ID NO: 45 - DNA, 60 bp, synthetic construct
  misc_feature 1..60: note = upstream PD1 locus polynucleotide sequence
  source 1..60: mol_type = other DNA
ggtggccggggaggctttgtggggccacccagccccttcctcacctctctccatctctca

SEQ ID NO: 46 - DNA, 60 bp, synthetic construct
  misc_feature 1..60: note = downstream PD1 locus polynucleotide sequence
  source 1..60: mol_type = other DNA
tgcccttccagagagaagggcagaagtgcccacagcccaccccagcccctcacccaggcc

SEQ ID NO: 47 - DNA, 759 bp, synthetic construct
  misc_feature 1..759: note = IL-12a polynucleotide
  source 1..759: mol_type = other DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 48 - DNA, 984 bp, synthetic construct
  misc_feature 1..984: note = IL12b polynucleotide
  source 1..984: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 49 - DNA, 399 bp, synthetic construct
  misc_feature 1..399: note = IL15 polynucleotide
  source 1..399: mol_type = other DNA
ggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 50 - DNA, 525 bp, synthetic construct
  misc_feature 1..525: note = sIL15ra polynucleotide
  source 1..525: mol_type = other DNA
atcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccact

SEQ ID NO: 51 - DNA, 1818 bp, synthetic construct
  misc_feature 1..1818: note = soluble GP130 polynucleotide
  source 1..1818: mol_type = other DNA
atgctgacactgcagacttggctggtgcaggcactgtttatttttctgactactgaatcaactggcgaactgctggacccttgtggctacatcagccctgagtccccagtggtgcagctgcacagcaacttcaccgccgtgtgcgtgctgaaggagaagtgtatggactactttcacgtgaacgccaattatatcgtgtggaaaaccaaccacttcacaatccccaaggagcagtacaccatcatcaataggacagccagctccgtgacctttacagacatcgcctccctgaacatccagctgacctgcaatatcctgacattcggccagctggagcagaacgtgtatggcatcaccatcatctctggcctgccccctgagaagcctaagaacctgagctgcatcgtgaatgagggcaagaagatgcggtgtgagtgggacggcggcagagagacacacctggagacaaacttcaccctgaagtccgagtgggccacacacaagtttgccgactgcaaggccaagcgcgataccccaacatcctgtaccgtggattactctacagtgtattttgtgaacatcgaagtgtgggtggaggccgagaatgccctgggcaaggtgacctccgaccacatcaacttcgatcccgtgtacaaggtgaagcctaacccaccccacaatctgagcgtgatcaattccgaggagctgtctagcatcctgaagctgacctggacaaacccatctatcaagagcgtgatcatcctgaagtacaatatccagtatcggaccaaggacgcctccacatggagccagatccctccagaggataccgccagcacaagatcctctttcaccgtgcaggacctgaagcccttcacagagtacgtgtttcggatcagatgtatgaaggaggacggcaagggctactggagcgattggtccgaggaggccagcggcatcacctatgaggacaggccttctaaggcccccagcttctggtacaagatcgatccatcccacacccagggctatcgcacagtgcagctggtgtggaaaaccctgccccctttcgaggccaacggcaagatcctggactacgaggtgaccctgacacggtggaagtcccacctgcagaactataccgtgaatgccaccaagctgacagtgaacctgacaaatgatcggtacctggccaccctgacagtgagaaacctggtgggcaagtctgacgccgccgtgctgaccatccctgcctgcgatttccaggccacacacccagtgatggacctgaaggcctttcccaaggataatatgctgtgggtggagtggaccacacctagagagtccgtgaagaagtacatcctggagtggtgcgtgctgtctgacaaggccccatgtatcaccgactggcagcaggaggatggcaccgtgcacaggacatatctgcgcggcaacctggccgagtctaagtgttacctgatcaccgtgacacccgtgtatgcagacggaccaggctctcctgagagcatcaaggcctacctgaagcaggcaccaccaagcaagggaccaaccgtgcggacaaagaaggtcggcaagaatgaggccgtgctggagtgggaccagctgcctgtggatgtgcagaacggcttcatcaggaattacaccatcttttatcgcacaatcatcggcaacgagacagccgtgaatgtggacagctcccacaccgagtatacactgtctagcctgacctccgatacactgtacatggtgaggatggccgcctatacagacgagggcggcaaggatggccccgagttt

SEQ ID NO: 52 - DNA, 72 bp, synthetic construct
  misc_feature 1..72: note = IgE signal sequence
  source 1..72: mol_type = other DNA
ggtaccgggtccgccaccatggactggacctggattctgttcctcgtggctgctgctacaagagtgcacagc

SEQ ID NO: 53 - DNA, 75 bp, synthetic construct
  misc_feature 1..75: note = F2A
  source 1..75: mol_type = other DNA
ggttctggcgtgaaacagactttgaattttgaccttctcaagttggcgggagacgtggagtccaacccagggccc

SEQ ID NO: 54 - DNA, 66 bp, synthetic construct
  misc_feature 1..66: note = P2A
  source 1..66: mol_type = other DNA
ggaagcggagctactaacttcagcctgctgaagcaggctggagacgtggaggagaaccctggacct

SEQ ID NO: 55 - DNA, 54 bp, synthetic construct
  misc_feature 1..54: note = T2A
  source 1..54: mol_type = other DNA
gagggcagaggcagcctgctgacctgcggcgacgtcgaggagaaccccgggccc

SEQ ID NO: 56 - DNA, 825 bp, synthetic construct
  misc_feature 1..825: note = LNGFR
  source 1..825: mol_type = other DNA
atgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtga

SEQ ID NO: 57 - protein, 253 aa, synthetic construct
  REGION 1..253: note = IL-12a polypeptide
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 58 - protein, 328 aa, synthetic construct; identical to SEQ ID NO: 11
  REGION 1..328: note = IL12b polypeptide

SEQ ID NO: 59 - protein, 133 aa, synthetic construct
  REGION 1..133: note = IL15 polypeptide
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 60 - protein, 175 aa, synthetic construct
  REGION 1..175: note = sIL15ra polypeptide
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 61 - protein, 606 aa, synthetic construct
  REGION 1..606: note = soluble gp130
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEF

SEQ ID NO: 62 - protein, 836 aa, synthetic construct
  REGION 1..836: note = soluble gp130 fused to a Fc
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFRSCDKTHTCPPCPAPEAEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 7711 bp, synthetic construct
  misc_feature 1..7711: note = Matrice TRAC locus_CubiCAR CD22 pCLS30056 full sequence
  source 1..7711: mol_type = other DNA
gtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggtctttcctgcgttatcccctgattctgtggataaccgtattaccgcctttgagtgagctgataccgctcgccgcagccgaacgaccgagcgcagcgagtcagtgagcgaggaagcggagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgtcaattaaccctcactaaagggaacaaaagctgttaattaattgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagtacccctacgacgtgcccgactacgcctccggtgagggcagaggaagtcttctaacatgcggtgacgtggaggagaatccgggccccggatccgctctgcccgtcaccgctctgctgctgccactggcactgctgctgcacgctgctaggcccggagggggaggcagctgcccctacagcaaccccagcctgtgcagcggaggcggcggcagcggcggagggggtagccaggtgcagctgcagcagagcggccctggcctggtgaagccaagccagacactgtccctgacctgcgccatcagcggcgattccgtgagctccaactccgccgcctggaattggatcaggcagtccccttctcggggcctggagtggctgggaaggacatactatcggtctaagtggtacaacgattatgccgtgtctgtgaagagcagaatcacaatcaaccctgacacctccaagaatcagttctctctgcagctgaatagcgtgacaccagaggacaccgccgtgtactattgcgccagggaggtgaccggcgacctggaggatgcctttgacatctggggccagggcacaatggtgaccgtgagctccggaggcggcggatctggcggaggaggaagtgggggcggcgggagtgatatccagatgacacagtccccatcctctctgagcgcctccgtgggcgacagagtgacaatcacctgtagggcctcccagaccatctggtcttacctgaactggtatcagcagaggcccggcaaggcccctaatctgctgatctacgcagcaagctccctgcagagcggagtgccatccagattctctggcaggggctccggcacagacttcaccctgaccatctctagcctgcaggccgaggacttcgccacctactattgccagcagtcttatagcatcccccagacatttggccagggcaccaagctggagatcaagtcggatcccggaagcggagggggaggcagctgcccctacagcaaccccagcctgtgcagcggaggcggcggcagcgagctgcccacccagggcaccttctccaacgtgtccaccaacgtgagcccagccaagcccaccaccaccgcctgtccttattccaatccttccctgtgtgctcccaccacaacccccgctccaaggccccctacccccgcaccaactattgcctcccagccactctcactgcggcctgaggcctgtcggcccgctgctggaggcgcagtgcatacaaggggcctcgatttcgcctgcgatatttacatctgggcacccctcgccggcacctgcggggtgcttctcctctccctggtgattaccctgtattgcagacggggccggaagaagctcctctacatttttaagcagcctttcatgcggccagtgcagacaacccaagaggaggatgggtgttcctgcagattccctgaggaagaggaaggcgggtgcgagctgagagtgaagttctccaggagcgcagatgcccccgcctatcaacagggccagaaccagctctacaacgagcttaacctcgggaggcgcgaagaatacgacgtgttggataagagaagggggcgggaccccgagatgggaggaaagccccggaggaagaaccctcaggagggcctgtacaacgagctgcagaaggataagatggccgaggcctactcagagatcgggatgaagggggagcggcgccgcgggaaggggcacgatgggctctaccaggggctgagcacagccacaaaggacacatacgacgccttgcacatgcaggcccttccaccccgggaatagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaggcgcgccactcgagcgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaggcgcgcccaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttggcctgtagtgggccatagccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttag

SEQ ID NO: 64 - DNA, 7502 bp, synthetic construct
  misc_feature 1..7502: note = Matrice CD25 locus_IL15_2A_sIL15Ra pCLS30519 full sequence
  source 1..7502: mol_type = other DNA
gtttattattcctgttccacagctattgtctgccatataaaaacttaggccaggcacagtggctcacacctgtaatcccagcactttggaaggccgaggcaggcagatcacaaggtcaggagttcgagaccagcctggccaacatagcaaaaccccatctctactaaaaatacaaaaattagccaggcatggtggcgtgtgcactggtttagagtgaggaccacatttttttggtgccgtgttacacatatgaccgtgactttgttacaccactacaggaggaagagtagaagaacaatcggttctggcgtgaaacagactttgaattttgaccttctcaagttggcgggagacgtggagtccaacccagggcccggtaccgggtccgccaccatggactggacctggattctgttcctcgtggctgctgctacaagagtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggaagcggagctactaacttcagcctgctgaagcaggctggagacgtggaggagaaccctggacctgggaccggctctgcaaccatggattggacgtggatcctgtttctcgtggcagctgccacaagagttcacagtatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcatgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaggcgcgccactcgagcgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaggcgcgcccaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttggcctgtagtgggccatagccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggtctttcctgcgttatcccctgattctgtggataaccgtattaccgcctttgagtgagctgataccgctcgccgcagccgaacgaccgagcgcagcgagtcagtgagcgaggaagcggagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgtcaattaaccctcactaaagggaacaaaagctgttaattaa

SEQ ID NO: 65 - DNA, 7778 bp, synthetic construct
  misc_feature 1..7778: note = Matrice PD1 locus_IL15_2A_sIL15Ra pCLS30513 full sequence
  source 1..7778: mol_type = other DNA
gactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccggttctggcgtgaaacagactttgaattttgaccttctcaagttggcgggagacgtggagtccaacccagggcccggtaccgggtccgccaccatggactggacctggattctgttcctcgtggctgctgctacaagagtgcacagcggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggaagcggagctactaacttcagcctgctgaagcaggctggagacgtggaggagaaccctggacctgggaccggctctgcaaccatggattggacgtggatcctgtttctcgtggcagctgccacaagagttcacagtatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttggcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaggcgcgccactcgagcgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaggcgcgcccaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttggcctgtagtgggccatagccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggtctttcctgcgttatcccctgattctgtggataaccgtattaccgcctttgagtgagctgataccgctcgccgcagccgaacgaccgagcgcagcgagtcagtgagcgaggaagcggagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgtcaattaaccctcactaaagggaacaaaagctgttaattaa

SEQ ID NO: 66 - DNA, 8177 bp, synthetic construct
  misc_feature 1..8177: note = Matrice CD25 locus_IL12a_2A_IL12b pCLS30520 full sequence
  source 1..8177: mol_type = other DNA
gtttattattcctgttccacagctattgtctgccatataaaaacttaggccaggcacagtggctcacacctgtaatcccagcactttggaaggccgaggcaggcagatcacaaggtcaggagttcgagaccagcctggccaacatagcaaaaccccatctctactaaaaatacaaaaattagccaggcatggtggcgtgtgcactggtttagagtgaggaccacatttttttggtgccgtgttacacatatgaccgtgactttgttacaccactacaggaggaagagtagaagaacaatcggttctggcgtgaaacagactttgaattttgaccttctcaagttggcgggagacgtggagtccaacccagggcccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggaagcggagctactaacttcagcctgctgaagcaggctggagacgtggaggagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgaaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcatgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgccaccatggcttcttaccctggacaccagcatgcttctgcctttgaccaggctgccagatccaggggccactccaacaggagaactgccctaagacccagaagacagcaggaagccactgaggtgaggcctgagcagaagatgccaaccctgctgagggtgtacattgatggacctcatggcatgggcaagaccaccaccactcaactgctggtggcactgggctccagggatgacattgtgtatgtgcctgagccaatgacctactggagagtgctaggagcctctgagaccattgccaacatctacaccacccagcacaggctggaccagggagaaatctctgctggagatgctgctgtggtgatgacctctgcccagatcacaatgggaatgccctatgctgtgactgatgctgttctggctcctcacattggaggagaggctggctcttctcatgcccctccacctgccctgaccctgatctttgacagacaccccattgcagccctgctgtgctacccagcagcaaggtacctcatgggctccatgaccccacaggctgtgctggcttttgtggccctgatccctccaaccctccctggcaccaacattgttctgggagcactgcctgaagacagacacattgacaggctggcaaagaggcagagacctggagagagactggacctggccatgctggctgcaatcagaagggtgtatggactgctggcaaacactgtgagatacctccagtgtggaggctcttggagagaggactggggacagctctctggaacagcagtgccccctcaaggagctgagccccagtccaatgctggtccaagaccccacattggggacaccctgttcaccctgttcagagcccctgagctgctggctcccaatggagacctgtacaatgtgtttgcctgggctctggatgttctagccaagaggctgaggtccatgcatgtgttcatcctggactatgaccagtcccctgctggatgcagagatgctctgctgcaactaacctctggcatggtgcagacccatgtgaccacccctggcagcatccccaccatctgtgacctagccagaacctttgccagggagatgggagaggccaactaaggcgcgccactcgagcgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaggcgcgcccaattcgccctatagtgagtcgtattacgtcgcgctcactggccgtcgttttacaacgtcgtgactgggaaaaccctggcgttacccaacttaatcgccttgcagcacatccccctttcgccagctggcgtaatagcgaagaggcccgcaccgaaacgcccttcccaacagttgcgcagcctgaatggcgaatgggagcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttggcctgtagtgggccatagccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggtctttcctgcgttatcccctgattctgtggataaccgtattaccgcctttgagtgagctgataccgctcgccgcagccgaacgaccgagcgcagcgagtcagtgagcgaggaagcggagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgtcaattaaccctcactaaagggaacaaaagctgttaattaa

SEQ ID NO: 67 - DNA, 6349 bp, synthetic construct
  misc_feature 1..6349: note = Matrice PD1 locus_IL12a_2A_IL12b pCLS30511 full sequence
  source 1..6349: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatgactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccggttctggcgtgaaacagactttgaattttgaccttctcaagttggcgggagacgtggagtccaacccagggcccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggaagcggagctactaacttcagcctgctgaagcaggctggagacgtggaggagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgagggcagaggcagcctgctgacctgcggcgacgtcgaggagaaccccgggcccatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgatgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgactagtggcgaattcggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 68 - protein, 14 aa, Human immunodeficiency virus 1
  VARIANT 5: note = MISC_FEATURE - Xaa is Ile or Val
  VARIANT 13: note = MISC_FEATURE - Xaa is Lys, Arg, or Gln
LQARXLAVERYLXD

SEQ ID NO: 69 - protein, 14 aa, Human immunodeficiency virus 2
  VARIANT 13: note = MISC_FEATURE - Xaa is Lys, Ala, or Gln
  VARIANT 14: note = MISC_FEATURE - Xaa is Asp or His
LQARVTAIEKYLXX

SEQ ID NO: 70 - protein, 14 aa, Simian immunodeficiency virus
LQARLLAVERYLKD

SEQ ID NO: 71 - protein, 14 aa, unidentified
  source 1..14: note = Moloney murine leukemia virus
LQNRRGLDLLFLKE

SEQ ID NO: 72 - protein, 14 aa, unidentified
  source 1..14: note = Human T-cell lymphotropic virus
AQNRRGLDLLFWEQ

SEQ ID NO: 73 - protein, 14 aa, Mason-Pfizer monkey virus
LQNRRGLDLLTAEQ

SEQ ID NO: 74 - protein, 14 aa, Homo sapiens
LQNRRALDLLTAER

SEQ ID NO: 75 - protein, 14 aa, Homo sapiens
LQNRRGLDMLTAAQ

SEQ ID NO: 76 - protein, 14 aa, unidentified
  source 1..14: note = Human endogenous retrovirus K
LANQINDLRQTVIW

SEQ ID NO: 77 - protein, 14 aa, Feline leukemia virus
LQNRRGLDILFLQE

SEQ ID NO: 78 - protein, 9 aa, Human immunodeficiency virus 1
GALFLGFLG

SEQ ID NO: 79 - protein, 9 aa, synthetic construct
  REGION 1..9: note = Synthetic peptide
AGFGLLLGF

SEQ ID NO: 80 - protein, 9 aa, synthetic construct
  REGION 1..9: note = Synthetic peptide
AGLFLGFLG

(19) United States
(12) Patent Application Publication
(10) Pub. No.: US 2024/0141293 A1
(43) Pub. Date: May 2, 2024

(54) TARGETED GENE INSERTION FOR IMPROVED IMMUNE CELLS THERAPY

(71) Applicant: CELLECTIS, Paris (FR)

(72) Inventors: Brian BUSSER, New York, NY (US); Philippe DUCHATEAU, Draveil (FR); Alexandre JUILLERAT, New York, NY (US); Laurent POIROT, Paris (FR); Julien VALTON, New York, NY (US)

(21) Appl. No.: 18/540,309

(22) Filed: Dec. 14, 2023

Related U.S. Application Data
(62) Division of application No. 16/340,222, filed on Apr. 8, 2019, now Pat. No. 11,873,511.
(60) Provisional application No. 62/410,187, filed on Oct. 19, 2016.

(30) Foreign Application Priority Data
Oct. 27, 2016 (DK) PA201670840

(51) Int. Cl. C12N 5/0783; A61K 35/17; C12N 9/22; C12N 15/90

(52) U.S. Cl. CPC C12N 5/0638; A61K 35/17; C12N 9/22; C12N 15/907; C12N 2510/00; C12N 2750/14143; C12N 2830/008

(57) ABSTRACT

The invention pertains to the field of adaptive cell immunotherapy. It provides with the genetic insertion of exogenous coding sequence(s) that help the immune cells to direct their immune response against infected or malignant cells. These exogenous coding sequences are more particularly inserted under the transcriptional control of endogenous gene promoters that are sensitive to immune cells activation. Such method allows the production of safer immune primary cells of higher therapeutic potential.

### CROSS-REFERENCE TO RELATED APPLICATIONS

This application is a divisional of U.S. application Ser. No. 16/340,222 filed on Apr. 8, 2019, which is a U.S. Natl. Stage of International Application PCT/EP2017/076798 filed Oct. 19, 2017, which claims the benefit of U.S. provisional application 62/410,187 filed Oct. 19, 2016, and Danish Application PA201670840 filed Oct. 27, 2016.

### REFERENCE TO SEQUENCE USING SUBMITTED ELECTRONICALLY

The instant application contains a Sequence Listing which has been submitted electronically in XML file format and is hereby incorporated by reference in its entirety. Said XML copy, created on Dec. 13, 2023, is named D12016-11US2_SL.xml and is 215,538 bytes in size.

### FIELD OF THE INVENTION

The invention pertains to the field of adaptive cell immunotherapy. It aims to enhance the functionality of primary immune cells against pathologies that develop immune resistance, such as tumors, thereby improving the therapeutic potential of these immune cells. The method of the invention provides with the genetic insertion of exogenous coding sequence(s) that help the immune cells to direct their immune response against infected or malignant cells. These exogenous coding sequences are more particularly inserted under the transcriptional control of endogenous gene promoters that are up or downregulated upon immune cells activation, upon tumor microenvironment or life threatening inflammatory conditions or promoters that are insensitive to immune cells activation. The invention also provides with sequence-specific endonuclease reagents and donor DNA vectors, such as AAV vectors, to perform such targeted insertions at said particular loci. The method of the invention contributes to improving the therapeutic potential and safety of engineered primary immune cells for their efficient use in cell therapy

### BACKGROUND OF THE INVENTION

Effective clinical application of primary immune cell populations including hematopoietic cell lineages has been established by a number of clinical trials over a decade against a range of pathologies, in particular HIV infection and Leukemia (Tristen S. J. et al. (2011) Treating cancer with genetically engineered T cells. Trends in Biotechnology. 29(11):550-557).

However, most of these clinical trials have used immune cells, mainly NK and T-cells, obtained from the patients themselves or from compatible donors, bringing some limitations with respect to the number of available immune cells, their fitness, and their efficiency to overcome diseases that have already developed strategies to get around or reduce patient's immune system.

As a primary advance into the procurement of allogeneic immune cells, universal immune cells, available as “off-the-shelf” therapeutic products, have been produced by gene editing (Poirot et al. (2015) Multiplex Genome-Edited T-cell Manufacturing Platform for “Off-the-Shelf” Adoptive T-cell Immunotherapies Cancer Res. 75: 3853-64). These universal immune cells are obtainable by expressing specific rare-cutting endonuclease into immune cells originating from donors, with the effect of disrupting, by double strand-break, their self-recognition genetic determinants.

Since the emergence of the first programmable sequence-specific reagents by the turn of the century, initially referred to as Meganucleases (Smith et al. (2006) A combinatorial approach to create artificial homing endonucleases cleaving chosen sequences. Nucl. Acids Res. 34 (22):e149), different types of sequence-specific endonucleases reagents have been developed offering improved specificity, safety and reliability.

TALE-nucleases (WO2011072246), which are fusions of a TALE binding domain with a cleavage catalytic domain have been successfully applied to primary immune cells, in particular T-cells from peripheral blood mononuclear cell (PBMC). Such TALE-nucleases, marketed under the name TALEN®, are those currently used to simultaneously inactivate gene sequences in T-cells originating from donors, in particular to produce allogeneic therapeutic T-Cells in which the genes encoding TCR (T-cell receptor) and CD52 are disrupted. These cells can be endowed with chimeric antigen receptors (CAR) for treating cancer patients (US2013/0315884). TALE-nucleases are very specific reagents because they need to bind DNA by pairs under obligatory heterodimeric form to obtain dimerization of the cleavage domain Fok-1. Left and right heterodimer members each recognizes a different nucleic sequences of about 14 to 20 bp, together spanning target sequences of 30 to 50 bp overall specificity.

Other endonucleases reagents have been developed based on the components of the type II prokaryotic CRISPR (Clustered Regularly Interspaced Short palindromic Repeats) adaptive immune system of the bacteria S. pyogenes. This multi-component system referred to as RNA-guided nuclease system (Gasiunas, Barrangou et al. 2012; Jinek, Chylinski et al. 2012), involves members of Cas9 or Cpf1 endonuclease families coupled with a guide RNA molecules that have the ability to drive said nuclease to some specific genome sequences (Zetsche et al. (2015). Cpf1 is a single RNA-guided endonuclease that provides immunity in bacteria and can be adapted for genome editing in mammalian cells. Cell 163:759-771). Such programmable RNA-guided endonucleases are easy to produce because the cleavage specificity is determined by the sequence of the RNA guide, which can be easily designed and cheaply produced. The specificity of CRISPR/Cas9 although stands on shorter sequences than TAL-nucleases of about 10 pb, which must be located near a particular motif (PAM) in the targeted genetic sequence. Similar systems have been described using a DNA single strand oligonucleotide (DNA guide) in combination with Argonaute proteins (Gao, F. et al. DNA-guided genome editing using the Natronobacterum gregoryi Argonaute (2016) doi:10.1038/nbt.3547).

Other endonuclease systems derived from homing endonucleases (ex: I-OnuI, or I-CreI), combined or not with TAL-nuclease (ex: MegaTAL) or zing-finger nucleases have also proven specificity, but to a lesser extend so far.

In parallel, novel specificities can be conferred to immune cells through the genetic transfer of transgenic T-cell receptors or so-called chimeric antigen receptors (CARs) (Jena et al. (2010) Redirecting T-cell specificity by introducing a tumor-specific chimeric antigen receptor. Blood. 116:1035-1044). CARs are recombinant receptors comprising a targeting moiety that is associated with one or more signaling domains in a single fusion molecule. In general, the binding moiety of a CAR consists of an antigen-binding domain of a single-chain antibody (scFv), comprising the light and heavy variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Signaling domains from co-stimulatory molecules including CD28, OX-40 (CD134), ICOS and 4-1BB (CD137) have been added alone (second generation) or in combination (third generation) to enhance survival and increase proliferation of CAR modified T cells. CARs have successfully allowed T cells to be redirected against antigens expressed at the surface of tumor cells from various malignancies including lymphomas and solid tumors.

Recently engineered T-cells disrupted in their T-cell receptor (TCR) using TALE-nucleases, endowed with chimeric antigen receptor (CAR) targeting CD19 malignant antigen, referred to as “UCART19” product, have shown therapeutic potential in at least two infants who had refractory leukemia (Leukaemia success heralds wave of gene-editing therapies (2015) Nature 527:146-147). To obtain such UCART19 cells, the TALE-nuclease was transiently expressed into the cells upon electroporation of capped mRNA to operate TCR gene disruption, whereas a cassette encoding the chimeric antigen receptor (CAR CD19) was introduced randomly into the genome using a retroviral vector.

In this later approach, the steps of gene inactivation and of expressing the chimeric antigen receptor are independently performed after inducing activation of the T-Cell “ex-vivo”.

However, engineering primary immune cells is not without any consequences on the growth/physiology of such cells. In particular one major challenge is to ovoid cells exhaustion/anergy that significantly reduces their immune reaction and life span. This is more likely to happen when the cells are artificially activated ahead of their infusion into the patient. It is also the case when a cell is endowed with a CAR that is too reactive.

To avoid these pitfalls, the inventors have thought about taking advantage of the transcriptional regulation of some key genes during T-cell activation to express exogenous genetic sequences increasing the therapeutic potential of the immune cells. The exogenous genetic sequences to be expressed or co-expressed upon immune cell activation are introduced by gene targeted insertion using sequence-specific endonuclease reagents, so that their coding sequences are transcribed under the control of the endogenous promoters present at said loci. Alternatively, loci that are not expressed during immune cell activation can be used as “safe-harbor loci” for the integration of expression cassettes without any adverse consequences on the genome.

These cell engineering strategies, as per the present invention, tend to reinforce the therapeutic potential of primary immune cells in general, in particular by increasing their life span, persistence and immune activity, as well as by limiting cell exhaustion. The invention may be carried out on primary cells originating from patients as part of autologous treatment strategies, as well as from donors, as part of allogeneic treatment strategies.

### SUMMARY OF THE INVENTION

Non-homologous end-joining (NHEJ) and homology-directed repair (HDR) are the two major pathways used to repair in vivo DNA breaks. The latter pathway repairs the break in a template-dependent manner (HDR naturally utilizes the sister chromatid as a DNA repair template). Homologous recombination has been used for decades to precisely edit genomes with targeted DNA modifications using exogenously supplied donor template. The artificial generation of a double strand break (DSB) at the target location using rare-cutting endonucleases considerably enhances the efficiency of homologous recombination (e.g. U.S. Pat. No. 8,921,332). Also the co-delivery of a rare-cutting endonuclease along with a donor template containing DNA sequences homologous to the break site enables HDR-based gene editing such as gene correction or gene insertion. However, such techniques have not been widely used in primary immune cells, especially CAR T-cells, due to several technical limitations: difficulty of transfecting DNA into such types of cells leading to apoptosis, immune cells have a limited life span and number of generations, homologous recombination occurs at a low frequency in general.

So far, sequence specific endonuclease reagents have been mainly used in primary immune cells for gene inactivation (e.g. WO2013176915) using the NHEJ pathway.

In a general aspect, the present invention relies on performing site directed gene editing, in particular gene insertion (or multi gene insertions) in a target cell in order to have the integrated gene transcription be under the control of an endogenous promoter.

In a general aspect the invention relies on performing gene editing in primary immune cells to have integrated genes transcription be under the control of an endogenous promoter while maintaining the expression of the native gene through the use of cis-regulatory elements (e.g. 2A cis-acting hydrolase elements) or of internal ribosome entry site (IRES) in the donor template.

In a general aspect the invention relies, as non-limiting examples, on controlling the expression, in primary T-cells, of chimeric antigen receptors (CAR), of critical cytokines to drive an anti-tumor response, of stimulatory cytokines to increase proliferative potential, of chemokine receptors to encourage trafficking to the tumor, or of different protective or inhibitory genes to block the immune inhibition provided by the tumor. Indeed, one major advantage of the present invention is to place such exogenous sequences under control of endogenous promoters, which transcriptional activity is not reduced by the effects of the immune cells activation.

By contrast to previous method for engineering therapeutic immune cells, where for instance an exogenous coding sequence was integrated and expressed at the TCR locus for constitutive gene expression, the inventors have integrated coding sequence at loci, which are specifically transcribed during T-cells activation, preferably on a CAR dependent fashion.

In one aspect, the invention relies on expressing a chimeric antigen receptor (CAR) at selected gene loci that are upregulated upon immune cells activation. The exogenous sequence(s) encoding the CAR and the endogenous gene coding sequence (s) may be co-transcribed, for instance by being separated by cis-regulatory elements (e.g. 2A cis-acting hydrolase elements) or by an internal ribosome entry site (IRES), which are also introduced. For instance, the exogenous sequences encoding a CAR can be placed under transcriptional control of the promoter of endogenous genes that are activated by the tumor microenvironment, such as HIF1a, transcription factor hypoxia-inducible factor, or the aryl hydrocarbon receptor (AhR), which are gene sensors respectively induced by hypoxia and xenobiotics in the close environment of tumors.

The present invention is thus useful to improve the therapeutic outcome of CAR T-cell therapies by integrating exogenous genetic attributes/circuits under the control of endogenous T-cell promoters influenced by tumor microenvironment (TME). TME features, including as non-limiting examples, arginine, cysteine, tryptophan and oxygen deprivation as well as extracellular acidosis (lactate build up), are known to upregulate specific endogenous genes. Pursuant to the invention, upregulation of endogenous genes can be “hijacked” to re-express relevant exogenous coding sequences to improve the antitumor activity of CAR T-cells in certain tumor microenvironment.

In preferred embodiments, the method of the invention comprises the step of generating a double-strand break at a locus highly transcribed under tumor microenvironment, by expressing sequence-specific nuclease reagents, such as TALEN, ZFN or RNA-guided endonucleases as non-limiting examples, in the presence of a DNA repair matrix preferably set into an AAV6 based vector. This DNA donor template generally includes two homology arms embedding unique or multiple Open Reading Frames and regulatory genetic elements (stop codon and polyA sequences) referred to herein as exogenous coding sequences.

In another aspect, said exogenous sequence is introduced into the genome by deleting or modifying the endogenous coding sequence(s) present at said locus (knock-out by knock-in), so that a gene inactivation is combined with transgenesis.

Depending on the locus targeted and its involvement in immune cells activity, the targeted endogenous gene may be inactivated or maintained in its original function. Should the targeted gene be essential for immune cells activity, this insertion procedure can generate a single knock-in (KI) without gene inactivation. In the opposite, if the targeted gene is deemed involved in immune cells inhibition/exhaustion, the insertion procedure is designed to prevent expression of the endogenous gene, preferably by knocking-out the endogenous sequence, while enabling expression of the introduced exogenous coding sequence(s).

In more specific aspects, the invention relies on up-regulating, with various kinetics, the target gene expression upon activation of the CAR signalling pathway by targeted integration (with or without the native gene disruption) at the specific loci such as, as non-limiting example, PD1, PDL1, CTLA-4, TIM3, LAG3, TNFa or IFNg.

In an even more specific aspect, it is herein described engineered immune cells, and preferably primary immune cells for infusion into patients, comprising exogenous sequences encoding IL-15 or IL-12 polypeptide(s), which are integrated at the PD1, CD25 or CD69 endogenous locus for their expression under the control of the endogenous promoters present at these loci.

The immune cells according to the present invention can be [CAR]^{positive}, [CAR]^{negative}, [TCR]^{positive}, or [TCR]^{negative}, depending on the therapeutic indications and recipient patients. In one preferred aspect, the immune cells are further made [TCR]^{negative} for allogeneic transplantation. This can be achieved especially by genetic disruption of at least one endogenous sequence encoding at least one component of TCR, such as TRAC (locus encoding TCRalpha), preferably by integration of an exogenous sequence encoding a chimeric antigen receptor (CAR) or a recombinant TCR, or component(s) thereof.

According to a further aspect of the invention, the immune cells are transfected with an exogenous sequence coding for a polypeptide which can associate and preferably interfere with a cytokine receptor of the IL-6 receptor family, such as a mutated GP130, In particular, the invention provides immune cells, preferably T-cells, which secrete soluble mutated GP130, aiming at reducing cytokine release syndrome (CRS) by interfering, and ideally block, interleukine-6 (IL-6) signal transduction. CRS is a well-known complication of cell immunotherapy leading to auto immunity that appears when the transduced immune cells start to be active in-vivo. Following binding of IL-6 to its receptor IL-6R, the complex associate with the GP130 subunit, initiating signal transduction and a cascade of inflammatory responses. According to a particular aspect, a dimeric protein comprising the extracellular domain of GP130 fused to the Fc portion of an IgG1 antibody (sgp130Fc) is expressed in the engineered immune cells to bind specifically soluble IL-R/IL-6 complex to achieve partial or complete blockade of IL-6 trans signaling. The present invention thus refers to a method for limiting CRS in immunotherapy, wherein immune cells are genetically modified to express a soluble polypeptide which can associate and preferably interfere with a cytokine receptor of the IL-6 receptor family, such as sgp130Fc. According to a preferred aspect, this sequence encoding said soluble polypeptide which can associate and preferably interfere with a cytokine receptor of the IL-6 receptor family, is integrated under control of an endogenous promoter, preferably at one locus responsive to T-cells activation, such as one selected from Tables 6, 8 or 9, more especially PD1, CD25 or CD69. Polynucleotide sequences of the vectors, donor templates comprising the exogenous coding sequences and/or sequences homologous to the endogenous loci, the sequences pertaining to the resulting engineered cells, as well as those permitting the detection of said engineered cells are all part of the present disclosure.

In a general aspect the invention relies, as non-limiting examples, on controlling the expression of components of biological “logic gates” (“AND” or “OR” or “NOT” or any combination of these) by targeted integration of genes. Similar to the electronic logic gates, cellular components expressed at different loci can exchange negative and positive signals that rule, for instance, the conditions of activation of an immune cell. Such component encompasses as non-limiting examples positive and negative chimeric antigen receptors that may be used to control T-cell activation and the resulting cytotoxicity of the engineered T-cells in which they are expressed.

According to a preferred embodiment, the invention relies on introducing the sequence specific endonuclease reagent and/or the donor template containing the gene of interest and sequences homologous to the target gene by transfecting ssDNA (oligonucleotides as non-limiting example), dsDNA (plasmid DNA as non-limiting example), and more particularly adeno-associated virus (AAV) as non-limiting example.

The invention also relates to the vectors, donor templates, reagents and resulting engineered cells pertaining to the above methods, as well as their use in therapy.

### BRIEF DESCRIPTION OF THE FIGURES AND TABLES

FIG. 1: Strategies for engineering hematopoietic stem cells (HSCs) by introducing exogenous sequences at specific loci under transcriptional control of endogenous promoters specifically activated in specific immune cell types. The figure lists examples of specific endogenous genes, at which loci the exogenous coding sequence(s) can be inserted for expression in the desired hematopoietic lineages as per the present invention. The goal is to produce ex-vivo engineered HSCs to be engrafted into patients, in order for them to produce immune cells in-vivo, which will express selected transgenes while they get differentiated into a desired lineage.

FIG. 2: Schematic representation of the donor sequences used in the experimental section to insert IL-15 exogenous coding sequence at the CD25 and PD1 loci and also the anti-CD22 CAR exogenous coding sequence at the TRAC locus. A: donor template (designated IL-15m-CD25) designed for site directed insertion of IL-15 at the CD25 locus for obtaining co-transcription of CD25 and IL-15 polypeptides by the immune cell. Sequences are detailed in the examples. B: donor template (designated IL-15m-PD1) designed for site directed insertion of IL-15 at the PD1 locus for obtaining transcription of IL-15 under the transcriptional activity of the promoter of PD1 endogenous gene. The PD1 right and Left border sequences can be selected so as to keep the PD1 endogenous coding sequence intact or disrupted. In this later case, PD1 is knocked-out while IL-15 is Knocked-in and transcribed. C: donor template designed for site directed insertion of a chimeric antigen receptor (ex: anti-CD22 CAR) into the TCR locus (ex: TRAC). In general, the left and right borders are chosen so as to disrupt the TCR in order to obtain [TCR]^{neg}[CAR]^{pos} engineered immune cells suitable for allogeneic transplant into patients.

FIG. 3: Flow cytometry measures of the frequency of targeted integration of IL-15m at either the PD1 or CD25 locus by using respectively PD1 or CD25 TALEN®, in a context where an anti-CD22 CAR is also integrated at the TRAC locus using TRAC TALEN®. These results show efficient targeted integration of both the CAR anti-CD22 at the TRAC locus together and the IL-15 coding sequence at the PD1 or CD25 loci. A: mock transfected primary T-cells. B: primary T-cells transfected with the donor sequences described in FIGS. 1 (B and C) and specific TALEN® for the double integration at the TCR and PDI loci. C: primary T-cells transfected with the donor sequences described in FIG. 1 (A and C) and specific TALEN® for the double integration at the TCR and CD25 loci.

FIG. 4: Schematic representation of the exogenous sequences used in the experimental section to transfect the primary immune cells to obtain the results shown in FIGS. 5 and 6.

FIGS. 5 and 6: Flow cytometry measures for LNGFR expression among viable T-cells transfected with donor templates of FIG. 4 and specific TALEN® (TCR and CD25), upon antiCD3/CD28 non-specific activation (Dynabeads®) and upon CAR dependent tumor cell activation (raji tumor cells). As shown in FIG. 6, LNGFR expression was specifically induced in [CAR anti-CD22]^{positive} cells upon CAR/tumor engagement.

FIGS. 7 and 8: Flow cytometry measures for CD25 expression among viable T-cells transfected with donor templates of FIG. 4 and specific TALEN® (TCR and CD25) upon antiCD3/CD28 non-specific activation (Dynabeads®) and Tumor cell activation (raji tumor cells). As shown in FIG. 8, CD25 expression was specifically induced in [CAR anti-CD22]^{positive} cells upon CAR/tumor engagement.

FIG. 9: Schematic representation of the exogenous sequences used in the experimental section to transfect the primary immune cells to obtain the results shown in FIGS. 11 and 12.

FIGS. 10 and 11: Flow cytometry measures for LNGFR expression among viable T-cells transfected with donor templates of FIG. 9 and specific TALEN® (TCR and PD1) upon antiCD3/CD28 non-specific activation (Dynabeads®) and Tumor cell activation (raji tumor cells). As shown in FIG. 11, LNGFR expression was specifically induced in [CAR anti-CD22]^{positive} cells upon CAR/tumor engagement.

FIG. 12: Flow cytometry measures for endogenous PD1 expression among viable T-cells transfected with donor templates of FIG. 9 upon antiCD3/CD28 non-specific activation (Dynabeads®) and Tumor cell activation (raji tumor cells) with and without using TALEN® (TCR and PD1). PD1 was efficiently Knocked-out by TALEN treatment (8% remaining expression of PD1 out of 54%).

FIG. 13: Diagram showing IL-15 production in [CAR]^{positive} (CARm) and [CAR]^{negative} engineered immune cells according to the invention transfected with the donor template described in FIG. 2 (B) and TALEN® for insertion of IL-15 exogenous coding sequences into the PD1 locus. IL15, which transcription was under control of endogenous PD1 promoter, was efficiently induced upon antiCD3/CD28 non-specific activation (Dynabeads®) and Tumor cell activation (raji tumor cells) and secreted in the culture media.

FIG. 14: Graph showing the amount of IL-15 secreted over time (days) post activation by the immune cells engineered according to the invention. A: Cells engineered by integration of the IL-15 coding sequence at the CD25 locus using the DNA donor templates described in FIGS. 2A (IL-15m_CD25) and/or 2C (CARm). B: Cells engineered by integration of the IL-15 coding sequence at the PD1 locus using the DNA donor templates described in FIGS. 2B (IL-15m_PD1) and/or 2C (CARm). Integrations at both loci show similar IL-15 secretion profiles. Secretion of IL-15 is significant increased by tumor specific activation of CAR.

FIG. 15: Graph reporting number of Raji-Luc tumor cells expressing CD22 antigen (luciferase signal) over time in a survival assay (serial killing assay) as described in Example 2. The immune cells (PBMCs) have been engineered to integrate IL-15 coding sequences at the PD1 (A) or CD25 locus (B) and to express anti-CD22-CAR at the TCR locus (thereby disrupting TCR expression). In this assay, tumor cells are regularly added to the culture medium, while being partially or totally eliminated by the CAR positive cells. The re-expression of IL-15 at either PD1 or CD25 cells dramatically helps the elimination of the tumor cells by the CAR positive cells.

FIG. 16: Schematic representation of the donor sequences used in the experimental section to insert at the PD1 locus the exogenous sequences encoding IL-12 and gp130Fc. A: donor template (designated IL-12m-PD1) designed for site directed insertion of IL-12a and IL-12b coding sequences (SEQ ID NO:47 and 48) at the PD1 locus for obtaining co-transcription of IL-12a and IL-12b, while disrupting PD1 endogenous coding sequence. The right and left border sequences homologous to the PD1 locus sequences are at least 100 pb long, preferably at least 200 pb long, and more preferably at least 300 pb long and comprising SEQ ID NO:45 and 46. Sequences are detailed in Table 5. B: donor template (designated gp130Fcm-PD1) designed for site directed insertion of gp130Fc coding sequences (SEQ ID NO:51) for obtaining transcription at the PD1 locus under PD1 promoter, while disrupting PD1 endogenous coding sequence. The right and left border sequences homologous to the PD1 locus sequences are at least 100 pb long, preferably at least 200 pb long, and more preferably at least 300 pb long and comprising SEQ ID NO:45 and 46. Sequences are detailed in Table 5.

Table 1: ISU domain variants from diverse viruses.

Table 2: Aminoacid sequences of FP polypeptide from natural and artificial origins.

Table 3: List of genes involved into immune cells inhibitory pathways, which can be advantageously modified or inactivated by inserting exogenous coding sequence according to the invention.

Table 4: sequences referred to in example 1.

Table 5: sequences referred to in example 2.

Table 6: List of human genes that are up-regulated upon T-cell activation (CAR activation sensitive promoters), in which gene targeted insertion is sought according to the present invention to improve immune cells therapeutic potential.

Table 7: Selection of genes that are steadily transcribed during immune cell activation (dependent or independent from T-cell activation).

Table 8: Selection of genes that are transiently upregulated upon T-cell activation.

Table 9: Selection of genes that are upregulated over more than 24 hours upon T-cell activation.

Table 10: Selection of genes that are down-regulated upon immune cell activation.

Table 11: Selection of genes that are silent upon T-cell activation (safe harbor gene targeted integration loci).

Table 12: List of gene loci upregulated in tumor exhausted infiltrating lymphocytes (compiled from multiple tumors) useful for gene integration of exogenous coding sequences as per the present invention.

Table 13: List of gene loci upregulated in hypoxic tumor conditions useful for gene integration of exogenous coding sequences as per the present invention.

### DETAILED DESCRIPTION OF THE INVENTION

Unless specifically defined herein, all technical and scientific terms used herein have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

The present invention is drawn to a general method of preparing primary immune cells for cell immunotherapy involving gene targeted integration of an exogenous coding sequence into the chromosomal DNA of said immune cells. According to some aspects, this integration is performed in such a way that said coding sequence is placed under the transcriptional control of at least one promoter endogenous to said cells, said endogenous promoter being preferably not a constitutive promoter, such as the one transcribing T-cell receptor alpha constant (TRAC—NCBI Gene ID #28755) A constitutive promoter as per the present invention is for instance a promoter that is active independently from CAR activation—ex: when T-cells are not yet activated.

### Improving the Therapeutic Potential of Immune Cells by Gene Targeted Integration

Gene editing techniques using polynucleotide sequence-specific reagents, such as rare-cutting endonucleases, have become the state of the art for the introduction of genetic modifications into primary cells. However, they have not been used so far in immune cells to introduce exogenous coding sequences under the transcriptional control of endogenous promoters.

The present invention aims to improve the therapeutic potential of immune cells through gene editing techniques, especially by gene targeted integration.

By “gene targeting integration” is meant any known site-specific methods allowing to insert, replace or correct a genomic sequence into a living cell. According to a preferred aspect of the present invention, said gene targeted integration involves homologous gene recombination at the locus of the targeted gene to result the insertion or replacement of at least one exogenous nucleotide, preferably a sequence of several nucleotides (i.e. polynucleotide), and more preferably a coding sequence.

By “sequence-specific reagent” is meant any active molecule that has the ability to specifically recognize a selected polynucleotide sequence at a genomic locus, preferably of at least 9 bp, more preferably of at least 10 bp and even more preferably of at least 12 pb in length, in view of modifying said genomic locus. According to a preferred aspect of the invention, said sequence-specific reagent is preferably a sequence-specific nuclease reagent.

By “immune cell” is meant a cell of hematopoietic origin functionally involved in the initiation and/or execution of innate and/or adaptative immune response, such as typically CD3 or CD4 positive cells. The immune cell according to the present invention can be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and from tumors, such as tumor infiltrating lymphocytes. In some embodiments, said immune cell can be derived from a healthy donor, from a patient diagnosed with cancer or from a patient diagnosed with an infection. In another embodiment, said cell is part of a mixed population of immune cells which present different phenotypic characteristics, such as comprising CD4, CD8 and CD56 positive cells.

By “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells. Primary cells are generally used in cell therapy as they are deemed more functional and less tumorigenic.

In general, primary immune cells are provided from donors or patients through a variety of methods known in the art, as for instance by leukapheresis techniques as reviewed by Schwartz J. et al. (Guidelines on the use of therapeutic apheresis in clinical practice-evidence-based approach from the Writing Committee of the American Society for Apheresis: the sixth special issue (2013) J Clin Apher. 28(3):145-284).

The primary immune cells according to the present invention can also be differentiated from stem cells, such as cord blood stem cells, progenitor cells, bone marrow stem cells, hematopoietic stem cells (HSC) and induced pluripotent stem cells (iPS).

By “nuclease reagent” is meant a nucleic acid molecule that contributes to an nuclease catalytic reaction in the target cell, preferably an endonuclease reaction, by itself or as a subunit of a complex such as a guide RNA/Cas9, preferably leading to the cleavage of a nucleic acid sequence target.

The nuclease reagents of the invention are generally “sequence-specific reagents”, meaning that they can induce DNA cleavage in the cells at predetermined loci, referred to by extension as “targeted gene”. The nucleic acid sequence which is recognized by the sequence specific reagents is referred to as “target sequence”. Said target sequence is usually selected to be rare or unique in the cell's genome, and more extensively in the human genome, as can be determined using software and data available from human genome databases, such as ensembl. org/index. html.

“Rare-cutting endonucleases” are sequence-specific endonuclease reagents of choice, insofar as their recognition sequences generally range from 10 to 50 successive base pairs, preferably from 12 to 30 bp, and more preferably from 14 to 20 bp.

According to a preferred aspect of the invention, said endonuclease reagent is a nucleic acid encoding an “engineered” or “programmable” rare-cutting endonuclease, such as a homing endonuclease as described for instance by Arnould S., et al. (WO2004067736), a zing finger nuclease (ZFN) as described, for instance, by Umov F., et al. (Highly efficient endogenous human gene correction using designed zinc-finger nucleases (2005) Nature 435:646-651), a TALE-Nuclease as described, for instance, by Mussolino et al. (A novel TALE nuclease scaffold enables high genome editing activity in combination with low toxicity (2011) Nucl. Acids Res. 39(21):9283-9293), or a MegaTAL nuclease as described, for instance by Boissel et al. (MegaTALs: a rare-cleaving nuclease architecture for therapeutic genome engineering (2013) Nucleic Acids Research 42 (4):2591-2601).

According to another embodiment, the endonuclease reagent is a RNA-guide to be used in conjunction with a RNA guided endonuclease, such as Cas9 or Cpf1, as per, inter alia, the teaching by Doudna, J., and Chapentier, E., (The new frontier of genome engineering with CRISPR-Cas9 (2014) Science 346 (6213):1077), which is incorporated herein by reference.

According to a preferred aspect of the invention, the endonuclease reagent is transiently expressed into the cells, meaning that said reagent is not supposed to integrate into the genome or persist over a long period of time, such as be the case of RNA, more particularly mRNA, proteins or complexes mixing proteins and nucleic acids (eg: Ribonucleoproteins).

In general, 80% the endonuclease reagent is degraded by 30 hours, preferably by 24, more preferably by 20 hours after transfection.

An endonuclease under mRNA form is preferably synthetized with a cap to enhance its stability according to techniques well known in the art, as described, for instance, by Kore A. L., et al. (Locked nucleic acid (LNA)-modified dinucleotide mRNA cap analogue: synthesis, enzymatic incorporation, and utilization (2009) J Am Chem Soc. 131(18):6364-5).

In general, electroporation steps that are used to transfect immune cells are typically performed in closed chambers comprising parallel plate electrodes producing a pulse electric field between said parallel plate electrodes greater than 100 volts/cm and less than 5,000 volts/cm, substantially uniform throughout the treatment volume such as described in WO/2004/083379, which is incorporated by reference, especially from page 23, line 25 to page 29, line 11. One such electroporation chamber preferably has a geometric factor (cm⁻¹) defined by the quotient of the electrode gap squared (cm2) divided by the chamber volume (cm³), wherein the geometric factor is less than or equal to 0.1 cm⁻¹, wherein the suspension of the cells and the sequence-specific reagent is in a medium which is adjusted such that the medium has conductivity in a range spanning 0.01 to 1.0 milliSiemens. In general, the suspension of cells undergoes one or more pulsed electric fields. With the method, the treatment volume of the suspension is scalable, and the time of treatment of the cells in the chamber is substantially uniform.

Due to their higher specificity, TALE-nuclease have proven to be particularly appropriate sequence specific nuclease reagents for therapeutic applications, especially under heterodimeric forms—i.e. working by pairs with a “right” monomer (also referred to as “5′” or “forward”) and ‘left” monomer (also referred to as “3″” or “reverse”) as reported for instance by Mussolino et al. (TALEN® facilitate targeted genome editing in human cells with high specificity and low cytotoxicity (2014) Nucl. Acids Res. 42(10): 6762-6773).

As previously stated, the sequence specific reagent is preferably under the form of nucleic acids, such as under DNA or RNA form encoding a rare cutting endonuclease a subunit thereof, but they can also be part of conjugates involving polynucleotide(s) and polypeptide(s) such as so-called “ribonucleoproteins”. Such conjugates can be formed with reagents as Cas9 or Cpf1 (RNA-guided endonucleases) or Argonaute (DNA-guided endonucleases) as recently respectively described by Zetsche, B. et al. (Cpf1 Is a Single RNA-Guided Endonuclease of a Class 2 CRISPR-Cas System (2015) Cell 163(3): 759-771) and by Gao F. et al. (DNA-guided genome editing using the Natronobacterum gregoryi Argonaute (2016) Nature Biotech), which involve RNA or DNA guides that can be complexed with their respective nucleases.

“Exogenous sequence” refers to any nucleotide or nucleic acid sequence that was not initially present at the selected locus. This sequence may be homologous to, or a copy of, a genomic sequence, or be a foreign sequence introduced into the cell. By opposition “endogenous sequence” means a cell genomic sequence initially present at a locus. The exogenous sequence preferably codes for a polypeptide which expression confers a therapeutic advantage over sister cells that have not integrated this exogenous sequence at the locus. A endogenous sequence that is gene edited by the insertion of a nucleotide or polynucleotide as per the method of the present invention, in order to express a different polypeptide is broadly referred to as an exogenous coding sequence The method of the present invention can be associated with other methods involving physical of genetic transformations, such as a viral transduction or transfection using nanoparticles, and also may be combined with other gene inactivation and/or transgene insertions.

According to one aspect, the method according to the invention comprises the steps of:
providing a population of primary immune cells;
introducing into a proportion of said primary immune cells:
   i) At least one nucleic acid comprising an exogenous nucleotide or polynucleotide sequence to be integrated at a selected endogenous locus to encode at least one molecule improving the therapeutic potential of said immune cells population;
   ii) At least one sequence-specific reagent that specifically targets said selected endogenous locus,
      wherein said exogenous nucleotide or polynucleotide sequence is inserted by targeted gene integration into said endogenous locus, so that said exogenous nucleotide or polynucleotide sequence forms an exogenous coding sequence under transcriptional control of an endogenous promoter present at said locus.

According to one aspect of the method, the sequence specific reagent is a nuclease and the targeted gene integration is operated by homologous recombination or NHEJ into said immune cells.

According to a further aspect of the invention, said endogenous promoter is selected to be active during immune cell activation and preferably up-regulated.

More specifically, the invention is drawn to a method for preparing engineered primary immune cells for cell immunotherapy, said method comprising:
providing a population of primary immune cells;
introducing into a proportion of said primary immune cells:
   i) At least one exogenous nucleic acid comprising an exogenous coding sequence encoding at least one molecule improving the therapeutic potential of said immune cells population;
   ii) At least one sequence-specific nuclease reagent that specifically targets a gene which is under control of an endogenous promoter active during immune cell activation;
      wherein said coding sequence is introduced into the primary immune cells genome by targeted homologous recombination, so that said coding sequence is placed under the transcriptional control of at least one endogenous promoter of said gene.

By “improving therapeutic potential” is meant that the engineered immune cells gain at least one advantageous property for their use in cell therapy by comparison to their sister non-engineered immune cells. The therapeutic properties sought by the invention maybe any measurable one as referred to in the relevant scientific literature.

Improved therapeutic potential can be more particularly reflected by a resistance of the immune cells to a drug, an increase in their persistence in-vitro or in-vivo, or a safer/more convenient handling during manufacturing of therapeutic compositions and treatments.

In general said molecule improving the therapeutic potential is a polypeptide, but it can also be a nucleic acid able to direct or repress expression of other genes, such as interference RNAs or guide-RNAs. The polypeptides may act directly or indirectly, such as signal transducers or transcriptional regulators.

According to one embodiment of the present method, the exogenous sequence is introduced into the endogenous chromosomal DNA by targeted homologous recombination. Accordingly, the exogenous nucleic acid introduced into the immune cell comprises at least one coding sequence(s), along with sequences that can hybridize endogenous chromosomal sequences under physiological conditions. In general, such homologous sequences show at least 70%, preferably 80% and more preferably 90% sequence identity with the endogenous gene sequences located at the insertion locus. These homologous sequences may flank the coding sequence to improve the precision of recombination as already taught for instance in U.S. Pat. No. 6,528,313. Using available software and on-line genome databases, it is possible to design vectors that includes said coding sequence (s), in such a way that said sequence(s) is (are) introduced at a precise locus, under transcriptional control of at least one endogenous promoter, which is a promoter of an endogenous gene. The exogenous coding sequence(s) is (are) then preferably inserted “in frame” with said endogenous gene. The sequences resulting from the integration of the exogenous polynucleotide sequence(s) can encode many different types of proteins, including fusion proteins, tagged protein or mutated proteins. Fusion proteins allow adding new functional domains to the proteins expressed in the cell, such as a dimerization domain that can be used to switch-on or switch-off the activity of said protein, such as caspase-9 switch. Tagged proteins can be advantageous for the detection of the engineered immune cells and the follow-up of the patients treated with said cells. Introducing mutation into proteins can confer resistance to drugs or immune depletion agents as further described below.

### Conferring Resistance to Drugs or Immune Depletion Agents

According to one aspect of the present method, the exogenous sequence that is integrated into the immune cells genomic locus encodes a molecule that confers resistance of said immune cells to a drug.

Examples of preferred exogenous sequences are variants of dihydrofolate reductase (DHFR) conferring resistance to folate analogs such as methotrexate, variants of inosine monophosphate dehydrogenase 2 (IMPDH2) conferring resistance to IMPDH inhibitors such as mycophenolic acid (MPA) or its prodrug mycophenolate mofetil (MMF), variants of calcineurin or methylguanine transferase (MGMT) conferring resistance to calcineurin inhibitor such as FK506 and/or CsA, variants of mTOR such as mTORmut conferring resistance to rapamycin) and variants of Lck, such as Lckmut conferring resistance to Imatinib and Gleevec.

The term “drug” is used herein as referring to a compound or a derivative thereof, preferably a standard chemotherapy agent that is generally used for interacting with a cancer cell, thereby reducing the proliferative or living status of the cell. Examples of chemotherapeutic agents include, but are not limited to, alkylating agents (e.g., cyclophosphamide, ifosamide), metabolic antagonists (e.g., purine nucleoside antimetabolite such as clofarabine, fludarabine or 2′-deoxyadenosine, methotrexate (MTX), 5-fluorouracil or derivatives thereof), antitumor antibiotics (e.g., mitomycin, adriamycin), plant-derived antitumor agents (e.g., vincristine, vindesine, Taxol), cisplatin, carboplatin, etoposide, and the like. Such agents may further include, but are not limited to, the anti-cancer agents TRIMETHOTRIXATE™ (TMTX), TEMOZOLOMIDE™, RALTRITREXED™, S-(4-Nitrobenzyl)-6-thioinosine (NBMPR), 6-benzyguanidine (6-BG), bis-chloronitrosourea (BCNU) and CAMPTOTHECIN™, or a therapeutic derivative of any thereof.

As used herein, an immune cell is made “resistant or tolerant” to a drug when said cell, or population of cells is modified so that it can proliferate, at least in-vitro, in a culture medium containing half maximal inhibitory concentration (IC50) of said drug (said IC50 being determined with respect to an unmodified cell(s) or population of cells).

In a particular embodiment, said drug resistance can be conferred to the immune cells by the expression of at least one “drug resistance coding sequence”. Said drug resistance coding sequence refers to a nucleic acid sequence that confers “resistance” to an agent, such as one of the chemotherapeutic agents referred to above. A drug resistance coding sequence of the invention can encode resistance to anti-metabolite, methotrexate, vinblastine, cisplatin, alkylating agents, anthracyclines, cytotoxic antibiotics, anti-immunophilins, their analogs or derivatives, and the like (Takebe, N., S. C. Zhao, et al. (2001) “Generation of dual resistance to 4-hydroperoxycyclophosphamide and methotrexate by retroviral transfer of the human aldehyde dehydrogenase class 1 gene and a mutated dihydrofolate reductase gene”. Mol. Ther. 3(1): 88-96), (Zielske, S. P., J. S. Reese, et al. (2003) “In vivo selection of MGMT(P140K) lentivirus-transduced human NOD/SCID repopulating cells without pretransplant irradiation conditioning.” J. Clin. Invest. 112(10): 1561-70) (Nivens, M. C., T. Felder, et al. (2004) “Engineered resistance to camptothecin and antifolates by retroviral coexpression of tyrosyl DNA phosphodiesterase-I and thymidylate synthase” Cancer Chemother Pharmacol 53(2): 107-15), (Bardenheuer, W., K. Lehmberg, et al. (2005). “Resistance to cytarabine and gemcitabine and in vitro selection of transduced cells after retroviral expression of cytidine deaminase in human hematopoietic progenitor cells”. Leukemia 19(12): 2281-8), (Kushman, M. E., S. L. Kabler, et al. (2007) “Expression of human glutathione S-transferase P1 confers resistance to benzo[a]pyrene or benzo[a]pyrene-7,8-dihydrodiol mutagenesis, macromolecular alkylation and formation of stable N2-Gua-BPDE adducts in stably transfected V79MZ cells co-expressing hCYP1A1” Carcinogenesis 28(1): 207-14).

The expression of such drug resistance exogenous sequences in the immune cells as per the present invention more particularly allows the use of said immune cells in cell therapy treatment schemes where cell therapy is combined with chemotherapy or into patients previously treated with these drugs.

Several drug resistance coding sequences have been identified that can potentially be used to confer drug resistance according to the invention. One example of drug resistance coding sequence can be for instance a mutant or modified form of Dihydrofolate reductase (DHFR). DHFR is an enzyme involved in regulating the amount of tetrahydrofolate in the cell and is essential to DNA synthesis. Folate analogs such as methotrexate (MTX) inhibit DHFR and are thus used as anti-neoplastic agents in clinic. Different mutant forms of DHFR which have increased resistance to inhibition by anti-folates used in therapy have been described. In a particular embodiment, the drug resistance coding sequence according to the present invention can be a nucleic acid sequence encoding a mutant form of human wild type DHFR (GenBank: AAH71996.1), which comprises at least one mutation conferring resistance to an anti-folate treatment, such as methotrexate. In particular embodiment, mutant form of DHFR comprises at least one mutated amino acid at position G15, L22, F31 or F34, preferably at positions L22 or F31 (Schweitzer et al. (1990) “Dihydrofolate reductase as a therapeutic target” Faseb J 4(8): 2441-52; International application WO94/24277; and U.S. Pat. No. 6,642,043). In a particular embodiment, said DHFR mutant form comprises two mutated amino acids at position L22 and F31. Correspondence of amino acid positions described herein is frequently expressed in terms of the positions of the amino acids of the form of wild-type DHFR polypeptide. In a particular embodiment, the serine residue at position 15 is preferably replaced with a tryptophan residue. In another particular embodiment, the leucine residue at position 22 is preferably replaced with an amino acid which will disrupt binding of the mutant DHFR to antifolates, preferably with uncharged amino acid residues such as phenylalanine or tyrosine. In another particular embodiment, the phenylalanine residue at positions 31 or 34 is preferably replaced with a small hydrophilic amino acid such as alanine, serine or glycine.

Another example of drug resistance coding sequence can also be a mutant or modified form of ionisine-5′-monophosphate dehydrogenase II (IMPDH2), a rate-limiting enzyme in the de novo synthesis of guanosine nucleotides. The mutant or modified form of IMPDH2 is a IMPDH inhibitor resistance gene. IMPDH inhibitors can be mycophenolic acid (MPA) or its prodrug mycophenolate mofetil (MMF). The mutant IMPDH2 can comprises at least one, preferably two mutations in the MAP binding site of the wild type human IMPDH2 (Genebank: NP_000875.2) leading to a significantly increased resistance to IMPDH inhibitor. Mutations in these variants are preferably at positions T333 and/or S351 (Yam, P., M. Jensen, et al. (2006) “Ex vivo selection and expansion of cells based on expression of a mutated inosine monophosphate dehydrogenase 2 after HIV vector transduction: effects on lymphocytes, monocytes, and CD34+ stem cells” Mol. Ther. 14(2): 236-44)(Jonnalagadda, M., et al. (2013) “Engineering human T cells for resistance to methotrexate and mycophenolate mofetil as an in vivo cell selection strategy.” PLoS One 8(6): e65519).

Another drug resistance coding sequence is the mutant form of calcineurin. Calcineurin (PP2B—NCBI: ACX34092.1) is an ubiquitously expressed serine/threonine protein phosphatase that is involved in many biological processes and which is central to T-cell activation. Calcineurin is a heterodimer composed of a catalytic subunit (CnA; three isoforms) and a regulatory subunit (CnB; two isoforms). After engagement of the T-cell receptor, calcineurin dephosphorylates the transcription factor NFAT, allowing it to translocate to the nucleus and active key target gene such as IL2. FK506 in complex with FKBP12, or cyclosporine A (CsA) in complex with CyPA block NFAT access to calcineurin's active site, preventing its dephosphorylation and thereby inhibiting T-cell activation (Brewin et al. (2009) “Generation of EBV-specific cytotoxic T cells that are resistant to calcineurin inhibitors for the treatment of posttransplantation lymphoproliferative disease” Blood 114(23): 4792-803). In a particular embodiment, said mutant form can comprise at least one mutated amino acid of the wild type calcineurin heterodimer a at positions: V314, Y341, M347, T351, W352, L354, K360, preferably double mutations at positions T351 and L354 or V314 and Y341. In a particular embodiment, the valine residue at position 341 can be replaced with a lysine or an arginine residue, the tyrosine residue at position 341 can be replaced with a phenylalanine residue; the methionine at position 347 can be replaced with the glutamic acid, arginine or tryptophane residue; the threonine at position 351 can be replaced with the glutamic acid residue; the tryptophane residue at position 352 can be replaced with a cysteine, glutamic acid or alanine residue, the serine at position 353 can be replaced with the histidine or asparagines residue, the leucine at position 354 can be replaced with an alanine residue; the lysine at position 360 can be replaced with an alanine or phenylalanine residue. In another particular embodiment, said mutant form can comprise at least one mutated amino acid of the wild type calcineurin heterodimer b at positions: V120, N123, L124 or K125, preferably double mutations at positions L124 and K125. In a particular embodiment, the valine at position 120 can be replaced with a serine, an aspartic acid, phenylalanine or leucine residue; the asparagines at position 123 can be replaced with a tryptophan, lysine, phenylalanine, arginine, histidine or serine; the leucine at position 124 can be replaced with a threonine residue; the lysine at position 125 can be replaced with an alanine, a glutamic acid, tryptophan, or two residues such as leucine-arginine or isoleucine-glutamic acid can be added after the lysine at position 125 in the amino acid sequence. Correspondence of amino acid positions described herein is frequently expressed in terms of the positions of the amino acids of the form of wild-type human calcineurin heterodimer b polypeptide (NCBI: ACX34095.1).

Another drug resistance coding sequence is 0(6)-methylguanine methyltransferase (MGMT—UniProtKB: P16455) encoding human alkyl guanine transferase (hAGT). AGT is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG, but retain their ability to repair DNA damage (Maze, R. et al. (1999) “Retroviral-mediated expression of the P140A, but not P140A/G156A, mutant form of O6-methylguanine DNA methyltransferase protects hematopoietic cells against O6-benzylguanine sensitization to chloroethylnitrosourea treatment” J. Pharmacol. Exp. Ther. 290(3): 1467-74). In a particular embodiment, AGT mutant form can comprise a mutated amino acid of the wild type AGT position P140. In a preferred embodiment, said proline at position 140 is replaced with a lysine residue.

Another drug resistance coding sequence can be multidrug resistance protein (MDR1) gene. This gene encodes a membrane glycoprotein, known as P-glycoprotein (P-GP) involved in the transport of metabolic byproducts across the cell membrane. The P-Gp protein displays broad specificity towards several structurally unrelated chemotherapy agents. Thus, drug resistance can be conferred to cells by the expression of nucleic acid sequence that encodes MDR-1 (Genebank NP_000918).

Another drug resistance coding sequence can contribute to the production of cytotoxic antibiotics, such as those from ble or mcrA genes. Ectopic expression of ble gene or mcrA in an immune cell gives a selective advantage when exposed to the respective chemotherapeutic agents bleomycine and mitomycin C (Belcourt, M. F. (1999) “Mitomycin resistance in mammalian cells expressing the bacterial mitomycin C resistance protein MCRA”. PNAS. 96(18):10489-94).

Another drug resistance coding sequence can come from genes encoded mutated version of drug targets, such as mutated variants of mTOR (mTOR mut) conferring resistance to rapamycin such as described by Lorenz M. C. et al. (1995) “TOR Mutations Confer Rapamycin Resistance by Preventing Interaction with FKBP12-Rapamycin” The Journal of Biological Chemistry 270, 27531-27537, or certain mutated variants of Lck (Lckmut) conferring resistance to Gleevec as described by Lee K. C. et al. (2010) “Lck is a key target of imatinib and dasatinib in T-cell activation”, Leukemia, 24: 896-900.

As described above, the genetic modification step of the method can comprise a step of introduction into cells of an exogeneous nucleic acid comprising at least a sequence encoding the drug resistance coding sequence and a portion of an endogenous gene such that homologous recombination occurs between the endogenous gene and the exogeneous nucleic acid. In a particular embodiment, said endogenous gene can be the wild type “drug resistance” gene, such that after homologous recombination, the wild type gene is replaced by the mutant form of the gene which confers resistance to the drug.

### Enhancing Persistence of the Immune Cells In-Vivo

According to one aspect of the present method, the exogenous sequence that is integrated into the immune cells genomic locus encodes a molecule that enhances persistence of the immune cells, especially in-vivo persistence in a tumor environment.

By “enhancing persistence” is meant extending the survival of the immune cells in terms of life span, especially once the engineered immune cells are injected into the patient. For instance, persistence is enhanced, if the mean survival of the modified cells is significantly longer than that of non-modified cells, by at least 10%, preferably 20%, more preferably 30%, even more preferably 50%.

This especially relevant when the immune cells are allogeneic. This may be done by creating a local immune protection by introducing coding sequences that ectopically express and/or secrete immunosuppressive polypeptides at, or through, the cell membrane. A various panel of such polypeptides in particular antagonists of immune checkpoints, immunosuppressive peptides derived from viral envelope or NKG2D ligand can enhance persistence and/or an engraftment of allogeneic immune cells into patients.

According to one embodiment, the immunosuppressive polypeptide to be encoded by said exogenous coding sequence is a ligand of Cytotoxic T-Lymphocyte Antigen 4 (CTLA-4 also known as CD152, GenBank accession number AF414120.1). Said ligand polypeptide is preferably an anti-CTLA-4 immunoglobulin, such as CTLA-4a Ig and CTLA-4b Ig or a functional variant thereof.

According to one embodiment, the immunosuppressive polypeptide to be encoded by said exogenous coding sequence is an antagonist of PD1, such as PD-L1 (other names: CD274, Programmed cell death 1 ligand; ref. UniProt for the human polypeptide sequence Q9NZQ7), which encodes a type I transmembrane protein of 290 amino acids consisting of a Ig V-like domain, a Ig C-like domain, a hydrophobic transmembrane domain and a cytoplasmic tail of 30 amino acids. Such membrane-bound form of PD-L1 ligand is meant in the present invention under a native form (wild-type) or under a truncated form such as, for instance, by removing the intracellular domain, or with one or more mutation(s) (Wang S et al., 2003, J Exp Med. 2003; 197(9): 1083-1091). Of note, PD1 is not considered as being a membrane-bound form of PD-L1 ligand according to the present invention. According to another embodiment, said immunosuppressive polypeptide is under a secreted form. Such recombinant secreted PD-L1 (or soluble PD-L1) may be generated by fusing the extracellular domain of PD-L1 to the Fc portion of an immunoglobulin (Haile S T et al., 2014, Cancer Immunol. Res. 2(7): 610-615; Song M Y et al., 2015, Gut. 64(2):260-71). This recombinant PD-L1 can neutralize PD-1 and abrogate PD-1-mediated T-cell inhibition. PD-L1 ligand may be co-expressed with CTLA4 Ig for an even enhanced persistence of both.

According to another embodiment, the exogenous sequence encodes a polypeptide comprising a viral env immusuppressive domain (ISU), which is derived for instance from HIV-1, HIV-2, SIV, MoMuLV, HTLV-I, -II, MPMV, SRV-1, Syncitin 1 or 2, HERV-K or FELV.

The following Table 1 shows variants of ISU domain from diverse virus which can be expressed within the present invention.

TABLE 1
ISU domain variants from diverse viruses
ISU Amino acids sequences

| | | | | | | | | | | | | | | Virus | |
|---|---|---|---|---|---|---|---|---|---|---|---|---|---|---|---|
| Amino acid positions | | | | | | | | | | | | | | origin | |
| 1 | 2 | 3 | 4 | 5 | 6 | 7 | 8 | 9 | 10 | 11 | 12 | 13 | 14 | Origin | SEQ ID NO |
| L | Q | A | R | I/V | L | A | V | E | R | Y | L | K/R/Q | D | HIV-1 | SEQ ID NO: 68 |
| L | Q | A | R | V | T | A | I | E | K | Y | L | K/A/Q | D/H | HIV-2 | SEQ ID NO: 69 |
| L | Q | A | R | L | L | A | V | E | R | Y | L | K | D | SIV | SEQ ID NO: 70 |
| L | Q | N | R | R | G | L | D | L | L | F | L | K | E | MoMuLV | SEQ ID NO: 71 |
| A | Q | N | R | R | G | L | D | L | L | F | W | E | Q | HTLV-I, | SEQ ID NO: 72 |
| | | | | | | | | | | | | | | -II | |
| L | Q | N | R | R | G | L | D | L | L | T | A | E | Q | MPMV, | SEQ ID NO: 73 |
| | | | | | | | | | | | | | | SRV-1 | |
| L | Q | N | R | R | A | L | D | L | L | T | A | E | R | Syncitin 1 | SEQ ID NO: 74 |
| L | Q | N | R | R | G | L | D | M | L | T | A | A | Q | Syncitin 2 | SEQ ID NO: 75 |
| L | A | N | Q | I | N | D | L | R | Q | T | V | I | W | HERV-K | SEQ ID NO: 76 |
| L | Q | N | R | R | G | L | D | I | L | F | L | Q | E | FELV | SEQ ID NO: 77 |

According to another embodiment, the exogenous sequence encodes a FP polypeptide such as gp41. The following Table 2 represents several FP polypeptide from natural and artificial origins.

TABLE 2
Amino acid sequences of FP polypeptide from
natural and artificial origins
FP Amino acids sequences

| Amino acid positions | | | | | | | | | | SEQ ID |
|---|---|---|---|---|---|---|---|---|---|---|
| 1 | 2 | 3 | 4 | 5 | 6 | 7 | 8 | 9 | Origin | NO |
| G | A | L | F | L | G | F | L | G | HIV-1 gp41 | SEQ ID |
| | | | | | | | | | | NO: 78 |
| A | G | F | G | L | L | L | G | F | Synthetic | SEQ ID |
| | | | | | | | | | | NO: 79 |
| A | G | L | F | L | G | F | L | G | Synthetic | SEQ ID |
| | | | | | | | | | | NO: 80 |

According to another embodiment, the exogenous sequence encodes a non-human MHC homolog, especially a viral MHC homolog, or a chimeric β2m polypeptide such as described by Margalit A. et al. (2003) “Chimeric β2 microglobulin/CD3ζ polypeptides expressed in T cells convert MHC class I peptide ligands into T cell activation receptors: a potential tool for specific targeting of pathogenic CD8+ T cells” Int. Immunol. 15 (11): 1379-1387.

According to one embodiment, the exogenous sequence encodes NKG2D ligand. Some viruses such as cytomegaloviruses have acquired mechanisms to avoid NK cell mediate immune surveillance and interfere with the NKG2D pathway by secreting a protein able to bind NKG2D ligands and prevent their surface expression (Welte, S. A et al. (2003) “Selective intracellular retention of virally induced NKG2D ligands by the human cytomegalovirus UL16 glycoprotein”. Eur. J. Immunol., 33, 194-203). In tumors cells, some mechanisms have evolved to evade NKG2D response by secreting NKG2D ligands such as ULBP2, MICB or MICA (Salih H R, Antropius H, Gieseke F, Lutz S Z, Kanz L, et al. (2003) Functional expression and release of ligands for the activating immunoreceptor NKG2D in leukemia. Blood 102: 1389-1396)

According to one embodiment, the exogenous sequence encodes a cytokine receptor, such as an IL-12 receptor. IL-12 is a well known activator of immune cells activation (Curtis J. H. (2008) “IL-12 Produced by Dendritic Cells Augments CD8+ T Cell Activation through the Production of the Chemokines CCL1 and CCL171”. The Journal of Immunology. 181 (12): 8576-8584.

According to one embodiment the exogenous sequence encodes an antibody that is directed against inhibitory peptides or proteins. Said antibody is preferably be secreted under soluble form by the immune cells. Nanobodies from shark and camels are advantageous in this respect, as they are structured as single chain antibodies (Muyldermans S. (2013) “Nanobodies: Natural Single-Domain Antibodies” Annual Review of Biochemistry 82: 775-797). Same are also deemed more easily to fuse with secretion signal polypeptides and with soluble hydrophilic domains.

The different aspects developed above to enhance persistence of the cells are particularly preferred, when the exogenous coding sequence is introduced by disrupting an endogenous gene encoding P2m or another MHC component, as detailed further on.

### Enhancing the Therapeutic Activity of Immune Cells

According to one aspect of the present method, the exogenous sequence that is integrated into the immune cells genomic locus encodes a molecule that enhances the therapeutic activity of the immune cells.

By “enhancing the therapeutic activity” is meant that the immune cells, or population of cells, engineered according to the present invention, become more aggressive than non-engineered cells or population of cells with respect to a selected type of target cells. Said target cells generally belong to a defined type of cells, or population of cells, preferably characterized by common surface marker(s). In the present specification, “therapeutic potential” reflects the therapeutic activity, as measured through in-vitro experiments. In general sensitive cancer cell lines, such as Daudi cells, are used to assess whether the immune cells are more or less active towards said cells by performing cell lysis or growth reduction measurements. This can also be assessed by measuring levels of degranulation of immune cells or chemokines and cytokines production. Experiments can also be performed in mice with injection of tumor cells, and by monitoring the resulting tumor expansion. Enhancement of activity is deemed significant when the number of developing cells in these experiments is reduced by the immune cells by more than 10%, preferably more than 20%, more preferably more than 30%, even more preferably by more than 50%.

According to one aspect of the invention, said exogenous sequence encodes a chemokine or a cytokine, such as IL-12. It is particularly advantageous to express IL-12 as this cytokine is extensively referred to in the literature as promoting immune cell activation (Colombo M. P. et al. (2002) “Interleukin-12 in anti-tumor immunity and immunotherapy” Cytokine Growth Factor Rev. 13(2):155-68).

According to a preferred aspect of the invention the exogenous coding sequence encodes or promote secreted factors that act on other populations of immune cells, such as T-regulatory cells, to alleviate their inhibitory effect on said immune cells.

According to one aspect of the invention, said exogenous sequence encodes an inhibitor of regulatory T-cell activity is a polypeptide inhibitor of forkhead/winged helix transcription factor 3 (FoxP3), and more preferably is a cell-penetrating peptide inhibitor of FoxP3, such as that referred as P60 (Casares N. et al. (2010) “A peptide inhibitor of FoxP3 impairs regulatory T cell activity and improves vaccine efficacy in mice.” J Immunol 185(9):5150-9).

By “inhibitor of regulatory T-cells activity” is meant a molecule or precursor of said molecule secreted by the T-cells and which allow T-cells to escape the down regulation activity exercised by the regulatory T-cells thereon. In general, such inhibitor of regulatory T-cell activity has the effect of reducing FoxP3 transcriptional activity in said cells.

According to one aspect of the invention, said exogenous sequence encodes a secreted inhibitor of Tumor Associated Macrophages (TAM), such as a CCR2/CCL2 neutralization agent. Tumor-associated macrophages (TAMs) are critical modulators of the tumor microenvironment. Clinicopathological studies have suggested that TAM accumulation in tumors correlates with a poor clinical outcome. Consistent with that evidence, experimental and animal studies have supported the notion that TAMs can provide a favorable microenvironment to promote tumor development and progression. (Theerawut C. et al. (2014) “Tumor-Associated Macrophages as Major Players in the Tumor Microenvironment” Cancers (Basel) 6(3): 1670-1690). Chemokine ligand 2 (CCL2), also called monocyte chemoattractant protein 1 (MCP1—NCBI NP_002973.1), is a small cytokine that belongs to the CC chemokine family, secreted by macrophages, that produces chemoattraction on monocytes, lymphocytes and basophils. CCR2 (C-C chemokine receptor type 2—NCBI NP_001116513.2), is the receptor of CCL2.

### Enhancing Specificity and Safety of Immune Cells

Expressing chimeric antigen receptors (CAR) have become the state of the art to direct or improve the specificity of primary immune cells, such as T-Cells and NK-cells for treating tumors or infected cells. CARs expressed by these immune cells specifically target antigen markers at the surface of the pathological cells, which further help said immune cells to destroy these cells in-vivo (Sadelain M. et al. “The basic principles of chimeric antigen receptor design” (2013) Cancer Discov. 3(4):388-98). CARs are usually designed to comprise activation domains that stimulate immune cells in response to binding to a specific antigen (so-called positive CAR), but they may also comprise an inhibitory domain with the opposite effect (so-called negative CAR)(Fedorov, V. D. (2014) “Novel Approaches to Enhance the Specificity and Safety of Engineered T Cells” Cancer Joumal 20 (2):160-165. Positive and negative CARs may be combined or co-expressed to finely tune the cells immune specificity depending of the various antigens present at the surface of the target cells.

The genetic sequences encoding CARs are generally introduced into the cells genome using retroviral vectors that have elevated transduction efficiency but integrate at random locations. Here, according to the present invention, components of chimeric antigen receptor (CAR) car be introduced at selected loci, more particularly under control of endogenous promoters by targeted gene recombination.

According to one aspect, while a positive CAR is introduced into the immune cell by a viral vector, a negative CAR can be introduced by targeted gene insertion and vice-versa, and be active preferably only during immune cells activation. Accordingly, the inhibitory (i.e. negative) CAR contributes to an improved specificity by preventing the immune cells to attack a given cell type that needs to be preserved. Still according to this aspect, said negative CAR can be an apoptosis CAR, meaning that said CAR comprise an apoptosis domain, such as FasL (CD95—NCBI: NP_000034.1) or a functional variant thereof, that transduces a signal inducing cell death (Eberstadt M; et al. “NMR structure and mutagenesis of the FADD (Mort1) death-effector domain” (1998) Nature. 392 (6679): 941-5).

Accordingly, the exogenous coding sequence inserted according to the invention can encode a factor that has the capability to induce cell death, directly, in combination with, or by activating other compound(s).

As another way to enhance the safety of us of the primary immune cells, the exogenous coding sequence can encodes molecules that confer sensitivity of the immune cells to drugs or other exogenous substrates. Such molecules can be cytochrome(s), such as from the P450 family (Preissner S et al. (2010) “SuperCYP: a comprehensive database on Cytochrome P450 enzymes including a tool for analysis of CYP-drug interactions”. Nucleic Acids Res 38 (Database issue): D237-43), such as CYP2D6-1 (NCBI—NP_000097.3), CYP2D6-2 (NCBI—NP_001020332.2), CYP2C9( ), CYP3A4 (NCBI—NP_000762.2), CYP2C19 (NCBI—NP_000760.1) or CYP1A2 (NCBI—NP_000752.2), conferring hypersensitivity of the immune cells to a drug, such as cyclophosphamide and/or isophosphamide.

According to a further aspect of the invention, an exogenous sequence is introduced in the immune cells for its expression, especially in vivo, to reduce IL-6 or IL-8 trans signalling in view of controlling potential Cyokine Release Syndrome (CRS).

Such an exogenous sequence can encode for instance antibodies directed against IL-6 or IL-8 or against their receptors IL-6R or IL-8R.

According to a preferred aspect said exogenous sequence can encode soluble extracellular domain of GP130, such as one showing at least 80% identity with SEQ ID NO:61.

Such soluble extracellular domain of GP130 is described for instance by Rose-John S. [The Soluble Interleukine Receptor Advanced Therapeutic Options in Inflammation (2017) Clinical Pharmacology & Therapeutics, 102(4):591-598] can be fused with fragments of immunoglobulins, such as sgp130Fc (SEQ ID NO:62). As stated before, said exogenous sequence can be stably integrated into the genome by site directed mutagenesis (i.e. using sequence specific nuclease reagents) and be placed under the transcriptional activity of an endogenous promoter at a locus which is active during immune cell activation, such as one listed in Tables 6, 8 or 9, and preferably up-regulated upon CAR activation or being CAR dependent.

According to a more preferred embodiment, the exogenous sequence is introduced into a CAR positive immune cell, such as one expressing an anti-CD22 CAR T-cell polynucleotide sequence such as SEQ ID NO:31. According to some more specific embodiments, said exogenous sequence coding for a polypeptide which can associate, and preferably interfere, with a cytokine receptor of the IL-6 receptor family, such as said soluble extracellular domain of GP130, is integrated at a PD1, CD25 or CD69 locus. As per the present invention, the endogenous sequence encoding PD1 locus is preferably disrupted by said exogenous sequence.

The invention thus provides with a method for treating or reducing CRS in cell immunotherapy, wherein cells or a therapeutic composition thereof are administered to patients, said cells being genetically modified to secrete polypeptide(s) comprising a soluble extracellular domain of GP130, sGP130Fc, an anti-IL-6 or anti-IL6R antibody, an anti-IL-8 or anti-IL8R antibody, or any fusion thereof.

Examples of preferred genotypes of the engineered immune cells are:
[CAR]^{positive}[GP130]^{positive}
[CAR]^{positive}[GP130]^{positive}
[CAR]^{positive}[TCR]^{negative}[GP130]^{positive}[PD1]^{negative}
[CAR]^{positive}[TCR]^{negative}[GP130]^{positive}[PD1]^{negative}
[CAR]^{positive}[GP130]^{positive}[CD25]^{negative}
[CAR]^{positive}[TCR]^{negative}[GP130]^{positive}[CD25]^{negative}

### Improving the Efficiency of Gene Targeted Insertion in Primary Immune Cells Using AAV Vectors

The present specification provides with donor templates and sequence specific reagents as illustrated in the figures that are useful to perform efficient insertion of a coding sequence in frame with endogenous promoters, in particular PD1 and CD25, as well as means and sequences for detecting proper insertion of said exogenous sequences at said loci.

The donor templates according to the present invention are generally polynucleotide sequences which can be included into a variety of vectors described in the art prompt to deliver the donor templates into the nucleus at the time the endonuclease reagents get active to obtain their site directed insertion into the genome generally by NHEJ or homologous recombination,

Specifically, the present invention provides specific donor polynucleotides for expression of IL-15 (SEQ ID NO:59) at the PD1 locus comprising one or several of the following sequences:
Sequence encoding IL-15, such as one presenting identity with SEQ ID NO:50;
Upstream and downstream (also referred to left and right) sequences homologous to the PD1 locus, comprising preferably polynucleotide sequences SEQ ID NO:45 and SEQ ID NO:46;
optionally, a sequence encoding soluble form of an IL-15 receptor (sIL-15R), such as one presenting identity with SEQ ID NO:50;
optionally, at least one_2A peptide cleavage site such as one of SEQ ID NO:53 (F2A), SEQ ID NO:54 (P2A) and/or SEQ ID NO:55 (T2A),
Specifically, the present invention provides specific donor polynucleotides for expression of IL-12 (SEQ ID NO:58) at the PD1 locus comprising one or several of the following sequences:
   Sequence encoding IL-12a, such as one presenting identity with SEQ ID NO:47;
   Upstream and downstream (also referred to left and right) sequences homologous to the PD1 locus, comprising preferably polynucleotide sequences SEQ ID NO:45 and SEQ ID NO:46;
   optionally, a sequence encoding IL-12b, such as one presenting identity with SEQ ID NO:48;
   optionally, at least one 2A peptide cleavage site such as one of SEQ ID NO:53 (F2A), SEQ ID NO:54 (P2A) and/or SEQ ID NO:55 (T2A),

Specifically, the present invention provides specific donor polynucleotides for expression of soluble GP130 (comprising SEQ ID NO:61) at the PD1 locus comprising one or several of the following sequences:
Sequence encoding soluble GP130, preferably a soluble gp130 fused to a Fc, such as one presenting identity with SEQ ID NO:62;
Upstream and downstream (also referred to left and right) sequences homologous to the PD1 locus, comprising preferably polynucleotide sequences SEQ ID NO:45 and SEQ ID NO:46;
optionally, at least one_2A peptide cleavage site such as one of SEQ ID NO:53 (F2A), SEQ ID NO:54 (P2A) and/or SEQ ID NO:55 (T2A),

Specifically, the present invention provides specific donor polynucleotides for expression of IL-15 (SEQ ID NO:59) at the CD25 locus comprising one or several of the following sequences:
Sequence encoding IL-15, such as one presenting identity with SEQ ID NO:50;
Upstream and downstream (also referred to left and right) sequences homologous to the CD25 locus, comprising preferably polynucleotide sequences SEQ ID NO:43 and SEQ ID NO:44;
optionally, a sequence encoding soluble form of an IL-15 receptor (sIL-15R), such as one presenting identity with SEQ ID NO:50;
optionally, at least one 2A peptide cleavage site such as one of SEQ ID NO:53 (F2A), SEQ ID NO:54 (P2A) and/or SEQ ID NO:55 (T2A),

Specifically, the present invention provides specific donor polynucleotides for expression of IL-12 (SEQ ID NO:58) at the CD25 locus comprising one or several of the following sequences:
Sequence encoding IL-12a, such as one presenting identity with SEQ ID NO:47;
Upstream and downstream (also referred to left and right) sequences homologous to the CD25 locus, comprising preferably polynucleotide sequences SEQ ID NO:43 and SEQ ID NO:44;
optionally, a sequence encoding IL-12b, such as one presenting identity with SEQ ID NO:48;
optionally, at least one_2A peptide cleavage site such as one of SEQ ID NO:53 (F2A), SEQ ID NO:54 (P2A) and/or SEQ ID NO:55 (T2A),

Specifically, the present invention provides specific donor polynucleotides for expression of soluble GP130 (comprising SEQ ID NO:61) at the CD25 locus comprising one or several of the following sequences:
Sequence encoding soluble GP130, preferably a soluble gp130 fused to a Fc, such as one presenting identity with SEQ ID NO:62;
   Upstream and downstream (also referred to left and right) sequences homologous to the CD25 locus, comprising preferably polynucleotide sequences SEQ ID NO:43 and SEQ ID NO:44;
   optionally, at least one_2A peptide cleavage site such as one of SEQ ID NO:53 (F2A), SEQ ID NO:54 (P2A) and/or SEQ ID NO:55 (T2A), As illustrated in the examples herein, the inventors have significantly improved the rate of gene targeted insertion into human cells by using AAV vectors, especially vectors from the AAV6 family.

One broad aspect of the present invention is thus the transduction of AAV vectors in human primary immune cells, in conjunction with the expression of sequence specific endonuclease reagents, such as TALE endonucleases, more preferably introduced under mRNA form, to increase homologous recombination events in these cells.

According to one aspect of this invention, sequence specific endonuclease reagents can be introduced into the cells by transfection, more preferably by electroporation of mRNA encoding said sequence specific endonuclease reagents, such as TALE nucleases.

Still according to this broad aspect, the invention more particularly provides a method of insertion of an exogenous nucleic acid sequence into an endogenous polynucleotide sequence in a cell, comprising at least the steps of
transducing into said cell an AAV vector comprising said exogenous nucleic acid sequence and sequences homologous to the targeted endogenous DNA sequence, and
Inducing the expression of a sequence specific endonuclease reagent to cleave said endogenous sequence at the locus of insertion.

The obtained insertion of the exogenous nucleic acid sequence may result into the introduction of genetic material, correction or replacement of the endogenous sequence, more preferably “in frame” with respect to the endogenous gene sequences at that locus.

According to another aspect of the invention, from 10⁵ to 10⁷ preferably from 10⁶ to 10⁷, more preferably about 5·10⁶ viral genomes are transduced per cell.

According to another aspect of the invention, the cells can be treated with proteasome inhibitors, such as Bortezomib to further help homologous recombination.

As one object of the present invention, the AAV vector used in the method can comprise a promoterless exogenous coding sequence as any of those referred to in this specification in order to be placed under control of an endogenous promoter at one loci selected among those listed in the present specification.

As one object of the present invention, the AAV vector used in the method can comprise a 2A peptide cleavage site followed by the cDNA (minus the start codon) forming the exogenous coding sequence.

As one object of the present invention, said AAV vector comprises an exogenous sequence coding for a chimeric antigen receptor, especially an anti-CD19 CAR, an anti-CD22 CAR, an anti-CD123 CAR, an anti-CS1 CAR, an anti-CCL1 CAR, an anti-HSP70 CAR, an anti-GD3 CAR or an anti-ROR1 CAR.

The invention thus encompasses any AAV vectors designed to perform the method herein described, especially vectors comprising a sequence homologous to a locus of insertion located in any of the endogenous gene responsive to T-cell activation referred to in Table 4.

Many other vectors known in the art, such as plasmids, episomal vectors, linear DNA matrices, etc. . . . can also be used following the teachings to the present invention.

As stated before, the DNA vector used according to the invention preferably comprises: (1) said exogenous nucleic acid comprising the exogenous coding sequence to be inserted by homologous recombination, and (2) a sequence encoding the sequence specific endonuclease reagent that promotes said insertion. According to a more preferred aspect, said exogenous nucleic acid under (1) does not comprise any promoter sequence, whereas the sequence under (2) has its own promoter. According to an even more preferred aspect, the nucleic acid under (1) comprises an Internal Ribosome Entry Site (IRES) or “self-cleaving” 2A peptides, such as T2A, P2A, E2A or F2A, so that the endogenous gene where the exogenous coding sequence is inserted becomes multi-cistronic. The IRES of 2A Peptide can precede or follow said exogenous coding sequence.

Preferred vectors of the present invention are vectors derived from AAV6, comprising donor polynucleotides as previously described herein or illustrated in the experimental section and figures. Examples of vectors according to the invention comprise or consist of polynucleotides having identity with sequences SEQ ID NO:37 (matrix for integration of sequence coding for IL-15 into the CD25 locus), SEQ ID NO:38 (matrix for integration of sequence coding for IL-15 into the PD1 locus) SEQ ID NO:39 (matrix for integration of sequence coding for IL-12 into the CD25 locus) and SEQ ID NO:40 (matrix for integration of sequence coding for IL-12 into the PD1 locus).

### Gene Targeted Integration in Immune Cells Under Transcriptional Control of Endogenous Promoters

The present invention, in one of its main aspects, is taking advantage of the endogenous transcriptional activity of the immune cells to express exogenous sequences that improve their therapeutic potential.

The invention provides with several embodiments based on the profile of transcriptional activity of the endogenous promoters and on a selection of promoter loci useful to carry out the invention. Preferred loci are those, which transcription activity is generally high upon immune cell activation, especially in response to CAR activation (CAR-sensitive promoters) when the cells are endowed with CARs.

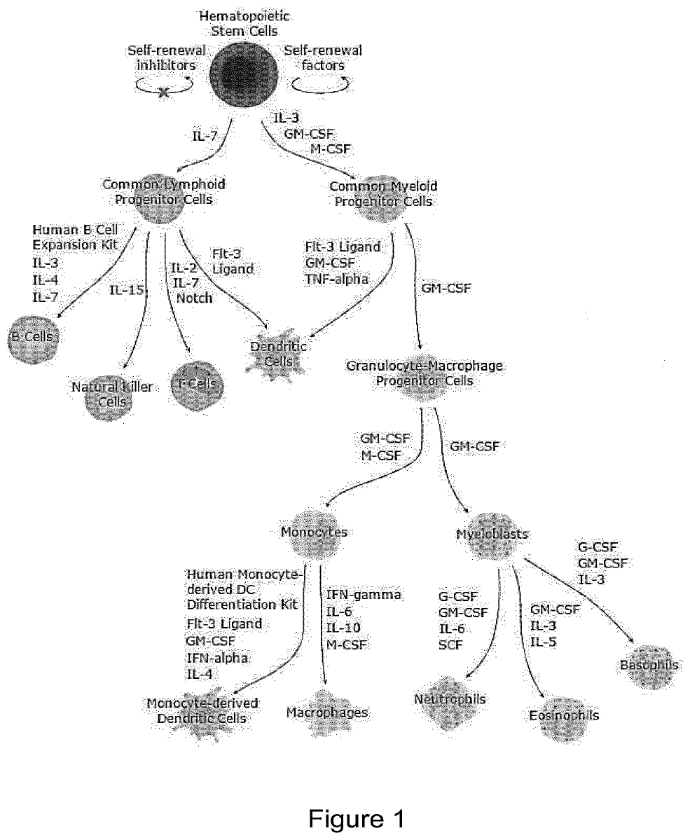

Accordingly, the invention provides with a method for producing allogeneic therapeutic immune cells by expressing a first exogenous sequence encoding a CAR at the TCR locus, thereby disrupting TCR expression, and expressing a second exogenous coding sequence under transcriptional activity of an endogenous locus, preferably dependent from either:
CD3/CD28 activation, such as dynabeads, which is useful for instance for promoting cells expansion;
CAR activation, such as through the CD3zeta pathway, which is useful for instance to activate immune cells functions on-target;
Transcriptional activity linked to the appearance of disease symptom or molecular marker. which is useful for instance for activating the cells in-situ in ill organs.
Cell differentiation, which is useful for conferring therapeutic properties to cells at a given level of differentiation or to express protein into a particular lineage (see FIG. 1), for instance at the time hematopoietic cells gain their immune functions; or/and
TME (Tumor microoenvironment), which is useful for redirect cells activity and their amplification to specific tumor conditions (hypoxia, low glucose . . . ), or for preventing exhaustion and/or sustaining activation;
CRS (cytokine release syndrome), which is useful to mitigate adverse events related to CAR T-cell activity

The inventors have established a first list of endogenous genes (Table 6) which have been found to be particularly appropriate for applying the targeted gene recombination as per the present invention. To draw this list, they have come across several transcriptome murine databases, in particular that from the Immunological Genome Project Consortium referred to in Best J. A. et al. (2013) “Transcriptional insights into the CD8(+) T cell response to infection and memory T cell formation” Nat. Immunol. 14(4):404-12., which allows comparing transcription levels of various genes upon T-cell activation, in response to ovalbumin antigens. Also, because very few data is available with respect to human T-cell activation, they had to make some extrapolations and analysis from these data and compare with the human situation by studying available literature related to the human genes. The selected loci are particularly relevant for the insertion of sequences encoding CARs. Based on the first selection of Table 6, they made subsequent selections of genes based on their expected expression profiles (Tables 7 to 10).

On another hand, the inventors have identified a selection of transcriptional loci that are mostly inactive, which would be most appropriate to insert expression cassette(s) to express exogenous coding sequence under the transcriptional control of exogenous promoters. These loci are referred to as “safe harbor loci” as those being mostly transcriptionally inactive, especially during T-Cell activation. They are useful to integrate a coding sequence by reducing at the maximum the risk of interfering with genome expression of the immune cells.

Gene Targeted Insertion Under Control of Endogenous Promoters that are Steadily Active During Immune Cell Activation

A selection of endogenous gene loci related to this embodiment is listed in Table 7.

Accordingly the method of the present invention provides with the step of performing gene targeted insertion under control of an endogenous promoter that is constantly active during immune cell activation, preferably from of an endogenous gene selected from CD3G, Rn28s1, Rn18s, Rn7sk, Actg1, β2m, Rpl18a, Pabpc1, Gapdh, Rpl17, Rpl19, Rplp0, Cfl1 and Pfn1.

By “steadily active” means that the transcriptional activity observed for these promoters in the primary immune cell is not affected by a negative regulation upon the activation of the immune cell.

As reported elsewhere (Acuto, O. (2008) “Tailoring T-cell receptor signals by proximal negative feedback mechanisms”. Nature Reviews Immunology 8:699-712), the promoters present at the TCR locus are subjected to different negative feedback mechanisms upon TCR engagement and thus may not be steadily active or up regulated during for the method of the present invention. The present invention has been designed to some extend to avoid using the TCR locus as a possible insertion site for exogenous coding sequences to be expressed during T-cell activation. Therefore, according to one aspect of the invention, the targeted insertion of the exogenous coding sequence is not performed at a TCRalpha or TCRbeta gene locus.

Examples of exogenous coding sequence that can be advantageously introduced at such loci under the control of steadily active endogenous promoters, are those encoding or positively regulating the production of a cytokine, a chemokine receptor, a molecule conferring resistance to a drug, a co-stimulation ligand, such as 4-1BRL and OX40L, or of a secreted antibody.

Gene Integration Under Endogenous Promoters that are Dependent from Immune Cell Activation or Dependent from CAR Activation

As stated before, the method of the present invention provides with the step of performing gene targeted insertion under control of an endogenous promoter, which transcriptional activity is preferably up-regulated upon immune cell activation, either transiently or over more than 10 days.

By “immune cell activation” is meant production of an immune response as per the mechanisms generally described and commonly established in the literature for a given type of immune cells. With respect to T-cell, for instance, T-cell activation is generally characterized by one of the changes consisting of cell surface expression by production of a variety of proteins, including CD69, CD71 and CD25 (also a marker for Treg cells), and HLA-DR (a marker of human T cell activation), release of perforin, granzymes and granulysin (degranulation), or production of cytokine effectors IFN-γ, TNF and LT-alpha.

According to a preferred embodiment of the invention, the transcriptional activity of the endogenous gene is up-regulated in the immune cell, especially in response to an activation by a CAR. The CAR can be independently expressed in the immune cell. By “independently expressed” is meant that the CAR can be transcribed in the immune cell from an exogenous expression cassette introduced, for instance, using a retroviral vector, such as a lentiviral vector, or by transfecting capped messenger RNAs by electroporation encoding such CAR Many methods are known in the art to express a CAR into an immune cell as described for instance by (REF.)

Said endogenous gene whose transcriptional activity is up regulated are particularly appropriate for the integration of exogenous sequences to encode cytokine(s), such as IL-12 and IL-15, immunogenic peptide(s), or a secreted antibody, such as an anti-IDO1, anti-IL10, anti-PD1, anti-PDL1, anti-IL6 or anti-PGE2 antibody.

According to a preferred embodiment of the invention, the endogenous promoter is selected for its transcriptional activity being responsive to, and more preferably being dependent from CAR activation.

As shown herein, CD69, CD25 and PD1 are such loci, which are particularly appropriate for the insertion of expression of an exogenous coding sequences to be expressed when the immune cells get activated, especially into CAR positive immune cells.

The present invention thus combines any methods of expressing a CAR into an immune cell with the step of performing a site directed insertion of an exogenous coding sequence at a locus, the transcriptional activity of which is responsive to or dependent from the engagement of said CAR with a tumor antigen. Especially, the method comprises the step of introducing into a CAR positive or Recombinant TCR positive immune cell an exogenous sequence encoding IL-12 or IL-15 under transcriptional control of one promoter selected from PD1, CD25 and CD69 promoters.

In particular, CAR positive cells can obtained by following the steps of co-expressing into an immune cell, preferably a primary cell, and more preferably into a primary T-cell, at least one exogenous sequence encoding a CAR and another exogenous sequence placed under an endogenous promoter dependent, which transcriptional activity is dependent from said CAR, such a PD1, CD25 or CD71.

The expression “dependent from said CAR” means that the transcriptional activity of said endogenous promoter is necessary increased by more than 10%, preferably by more than 20%, more preferably by more than 50% and even more preferably more than 80%, as a result of the engagement of the CAR with its cognate antigen, in a situation where, in general, the antigens are exceeding the number of CARs present at the cell surface and the number of CARs expressed at the cell surface is more than 10 per cell, preferably more than 100, and more preferably more than 1000 molecules per cells.

The present invention thus teaches the expression of a CAR sequence, preferably inserted at the TCR locus and constitutively expressed, whereas another exogenous sequence integrated at another locus is co-expressed, in response to, or dependent from, the engagement of said CAR with its cognate antigen. Said another locus is for instance CD25, PD1 or CD71 or any loci being specifically transcribed upon CAR activation.

In other words, the invention provides the co-expression of a CAR and at least one exogenous coding sequence, the expression of said exogenous sequence being under control of an endogenous promoter the transcriptional activity of which is influenced by the CAR activity, this being done in view of obtaining engineered immune cells offering a better immune response.

As previously described, this can be performed by transfecting the cells with sequence-specific nuclease reagents targeting the coding regions of such loci being specifically CAR dependent, along with donor templates comprising sequences homologous to said genomic regions. The sequence specific nuclease reagents help the donor templates to be integrated by homologous recombination or NHEJ.

According to a preferred embodiment, the exogenous coding sequence is integrated in frame with the endogenous gene, so that the expression of said endogenous gene is preserved. This is the case for instance with respect to CD25 and CD69 in at least one example of the experimental section herein.

According to a preferred embodiment, the exogenous sequence disrupts the endogenous coding sequence of the gene to prevent its expression of one endogenous coding sequence, especially when this expression has a negative effect on the immune cell functions, as it the case for instance with PD1 in the experimental section herein.

According to an even more preferred embodiments, the exogenous coding sequence, which disrupts the endogenous gene sequence is placed in frame with the endogenous promoter, so that its expression is made dependent from the endogenous promoter as also shown in the experimental section.

The present invention is also drawn to the polynucleotide and polypeptide sequences encoding the different TAL-nucleases exemplified in the present patent application, especially those permitting the site directed insertion at the CD25 locus (SEQ ID NO:18 and 19), as well as their respective target and RVD sequences.

The present invention also encompasses kits for immune cells transfection comprising polynucleotides encoding the sequence-specific endonuclease reagents and the donor sequences designed to integrate the exogenous sequence at the locus targeted by said reagents. Examples of such kits are a kit comprising mRNA encoding rare-cutting endonuclease targeting PD1 locus (ex: PD1 TALEN®) and an AAV vector comprising an exogenous sequence encoding IL-12, a kit comprising mRNA encoding rare-cutting endonuclease targeting PD1 locus (ex: PD1 TALEN®) and an AAV vector comprising an exogenous sequence encoding IL-15, a kit comprising mRNA encoding rare-cutting endonuclease targeting CD25 locus (ex: CD25 TALEN®) and an AAV vector comprising an exogenous sequence encoding IL-12, a kit comprising mRNA encoding rare-cutting endonuclease targeting CD25 locus (ex: CD25 TALEN®) and an AAV vector comprising an exogenous sequence encoding IL-15, a kit comprising mRNA encoding rare-cutting endonuclease targeting PD1 locus (ex: PD1 TALEN®) and an AAV vector comprising an exogenous sequence encoding soluble gp130, a kit comprising mRNA encoding rare-cutting endonuclease targeting CD25 locus (ex: CD25 TALEN®) and an AAV vector comprising an exogenous sequence encoding soluble gp130, and any kits involving endonuclease reagents targeting a gene listed in table 6, and a donor matrix for introducing a coding sequence referred to in the present specification.

According to one aspect of the invention, the endogenous gene is selected for a weak up-regulation. The exogenous coding sequence introduced into said endogenous gene whose transcriptional activity is weakly up regulated, can be advantageously a constituent of an inhibitory CAR, or of an apoptotic CAR, which expression level has generally to remain lower than that of a positive CAR. Such combination of CAR expression, for instance one transduced with a viral vector and the other introduced according to the invention, can greatly improve the specificity or safety of CAR immune cells

Some endogenous promoters are transiently up-regulated, sometimes over less than 12 hours upon immune cell activation, such as those selected from the endogenous gene loci Spata6, Itga6, Rcbtb2, Cdld1, St8sia4, Itgae and Fam214a (Table 8). Other endogenous promoters are up-regulated over less than 24 hours upon immune cell activation, such as those selected from the endogenous gene loci IL3, IL2, Ccl4, IL21, Gp49a, Nr4a3, Lirb4, Cd200, Cdkn1a, Gzmc, Nr4a2, Cish, Ccr8, Lad1 and Crabp2 (Table 9) and others over more than 24 hours, more generally over more than 10 days, upon immune cell activation. Such as those selected from Gzmb, Tbx2l, Plek, Chek1, Slamf7, Zbtb32, Tigit, Lag3, Gzma, Wee1, IL12rb2, Eea1 and DtU (Table 9).

Alternatively, the inventors have found that endogenous gene under transcriptional control of promoters that are down-regulated upon immune cell activation, could also be of interest for the method according to the present invention. Indeed they have conceived that exogenous coding sequences encoding anti-apoptotic factors, such as of Bcl2 family, BcIXL, NF-kB, Survivin, or anti-FAP (fibroblast activation protein), such as a constituent of a CAR anti-FAP, could be introduced at said loci. Said endogenous gene under transcriptional control of promoters that are down-regulated upon immune cell activation can be more particularly selected from Slc6a19, Cd55, Xkrx, Mtum, H2-Ob, Cnr2, Itgae, Raver2, Zbtb20, Arrb1, Abca1, Tet1, Sic16a5 and Ampd3 (Table 10)

### Gene Integration Under Endogenous Promoters Activated Under Tumor Microenvironment (TME) Conditions

One aspect of the present invention more particularly concerns methods to prevent immune cells exhaustion in tumor microenvironment (TME) conditions. Immune cells often get exhausted in response to nutrient depletion or molecular signals found in the microoenvironment of tumors, which helps tumor resistance. The method comprises the steps of engineering immune cells by integrating exogenous coding sequences under control of endogenous promoters which are up-regulated under arginine, cysteine, tryptophan and oxygen deprivation as well as extracellular acidosis (lactate build up).

Such exogenous sequences may encode chimeric antigen receptors, interleukins, or any polypeptide given elsewhere in this specification to bolster immune cells function or activation and/or confer a therapeutic advantage.

The inventors have listed a number of loci which have been found to be upregulated in a large number of exhausted tumor infiltrating lymphocytes (TIL), which are listed in tables 12 and 13. The invention provides with the step of integrating exogenous coding sequences at these preferred loci to prevent exhaustion of the immune cells, in particular T-cells, in tumor microoenvironment.

For instance, the exogenous sequences encoding a CAR can be placed under transcriptional control of the promoter of endogenous genes that are activated by the tumor microenvironment, such as HIF1a, transcription factor hypoxia-inducible factor, or the aryl hydrocarbon receptor (AhR), These gene are sensors respectively induced by hypoxia and xenobiotics in the close environment of tumors.

The present invention is thus useful to improve the therapeutic outcome of CAR T-cell therapies by integrating exogenous coding sequences, and more generally genetic attributes/circuits, under the control of endogenous T-cell promoters influenced by tumor microenvironment (TME).

Pursuant to the invention, upregulation of endogenous genes can be “hijacked” to re-express relevant exogenous coding sequences to improve the antitumor activity of CAR T-cells in certain tumor microenvironment

### Gene Targeted Insertion and Expression in Hematopoietic Stem Cells (HSCs)

One aspect of the present invention more particularly concerns the insertion of transgenes into hematopoietic stem cells (HSCs).

Hematopoietic stem cells (HSCs) are multipotent, self-renewing progenitor cells from which all differentiated blood cell types arise during the process of hematopoiesis. These cells include lymphocytes, granulocytes, and macrophages of the immune system as well as circulating erythrocytes and platelets. Classically, HSCs are thought to differentiate into two lineage-restricted, lymphoid and myelo-erythroid, oligopotent progenitor cells. The mechanisms controlling HSC self-renewal and differentiation are thought to be influenced by a diverse set of cytokines, chemokines, receptors, and intracellular signaling molecules. Differentiation of HSCs is regulated, in part, by growth factors and cytokines including colony-stimulating factors (CSFs) and interleukins (ILs) that activate intracellular signaling pathways. The factors depicted below are known to influence HSC multipotency, proliferation, and lineage commitment. HSCs and their differentiated progeny can be identified by the expression of specific cell surface lineage markers such as cluster of differentiation (CD) proteins and cytokine receptors into hematopoietic stem cells.

Gene therapy using HSCs has enormous potential to treat diseases of the hematopoietic system including immune diseases. In this approach, HSCs are collected from a patient, gene-modified ex-vivo using integrating retroviral vectors, and then infused into a patient To date retroviral vectors have been the only effective gene delivery system for HSC gene therapy. Gene delivery to HSCs using integrating vectors thereby allowing for efficient delivery to HSC-derived mature hematopoietic cells. However, the gene-modified cells that are infused into a patient are a polyclonal population, where the different cells have vector proviruses integrated at different chromosomal locations, which can result into many adverse mutations, which may be amplified due to some proliferative/survival advantage of these mutations (Powers and Trobridge (2013) “Identification of Hematopoietic Stem Cell Engraftment Genes in Gene Therapy Studies” J Stem Cell Res Ther S3:004. doi:10.4172/2157-7633.S3-00).

HSCs are commonly harvested from the peripheral blood after mobilization (patients receive recombinant human granulocyte-colony stimulating factor (G-CSF)). The patient's peripheral blood is collected and enriched for HSCs using the CD34+ marker. HSCs are then cultured ex vivo and exposed to viral vectors. The ex vivo culture period varies from 1 to 4 days. Prior to the infusion of gene-modified HSCs, patients may be treated with chemotherapy agents or irradiation to help enhance the engraftment efficiency. Gene-modified HSCs are re-infused into the patient intravenously. The cells migrate into the bone marrow before finally residing in the sinusoids and perivascular tissue. Both homing and hematopoiesis are integral aspects of engraftment. Cells that have reached the stem cell niche through homing will begin producing mature myeloid and lymphoid cells from each blood lineage. Hematopoiesis continues through the action of long-term HSCs, which are capable of self-renewal for life-long generation of the patient's mature blood cells, in particular the production of common lymphoid progenitor cells, such as T cells and NK cells, which are key immune cells for eliminating infected and malignant cells.

The present invention provides with performing gene targeted insertion in HSCs to introduce exogenous coding sequences under the control of endogenous promoters, especially endogenous promoters of genes that are specifically activated into cells of a particular hematopoietic lineage or at particular differentiation stage, preferably at a late stage of differentiation. The HSCs can be transduced with a polynucleotide vector (donor template), such as an AAV vector, during an ex-vivo treatment as referred to in the previous paragraph, whereas a sequence specific nuclease reagent is expressed as to promote the insertion of the coding sequences at the selected locus. The resulting engineered HSCs can be then engrafted into a patient in need thereof for a long term in-vivo production of engineered immune cells that will comprise said exogenous coding sequences. Depending on the activity of the selected endogenous promoter, the coding sequences will be selectively expressed in certain lineages or in response to the local environment of the immune cells in-vivo, thereby providing adoptive immunotherapy.

According to one preferred aspect of the invention, the exogenous coding sequences are placed under the control of promoters of a gene, which transcriptional activity is specifically induced in common lymphoid progenitor cells, such as CD34, CD43, Flt-3/Flk-2, IL-7 R alpha/CD127 and Neprilysin/CD10.

More preferably, the exogenous coding sequences are placed under the control of promoters of a gene, which transcriptional activity is specifically induced in NK cells, such as CD161, CD229/SLAMF3, CD96, DNAM-1/CD226, Fc gamma RII/CD32, Fc gamma RII/RIII (CD32/CD16), Fc gamma RIII (CD16), IL-2 R beta, Integrin alpha 2/CD49b, KIR/CD158, NCAM-1/CD56, NKG2A/CD159a, NKG2C/CD159c, NKG2D/CD314, NKp30/NCR3, NKp44/NCR2, NKp46/NCR1, NKp80/KLRF1, Siglec-7/CD328 and TIGIT, or induced in T-cells, such as CCR7, CD2, CD3, CD4, CD8, CD28, CD45, CD96, CD229/SLAMF3, DNAM-1/CD226, CD25/AL-2 R alpha, L-Selectin/CD62L and TIGIT.

The invention comprises as a preferred aspect the introduction of an exogenous sequence encoding a CAR, or a component thereof, into HSCs, preferably under the transcriptional control of a promoter of a gene that is not expressed in HSC, more preferably a gene that is only expressed in the hematopoietic cells produced by said HSC, and even more preferably of a gene that is only expressed in T-cells or NK cells.

### Conditional CAR Expression in HSCs to Overpass the Thymus Barrier

A particular aspect of the present invention concerns the in-vivo production by the above engineered HSCs of hematopoietic immune cells, such as T-cells or NK-cells, expressing exogenous coding sequences, in particular a CAR or a component thereof.

One major bar of the production of hematopoietic CAR positive cells by engineered HSCs, for instance, is the rejection of the CAR positive cells by the immune system itself, especially by the thymus.

The blood-thymus barrier regulates exchange of substances between the circulatory system and thymus, providing a sequestered environment for immature T cells to develop. The barrier also prevents the immature T cells from contacting foreign antigens (since contact with antigens at this stage will cause the T cells to die by apoptosis).

One solution provided by the present invention is to place the sequences encoding the CAR components in the HSCs under the transcriptional control of promoters which are not significantly transcribed into the hematopoietic cells when they pass through the thymus barrier. One example of a gene that offers a conditional expression of the CAR into the hematopoietic cells with reduced or no significant transcriptional activity in the thymus is LCK (Uniprot P06239).

According to a preferred aspect of the invention the exogenous sequence encoding a CAR, or a component thereof, is introduced into the HSC under the transcriptional control of a gene that is described as being specifically expressed in T-cells or NK cells, preferably in these types of cells only.

The invention thereby provides with a method of producing HSCs comprising an exogenous coding sequences to be expressed exclusively in selected hematopoietic lineage(s), said coding sequences encoding preferably at least one component of a CAR or of an antigen in order to stimulate the immune system.

More broadly, the invention provides with a method of engineering HSCs by gene targeted insertion of an exogenous coding sequences to be selectively expressed in the hematopoietic cells produced by said HSCs. As a preferred embodiment, said hematopoietic cells produced by said engineered HSCs express said exogenous coding sequences in response to selected environmental factors or in-vivo stimuli to improve their therapeutic potential.

Combining Targeted Sequence Insertion(s) in Immune Cells with the Inactivation of Endogenous Genomic Sequences

One particular focus of the present invention is to perform gene inactivation in primary immune cells at a locus, by integrating exogenous coding sequence at said locus, the expression of which improves the therapeutic potential of said engineered cells. Examples of relevant exogenous coding sequences that can be inserted according to the invention have been presented above in connection with their positive effects on the therapeutic potential of the cells. Here below are presented the endogenous gene that are preferably targeted by gene targeted insertion and the advantages associated with their inactivation.

According to a preferred aspect of the invention, the insertion of the coding sequence has the effect of reducing or preventing the expression of genes involved into self and non-self recognition to reduce host versus graft disease (GVHD) reaction or immune rejection upon introduction of the allogeneic cells into a recipient patient. For instance, one of the sequence-specific reagents used in the method can reduce or prevent the expression of TCR in primary T-cells, such as the genes encoding TCR-alpha or TCR-beta.

As another preferred aspect, one gene editing step is to reduce or prevent the expression of the 182m protein and/or another protein involved in its regulation such as C2TA (Uniprot P33076) or in MHC recognition, such as HLA proteins. This permits the engineered immune cells to be less alloreactive when infused into patients.

By “allogeneic therapeutic use” is meant that the cells originate from a donor in view of being infused into patients having a different haplotype. Indeed, the present invention provides with an efficient method for obtaining primary cells, which can be gene edited in various gene loci involved into host-graft interaction and recognition.

Other loci may also be edited in view of improving the activity, the persistence of the therapeutic activity of the engineered primary cells as detailed here after

### Inactivation of Checkpoint Receptors and Immune Cells Inhibitory Pathways:

According to a preferred aspect of the invention, the inserted exogenous coding sequence has the effect of reducing or preventing the expression of a protein involved in immune cells inhibitory pathways, in particular those referred to in the literature as “immune checkpoint” (Pardoll, D. M. (2012) The blockade of immune checkpoints in cancer immunotherapy, Nature Reviews Cancer, 12:252-264). In the sense of the present invention, “immune cells inhibitory pathways” means any gene expression in immune cells that leads to a reduction of the cytotoxic activity of the lymphocytes towards malignant or infected cells. This can be for instance a gene involved into the expression of FOXP3, which is known to drive the activity of Tregs upon T cells (moderating T-cell activity).

“Immune checkpoints” are molecules in the immune system that either turn up a signal (co-stimulatory molecules) or turn down a signal of activation of an immune cell. As per the present invention, immune checkpoints more particularly designate surface proteins involved in the ligand-receptor interactions between T cells and antigen-presenting cells (APCs) that regulate the T cell response to antigen (which is mediated by peptide-major histocompatibility complex (MHC) molecule complexes that are recognized by the T cell receptor (TCR)). These interactions can occur at the initiation of T cell responses in lymph nodes (where the major APCs are dendritic cells) or in peripheral tissues or tumours (where effector responses are regulated). One important family of membrane-bound ligands that bind both co-stimulatory and inhibitory receptors is the B7 family. All of the B7 family members and their known ligands belong to the immunoglobulin superfamily. Many of the receptors for more recently identified B7 family members have not yet been identified. Tumour necrosis factor (TNF) family members that bind to cognate TNF receptor family molecules represent a second family of regulatory ligand-receptor pairs. These receptors predominantly deliver co-stimulatory signals when engaged by their cognate ligands. Another major category of signals that regulate the activation of T cells comes from soluble cytokines in the microenvironment. In other cases, activated T cells upregulate ligands, such as CD40L, that engage cognate receptors on APCs. A2aR, adenosine A2a receptor; B7RP1, B7-related protein 1; BTLA, B and T lymphocyte attenuator; GAL9, galectin 9; HVEM, herpesvirus entry mediator; ICOS, inducible T cell co-stimulator; IL, interleukin; KIR, killer cell immunoglobulin-like receptor; LAG3, lymphocyte activation gene 3; PD1, programmed cell death protein 1; PDL, PD1 ligand; TGFβ, transforming growth factor-β; TIM3, T cell membrane protein 3.

Examples of further endogenous genes, which expression could be reduced or suppressed to turn-up activation in the engineered immune cells according the present invention are listed in Table 3.

For instance, the inserted exogenous coding sequence(s) can have the effect of reducing or preventing the expression, by the engineered immune cell of at least one protein selected from PD1 (Uniprot Q15116), CTLA4 (Uniprot P16410), PPP2CA (Uniprot P67775), PPP2CB (Uniprot P62714), PTPN6 (Uniprot P29350), PTPN22 (Uniprot Q9Y2R2), LAG3 (Uniprot P18627), HAVCR2 (Uniprot Q8TDQ0), BTLA (Uniprot Q7Z6A9), CD160 (Uniprot 095971), TIGIT (Uniprot Q495A1), CD96 (Uniprot P40200), CRTAM (Uniprot 095727), LAIR1 (Uniprot Q6GTX8), SIGLEC7 (Uniprot Q9Y286), SIGLEC9 (Uniprot Q9Y336), CD244 (UniprotQ9BZWC), TNFRSF1B (Uniprot014763), TNFRSF10A (Uniprot000220), CASP8 (Uniprot Q14790), CASP10 (Uniprot Q92851), CASP3 (Uniprot P42574), CASP6 (Uniprot P55212), CASP7 (Uniprot P55210), FADD (Uniprot Q13158), FAS (Uniprot P25445), TGFBRII (Uniprot P37173), TGFRBRI (Uniprot Q15582), SMAD2 (Uniprot Q15796), SMAD3 (Uniprot P84022), SMAD4 (Uniprot Q13485), SMAD10 (Uniprot B7ZSB5), SKI (Uniprot P12755), SKIL (Uniprot P12757), TGIF1 (Uniprot Q15583), IL10RA (Uniprot Q13651), IL10RB (Uniprot Q08334), HMOX2 (Uniprot P30519), IL6R (Uniprot P08887), IL6ST (Uniprot P40189), EIF2AK4 (Uniprot Q9P2K8), CSK (Uniprot P41240), PAG1 (Uniprot Q9NWQ8), SIT1 (Uniprot Q9Y3P8), FOXP3 (Uniprot Q9BZS1), PRDM1 (Uniprot Q60636), BATF (Uniprot Q16520), GUCY1A2 (Uniprot P33402), GUCY1A3 (Uniprot Q02108), GUCY1B2 (Uniprot Q8BXH3) and GUCYB3 (Uniprot Q02153). The gene editing introduced in the genes encoding the above proteins is preferably combined with an inactivation of TCR in CAR T cells.

Preference is given to inactivation of PD1 and/or CTLA4, in combination with the expression of non-endogenous immunosuppressive polypeptide, such as a PD-L1 ligand and/or CTLA-4 Ig (see also peptides of Table 1 and 2).

TABLE 3
List of genes involved into immune cells inhibitory pathways

| | | Genes that can be inactivated |
|---|---|---|
| Pathway | | In the pathway |
| Co-inhibitory | CTLA4 (CD152) | CTLA4, PPP2CA, PPP2CB, |
| receptors | | PTPN6, PTPN22 |
| | PDCD1 (PD-1, CD279) | PDCD1 |
| | CD223 (lag3) | LAG3 |
| | HAVCR2 (tim3) | HAVCR2 |
| | BTLA(cd272) | BTLA |
| | CD160(by55) | CD160 |
| | IgSF family | TIGIT |
| | | CD96 |
| | | CRTAM |
| | LAIR1(cd305) | LAIR1 |
| | SIGLECs | SIGLEC7 |
| | | SIGLEC9 |
| | CD244(2b4) | CD244 |
| Death receptors | TRAIL | TNFRSF10B, TNFRSF10A, CASP8, |
| | | CASP10, CASP3, CASP6, CASP7 |
| | FAS | FADD, FAS |
| Cytokine signalling | TGF-beta signaling | TGFBRII, TGFBRI, SMAD2, SMAD3, |
| | | SMAD4, SMAD10, SKI, SKIL, TGIF1 |
| | IL10 signalling | IL10RA, IL10RB, HMOX2 |
| | IL6 signalling | IL6R, IL6ST |
| Prevention of TCR | | CSK, PAG1 |
| signalling | | SIT1 |
| Induced Treg | induced Treg | FOXP3 |
| Transcription | transcription factors | PRDM1 |
| factors controlling | controlling exhaustion | BATF |
| exhaustion | | |
| Hypoxia mediated | iNOS induced guanylated | GUCY1A2, GUCY1A3, GUCY1B2, |
| tolerance | cyclase | GUCY1B3 |

### Inhibiting Suppressive Cytokines/Metabolites

According to another aspect of the invention, the inserted exogenous coding sequence has the effect of reducing or preventing the expression of genes encoding or positively regulating suppressive cytokines or metabolites or receptors thereof, in particular TGFbeta (Uniprot:P01137), TGFbR (UniprotP37173), IL10 (Uniprot:P22301), IL10R (Uniprot: Q13651 and/or Q08334), A2aR (Uniprot: P29274), GCN2 (Uniprot: P15442) and PRDM1 (Uniprot: 075626).

Preference is given to engineered immune cells in which a sequence encoding IL-2, IL-12 or IL-15 replaces the sequence of at least one of the above endogenous genes.

### Inducing Resistance to Chemotherapy Drugs

According to another aspect of the present method, the inserted exogenous coding sequence has the effect of reducing or preventing the expression of a gene responsible for the sensitivity of the immune cells to compounds used in standard of care treatments for cancer or infection, such as drugs purine nucleotide analogs (PNA) or 6-Mercaptopurine (6MP) and 6 thio-guanine (6TG) commonly used in chemotherapy. Reducing or inactivating the genes involved into the mode of action of such compounds (referred to as “drug sensitizing genes”) improves the resistance of the immune cells to same.

Examples of drug sensitizing gene are those encoding DCK (Uniprot P27707) with respect to the activity of PNA, such a clorofarabine et fludarabine, HPRT (Uniprot P00492) with respect to the activity of purine antimetabolites such as 6MP and 6TG, and GGH (Uniprot Q92820) with respect to the activity of antifolate drugs, in particular methotrexate.

This enables the cells to be used after or in combination with conventional anti-cancer chemotherapies.

### Resistance to Immune-Suppressive Treatments

According to another aspect of the present invention, the inserted exogenous coding sequence has the effect of reducing or preventing the expression of receptors or proteins, which are drug targets, making said cells resistant to immune-depletion drug treatments. Such target can be glucocorticoids receptors or antigens, to make the engineered immune cells resistant to glucocorticoids or immune depletion treatments using antibodies such as Alemtuzumab, which is used to deplete CD52 positive immune cells in many cancer treatments.

Also the method of the invention can comprise gene targeted insertion in endogenous gene(s) encoding or regulating the expression of CD52 (Uniprot P31358) and/or GR (Glucocorticoids receptor also referred to as NR3C1—Uniprot P04150).

### Improving CAR Positive Immune Cells Activity and Survival

According to another aspect of the present invention, the inserted exogenous coding sequence can have the effect of reducing or preventing the expression of a surface antigen, such as BCMA, CS1 and CD38, wherein such antigen is one targeted by a CAR expressed by said immune cells.

This embodiment can solve the problem of CAR targeting antigens that are present at the surface of infected or malignant cells, but also to some extent expressed by the immune cell itself.

According to a preferred embodiment the exogenous sequence encoding the CAR or one of its constituents is integrated into the gene encoding the antigen targeted by said CAR to avoid self-destruction of the immune cells.

Engineered Immune Cells and Populations of Immune Cells

The present invention is also drawn to the variety of engineered immune cells obtainable according to one of the method described previously under isolated form or as part of populations of cells.

According to a preferred aspect of the invention the engineered cells are primary immune cells, such as NK cells or T-cells, which are generally part of populations of cells that may involve different types of cells. In general, population deriving from patients or donors isolated by leukapheresis from PBMC (peripheral blood mononuclear cells).

According to a preferred aspect of the invention, more than 50% of the immune cells comprised in said population are TCR negative T-cells. According to a more preferred aspect of the invention, more than 50% of the immune cells comprised in said population are CAR positive T-cells.

The present invention encompasses immune cells comprising any combinations of the different exogenous coding sequences and gene inactivation, which have been respectively and independently described above. Among these combinations are particularly preferred those combining the expression of a CAR under the transcriptional control of an endogenous promoter that is steadily active during immune cell activation and preferably independently from said activation, and the expression of an exogenous sequence encoding a cytokine, such as IL-2, IL-12 or IL-15, under the transcriptional control of a promoter that is up-regulated during the immune cell activation.

Another preferred combination is the insertion of an exogenous sequence encoding a CAR or one of its constituents under the transcription control of the hypoxia-inducible factor 1 gene promoter (Uniprot: Q16665).

The invention is also drawn to a pharmaceutical composition comprising an engineered primary immune cell or immune cell population as previously described for the treatment of infection or cancer, and to a method for treating a patient in need thereof, wherein said method comprises:
preparing a population of engineered primary immune cells according to the method of the invention as previously described;
optionally, purifying or sorting said engineered primary immune cells;
activating said population of engineered primary immune cells upon or after infusion of said cells into said patient.

Activation and Expansion of T Cells

Whether prior to or after genetic modification, the immune cells according to the present invention can be activated or expanded, even if they can activate or proliferate independently of antigen binding mechanisms. T-cells, in particular, can be activated and expanded using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo. T cells are generally expanded by contact with an agent that stimulates a CD3 TCR complex and a co-stimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, -10, -2, IL-15, TGFp, and TNF- or any other additives for the growth of cells known to the skilled artisan. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02). T cells that have been exposed to varied stimulation times may exhibit different characteristics

In another particular embodiment, said cells can be expanded by co-culturing with tissue or cells. Said cells can also be expanded in vivo, for example in the subject's blood after administrating said cell into the subject.

Therapeutic Compositions and Applications

The method of the present invention described above allows producing engineered primary immune cells within a limited time frame of about 15 to 30 days, preferably between 15 and 20 days, and most preferably between 18 and 20 days so that they keep their full immune therapeutic potential, especially with respect to their cytotoxic activity.

These cells form a population of cells, which preferably originate from a single donor or patient. These populations of cells can be expanded under closed culture recipients to comply with highest manufacturing practices requirements and can be frozen prior to infusion into a patient, thereby providing “off the shelf” or “ready to use” therapeutic compositions.

As per the present invention, a significant number of cells originating from the same Leukapheresis can be obtained, which is critical to obtain sufficient doses for treating a patient. Although variations between populations of cells originating from various donors may be observed, the number of immune cells procured by a leukapheresis is generally about from 10⁸ to 10¹⁰ cells of PBMC. PBMC comprises several types of cells: granulocytes, monocytes and lymphocytes, among which from 30 to 60% of T-cells, which generally represents between 10⁸ to 10⁹ of primary T-cells from one donor. The method of the present invention generally ends up with a population of engineered cells that reaches generally more than about 10⁸ T-cells, more generally more than about 10⁹ T-cells, even more generally more than about 10¹⁰ T-cells, and usually more than 10¹¹ T-cells.

The invention is thus more particularly drawn to a therapeutically effective population of primary immune cells, wherein at least 30%, preferably 50%, more preferably 80% of the cells in said population have been modified according to any one the methods described herein. Said therapeutically effective population of primary immune cells, as per the present invention, comprises immune cells that have integrated at least one exogenous genetic sequence under the transcriptional control of an endogenous promoter from at least one of the genes listed in Table 6.

Such compositions or populations of cells can therefore be used as medicaments; especially for treating cancer, particularly for the treatment of lymphoma, but also for solid tumors such as melanomas, neuroblastomas, gliomas or carcinomas such as lung, breast, colon, prostate or ovary tumors in a patient in need thereof.

The invention is more particularly drawn to populations of primary TCR negative T-cells originating from a single donor, wherein at least 20%, preferably 30%, more preferably 50% of the cells in said population have been modified using sequence-specific reagents in at least two, preferably three different loci.

In another aspect, the present invention relies on methods for treating patients in need thereof, said method comprising at least one of the following steps:
(a) Determining specific antigen markers present at the surface of patients tumors biopsies;
(b) providing a population of engineered primary immune cells engineered by one of the methods of the present invention previously described expressing a CAR directed against said specific antigen markers;
(c) Administrating said engineered population of engineered primary immune cells to said patient,

Generally, said populations of cells mainly comprises CD4 and CD8 positive immune cells, such as T-cells, which can undergo robust in vivo T cell expansion and can persist for an extended amount of time in-vitro and in-vivo.

The treatments involving the engineered primary immune cells according to the present invention can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

In another embodiment, said isolated cell according to the invention or cell line derived from said isolated cell can be used for the treatment of liquid tumors, and preferably of T-cell acute lymphoblastic leukemia.

Adult tumors/cancers and pediatric tumors/cancers are also included.

The treatment with the engineered immune cells according to the invention may be in combination with one or more therapies against cancer selected from the group of antibodies therapy, chemotherapy, cytokines therapy, dendritic cell therapy, gene therapy, hormone therapy, laser light therapy and radiation therapy.

According to a preferred embodiment of the invention, said treatment can be administrated into patients undergoing an immunosuppressive treatment. Indeed, the present invention preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the invention within the patient.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁸ cells/kg body weight including all integer values of cell numbers within those ranges. The present invention thus can provide more than 10, generally more than 50, more generally more than 100 and usually more than 1000 doses comprising between 10⁸ to 10⁸ gene edited cells originating from a single donor's or patient's sampling.

The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or nataliziimab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Henderson, Naya et al. 1991; Liu, Albers et al. 1992; Bierer, Hollander et al. 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

When CARs are expressed in the immune cells or populations of immune cells according to the present invention, the preferred CARs are those targeting at least one antigen selected from CD22, CD38, CD123, CS1, HSP70, ROR1, GD3, and CLL1.

The engineered immune cells according to the present invention endowed with a CAR or a modified TCR targeting CD22 are preferably used for treating leukemia, such as acute lymphoblastic leukemia (ALL), those with a CAR or a modified TCR targeting CD38 are preferably used for treating leukemia such as T-cell acute lymphoblastic leukemia (T-ALL) or multiple myeloma (MM), those with a CAR or a modified TCR targeting CD123 are preferably used for treating leukemia, such as acute myeloid leukemia (AML), and blastic plasmacytoid dendritic cells neoplasm (BPDCN), those with a CAR or a modified TCR targeting CS1 are preferably used for treating multiple myeloma (MM).

The present invention also encompasses means for detecting the engineered cells of the present invention comprising the desired genetic insertions, especially by carrying out steps of using PCR methods for detecting insertions of exogenous coding sequences at the endogenous loci referred to in the present specification, especially at the PD1, CD25, CD69 and TCR loci, by using probes or primers hybridizing any sequences represented by SEQ ID NO:36 to 40.

Immunological assays may also be performed for detecting the expression by the engineered cells of CARs, GP130, and to check absence or reduction of the expression of TCR, PD1, IL-6 or IL-8 in the cells where such genes have been knocked-out or their expression reduced.

### Other Definitions

Amino acid residues in a polypeptide sequence are designated herein according to the one-letter code, in which, for example, Q means Gln or Glutamine residue, R means Arg or Arginine residue and D means Asp or Aspartic acid residue.

Amino acid substitution means the replacement of one amino acid residue with another, for instance the replacement of an Arginine residue with a Glutamine residue in a peptide sequence is an amino acid substitution.

Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.

“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

The term “endonuclease” refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites”. Endonucleases can be classified as rare-cutting endonucleases when having typically a polynucleotide recognition site greater than 10 base pairs (bp) in length, more preferably of 14-55 bp. Rare-cutting endonucleases significantly increase homologous recombination by inducing DNA double-strand breaks (DSBs) at a defined locus thereby allowing gene repair or gene insertion therapies (Pingoud, A. and G. H. Silva (2007). Precision genome surgery. Nat. Biotechnol. 25(7): 743-4).

By “DNA target”, “DNA target sequence”, “target DNA sequence”, “nucleic acid target sequence”, “target sequence”, or “processing site” is intended a polynucleotide sequence that can be targeted and processed by a rare-cutting endonuclease according to the present invention. These terms refer to a specific DNA location, preferably a genomic location in a cell, but also a portion of genetic material that can exist independently to the main body of genetic material such as plasmids, episomes, virus, transposons or in organelles such as mitochondria as non-limiting example. As non-limiting examples of RNA guided target sequences, are those genome sequences that can hybridize the guide RNA which directs the RNA guided endonuclease to a desired locus.

By “mutation” is intended the substitution, deletion, insertion of up to one, two, three, four, five, six, seven, eight, nine, ten, eleven, twelve, thirteen, fourteen, fifteen, twenty, twenty five, thirty, forty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.

By “vector” is meant a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e. g. adenoassociated viruses (AAV), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picomavirus and alphavirus, and double-stranded DNA viruses including adenovirus, herpesvirus (e. g., Herpes Simplex virus types 1 and 2, Epstein-Barr virus, cytomegalovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

As used herein, the term “locus” is the specific physical location of a DNA sequence (e.g. of a gene) into a genome. The term “locus” can refer to the specific physical location of a rare-cutting endonuclease target sequence on a chromosome or on an infection agent's genome sequence. Such a locus can comprise a target sequence that is recognized and/or cleaved by a sequence-specific endonuclease according to the invention. It is understood that the locus of interest of the present invention can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.

The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA/RNA hybrid cleavage can result in the production of either blunt ends or staggered ends.

“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.

The term “subject” or “patient” as used herein includes all members of the animal kingdom including non-human primates and humans.

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

Having generally described this invention, a further understanding can be obtained by reference to certain specific examples, which are provided herein for purposes of illustration only, and are not intended to limit the scope of the claimed invention.

### EXAMPLES

### Example 1: AAV Driven Homologous Recombination in Human Primary T-Cells at Various Loci Under Control of Endogenous Promoters with Knock-Out of the Endogenous Gene

Introduction

Sequence specific endonuclease reagents, such as TALEN® (Cellectis, 8 rue de la Croix Jarry, 75013 PARIS) enable the site-specific induction of double-stranded breaks (DSBs) in the genome at desired loci. Repair of DSBs by cellular enzymes occurs mainly through two pathways: non-homologous end joining (NHEJ) and homology directed repair (HDR). HDR uses a homologous piece of DNA (template DNA) to repair the DSB by recombination and can be used to introduce any genetic sequence comprised in the template DNA. As shown therein, said template DNA can be delivered by recombinant adeno-associated virus (rAAV) along with an engineered nuclease such as TALEN® to introduce a site-specific DSB.

Design of the Integration Matrices

1.1. Insertion of an Apoptosis CAR in an Upregulated Locus with Knock-Out of the Endogenous PD1 Gene Coding Sequence

The location of the TALEN target site has been designed to be located in the targeted endogenous PDCD1 gene (Programmed cell death protein 1 referred to as PD1—Uniprot #Q15116). The sequence encompassing 1000 bp upstream and downstream the TALEN targets is given in SEQ ID NO:1 and SEQ ID NO:2. Target sequences of the TALEN (SEQ ID NO:3 and SEQ ID NO:4) is given in SEQ ID NO:5. The integration matrix is designed to be composed of a sequence (300 bp) homologous to the endogenous gene upstream of the TALEN site (SEQ ID NO:1), followed by a 2A regulatory element (SEQ ID NO:6), followed by a sequence encoding an apoptosis inducing CAR without the start codon (SEQ ID NO:7), followed by a STOP codon (TAG), followed by a polyadenylation sequence (SEQ ID NO:8), followed by a sequence (1000 bp) homologous to the endogenous gene downstream of the TALEN site (SEQ ID NO:2). The insertion matrix is subsequently cloned into a promoterless rAAV vector and used to produce AAV6.

1.2 Insertion of an Interleukin in an Upregulated Locus with Knock-Out of the Endogenous Gene

The location of the TALEN target site is designed to be located in the targeted endogenous PDCD1 gene (Programmed cell death protein 1, PD1). The sequence encompassing 1000 bp upstream and downstream the TALEN targets is given in SEQ ID NO:1 and SEQ ID NO:2. Target sequences of the TALEN (SEQ ID NO:3 and SEQ ID NO:4) is given in SEQ ID NO:5. The integration matrix is designed to be composed of a sequence (300 bp) homologous to the endogenous gene upstream of the TALEN site (SEQ ID NO:1), followed by a 2A regulatory element (SEQ ID NO:6), followed by a sequence encoding an engineered single-chained human IL-12 p35 (SEQ ID NO:9) and p40 (SEQ ID NO:10) subunit fusion protein, followed by a STOP codon (TAG), followed by a polyadenylation sequence (SEQ ID NO:8), followed by a sequence (1000 bp) homologous to the endogenous gene downstream of the TALEN site (SEQ ID NO:2). The insertion matrix is subsequently cloned into a promoterless rAAV vector and used to produce AAV6.

1.3 Insertion of an Apoptosis CAR in a Weakly Expressed Locus without Knocking Out the Endogenous Gene—N-Terminal Insertion

The location of the TALEN target site is designed to be located as close as possible to the start codon of the targeted endogenous LCK gene (LCK, LCK proto-oncogene, Src family tyrosine kinase [Homo sapiens (human)]). The sequence encompassing 1000 bp upstream and downstream the start codon is given in SEQ ID NO:11 and SEQ ID NO:12. The integration matrix is designed to be composed of a sequence (1000 bp) homologous to the endogenous gene upstream of the start codon, followed by a sequence encoding an apoptosis inducing CAR containing a start codon (SEQ ID NO:13), followed by a 2A regulatory element (SEQ ID NO:8), followed by a sequence (1000 bp) homologous to the endogenous gene downstream of the start codon (SEQ ID NO:12). The insertion matrix is subsequently cloned into a promoterless rAAV vector and used to produce AAV6.

1.4 Insertion of an Apoptosis CAR in a Weakly Expressed Locus without Knocking Out the Endogenous Gene—C-Terminal Insertion

The location of the TALEN target site is designed to be located as close as possible to the stop codon of the targeted endogenous LCK gene (LCK, LCK proto-oncogene, Src family tyrosine kinase [Homo sapiens (human)]). The sequence encompassing 1000 bp upstream and downstream the stop codon is given in SEQ ID NO:14 and SEQ ID NO:15. The integration matrix is designed to be composed of a sequence (1000 bp) homologous to the endogenous gene upstream of the stop codon, followed by a 2A regulatory element (SEQ ID NO:8), followed by a sequence encoding an apoptosis inducing CAR without the start codon (SEQ ID NO:7), followed by a STOP codon (TAG), followed by a sequence (1000 bp) homologous to the endogenous gene downstream of the stop codon (SEQ ID NO:15). The insertion matrix is subsequently cloned into a promoterless rAAV vector and used to produce AAV6.

Expression of the Sequence-Specific Nuclease Reagents in the Transduced Cells

TALEN® mRNA is synthesized using the mMessage mMachine T7 Ultra kit (Thermo Fisher Scientific, Grand Island, NY) as each TALEN is cloned downstream of a T7 promoter, purified using RNeasy columns (Qiagen, Valencia, CA) and eluted in “cytoporation medium T” (Harvard Apparatus, Holliston, MA). Human T-cells are collected and activated from whole peripheral blood provided by ALLCELLS (Alameda, CA) in X-Vivo-15 medium (Lonza, Basel, Switzerland) supplemented with 20 ng/ml human IL-2 (Miltenyi Biotech, San Diego, CA), 5% human AB serum (Gemini Bio-Products, West San Francisco, CA) and Dynabeads Human T-activator CD3/CD28 at a 1:1 bead:cell ratio (Thermo Fisher Scientific, Grand Island, NY). Beads are removed after 3 days and 5×10⁶ cells are electroporated with 10 μg mRNA of each of the two adequate TALEN® using Cytopulse (BTX Harvard Apparatus, Holliston, MA) by applying two 0.1 mS pulses at 3,000 V/cm followed by four 0.2 mS pulses at 325 V/cm in 0.4 cm gap cuvettes in a final volume of 200 μl of “cytoporation medium T” (BTX Harvard Apparatus, Holliston, Massachusetts). Cells are immediately diluted in X-Vivo-15 media with 20 ng/mL IL-2 and incubated at 37° C. with 5% CO₂. After two hours, cells are incubated with AAV6 particles at 3×10≡viral genomes (vg) per cell (37° C., 16 hours). Cells are passaged and maintained in X-Vivo-15 medium supplemented with 5% human AB serum and 20 ng/mL IL-2 until examined by flow cytometry for expression of the respective inserted gene sequences.

TABLE 4
Sequences referred to in example 1

| Sequence | Ref. | |
|---|---|---|
| name | sequences | Polynucleotide or polypeptide sequences |
| PD1 left | SEQ ID | CCAAGCCCTGACCCTGGCAGGCATATGTTTCAGGAGGTCCTTGTCTTGGGA |
| homology | NO: 1 | GCCCAGGGTCGGGGGCCCCGTGTCTGTCCACATCCGAGTCAATGGCCCAT |
| | | CTCGTCTCTGAAGCATCTTTGCTGTGAGCTCTAGTCCCCACTGTCTTGCTGG |
| | | AAAATGTGGAGGCCCCACTGCCCACTGCCCAGGGCAGCAATGCCCATACC |
| | | ACGTGGTCCCAGCTCCGAGCTTGTCCTGAAAAGGGGGCAAAGACTGGACC |
| | | CTGAGCCTGCCAAGGGGCCACACTCCTCCCAGGGCTGGGGTCTCCATGGG |
| | | CAGCCCCCCACCCACCCAGACCAGTTACACTCCCCTGTGCCAGAGCAGTGC |
| | | AGACAGGACCAGGCCAGGATGCCCAAGGGTCAGGGGCTGGGGATGGGT |
| | | AGCCCCCAAACAGCCCTTTCTGGGGGAACTGGCCTCAACGGGGAAGGGG |
| | | GTGAAGGCTCTTAGTAGGAAATCAGGGAGACCCAAGTCAGAGCCAGGTG |
| | | CTGTGCAGAAGCTGCAGCCTCACGTAGAAGGAAGAGGCTCTGCAGTGGA |
| | | GGCCAGTGCCCATCCCCGGGTGGCAGAGGCCCCAGCAGAGACTTCTCAAT |
| | | GACATTCCAGCTGGGGTGGCCCTTCCAGAGCCCTTGCTGCCCGAGGGATG |
| | | TGAGCAGGTGGCCGGGGAGGCTTTGTGGGGCCACCCAGCCCCTTCCTCAC |
| | | CTCTCTCCATCTCTCAGACTCCCCAGACAGGCCCTGGAACCCCCCCACCTTC |
| | | TCCCCAGCCCTGCTCGTGGTGACCGAAGGGGACAACGCCACCTTCACCTGC |
| | | AGCTTCTCCAACACATCGGAGAGCTTCGTGCTAAACTGGTACCGCATGAGC |
| | | CCCAGCAACCAGACGGACAAGCTGGCCGCCTTCCCCGAGGACCGCAGCCA |
| | | GCCCGGCCAGGACTGCCGCTTCCGTGTCACACAACTGCCCAACGGGCGTG |
| | | ACTTCCACATGAGCGTGGTCAGGGCCCGGCGCAATGACAGCGGCACC |
| PD1 right | SEQ ID | GCCTGCGGGCAGAGCTCAGGGTGACAGGTGCGGCCTCGGAGGCCCCGGG |
| homology | NO: 2 | GCAGGGGTGAGCTGAGCCGGTCCTGGGGTGGGTGTCCCCTCCTGCACAG |
| | | GATCAGGAGCTCCAGGGTCGTAGGGCAGGGACCCCCCAGCTCCAGTCCAG |
| | | GGCTCTGTCCTGCACCTGGGGAATGGTGACCGGCATCTCTGTCCTCTAGCT |
| | | CTGGAAGCACCCCAGCCCCTCTAGTCTGCCCTCACCCCTGACCCTGACCCTC |
| | | CACCCTGACCCCGTCCTAACCCCTGACCTTTGTGCCCTTCCAGAGAGAAGG |
| | | GCAGAAGTGCCCACAGCCCACCCCAGCCCCTCACCCAGGCCAGCCGGCCA |
| | | GTTCCAAACCCTGGTGGTTGGTGTCGTGGGCGGCCTGCTGGGCAGCCTGG |
| | | TGCTGCTAGTCTGGGTCCTGGCCGTCATCTGCTCCCGGGCCGCACGAGGTA |
| | | ACGTCATCCCAGCCCCTCGGCCTGCCCTGCCCTAACCCTGCTGGCGGCCCT |
| | | CACTCCCGCCTCCCCTTCCTCCACCCTTCCCTCACCCCACCCCACCTCCCCCC |
| | | ATCTCCCCGCCAGGCTAAGTCCCTGATGAAGGCCCCTGGACTAAGACCCCC |
| | | CACCTAGGAGCACGGCTCAGGGTCGGCCTGGTGACCCCAAGTGTGTTTCT |
| | | CTGCAGGGACAATAGGAGCCAGGCGCACCGGCCAGCCCCTGGTGAGTCTC |
| | | ACTCTTTTCCTGCATGATCCACTGTGCCTTCCTTCCTGGGTGGGCAGAGGT |
| | | GGAAGGACAGGCTGGGACCACACGGCCTGCAGGACTCACATTCTATTATA |
| | | GCCAGGACCCCACCTCCCCAGCCCCCAGGCAGCAACCTCAATCCCTAAAGC |
| | | CATGATCTGGGGCCCCAGCCCACCTGCGGTCTCCGGGGGTGCCCGGCCCA |
| | | TGTGTGTGCCTGCCTGCGGTCTCCAGGGGTGCCTGGCCCACGCGTGTGCC |
| | | CGCCTGCGGTCTCTGGGGGTGCCCGGCCCACATATGTGCC |
| PD1_T3C-L2 | SEQ ID | ATGGGCGATCCTAAAAAGAAACGTAAGGTCATCGATATCGCCGATCTACG |
| | NO: 3 | CACGCTCGGCTACAGCCAGCAGCAACAGGAGAAGATCAAACCGAAGGTTC |
| | | GTTCGACAGTGGCGCAGCACCACGAGGCACTGGTCGGCCACGGGTTTACA |
| | | CACGCGCACATCGTTGCGTTAAGCCAACACCCGGCAGCGTTAGGGACCGT |
| | | CGCTGTCAAGTATCAGGACATGATCGCAGCGTTGCCAGAGGCGACACACG |
| | | AAGCGATCGTTGGCGTCGGCAAACAGTGGTCCGGCGCACGCGCTCTGGA |
| | | GGCCTTGCTCACGGTGGCGGGAGAGTTGAGAGGTCCACCGTTACAGTTGG |
| | | ACACAGGCCAACTTCTCAAGATTGCAAAACGTGGCGGCGTGACCGCAGTG |
| | | GAGGCAGTGCATGCATGGCGCAATGCACTGACGGGTGCCCCGCTCAACTT |
| | | GACCCCCGAGCAAGTGGTGGCTATCGCTTCCAAGCTGGGGGGAAAGCAG |
| | | GCCCTGGAGACCGTCCAGGCCCTTCTCCCAGTGCTTTGCCAGGCTCACGGA |
| | | CTGACCCCTGAACAGGTGGTGGCAATTGCCTCACACGACGGGGGCAAGCA |
| | | GGCACTGGAGACTGTCCAGCGGCTGCTGCCTGTCCTCTGCCAGGCCCACG |
| | | GACTCACTCCTGAGCAGGTCGTGGCCATTGCCAGCCACGATGGGGGCAAA |
| | | CAGGCTCTGGAGACCGTGCAGCGCCTCCTCCCAGTGCTGTGCCAGGCTCAT |
| | | GGGCTGACCCCACAGCAGGTCGTCGCCATTGCCAGTAACGGCGGGGGGA |
| | | AGCAGGCCCTCGAAACAGTGCAGAGGCTGCTGCCCGTCTTGTGCCAAGCA |
| | | CACGGCCTGACACCCGAGCAGGTGGTGGCCATCGCCTCTCATGACGGCGG |
| | | CAAGCAGGCCCTTGAGACAGTGCAGAGACTGTTGCCCGTGTTGTGTCAGG |
| | | CCCACGGGTTGACACCCCAGCAGGTGGTCGCCATCGCCAGCAATGGCGGG |
| | | GGAAAGCAGGCCCTTGAGACCGTGCAGCGGTTGCTTCCAGTGTTGTGCCA |
| | | GGCACACGGACTGACCCCTCAACAGGTGGTCGCAATCGCCAGCTACAAGG |
| | | GCGGAAAGCAGGCTCTGGAGACAGTGCAGCGCCTCCTGCCCGTGCTGTGT |
| | | CAGGCTCACGGACTGACACCACAGCAGGTGGTCGCCATCGCCAGTAACGG |
| | | GGGCGGCAAGCAGGCTTTGGAGACCGTCCAGAGACTCCTCCCCGTCCTTT |
| | | GCCAGGCCCACGGGTTGACACCTCAGCAGGTCGTCGCCATTGCCTCCAAC |
| | | AACGGGGGCAAGCAGGCCCTCGAAACTGTGCAGAGGCTGCTGCCTGTGCT |
| | | GTGCCAGGCTCATGGGCTGACACCCCAGCAGGTGGTGGCCATTGCCTCTA |
| | | ACAACGGCGGCAAACAGGCACTGGAGACCGTGCAAAGGCTGCTGCCCGT |
| | | CCTCTGCCAAGCCCACGGGCTCACTCCACAGCAGGTCGTGGCCATCGCCTC |
| | | AAACAATGGCGGGAAGCAGGCCCTGGAGACTGTGCAAAGGCTGCTCCCT |
| | | GTGCTCTGCCAGGCACACGGACTGACCCCTCAGCAGGTGGTGGCAATCGC |
| | | TTCCAACAACGGGGGAAAGCAGGCCCTCGAAACCGTGCAGCGCCTCCTCC |
| | | CAGTGCTGTGCCAGGCACATGGCCTCACACCCGAGCAAGTGGTGGCTATC |
| | | GCCAGCCACGACGGAGGGAAGCAGGCTCTGGAGACCGTGCAGAGGCTGC |
| | | TGCCTGTCCTGTGCCAGGCCCACGGGCTTACTCCAGAGCAGGTCGTCGCCA |
| | | TCGCCAGTCATGATGGGGGGAAGCAGGCCCTTGAGACAGTCCAGCGGCT |
| | | GCTGCCAGTCCTTTGCCAGGCTCACGGCTTGACTCCCGAGCAGGTCGTGGC |
| | | CATTGCCTCAAACATTGGGGGCAAACAGGCCCTGGAGACAGTGCAGGCCC |
| | | TGCTGCCCGTGTTGTGTCAGGCCCACGGCTTGACACCCCAGCAGGTGGTC |
| | | GCCATTGCCTCTAATGGCGGCGGGAGACCCGCCTTGGAGAGCATTGTTGC |
| | | CCAGTTATCTCGCCCTGATCCGGCGTTGGCCGCGTTGACCAACGACCACCT |
| | | CGTCGCCTTGGCCTGCCTCGGCGGGCGTCCTGCGCTGGATGCAGTGAAAA |
| | | AGGGATTGGGGGATCCTATCAGCCGTTCCCAGCTGGTGAAGTCCGAGCTG |
| | | GAGGAGAAGAAATCCGAGTTGAGGCACAAGCTGAAGTACGTGCCCCACG |
| | | AGTACATCGAGCTGATCGAGATCGCCCGGAACAGCACCCAGGACCGTATC |
| | | CTGGAGATGAAGGTGATGGAGTTCTTCATGAAGGTGTACGGCTACAGGG |
| | | GCAAGCACCTGGGCGGCTCCAGGAAGCCCGACGGCGCCATCTACACCGTG |
| | | GGCTCCCCCATCGACTACGGCGTGATCGTGGACACCAAGGCCTACTCCGG |
| | | CGGCTACAACCTGCCCATCGGCCAGGCCGACGAAATGCAGAGGTACGTGG |
| | | AGGAGAACCAGACCAGGAACAAGCACATCAACCCCAACGAGTGGTGGAA |
| | | GGTGTACCCCTCCAGCGTGACCGAGTTCAAGTTCCTGTTCGTGTCCGGCCA |
| | | CTTCAAGGGCAACTACAAGGCCCAGCTGACCAGGCTGAACCACATCACCA |
| | | ACTGCAACGGCGCCGTGCTGTCCGTGGAGGAGCTCCTGATCGGCGGCGA |
| | | GATGATCAAGGCCGGCACCCTGACCCTGGAGGAGGTGAGGAGGAAGTTC |
| | | AACAACGGCGAGATCAACTTCGCGGCCGACTGATAA |
| PD1T3R | SEQ ID | ATGGGCGATCCTAAAAAGAAACGTAAGGTCATCGATATCGCCGATCTACG |
| | NO: 4 | CACGCTCGGCTACAGCCAGCAGCAACAGGAGAAGATCAAACCGAAGGTTC |
| | | GTTCGACAGTGGCGCAGCACCACGAGGCACTGGTCGGCCACGGGTTTACA |
| | | CACGCGCACATCGTTGCGTTAAGCCAACACCCGGCAGCGTTAGGGACCGT |
| | | CGCTGTCAAGTATCAGGACATGATCGCAGCGTTGCCAGAGGCGACACACG |
| | | AAGCGATCGTTGGCGTCGGCAAACAGTGGTCCGGCGCACGCGCTCTGGA |
| | | GGCCTTGCTCACGGTGGCGGGAGAGTTGAGAGGTCCACCGTTACAGTTGG |
| | | ACACAGGCCAACTTCTCAAGATTGCAAAACGTGGCGGCGTGACCGCAGTG |
| | | GAGGCAGTGCATGCATGGCGCAATGCACTGACGGGTGCCCCGCTCAACTT |
| | | GACCCCCGAGCAAGTCGTCGCAATCGCCAGCCATGATGGAGGGAAGCAA |
| | | GCCCTCGAAACCGTGCAGCGGTTGCTTCCTGTGCTCTGCCAGGCCCACGGC |
| | | CTTACCCCTCAGCAGGTGGTGGCCATCGCAAGTAACGGAGGAGGAAAGCA |
| | | AGCCTTGGAGACAGTGCAGCGCCTGTTGCCCGTGCTGTGCCAGGCACACG |
| | | GCCTCACACCAGAGCAGGTCGTGGCCATTGCCTCCCATGACGGGGGGAAA |
| | | CAGGCTCTGGAGACCGTCCAGAGGCTGCTGCCCGTCCTCTGTCAAGCTCAC |
| | | GGCCTGACTCCCCAACAAGTGGTCGCCATCGCCTCTAATGGCGGCGGGAA |
| | | GCAGGCACTGGAAACAGTGCAGAGACTGCTCCCTGTGCTTTGCCAAGCTC |
| | | ATGGGTTGACCCCCCAACAGGTCGTCGCTATTGCCTCAAACGGGGGGGGC |
| | | AAGCAGGCCCTTGAGACTGTGCAGAGGCTGTTGCCAGTGCTGTGTCAGGC |
| | | TCACGGGCTCACTCCACAACAGGTGGTCGCAATTGCCAGCAACGGCGGCG |
| | | GAAAGCAAGCTCTTGAAACCGTGCAACGCCTCCTGCCCGTGCTCTGTCAGG |
| | | CTCATGGCCTGACACCACAACAAGTCGTGGCCATCGCCAGTAATAATGGC |
| | | GGGAAACAGGCTCTTGAGACCGTCCAGAGGCTGCTCCCAGTGCTCTGCCA |
| | | GGCACACGGGCTGACCCCCGAGCAGGTGGTGGCTATCGCCAGCAATATTG |
| | | GGGGCAAGCAGGCCCTGGAAACAGTCCAGGCCCTGCTGCCAGTGCTTTGC |
| | | CAGGCTCACGGGCTCACTCCCCAGCAGGTCGTGGCAATCGCCTCCAACGG |
| | | CGGAGGGAAGCAGGCTCTGGAGACCGTGCAGAGACTGCTGCCCGTCTTGT |
| | | GCCAGGCCCACGGACTCACACCTGAACAGGTCGTCGCCATTGCCTCTCACG |
| | | ATGGGGGCAAACAAGCCCTGGAGACAGTGCAGCGGCTGTTGCCTGTGTTG |
| | | TGCCAAGCCCACGGCTTGACTCCTCAACAAGTGGTCGCCATCGCCTCAAAT |
| | | GGCGGCGGAAAACAAGCTCTGGAGACAGTGCAGAGGTTGCTGCCCGTCC |
| | | TCTGCCAAGCCCACGGCCTGACTCCCCAACAGGTCGTCGCCATTGCCAGCA |
| | | ACAACGGAGGAAAGCAGGCTCTCGAAACTGTGCAGCGGCTGCTTCCTGTG |
| | | CTGTGTCAGGCTCATGGGCTGACCCCCGAGCAAGTGGTGGCTATTGCCTCT |
| | | AATGGAGGCAAGCAAGCCCTTGAGACAGTCCAGAGGCTGTTGCCAGTGCT |
| | | GTGCCAGGCCCACGGGCTCACACCCCAGCAGGTGGTCGCCATCGCCAGTA |
| | | ACAACGGGGGCAAACAGGCATTGGAAACCGTCCAGCGCCTGCTTCCAGTG |
| | | CTCTGCCAGGCACACGGACTGACACCCGAACAGGTGGTGGCCATTGCATC |
| | | CCATGATGGGGGCAAGCAGGCCCTGGAGACCGTGCAGAGACTCCTGCCA |
| | | GTGTTGTGCCAAGCTCACGGCCTCACCCCTCAGCAAGTCGTGGCCATCGCC |
| | | TCAAACGGGGGGGGCCGGCCTGCACTGGAGAGCATTGTTGCCCAGTTATC |
| | | TCGCCCTGATCCGGCGTTGGCCGCGTTGACCAACGACCACCTCGTCGCCTT |
| | | GGCCTGCCTCGGCGGGCGTCCTGCGCTGGATGCAGTGAAAAAGGGATTG |
| | | GGGGATCCTATCAGCCGTTCCCAGCTGGTGAAGTCCGAGCTGGAGGAGAA |
| | | GAAATCCGAGTTGAGGCACAAGCTGAAGTACGTGCCCCACGAGTACATCG |
| | | AGCTGATCGAGATCGCCCGGAACAGCACCCAGGACCGTATCCTGGAGATG |
| | | AAGGTGATGGAGTTCTTCATGAAGGTGTACGGCTACAGGGGCAAGCACCT |
| | | GGGCGGCTCCAGGAAGCCCGACGGCGCCATCTACACCGTGGGCTCCCCCA |
| | | TCGACTACGGCGTGATCGTGGACACCAAGGCCTACTCCGGCGGCTACAAC |
| | | CTGCCCATCGGCCAGGCCGACGAAATGCAGAGGTACGTGGAGGAGAACC |
| | | AGACCAGGAACAAGCACATCAACCCCAACGAGTGGTGGAAGGTGTACCCC |
| | | TCCAGCGTGACCGAGTTCAAGTTCCTGTTCGTGTCCGGCCACTTCAAGGGC |
| | | AACTACAAGGCCCAGCTGACCAGGCTGAACCACATCACCAACTGCAACGG |
| | | CGCCGTGCTGTCCGTGGAGGAGCTCCTGATCGGCGGCGAGATGATCAAG |
| | | GCCGGCACCCTGACCCTGGAGGAGGTGAGGAGGAAGTTCAACAACGGCG |
| | | AGATCAACTTCGCGGCCGACTGATAA |
| PD1-T3 | SEQ ID | TACCTCTGTGGGGCCATCTCCCTGGCCCCCAAGGCGCAGATCAAAGAGA |
| | NO: 5 | |
| 2A-element | SEQ ID | TCCGGTGAGGGCAGAGGAAGTCTTCTAACATGCGGTGACGTGGAGGAGA |
| | NO: 6 | ATCCGGGCCCC |
| apoptosis CAR | SEQ ID | GCTTTGCCTGTCACTGCCTTGCTGCTTCCACTTGCTCTGTTGTTGCACGCCG |
| (without | NO: 7 | CAAGACCCGAGGTCAAGCTCCAGGAAAGCGGACCAGGGCTGGTGGCCCC |
| start codon) | | TAGTCAGTCATTGAGCGTCACTTGCACCGTCAGCGGCGTGTCTCTGCCCGA |
| | | TTACGGCGTGAGCTGGATCAGACAGCCCCCAAGGAAGGGACTGGAGTGG |
| | | CTGGGCGTCATCTGGGGGAGCGAGACTACCTACTACAACAGCGCCCTGAA |
| | | GAGCAGGCTGACCATCATTAAGGACAACTCCAAGTCCCAGGTCTTTCTGAA |
| | | AATGAACAGCCTGCAGACTGATGACACTGCCATCTACTACTGCGCCAAGCA |
| | | TTACTACTACGGGGGCAGCTACGCTATGGACTACTGGGGGCAGGGGACCT |
| | | CTGTCACAGTGTCAAGTGGCGGAGGAGGCAGTGGCGGAGGGGGAAGTG |
| | | GGGGCGGCGGCAGCGACATCCAGATGACCCAGACAACATCCAGCCTCTCC |
| | | GCCTCTCTGGGCGACAGAGTGACAATCAGCTGCCGGGCCAGTCAGGACAT |
| | | CAGCAAGTATCTCAATTGGTACCAGCAGAAACCAGACGGGACAGTGAAAT |
| | | TGCTGATCTACCACACATCCAGGCTGCACTCAGGAGTCCCCAGCAGGTTTT |
| | | CCGGCTCCGGCTCCGGGACAGATTACAGTCTGACCATTTCCAACCTGGAGC |
| | | AGGAGGATATTGCCACATACTTTTGCCAGCAAGGCAACACTCTGCCCTATA |
| | | CCTTCGGCGGAGGCACAAAACTGGAGATTACTCGGTCGGATCCCGAGCCC |
| | | AAATCTCCTGACAAAACTCACACATGCCCACCGTGCCCAGCACCTCCCGTG |
| | | GCCGGCCCGTCAGTGTTCCTCTTCCCCCCAAAACCCAAGGACACCCTCATG |
| | | ATCGCCCGGACCCCTGAGGTCACATGCGTGGTGGTGGACGTGAGCCACGA |
| | | GGACCCTGAGGTCAAGTTCAACTGGTACGTGGACGGCGTGGAGGTGCAT |
| | | AATGCCAAGACAAAGCCGCGGGAGGAGCAGTACAACAGCACGTACCGTG |
| | | TGGTCAGCGTCCTCACCGTCCTGCACCAGGACTGGCTGAATGGCAAGGAG |
| | | TACAAGTGCAAGGTGTCCAACAAAGCCCTCCCAGCCCCCATCGAGAAAAC |
| | | CATCTCCAAAGCCAAAGGGCAGCCCCGAGAACCACAGGTGTACACCCTGC |
| | | CCCCATCCCGGGATGAGCTGACCAAGAACCAGGTCAGCCTGACCTGCCTG |
| | | GTCAAAGGCTTCTATCCCAGCGACATCGCCGTGGAGTGGGAGAGCAATGG |
| | | GCAACCGGAGAACAACTACAAGACCACGCCTCCCGTGCTGGACTCCGACG |
| | | GCTCCTTCTTCCTCTACAGCAAGCTCACCGTGGACAAGAGCAGGTGGCAGC |
| | | AGGGGAACGTGTTCTCATGCTCCGTGATGCATGAGGCCCTGCACAATCACT |
| | | ATACCCAGAAATCTCTGAGTCTGAGCCCAGGCAAGAAGGATATTTTGGGG |
| | | TGGCTTTGCCTTCTTCTTTTGCCAATTCCACTAATTGTTTGGGTGAAGAGAA |
| | | AGGAAGTACAGAAAACATGCAGAAAGCACAGAAAGGAAAACCAAGGTTC |
| | | TCATGAATCTCCAACCTTAAATCCTGAAACAGTGGCAATAAATTTATCTGAT |
| | | GTTGACTTGAGTAAATATATCACCACTATTGCTGGAGTCATGACACTAAGT |
| | | CAAGTTAAAGGCTTTGTTCGAAAGAATGGTGTCAATGAAGCCAAAATAGA |
| | | TGAGATCAAGAATGACAATGTCCAAGACACAGCAGAACAGAAAGTTCAAC |
| | | TGCTTCGTAATTGGCATCAACTTCATGGAAAGAAAGAAGCGTATGACACAT |
| | | TGATTGCAGATCTCAAAAAAGCCAATCTTTGTACTCTTGCAGAGAAAATTC |
| | | AGACTATCATCCTCAAGGACATTACTAGTGACTCAGAAAATTCAAACTTCA |
| | | GAAATGAAATCCAGAGCTTGGTCGAA |
| BGH polyA | SEQ ID | TCTAGAGGGCCCGTTTAAACCCGCTGATCAGCCTCGACTGTGCCTTCTAGT |
| | NO: 8 | TGCCAGCCATCTGTTGTTTGCCCCTCCCCCGTGCCTTCCTTGACCCTGGAAG |
| | | GTGCCACTCCCACTGTCCTTTCCTAATAAAATGAGGAAATTGCATCGCATT |
| | | GTCTGAGTAGGTGTCATTCTATTCTGGGGGGTGGGGTGGGGCAGGACAG |
| | | CAAGGGGGAGGATTGGGAAGACAATAGCAGGCATGCTGGGGATGCGGT |
| | | GGGCTCTATGACTAGTGGCGAATTC |
| Interleukin- | SEQ ID | MCPARSLLLVATLVLLDHLSLARNLPVATPDPGMFPCLHHSQNLLRAVSNML |
| 12 subunit | NO: 9 | QKARQTLEFYPCTSEEIDHEDITKDKTSTVEACLPLELTKNESCLNSRETSF |
| alpha | | ITNGSCLASRKTSFMMALCLSSIYEDLKMYQVEFKTMNAKLLMDPKRQIFLD |
| | | QNMLAVIDELMQALNFNSETVPQKSSLEEPDFYKTKIKLCILLHAFRIRAVT |
| | | IDRVMSYLNAS |
| Interleukin- | SEQ ID | MCHQQLVISWFSLVFLASPLVAIWELKKDVYVVELDWYPDAPGEMVVLTCDT |
| 12 subunit | NO: 10 | PEEDGITWTLDQSSEVLGSGKTLTIQVKEFGDAGQYTCHKGGEVLSHSLLLLHK |
| beta | | KEDGIWSTDILKDQKEPKNKTFLRCEAKNYSGRFTCWWLTTISTDLTFSVKSSR |
| | | GSSDPQGVTCGAATLSAERVRGDNKEYEYSVECQEDSACPAAEESLPIEVMV |
| | | DAVHKLKYENYTSSFFIRDIIKPDPPKNLQLKPLKNSRQVEVSWEYPDTWSTPH |
| | | SYFSLTFCVQVQGKSKREKKDRVFTDKTSATVICRKNASISVRAQDRYYSSSWS |
| | | EWASVPCS |
| Lck left | SEQ ID | GGGATAGGGGGTGCCTCTGTGTGTGTGTGTGAGAGTGTGTGTGTGTAGG |
| homology | NO: 11 | GTGTGTATATGTATAGGGTGTGTGTGAGTGTGTGTGTGTGAGAGAGTGTG |
| | | TGTGTGGCAGAATAGACTGCGGAGGTGGATTTCATCTTGATATGAAAGGT |
| | | CTGGAATGCATGGTACATTAAACTTTGAGGACAGCGCTTTCCAAGCACTCT |
| | | GAGGAGCAGCCCTAGAGAAGGAGGAGCTGCAGGGACTCCGGGGGCTTCA |
| | | AAGTGAGGGCCCCACTCTGCTTCAGGCAAAACAGGCACACATTTATCACTT |
| | | TATCTATGGAGTTCTGCTTGATTTCATCAGACAAAAAATTTCCACTGCTAAA |
| | | ACAGGCAAATAAACAAAAAAAAAGTTATGGCCAACAGAGTCACTGGAGG |
| | | GTTTTCTGCTGGGGAGAAGCAAGCCCGTGTTTGAAGGAACCCTGTGAGAT |
| | | GACTGTGGGCTGTGTGAGGGGAACAGCGGGGGCTTGATGGTGGACTTCG |
| | | GGAGCAGAAGCCTCTTTCTCAGCCTCCTCAGCTAGACAGGGGAATTATAAT |
| | | AGGAGGTGTGGCGTGCACACCTCTCCAGTAGGGGAGGGTCTGATAAGTC |
| | | AGGTCTCTCCCAGGCTTGGGAAAGTGTGTGTCATCTCTAGGAGGTGGTCCT |
| | | CCCAACACAGGGTACTGGCAGAGGGAGAGGGAGGGGGCAGAGGCAGGA |
| | | AGTGGGTAACTAGACTAACAAAGGTGCCTGTGGCGGTTTGCCCATCCCAG |
| | | GTGGGAGGGTGGGGCTAGGGCTCAGGGGCCGTGTGTGAATTTACTTGTA |
| | | GCCTGAGGGCTCAGAGGGAGCACCGGTTTGGAGCTGGGACCCCCTATTTT |
| | | AGCTTTTCTGTGGCTGGTGAATGGGGATCCCAGGATCTCACAATCTCAGGT |
| | | ACTTTTGGAACTTTCCAGGGCAAGGCCCCATTATATCTGATGTTGGGGGAG |
| | | CAGATCTTGGGGGAGCCCCTTCAGCCCCCTCTTCCATTCCCTCAGGGACC |
| Ick right | SEQ ID | GGCTGTGGCTGCAGCTCACACCCGGAAGATGACTGGATGGAAAACATCGA |
| homology | NO: 12 | TGTGTGTGAGAACTGCCATTATCCCATAGTCCCACTGGATGGCAAGGGCA |
| | | CGGTAAGAGGCGAGACAGGGGCCTTGGTGAGGGAGTTGGGTAGAGAAT |
| | | GCAACCCAGGAGAAAGAAATGACCAGCACTACAGGCCCTTGAAAGAATA |
| | | GAGTGGCCCTCTCCCCTGAAATACAGAAAGGAAAAGAGGCCCAGAGAGG |
| | | GGAAGGGAATCTCCTAAGATCACACAGAAAGTAGTTGGTAAACTCAGGGA |
| | | TAACATCTAACCAGGCTGGAGAGGCTGAGAGCAGAGCAGGGGGGAAGG |
| | | GGGCCAGGGTCTGACCCAATCTTCTGCTTTCTGACCCCACCCTCATCCCCCA |
| | | CTCCACAGCTGCTCATCCGAAATGGCTCTGAGGTGCGGGACCCACTGGTTA |
| | | CCTACGAAGGCTCCAATCCGCCGGCTTCCCCACTGCAAGGTGACCCCAGGC |
| | | AGCAGGGCCTGAAAGACAAGGCCTGCGGATCCCTGGCTGTTGGCTTCCAC |
| | | CTCTCCCCCACCTACTTTCTCCCCGGTCTTGCCTTCCTTGTCCCCCACCCTGT |
| | | AACTCCAGGCTTCCTGCCGATCCCAGCTCGGTTCTCCCTGATGCCCCTTGTC |
| | | TTTACAGACAACCTGGTTATCGCTCTGCACAGCTATGAGCCCTCTCACGAC |
| | | GGAGATCTGGGCTTTGAGAAGGGGGAACAGCTCCGCATCCTGGAGCAGT |
| | | GAGTCCCTCTCCACCTTGCTCTGGCGGAGTCCGTGAGGGAGCGGCGATCT |
| | | CCGCGACCCGCAGCCCTCCTGCGGCCCTTGACCAGCTCGGGGTGGCCGCC |
| | | CTTGGGACAAAATTCGAGGCTCAGTATTGCTGAGCCAGGGTTGGGGGAG |
| | | GCTGGCTTAAGGGGTGGAGGGGTCTTTGAGGGAGGGTCTCAGGTCGACG |
| | | GCTGAGCGAGCCACACTGACCCACCTCCGTGGCGCAGGAGCGGCGAGTG |
| apoptosis CAR | SEQ ID | ATGGCTTTGCCTGTCACTGCCTTGCTGCTTCCACTTGCTCTGTTGTTGCACG |
| (with start | NO: 13 | CCGCAAGACCCGAGGTCAAGCTCCAGGAAAGCGGACCAGGGCTGGTGGC |
| codon) | | CCCTAGTCAGTCATTGAGCGTCACTTGCACCGTCAGCGGCGTGTCTCTGCC |
| | | CGATTACGGCGTGAGCTGGATCAGACAGCCCCCAAGGAAGGGACTGGAG |
| | | TGGCTGGGCGTCATCTGGGGGAGCGAGACTACCTACTACAACAGCGCCCT |
| | | GAAGAGCAGGCTGACCATCATTAAGGACAACTCCAAGTCCCAGGTCTTTCT |
| | | GAAAATGAACAGCCTGCAGACTGATGACACTGCCATCTACTACTGCGCCAA |
| | | GCATTACTACTACGGGGGCAGCTACGCTATGGACTACTGGGGGCAGGGG |
| | | ACCTCTGTCACAGTGTCAAGTGGCGGAGGAGGCAGTGGCGGAGGGGGAA |
| | | GTGGGGGCGGCGGCAGCGACATCCAGATGACCCAGACAACATCCAGCCTC |
| | | TCCGCCTCTCTGGGCGACAGAGTGACAATCAGCTGCCGGGCCAGTCAGGA |
| | | CATCAGCAAGTATCTCAATTGGTACCAGCAGAAACCAGACGGGACAGTGA |
| | | AATTGCTGATCTACCACACATCCAGGCTGCACTCAGGAGTCCCCAGCAGGT |
| | | TTTCCGGCTCCGGCTCCGGGACAGATTACAGTCTGACCATTTCCAACCTGG |
| | | AGCAGGAGGATATTGCCACATACTTTTGCCAGCAAGGCAACACTCTGCCCT |
| | | ATACCTTCGGCGGAGGCACAAAACTGGAGATTACTCGGTCGGATCCCGAG |
| | | CCCAAATCTCCTGACAAAACTCACACATGCCCACCGTGCCCAGCACCTCCC |
| | | GTGGCCGGCCCGTCAGTGTTCCTCTTCCCCCCAAAACCCAAGGACACCCTC |
| | | ATGATCGCCCGGACCCCTGAGGTCACATGCGTGGTGGTGGACGTGAGCCA |
| | | CGAGGACCCTGAGGTCAAGTTCAACTGGTACGTGGACGGCGTGGAGGTG |
| | | CATAATGCCAAGACAAAGCCGCGGGAGGAGCAGTACAACAGCACGTACC |
| | | GTGTGGTCAGCGTCCTCACCGTCCTGCACCAGGACTGGCTGAATGGCAAG |
| | | GAGTACAAGTGCAAGGTGTCCAACAAAGCCCTCCCAGCCCCCATCGAGAA |
| | | AACCATCTCCAAAGCCAAAGGGCAGCCCCGAGAACCACAGGTGTACACCC |
| | | TGCCCCCATCCCGGGATGAGCTGACCAAGAACCAGGTCAGCCTGACCTGC |
| | | CTGGTCAAAGGCTTCTATCCCAGCGACATCGCCGTGGAGTGGGAGAGCAA |
| | | TGGGCAACCGGAGAACAACTACAAGACCACGCCTCCCGTGCTGGACTCCG |
| | | ACGGCTCCTTCTTCCTCTACAGCAAGCTCACCGTGGACAAGAGCAGGTGGC |
| | | AGCAGGGGAACGTGTTCTCATGCTCCGTGATGCATGAGGCCCTGCACAAT |
| | | CACTATACCCAGAAATCTCTGAGTCTGAGCCCAGGCAAGAAGGATATTTTG |
| | | GGGTGGCTTTGCCTTCTTCTTTTGCCAATTCCACTAATTGTTTGGGTGAAGA |
| | | GAAAGGAAGTACAGAAAACATGCAGAAAGCACAGAAAGGAAAACCAAGG |
| | | TTCTCATGAATCTCCAACCTTAAATCCTGAAACAGTGGCAATAAATTTATCT |
| | | GATGTTGACTTGAGTAAATATATCACCACTATTGCTGGAGTCATGACACTA |
| | | AGTCAAGTTAAAGGCTTTGTTCGAAAGAATGGTGTCAATGAAGCCAAAAT |
| | | AGATGAGATCAAGAATGACAATGTCCAAGACACAGCAGAACAGAAAGTTC |
| | | AACTGCTTCGTAATTGGCATCAACTTCATGGAAAGAAAGAAGCGTATGAC |
| | | ACATTGATTGCAGATCTCAAAAAAGCCAATCTTTGTACTCTTGCAGAGAAA |
| | | ATTCAGACTATCATCCTCAAGGACATTACTAGTGACTCAGAAAATTCAAAC |
| | | TTCAGAAATGAAATCCAGAGCTTGGTCGAA |
| Lck left | SEQ ID | CTCATAACAATTCTATGAGGTAGGAACAGTTATTTACTCTATTTTCCAAATA |
| homology | NO: 14 | AGGAAACTGGGCTCGCCCAAGGTTCCACAACTAACATGTGTGTATTATTGA |
| | | GCATTTAATTTACACCAGGGAAGCAGGTTGTGGTGGTGTGCACCTGTTGTC |
| | | CAGCTATTTAGGAGGCTGAGGTGAAAGGATCACTTGAACGGAGGAGTTCA |
| | | AATTTGCAATGTGCTATGATTGTGCCTGTGAACAGCTGCTGCACTCCAGCC |
| | | TGGGCAACATAGTGAGATCCCTTATCTAAAACATTTTTTTTAAGTAAATAAT |
| | | CAGGTGGGCACGGTGGCTCACGCCTGTAATCCAGCACTTTGGGAGGCTGA |
| | | GGCGGGCGGATCACCTGAGGTCAGGAGTTCAAGACCAGCCTGACCAACAT |
| | | GGAGAAACCCGTCTCTACTAAAAATACAAAATTAGCTTGGCGTGGTGGTG |
| | | CATGCCTGTAATCCCAGCTACTCGAGAAGCTGAGGCAGGAGAATTGTTTG |
| | | AACCTGGGAGGTGGAGGTTGCGGTGAGCCGAGATCGCACCATTGCACTCC |
| | | AGCCTGGGCAACAAGAGTGAAATTGCATCTCAAAAAAAAAGAAAAGGAA |
| | | ATAATCTATACCAGGCACTCCAAGTGGTGTGACTGATATTCAACAAGTACC |
| | | TCTAGTGTGACCTTACCATTGATGAAGACCAAGATTCTTTTGGATTGGTGC |
| | | TCACACTGTGCCAGTTAAATATTCCGAACATTACCCTTGCCTGTGGGCTTCC |
| | | AGTGCCTGACCTTGATGTCCTTTCACCCATCAACCCGTAGGGATGACCAAC |
| | | CCGGAGGTGATTCAGAACCTGGAGCGAGGCTACCGCATGGTGCGCCCTGA |
| | | CAACTGTCCAGAGGAGCTGTACCAACTCATGAGGCTGTGCTGGAAGGAGC |
| | | GCCCAGAGGACCGGCCCACCTTTGACTACCTGCGCAGTGTGCTGGAGGAC |
| | | TTCTTCACGGCCACAGAGGGCCAGTACCAGCCTCAGCCT |
| Ick right | SEQ ID | GAGGCCTTGAGAGGCCCTGGGGTTCTCCCCCTTTCTCTCCAGCCTGACTTG |
| homology | NO: 15 | GGGAGATGGAGTTCTTGTGCCATAGTCACATGGCCTATGCACATATGGAC |
| | | TCTGCACATGAATCCCACCCACATGTGACACATATGCACCTTGTGTCTGTAC |
| | | ACGTGTCCTGTAGTTGCGTGGACTCTGCACATGTCTTGTACATGTGTAGCC |
| | | TGTGCATGTATGTCTTGGACACTGTACAAGGTACCCCTTTCTGGCTCTCCCA |
| | | TTTCCTGAGACCACAGAGAGAGGGGAGAAGCCTGGGATTGACAGAAGCT |
| | | TCTGCCCACCTACTTTTCTTTCCTCAGATCATCCAGAAGTTCCTCAAGGGCC |
| | | AGGACTTTATCTAATACCTCTGTGTGCTCCTCCTTGGTGCCTGGCCTGGCAC |
| | | ACATCAGGAGTTCAATAAATGTCTGTTGATGACTGTTGTACATCTCTTTGCT |
| | | GTCCACTCTTTGTGGGTGGGCAGTGGGGGTTAAGAAAATGGTAATTAGGT |
| | | CACCCTGAGTTGGGGTGAAAGATGGGATGAGTGGATGTCTGGAGGCTCT |
| | | GCAGACCCCTTCAAATGGGACAGTGCTCCTCACCCCTCCCCAAAGGATTCA |
| | | GGGTGACTCCTACCTGGAATCCCTTAGGGAATGGGTGCGTCAAAGGACCT |
| | | TCCTCCCCATTATAAAAGGGCAACAGCATTTTTTACTGATTCAAGGGCTATA |
| | | TTTGACCTCAGATTTTGTTTTTTTAAGGCTAGTCAAATGAAGCGGCGGGAA |
| | | TGGAGGAGGAACAAATAAATCTGTAACTATCCTCAGATTTTTTTTTTTTTTT |
| | | GAGACTGGGTCTCACTTTTTCATCCAGGCTGGAGTGCAGTCGCATGATCAC |
| | | GGCTCACTGTAGCCTCAACCTCTCCAGCTCAAATGCTCCTCCTGTCTCAGCC |
| | | TCCCGAGTACCTGGGACTACTTTCTTGAGGCCAGGAATTCAAGAACAGAG |
| | | TAAGATCCTGGTCTCCAAAAAAAGTTTTAAA |

### Example 2: TALEN®-Mediated Double Targeted Integration of IL-15 and CAR Encoding Matrices in T-Cells

### Materials

X-vivo-15 was obtained for Lonza (cat #BE04-418Q), IL-2 from Miltenyi Biotech (cat #130-097-748), human serum AB from Seralab (cat #GEM-100-318), human T activator CD3/CD28 from Life Technology (cat #11132D), QBEND10-APC from R&D Systems (cat #FAB7227A), vioblue-labeled anti-CD3, PE-labeled anti-LNGFR, APC-labeled anti-CD25 and PE-labeled anti-PD1 from Miltenyi (cat #130-094-363, 130-112-790, 130-109-021 and 130-104-892 respectively) 48 wells treated plates (CytoOne, cat #CC7682-7548), human IL-15 Quantikine ELISA kit from R&D systems (cat #S1500), ONE-Glo from Promega (cat #E6110). AAV6 batches containing the different matrices were obtained from Virovek, PBMC cells were obtained from Allcells, (cat #PB004F) and Raji-Luciferase cells were obtained after Firefly Luciferase-encoding lentiviral particles transduction of Raji cells from ATCC (cat #CCL-86).

### Methods

2.1-Transfection-Transduction

The double targeted integration at TRAC and PD1 or CD25 loci were performed as follows. PBMC cells were first thawed, washed, resuspended and cultivated in X-vivo-15 complete media (X-vivo-15, 5% AB serum, 20 ng/mL IL-2). One day later, cells were activated by Dynabeads human T activator CD3/CD28 (25 uL of beads/1E⁶ CD3 positive cells) and cultivated at a density of 1E⁶ cells/mL for 3 days in X-vivo complete media at 37° C. in the presence of 5% CO₂. Cells were then split in fresh complete media and transduced/transfected the next day according to the following procedure. On the day of transduction-transfection, cells were first de-beaded by magnetic separation (EasySep), washed twice in Cytoporation buffer T (BTX Harvard Apparatus, Holliston, Massachusetts) and resuspended at a final concentration of 28E⁶ cells/mL in the same solution. Cellular suspension was mixed with 5 μg mRNA encoding TRAC TALEN® arms (SEQ ID NO:16 and 17) in the presence or in the absence of 15 μg of mRNA encoding arms of either CD25 or PD1 TALEN® (SEQ ID NO:18 and 19 and SEQ ID NO:20 and 21 respectively) in a final volume of 200 μl. TALEN® is a standard format of TALE-nucleases resulting from a fusion of TALE with Fok-1 Transfection was performed using Pulse Agile technology, by applying two 0.1 mS pulses at 3,000 V/cm followed by four 0.2 mS pulses at 325 V/cm in 0.4 cm gap cuvettes and in a final volume of 200 μl of Cytoporation buffer T (BTX Harvard Apparatus, Holliston, Massachusetts). Electroporated cells were then immediately transferred to a 12-well plate containing 1 mL of prewarm X-vivo-15 serum-free media and incubated for 37° C. for 15 min. Cells were then concentrated to 8E⁶ cells/mL in 250 μL of the same media in the presence of AAV6 particles (MOI=3E⁵ vg/cells) comprising the donor matrices in 48 wells regular treated plates. After 2 hours of culture at 30° C., 250 μL of Xvivo-15 media supplemented by 10% AB serum and 40 ng/ml IL-2 was added to the cell suspension and the mix was incubated 24 hours in the same culture conditions. One day later, cells were seeded at 1E⁶ cells/mL in complete X-vivo-15 media and cultivated at 37° C. in the presence of 5% CO₂.

2.2-Activation-Dependent Expression of ΔLNGFR and Secretion of IL15

Engineered T-cells were recovered from the transfection-transduction process described earlier and seeded at 1E⁶ cells/mL alone or in the presence of Raji cells (E:T=1:1) or Dynabeads (12.5 uL/1E⁶ cells) in 100 μL final volume of complete X-vivo-15 media. Cells were cultivated for 48 hours before being recovered, labeled and analyzed by flow cytometry. Cells were labeled with two independent sets of antibodies. The first sets of antibodies, aiming at detecting the presence of ΔLNGFR, CAR and CD3 cells, consisted in QBEND10-APC (diluted 1/10), vioblue-labeled anti CD3 (diluted 1/25) and PE-labeled anti-ΔLNGFR (diluted 1/25). The second sets of antibodies, aiming at detecting expression of endogenous CD25 and PD1, consisted in APC-labeled anti-CD25 (diluted 1/25) and vioblue-labeled anti PD1 (diluted 1/25).

The same experimental set up was used to study IL-15 secretion in the media. Cells mixture were kept in co-culture for 2, 4, 7 and 10 days before collecting and analyzing supernatant using an IL-15 specific ELISA kit.

2.3-Serial Killing Assay

To assess the antitumor activity of engineered CAR T-cells, a serial killing assay was performed. The principle of this assay is to challenge CAR T-cell antitumor activity everyday by a daily addition of a constant amount of tumor cells. Tumor cell proliferation, control and relapse could be monitored via luminescence read out thanks to a Luciferase marker stably integrated in Tumor cell lines.

Typically, CAR T-cells are mixed to a suspension of 2.5×10⁵ Raji-luc tumor cells at variable E:T ratio (E:T=5:1 or 1:1) in a total volume of 1 mL of Xvivo 5% AB, 20 ng/uL IL-2. The mixture is incubated 24 hours before determining the luminescence of 25 uL of cell suspension using ONE-Glo reagent. Cells mixture are then spun down, the old media is discarded and substituted with 1 mL of fresh complete X-vivo-15 media containing 2.5×10⁵ Raji-Luc cells and the resulting cell mixture is incubated for 24 hours. This protocol is repeated 4 days.

### EXPERIMENTS AND RESULTS

This example describes methods to improve the therapeutic outcome of CAR T-cell therapies by integrating an IL-15/soluble IL-15 receptor alpha heterodimer (IL15/sIL15rα) expression cassette under the control of the endogenous T-cell promoters regulating PD1 and CD25 genes. Because both genes are known to be upregulated upon tumor engagement by CAR T-cells, they could be hijacked to re-express IL-IL15/sIL15rα only in vicinity of a tumor. This method aims to reduce the potential side effects of IL15/sIL15rα systemic secretion while maintaining its capacity to reduced activation induced T-cell death (AICD), promote T-cell survival, enhance T-cell antitumor activity and to reverse T-cell anergy.

The method developed to integrate IL15/sIL15rα at PD1 and CD25 loci consisted in generating a double-strand break at both loci using TALEN in the presence of a DNA repair matrix vectorized by AAV6. This matrix consists of two homology arms embedding IL15/sIL15rα coding regions separated by a 2A cis acting elements and regulatory elements (stop codon and polyA sequences). Depending on the locus targeted and its involvement in T-cell activity, the targeted endogenous gene could be inactivated or not via specific matrix design. When CD25 gene was considered as targeted locus, the insertion matrix was designed to knock-in (KI) IL15/sIL15rα without inactivating CD25 because the protein product of this gene is regarded as essential for T-cell function. By contrast, because PD1 is involved in T-cell inhibition/exhaustion of T-cells, the insertion matrix was designed to prevent its expression while enabling the expression and secretion of IL15/sIL15rα.

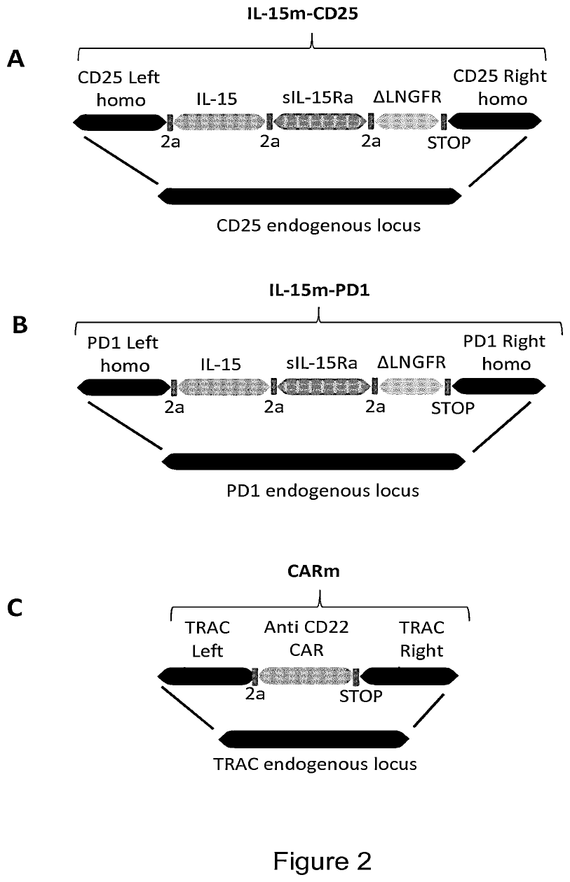

To illustrate this approach and demonstrate the feasibility of double targeted insertion in primary T-cells, three different matrices were designed (FIGS. 2A, 2B and 2C). The first one named CARm represented by SEQ ID NO:36 was designed to insert an anti-CD22 CAR cDNA at the TRAC locus in the presence of TRAC TALEN® (SEQ ID NO:16 and 17). The second one, IL-15_CD25m (SEQ ID NO:37) was designed to integrate IL15, sIL15rα and the surface marker named ΔLNGFR cDNAs separated by 2A cis-acting elements just before the stop codon of CD25 endogenous coding sequence using CD25 TALEN® (SEQ ID NO:18 and 19). The third one, IL-15_PD1m (SEQ ID NO:38), contained the same expression cassette and was designed to integrate in the middle of the PD1 open reading frame using PD1 TALEN® (SEQ ID NO:20 and 21). The three matrices contained an additional 2A cis-acting element located upstream expression cassettes to enable co-expression of IL15/sIL15rα and CAR with the endogenous gene targeted.

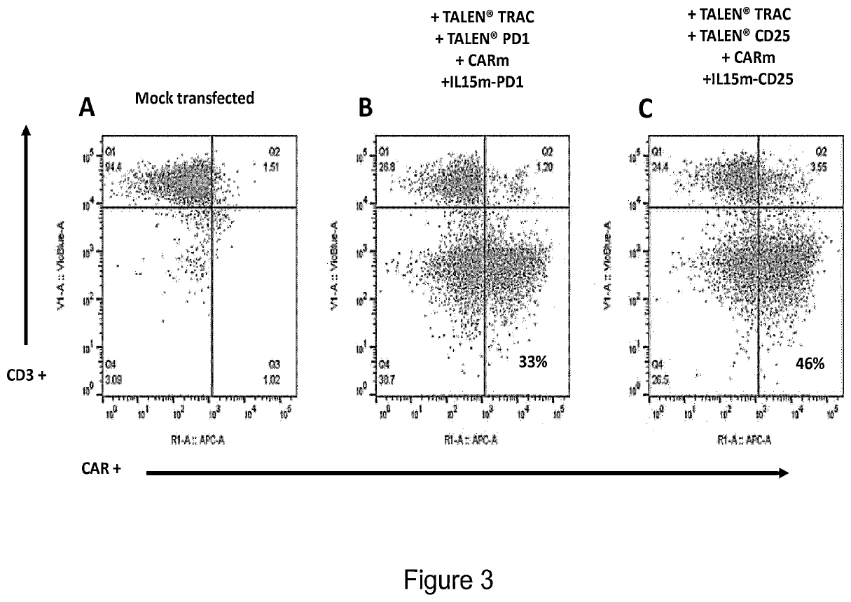

We first assessed the efficiency of double targeted insertion in T-cells by transducing them with one of the AAV6 encoding IL15/sIL15rα matrices (SEQ ID NO:41; pCLS30519) along with the one encoding the CAR and subsequently transfected the corresponding TALEN®. AAV6-assisted vectorization of matrices in the presence of mRNA encoding TRAC TALEN® (SEQ ID NO:22 and 23) and PD1 TALEN® (SEQ ID NO:24 and 25) or CD25 TALEN® (SEQ ID NO:26 and 27) enabled expression of the anti CD22 CAR in up to 46% of engineered T-cells (FIG. 3).

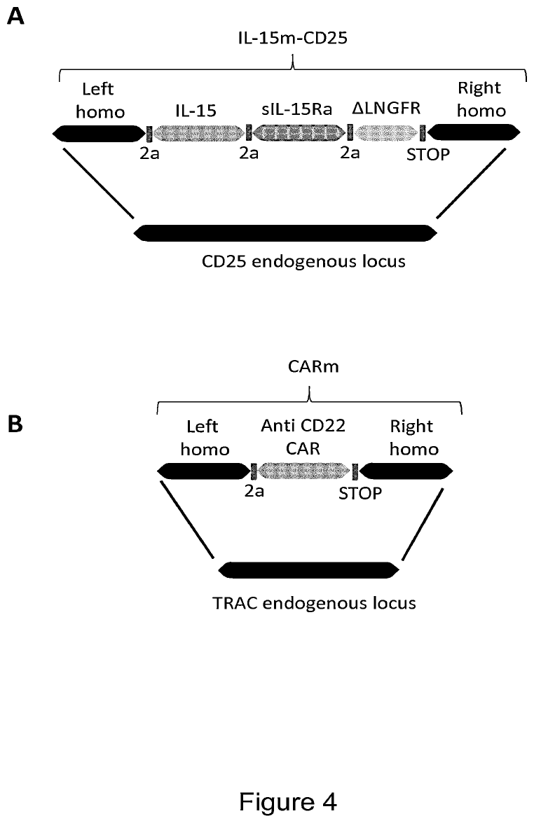

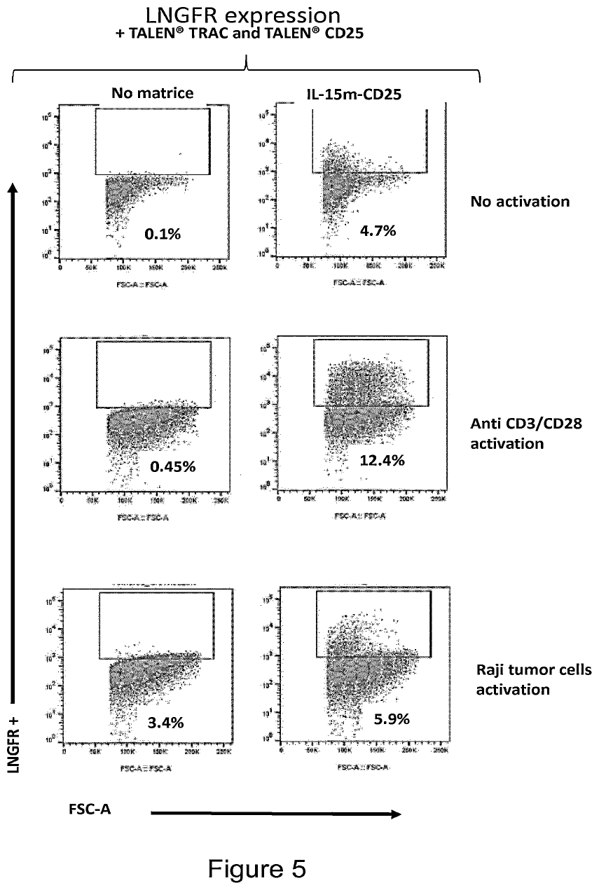

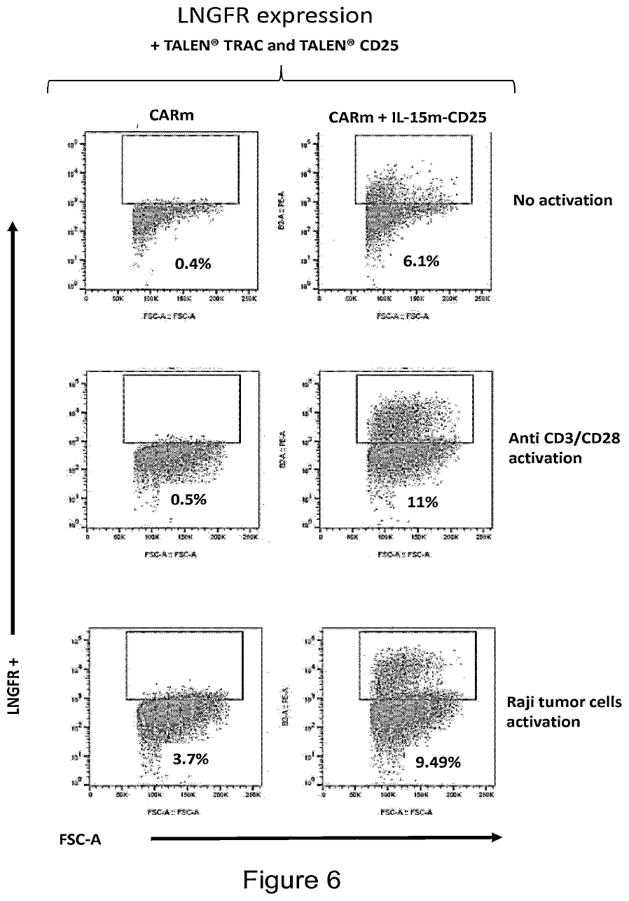

To determine the extent of IL15m integration at CD25 and PD1 locus, engineered T-cells were activated with either antiCD3/CD28 coated beads or with CD22 expressing Raji tumor cells. 2 days post activation, cells were recovered and analyzed by FACS using LNGFR expression as IL15/sIL15rα secretion surrogate (FIGS. 4 and 5). Our results showed that antiCD3/CD28 coated beads induced expression of ΔLNGFR by T-cells containing IL-15m_CD25 or IL-15m_PD1, independently of the presence of the anti CD22 CAR (FIG. 4A-B). Tumor cells however, only induced expression of ΔLNGFR by T-cell treated by both CARm and IL-15m. This indicated that expression of ΔLNGFR could be specifically induced through tumor cell engagement by the CAR (FIGS. 5 and 6).

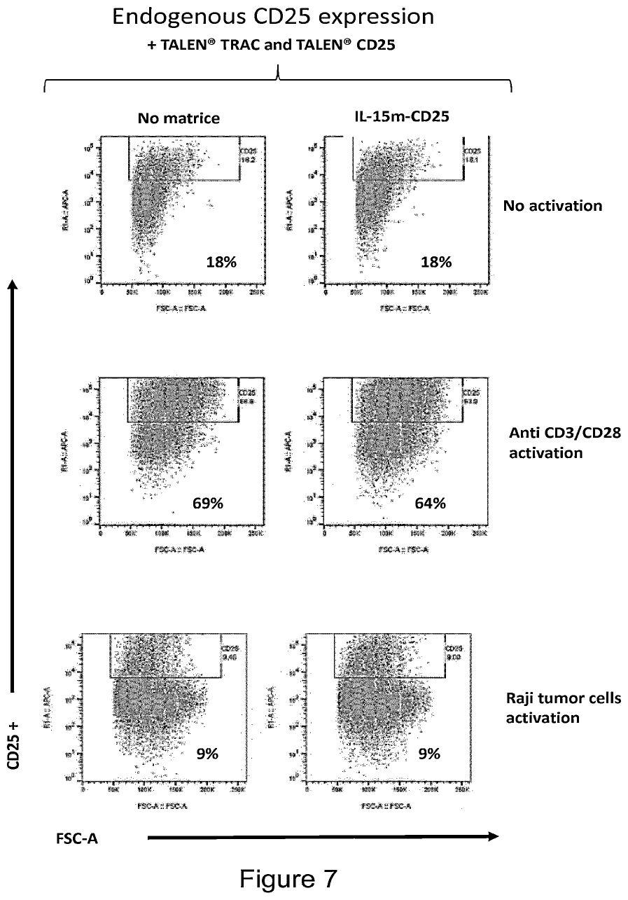

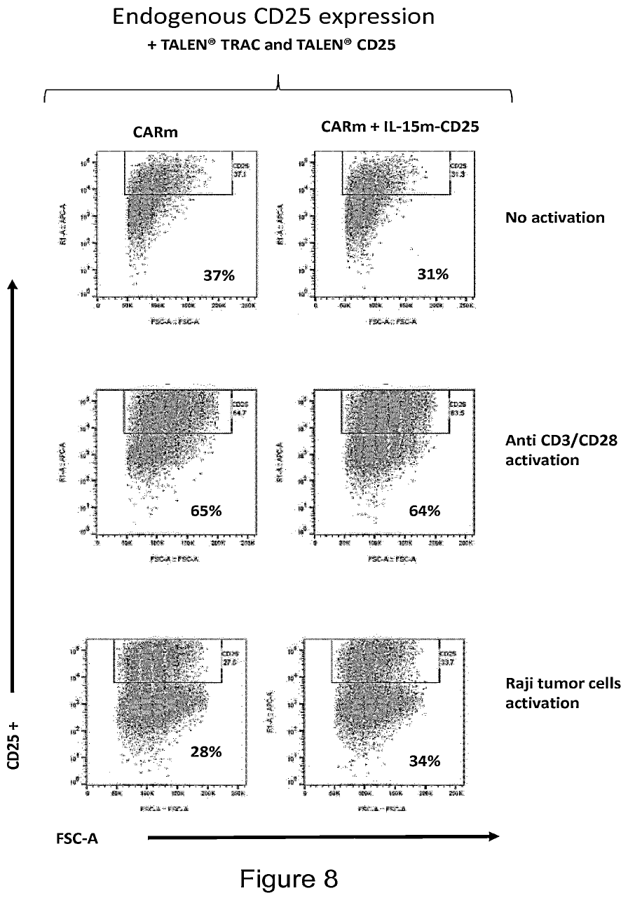

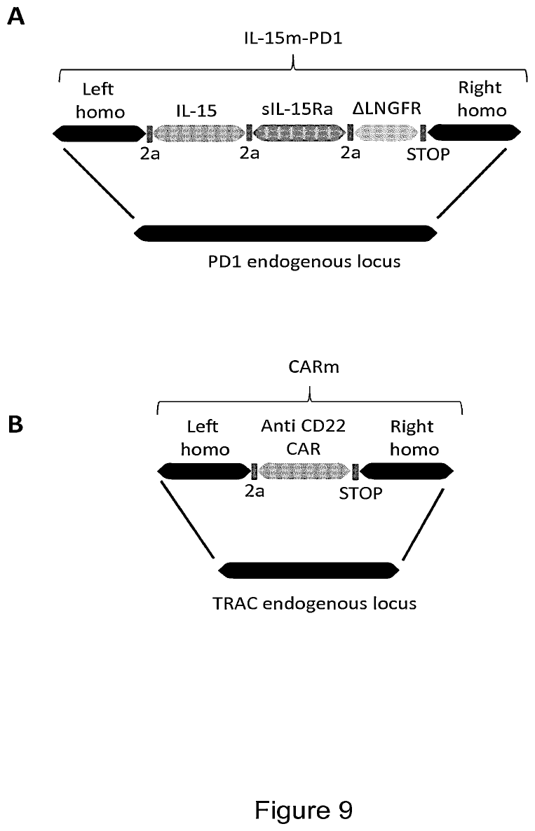

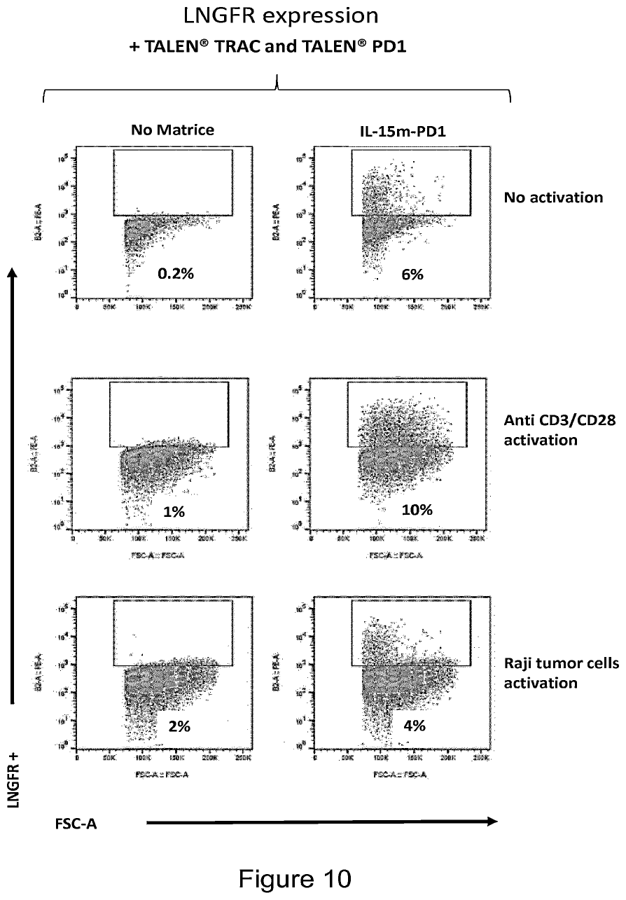

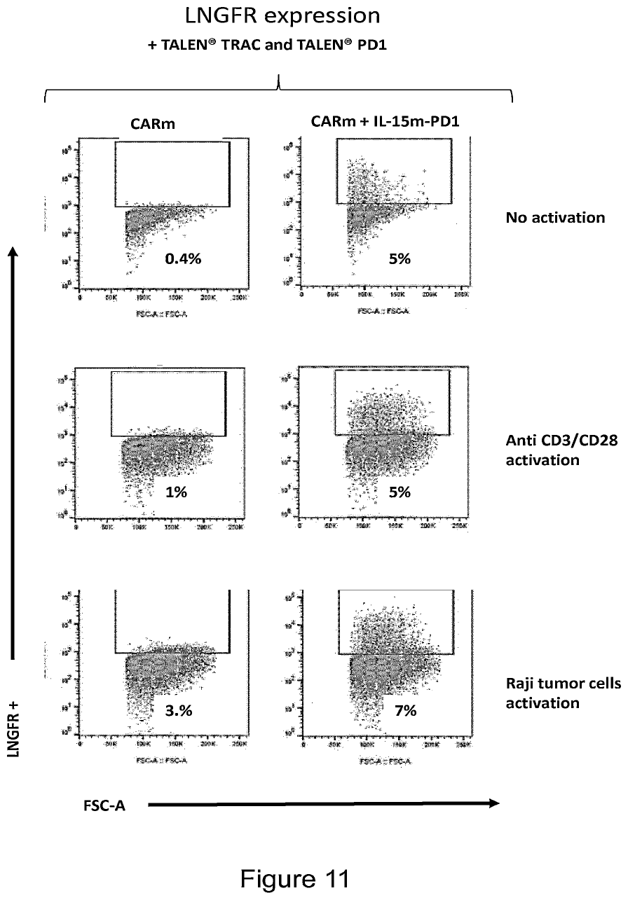

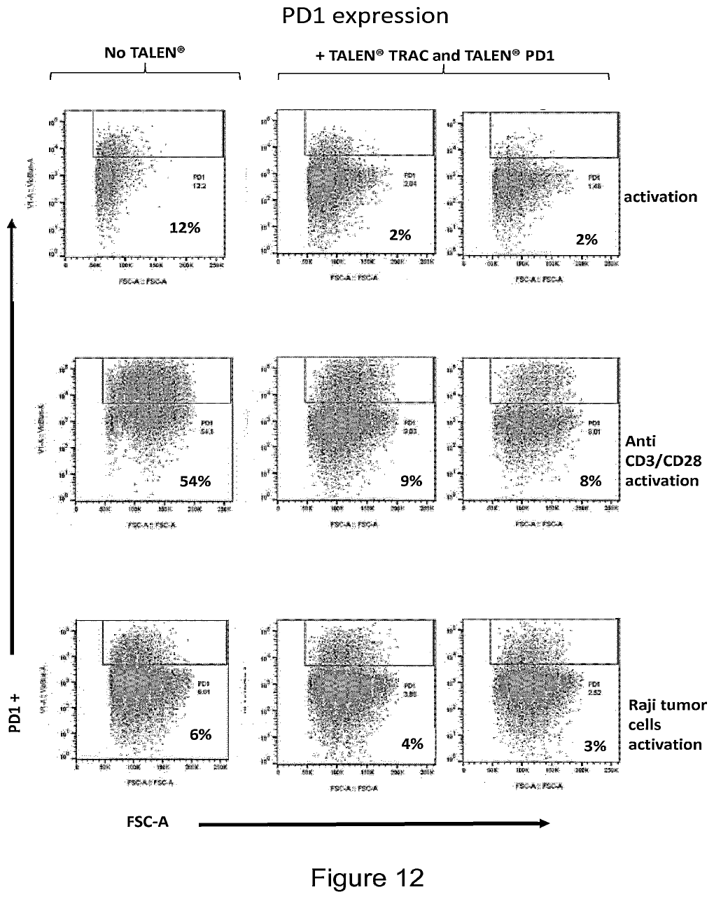

As expected the endogenous CD25 gene was still expressed in activated treated T-cells (FIGS. 7 and 8) while PD1 expression was strongly impaired (FIG. 12).

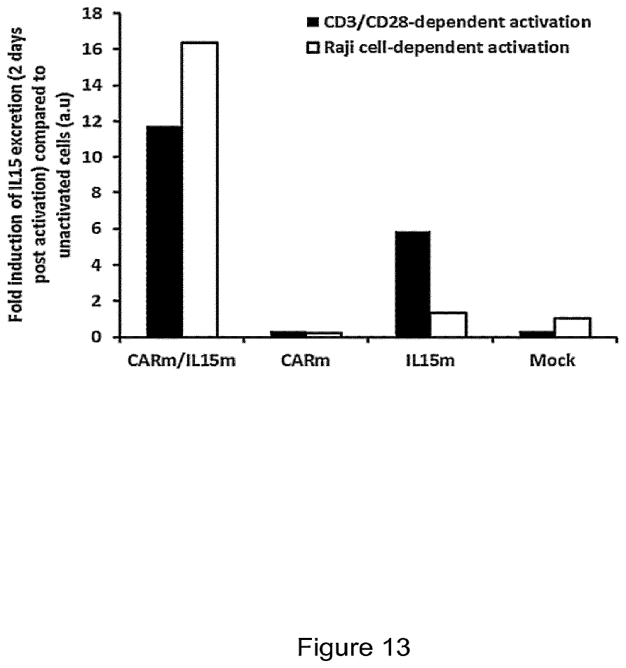

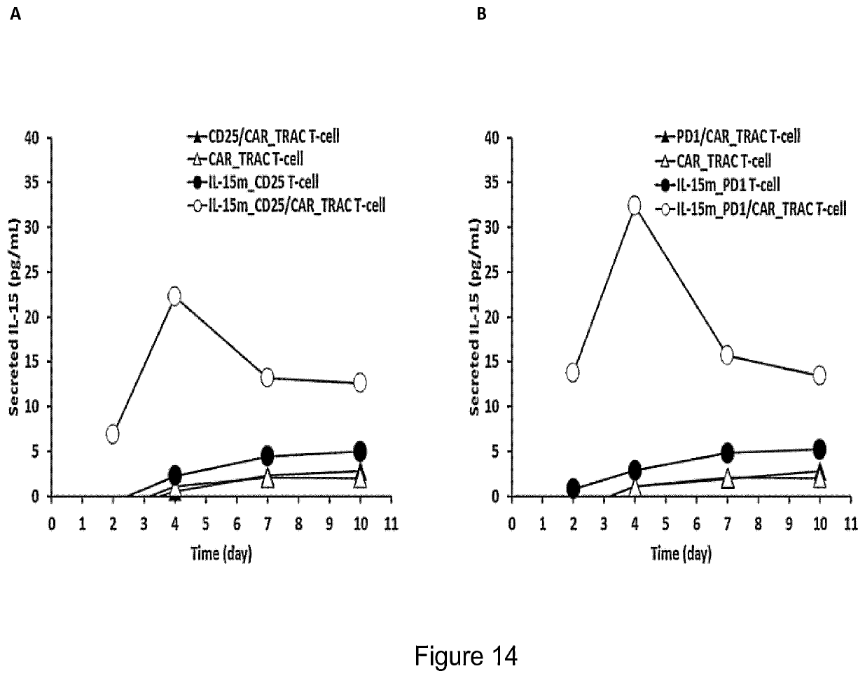

To verify that expression of ΔLNGFR correlated with secretion of IL15 in the media, T-cells expressing the anti-CD22 CAR and ΔLNGFR were incubated in the presence of CD22 expressing Raji tumor cells (E:T ratio=1:1) for a total of 10 days. Supernatant were recovered at day 2, 4, 7 and 10 and the presence of IL15 was quantified by ELISA assay. Our results showed that IL15 was secreted in the media only by T-cells that were co-treated by both CARm and IL15m matrices along with their corresponding TALEN® (FIG. 13). T-cell treated with either one of these matrices were unable to secrete any significant level of IL15 with respect to resting T-cells. Interestingly, IL-15 secretion level was found transitory, with a maximum peak centered at day 4 (FIG. 14).

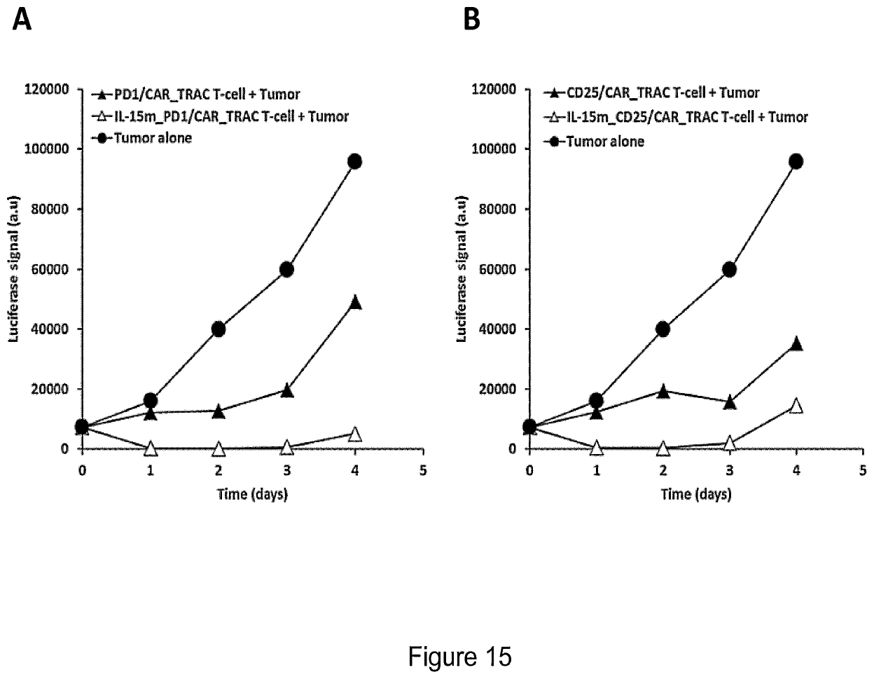

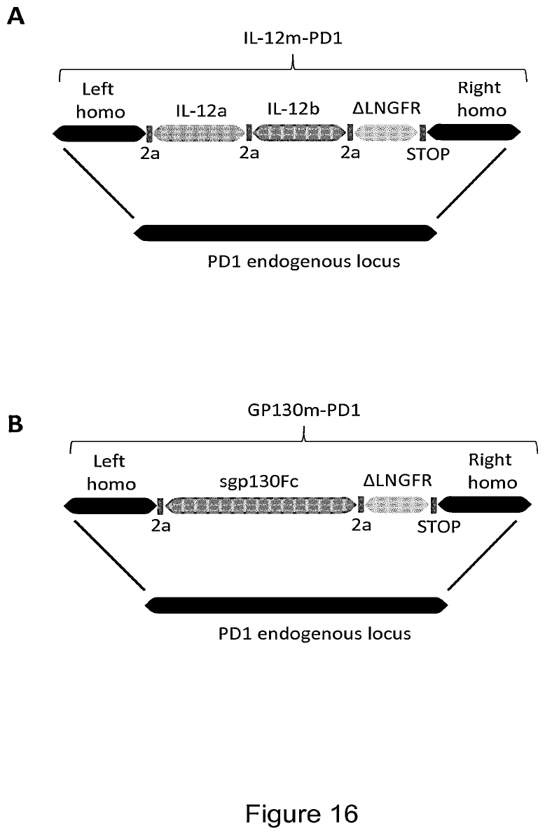

To assess whether the level of secreted IL-15 (SEQ ID NO:59) could impact CAR T-cell activity, CAR T-cell were co-cultured in the presence of tumor cells at E:T ratio of 5:1 for 4 days. Their antitumor activity was challenged everyday by pelleting and resuspended them in a culture media lacking IL-2 and containing fresh tumor cells. Antitumor activity of CAR T-cell was monitored everyday by measuring the luminescence of the remaining Raji tumor cells expressing luciferase. Our results showed that CAR T-cells co-expressing IL-15 had a higher antitumor activity than those lacking IL15 at all time points considered (FIG. 15).

Thus, together our results showed that we have developed a method allowing simultaneous targeted insertions of CAR and IL15 cDNA at TRAC and CD25 or PD1 loci. This double targeted insertion led to robust expression of an antiCD22 CAR and to the secretion of IL15 in the media. Levels of secreted IL15 were sufficient to enhance the activity of CAR T-cells.

TABLE 5
Sequences referred to in example 2.

| SEQ | | | |
|---|---|---|---|
| ID | Sequence | | |
| NO# | Name | Polypeptide sequence | RVD sequence |
| 16 | TALEN | MGDPKKKRKVIDYPYDVPDYAIDIADLRTLGYSQQQQEKIKPKVRSTVA | NG-NN-NG-HD- |
| | right TRAC | QHHEALVGHGFTHAHIVALSQHPAALGTVAVKYQDMIAALPEATHEAIV | HD-HD-NI-HD-NI- |
| | | GVGKQWSGARALEALLTVAGELRGPPLQLDTGQLLKIAKRGGVTAVEA | NN-NI-NG-NI-NG- |
| | | VHAWRNALTGAPLNLTPQQVVAIASNGGGKQALETVQRLLPVLCQAHG | HD-NG# |
| | | LTPQQVVAIASNNGGKQALETVQRLLPVLCQAHGLTPQQVVAIASNGG | |
| | | GKQALETVQRLLPVLCQAHGLTPEQVVAIASHDGGKQALETVQRLLPVL | |
| | | CQAHGLTPEQVVAIASHDGGKQALETVQRLLPVLCQAHGLTPEQVVAIA | |
| | | SHDGGKQALETVQRLLPVLCQAHGLTPEQVVAIASNIGGKQALETVQAL | |
| | | LPVLCQAHGLTPEQVVAIASHDGGKQALETVQRLLPVLCQAHGLTPEQ | |
| | | VVAIASNIGGKQALETVQALLPVLCQAHGLTPQQVVAIASNNGGKQALE | |
| | | TVQRLLPVLCQAHGLTPEQVVAIASNIGGKQALETVQALLPVLCQAHGL | |
| | | TPQQVVAIASNGGGKQALETVQRLLPVLCQAHGLTPEQVVAIASNIGGK | |
| | | QALETVQALLPVLCQAHGLTPQQVVAIASNGGGKQALETVQRLLPVLC | |
| | | QAHGLTPEQVVAIASHDGGKQALETVQRLLPVLCQAHGLTPQQVVAIAS | |
| | | NGGGRPALESIVAQLSRPDPALAALTNDHLVALACLGGRPALDAVKKGL | |
| | | GDPISRSQLVKSELEEKKSELRHKLKYVPHEYIELIEIARNSTQDRILEMK | |
| | | VMEFFMKVYGYRGKHLGGSRKPDGAIYTVGSPIDYGVIVDTKAYSGGY | |
| | | NLPIGQADEMQRYVEENQTRNKHINPNEWWKVYPSSVTEFKFLFVSGH | |
| | | FKGNYKAQLTRLNHITNCNGAVLSVEELLIGGEMIKAGTLTLEEVRRKFN | |
| | | NGEINFAAD | |
| 17 | TALEN | MGDPKKKRKVIDKETAAAKFERQHMDSIDIADLRTLGYSQQQQEKIKPK | HD-NG-HD-NI-NN- |
| | Left TRAC | VRSTVAQHHEALVGHGFTHAHIVALSQHPAALGTVAVKYQDMIAALPEA | HD-NG-NN-NN- |
| | | THEAIVGVGKQWSGARALEALLTVAGELRGPPLQLDTGQLLKIAKRGGV | NG-NI-HD-NI-HD- |
| | | TAVEAVHAWRNALTGAPLNLTPEQVVAIASHDGGKQALETVQRLLPVL | NN-NG# |
| | | CQAHGLTPQQVVAIASNGGGKQALETVQRLLPVLCQAHGLTPEQVVAI | |
| | | ASHDGGKQALETVQRLLPVLCQAHGLTPEQVVAIASNIGGKQALETVQA | |
| | | LLPVLCQAHGLTPQQVVAIASNNGGKQALETVQRLLPVLCQAHGLTPE | |
| | | QVVAIASHDGGKQALETVQRLLPVLCQAHGLTPQQVVAIASNGGGKQA | |
| | | LETVQRLLPVLCQAHGLTPQQVVAIASNNGGKQALETVQRLLPVLCQA | |
| | | HGLTPQQVVAIASNNGGKQALETVQRLLPVLCQAHGLTPQQVVAIASN | |
| | | GGGKQALETVQRLLPVLCQAHGLTPEQVVAIASNIGGKQALETVQALLP | |
| | | VLCQAHGLTPEQVVAIASHDGGKQALETVQRLLPVLCQAHGLTPEQVV | |
| | | AIASNIGGKQALETVQALLPVLCQAHGLTPEQVVAIASHDGGKQALETV | |
| | | QRLLPVLCQAHGLTPQQVVAIASNNGGKQALETVQRLLPVLCQAHGLT | |
| | | PQQVVAIASNGGGRPALESIVAQLSRPDPALAALTNDHLVALACLGGRP | |
| | | ALDAVKKGLGDPISRSQLVKSELEEKKSELRHKLKYVPHEYIELIEIARNS | |
| | | TQDRILEMKVMEFFMKVYGYRGKHLGGSRKPDGAIYTVGSPIDYGVIVD | |
| | | TKAYSGGYNLPIGQADEMQRYVEENQTRNKHINPNEWWKVYPSSVTE | |
| | | FKFLFVSGHFKGNYKAQLTRLNHITNCNGAVLSVEELLIGGEMIKAGTLT | |
| | | LEEVRRKFNNGEINFAAD | |
| 18 | TALEN | MGDPKKKRKVIDYPYDVPDYAIDIADLRTLGYSQQQQEKIKPKVRSTVA | NN-NG-NG-HD- |
| | right CD25 | QHHEALVGHGFTHAHIVALSQHPAALGTVAVKYQDMIAALPEATHEAIV | NG-NG-NG-NG- |
| | | GVGKQWSGARALEALLTVAGELRGPPLQLDTGQLLKIAKRGGVTAVEA | NN-NN-NG-NG- |
| | | VHAWRNALTGAPLNLTPQQVVAIASNNGGKQALETVQRLLPVLCQAHG | NG-NG-HD-NG# |
| | | LTPQQVVAIASNGGGKQALETVQRLLPVLCQAHGLTPQQVVAIASNGG | |
| | | GKQALETVQRLLPVLCQAHGLTPEQVVAIASHDGGKQALETVQRLLPVL | |
| | | CQAHGLTPQQVVAIASNGGGKQALETVQRLLPVLCQAHGLTPQQVVAI | |
| | | ASNGGGKQALETVQRLLPVLCQAHGLTPQQVVAIASNGGGKQALETVQ | |
| | | RLLPVLCQAHGLTPQQVVAIASNGGGKQALETVQRLLPVLCQAHGLTP | |
| | | QQVVAIASNNGGKQALETVQRLLPVLCQAHGLTPQQVVAIASNNGGKQ | |
| | | ALETVQRLLPVLCQAHGLTPQQVVAIASNGGGKQALETVQRLLPVLCQ | |
| | | AHGLTPQQVVAIASNGGGKQALETVQRLLPVLCQAHGLTPQQVVAIAS | |
| | | NGGGKQALETVQRLLPVLCQAHGLTPQQVVAIASNGGGKQALETVQRL | |
| | | LPVLCQAHGLTPEQVVAIASHDGGKQALETVQRLLPVLCQAHGLTPQQ | |
| | | VVAIASNGGGRPALESIVAQLSRPDPSGSGSGGDPISRSQLVKSELEEK | |
| | | KSELRHKLKYVPHEYIELIEIARNSTQDRILEMKVMEFFMKVYGYRGKHL | |
| | | GGSRKPDGAIYTVGSPIDYGVIVDTKAYSGGYNLPIGQADEMQRYVEEN | |
| | | QTRNKHINPNEWWKVYPSSVTEFKFLFVSGHFKGNYKAQLTRLNHITN | |
| | | CNGAVLSVEELLIGGEMIKAGTLTLEEVRRKFNNGEINFAAD | |
| 19 | TALEN left | MGDPKKKRKVIDYPYDVPDYAIDIADLRTLGYSQQQQEKIKPKVRSTVA | NI-HD-NI-NN-NN- |
| | CD25 | QHHEALVGHGFTHAHIVALSQHPAALGTVAVKYQDMIAALPEATHEAIV | NI-NN-NN-NI-NI- |
| | | GVGKQWSGARALEALLTVAGELRGPPLQLDTGQLLKIAKRGGVTAVEA | NN-NI-NN-NG-NI- |
| | | VHAWRNALTGAPLNLTPEQVVAIASNIGGKQALETVQALLPVLCQAHGL | NG# |
| | | TPEQVVAIASHDGGKQALETVQRLLPVLCQAHGLTPEQVVAIASNIGGK | |
| | | QALETVQALLPVLCQAHGLTPQQVVAIASNNGGKQALETVQRLLPVLC | |
| | | QAHGLTPQQVVAIASNNGGKQALETVQRLLPVLCQAHGLTPEQVVAIAS | |
| | | NIGGKQALETVQALLPVLCQAHGLTPQQVVAIASNNGGKQALETVQRLL | |
| | | PVLCQAHGLTPQQVVAIASNNGGKQALETVQRLLPVLCQAHGLTPEQV | |
| | | VAIASNIGGKQALETVQALLPVLCQAHGLTPEQVVAIASNIGGKQALETV | |
| | | QALLPVLCQAHGLTPQQVVAIASNNGGKQALETVQRLLPVLCQAHGLT | |
| | | PEQVVAIASNIGGKQALETVQALLPVLCQAHGLTPQQVVAIASNNGGKQ | |
| | | ALETVQRLLPVLCQAHGLTPQQVVAIASNGGGKQALETVQRLLPVLCQ | |
| | | AHGLTPEQVVAIASNIGGKQALETVQALLPVLCQAHGLTPQQVVAIASN | |
| | | GGGRPALESIVAQLSRPDPSGSGSGGDPISRSQLVKSELEEKKSELRH | |
| | | KLKYVPHEYIELIEIARNSTQDRILEMKVMEFFMKVYGYRGKHLGGSRK | |
| | | PDGAIYTVGSPIDYGVIVDTKAYSGGYNLPIGQADEMQRYVEENQTRNK | |
| | | HINPNEWWKVYPSSVTEFKFLFVSGHFKGNYKAQLTRLNHITNCNGAV | |
| | | LSVEELLIGGEMIKAGTLTLEEVRRKFNNGEINFAAD | |
| 20 | TALEN | MGDPKKKRKVIDYPYDVPDYAIDIADLRTLGYSQQQQEKIKPKVRSTVA | KL-HD-HD-NG-HD- |
| | right PD1 | QHHEALVGHGFTHAHIVALSQHPAALGTVAVKYQDMIAALPEATHEAIV | NG-YK-NG-NN- |
| | | GVGKQWSGARALEALLTVAGELRGPPLQLDTGQLLKIAKRGGVTAVEA | NN-NN-NN-HD- |
| | | VHAWRNALTGAPLNLTPEQVVAIASKLGGKQALETVQALLPVLCQAHGL | HD-NI-NG# |
| | | TPEQVVAIASHDGGKQALETVQRLLPVLCQAHGLTPEQVVAIASHDGG | |
| | | KQALETVQRLLPVLCQAHGLTPQQVVAIASNGGGKQALETVQRLLPVL | |
| | | CQAHGLTPEQVVAIASHDGGKQALETVQRLLPVLCQAHGLTPQQVVAI | |
| | | ASNGGGKQALETVQRLLPVLCQAHGLTPQQVVAIASYKGGKQALETVQ | |
| | | RLLPVLCQAHGLTPQQVVAIASNGGGKQALETVQRLLPVLCQAHGLTP | |
| | | QQVVAIASNNGGKQALETVQRLLPVLCQAHGLTPQQVVAIASNNGGKQ | |
| | | ALETVQRLLPVLCQAHGLTPQQVVAIASNNGGKQALETVQRLLPVLCQA | |
| | | HGLTPQQVVAIASNNGGKQALETVQRLLPVLCQAHGLTPEQVVAIASHD | |
| | | GGKQALETVQRLLPVLCQAHGLTPEQVVAIASHDGGKQALETVQRLLP | |
| | | VLCQAHGLTPEQVVAIASNIGGKQALETVQALLPVLCQAHGLTPQQVVA | |
| | | IASNGGGRPALESIVAQLSRPDPALAALTNDHLVALACLGGRPALDAVK | |
| | | KGLGDPISRSQLVKSELEEKKSELRHKLKYVPHEYIELIEIARNSTQDRIL | |
| | | EMKVMEFFMKVYGYRGKHLGGSRKPDGAIYTVGSPIDYGVIVDTKAYS | |
| | | GGYNLPIGQADEMQRYVEENQTRNKHINPNEWWKVYPSSVTEFKFLFV | |
| | | SGHFKGNYKAQLTRLNHITNCNGAVLSVEELLIGGEMIKAGTLTLEEVRR | |
| | | KFNNGEINFAAD | |
| 21 | TALEN | MGDPKKKRKVIDKETAAAKFERQHMDSIDIADLRTLGYSQQQQEKIKPK | HD-NG-HD-NG- |
| | Left PD1 | VRSTVAQHHEALVGHGFTHAHIVALSQHPAALGTVAVKYQDMIAALPEA | NG-NG-NN-NI-NG- |
| | | THEAIVGVGKQWSGARALEALLTVAGELRGPPLQLDTGQLLKIAKRGGV | HD-NG-NN-N-NN- |
| | | TAVEAVHAWRNALTGAPLNLTPEQVVAIASHDGGKQALETVQRLLPVL | HD-NG# |
| | | CQAHGLTPQQVVAIASNGGGKQALETVQRLLPVLCQAHGLTPEQVVAI | |
| | | ASHDGGKQALETVQRLLPVLCQAHGLTPQQVVAIASNGGGKQALETVQ | |
| | | RLLPVLCQAHGLTPQQVVAIASNGGGKQALETVQRLLPVLCQAHGLTP | |
| | | QQVVAIASNGGGKQALETVQRLLPVLCQAHGLTPQQVVAIASNNGGKQ | |
| | | ALETVQRLLPVLCQAHGLTPEQVVAIASNIGGKQALETVQALLPVLCQA | |
| | | HGLTPQQVVAIASNGGGKQALETVQRLLPVLCQAHGLTPEQVVAIASH | |
| | | DGGKQALETVQRLLPVLCQAHGLTPQQVVAIASNGGGKQALETVQRLL | |
| | | PVLCQAHGLTPQQVVAIASNNGGKQALETVQRLLPVLCQAHGLTPEQV | |
| | | VAIASNGGKQALETVQRLLPVLCQAHGLTPQQVVAIASNNGGKQALETV | |
| | | QRLLPVLCQAHGLTPEQVVAIASHDGGKQALETVQRLLPVLCQAHGLT | |
| | | PQQVVAIASNGGGRPALESIVAQLSRPDPALAALTNDHLVALACLGGRP | |
| | | ALDAVKKGLGDPISRSQLVKSELEEKKSELRHKLKYVPHEYIELIEIARNS | |
| | | TQDRILEMKVMEFFMKVYGYRGKHLGGSRKPDGAIYTVGSPIDYGVIVD | |
| | | TKAYSGGYNLPIGQADEMQRYVEENQTRNKHINPNEWWKVYPSSVTE | |
| | | FKFLFVSGHFKGNYKAQLTRLNHITNCNGAVLSVEELLIGGEMIKAGTLT | |
| | | LEEVRRKFNNGEINFAAD | |

| SEQ | | |
|---|---|---|
| ID | Sequence | |
| NO# | Name | Polynucleotide sequence |
| 22 | TALEN TRAC | ATGGGCGATCCTAAAAAGAAACGTAAGGTCATCGATTACCCATACGATGTTCCAGATTACGCTAT |
| | pCLS11370 | CGATATCGCCGATCTACGCACGCTCGGCTACAGCCAGCAGCAACAGGAGAAGATCAAACCGAA |
| | | GGTTCGTTCGACAGTGGCGCAGCACCACGAGGCACTGGTCGGCCACGGGTTTACACACGCGC |
| | | ACATCGTTGCGTTAAGCCAACACCCGGCAGCGTTAGGGACCGTCGCTGTCAAGTATCAGGACA |
| | | TGATCGCAGCGTTGCCAGAGGCGACACACGAAGCGATCGTTGGCGTCGGCAAACAGTGGTCC |
| | | GGCGCACGCGCTCTGGAGGCCTTGCTCACGGTGGCGGGAGAGTTGAGAGGTCCACCGTTACA |
| | | GTTGGACACAGGCCAACTTCTCAAGATTGCAAAACGTGGCGGCGTGACCGCAGTGGAGGCAGT |
| | | GCATGCATGGCGCAATGCACTGACGGGTGCCCCGCTCAACTTGACCCCCCAGCAGGTGGTGG |
| | | CCATCGCCAGCAATGGCGGTGGCAAGCAGGCGCTGGAGACGGTCCAGCGGCTGTTGCCGGTG |
| | | CTGTGCCAGGCCCACGGCTTGACCCCCCAGCAGGTGGTGGCCATCGCCAGCAATAATGGTGG |
| | | CAAGCAGGCGCTGGAGACGGTCCAGCGGCTGTTGCCGGTGCTGTGCCAGGCCCACGGCTTGA |
| | | CCCCCCAGCAGGTGGTGGCCATCGCCAGCAATGGCGGTGGCAAGCAGGCGCTGGAGACGGT |
| | | CCAGCGGCTGTTGCCGGTGCTGTGCCAGGCCCACGGCTTGACCCCGGAGCAGGTGGTGGCCA |
| | | TCGCCAGCCACGATGGCGGCAAGCAGGCGCTGGAGACGGTCCAGCGGCTGTTGCCGGTGCTG |
| | | TGCCAGGCCCACGGCTTGACCCCGGAGCAGGTGGTGGCCATCGCCAGCCACGATGGCGGCAA |
| | | GCAGGCGCTGGAGACGGTCCAGCGGCTGTTGCCGGTGCTGTGCCAGGCCCACGGCTTGACCC |
| | | CGGAGCAGGTGGTGGCCATCGCCAGCCACGATGGCGGCAAGCAGGCGCTGGAGACGGTCCA |
| | | GCGGCTGTTGCCGGTGCTGTGCCAGGCCCACGGCTTGACCCCGGAGCAGGTGGTGGCCATCG |
| | | CCAGCAATATTGGTGGCAAGCAGGCGCTGGAGACGGTGCAGGCGCTGTTGCCGGTGCTGTGC |
| | | CAGGCCCACGGCTTGACCCCGGAGCAGGTGGTGGCCATCGCCAGCCACGATGGCGGCAAGCA |
| | | GGCGCTGGAGACGGTCCAGCGGCTGTTGCCGGTGCTGTGCCAGGCCCACGGCTTGACCCCG |
| | | GAGCAGGTGGTGGCCATCGCCAGCAATATTGGTGGCAAGCAGGCGCTGGAGACGGTGCAGGC |
| | | GCTGTTGCCGGTGCTGTGCCAGGCCCACGGCTTGACCCCCCAGCAGGTGGTGGCCATCGCCA |
| | | GCAATAATGGTGGCAAGCAGGCGCTGGAGACGGTCCAGCGGCTGTTGCCGGTGCTGTGCCAG |
| | | GCCCACGGCTTGACCCCGGAGCAGGTGGTGGCCATCGCCAGCAATATTGGTGGCAAGCAGGC |
| | | GCTGGAGACGGTGCAGGCGCTGTTGCCGGTGCTGTGCCAGGCCCACGGCTTGACCCCCCAGC |
| | | AGGTGGTGGCCATCGCCAGCAATGGCGGTGGCAAGCAGGCGCTGGAGACGGTCCAGCGGCT |
| | | GTTGCCGGTGCTGTGCCAGGCCCACGGCTTGACCCCGGAGCAGGTGGTGGCCATCGCCAGCA |
| | | ATATTGGTGGCAAGCAGGCGCTGGAGACGGTGCAGGCGCTGTTGCCGGTGCTGTGCCAGGCC |
| | | CACGGCTTGACCCCCCAGCAGGTGGTGGCCATCGCCAGCAATGGCGGTGGCAAGCAGGCGCT |
| | | GGAGACGGTCCAGCGGCTGTTGCCGGTGCTGTGCCAGGCCCACGGCTTGACCCCGGAGCAG |
| | | GTGGTGGCCATCGCCAGCCACGATGGCGGCAAGCAGGCGCTGGAGACGGTCCAGCGGCTGTT |
| | | GCCGGTGCTGTGCCAGGCCCACGGCTTGACCCCTCAGCAGGTGGTGGCCATCGCCAGCAATG |
| | | GCGGCGGCAGGCCGGCGCTGGAGAGCATTGTTGCCCAGTTATCTCGCCCTGATCCGGCGTTG |
| | | GCCGCGTTGACCAACGACCACCTCGTCGCCTTGGCCTGCCTCGGCGGGCGTCCTGCGCTGGA |
| | | TGCAGTGAAAAAGGGATTGGGGGATCCTATCAGCCGTTCCCAGCTGGTGAAGTCCGAGCTGGA |
| | | GGAGAAGAAATCCGAGTTGAGGCACAAGCTGAAGTACGTGCCCCACGAGTACATCGAGCTGAT |
| | | CGAGATCGCCCGGAACAGCACCCAGGACCGTATCCTGGAGATGAAGGTGATGGAGTTCTTCAT |
| | | GAAGGTGTACGGCTACAGGGGCAAGCACCTGGGCGGCTCCAGGAAGCCCGACGGCGCCATCT |
| | | ACACCGTGGGCTCCCCCATCGACTACGGCGTGATCGTGGACACCAAGGCCTACTCCGGCGGC |
| | | TACAACCTGCCCATCGGCCAGGCCGACGAAATGCAGAGGTACGTGGAGGAGAACCAGACCAG |
| | | GAACAAGCACATCAACCCCAACGAGTGGTGGAAGGTGTACCCCTCCAGCGTGACCGAGTTCAA |
| | | GTTCCTGTTCGTGTCCGGCCACTTCAAGGGCAACTACAAGGCCCAGCTGACCAGGCTGAACCA |
| | | CATCACCAACTGCAACGGCGCCGTGCTGTCCGTGGAGGAGCTCCTGATCGGCGGCGAGATGA |
| | | TCAAGGCCGGCACCCTGACCCTGGAGGAGGTGAGGAGGAAGTTCAACAACGGCGAGATCAAC |
| | | TTCGCGGCCGACTGATAA |
| 23 | TALEN TRAC | ATGGGCGATCCTAAAAAGAAACGTAAGGTCATCGATAAGGAGACCGCCGCTGCCAAGTTCGAG |
| | pCLS11369 | AGACAGCACATGGACAGCATCGATATCGCCGATCTACGCACGCTCGGCTACAGCCAGCAGCAA |
| | | CAGGAGAAGATCAAACCGAAGGTTCGTTCGACAGTGGCGCAGCACCACGAGGCACTGGTCGG |
| | | CCACGGGTTTACACACGCGCACATCGTTGCGTTAAGCCAACACCCGGCAGCGTTAGGGACCGT |
| | | CGCTGTCAAGTATCAGGACATGATCGCAGCGTTGCCAGAGGCGACACACGAAGCGATCGTTGG |
| | | CGTCGGCAAACAGTGGTCCGGCGCACGCGCTCTGGAGGCCTTGCTCACGGTGGCGGGAGAGT |
| | | TGAGAGGTCCACCGTTACAGTTGGACACAGGCCAACTTCTCAAGATTGCAAAACGTGGCGGCG |
| | | TGACCGCAGTGGAGGCAGTGCATGCATGGCGCAATGCACTGACGGGTGCCCCGCTCAACTTG |
| | | ACCCCGGAGCAGGTGGTGGCCATCGCCAGCCACGATGGCGGCAAGCAGGCGCTGGAGACGG |
| | | TCCAGCGGCTGTTGCCGGTGCTGTGCCAGGCCCACGGCTTGACCCCCCAGCAGGTGGTGGCC |
| | | ATCGCCAGCAATGGCGGTGGCAAGCAGGCGCTGGAGACGGTCCAGCGGCTGTTGCCGGTGCT |
| | | GTGCCAGGCCCACGGCTTGACCCCGGAGCAGGTGGTGGCCATCGCCAGCCACGATGGCGGC |
| | | AAGCAGGCGCTGGAGACGGTCCAGCGGCTGTTGCCGGTGCTGTGCCAGGCCCACGGCTTGAC |
| | | CCCGGAGCAGGTGGTGGCCATCGCCAGCAATATTGGTGGCAAGCAGGCGCTGGAGACGGTGC |
| | | AGGCGCTGTTGCCGGTGCTGTGCCAGGCCCACGGCTTGACCCCCCAGCAGGTGGTGGCCATC |
| | | GCCAGCAATAATGGTGGCAAGCAGGCGCTGGAGACGGTCCAGCGGCTGTTGCCGGTGCTGTG |
| | | CCAGGCCCACGGCTTGACCCCGGAGCAGGTGGTGGCCATCGCCAGCCACGATGGCGGCAAG |
| | | CAGGCGCTGGAGACGGTCCAGCGGCTGTTGCCGGTGCTGTGCCAGGCCCACGGCTTGACCCC |
| | | CCAGCAGGTGGTGGCCATCGCCAGCAATGGCGGTGGCAAGCAGGCGCTGGAGACGGTCCAG |
| | | CGGCTGTTGCCGGTGCTGTGCCAGGCCCACGGCTTGACCCCCCAGCAGGTGGTGGCCATCGC |
| | | CAGCAATAATGGTGGCAAGCAGGCGCTGGAGACGGTCCAGCGGCTGTTGCCGGTGCTGTGCC |
| | | AGGCCCACGGCTTGACCCCCCAGCAGGTGGTGGCCATCGCCAGCAATAATGGTGGCAAGCAG |
| | | GCGCTGGAGACGGTCCAGCGGCTGTTGCCGGTGCTGTGCCAGGCCCACGGCTTGACCCCCCA |
| | | GCAGGTGGTGGCCATCGCCAGCAATGGCGGTGGCAAGCAGGCGCTGGAGACGGTCCAGCGG |
| | | CTGTTGCCGGTGCTGTGCCAGGCCCACGGCTTGACCCCGGAGCAGGTGGTGGCCATCGCCAG |
| | | CAATATTGGTGGCAAGCAGGCGCTGGAGACGGTGCAGGCGCTGTTGCCGGTGCTGTGCCAGG |
| | | CCCACGGCTTGACCCCGGAGCAGGTGGTGGCCATCGCCAGCCACGATGGCGGCAAGCAGGC |
| | | GCTGGAGACGGTCCAGCGGCTGTTGCCGGTGCTGTGCCAGGCCCACGGCTTGACCCCGGAGC |
| | | AGGTGGTGGCCATCGCCAGCAATATTGGTGGCAAGCAGGCGCTGGAGACGGTGCAGGCGCTG |
| | | TTGCCGGTGCTGTGCCAGGCCCACGGCTTGACCCCGGAGCAGGTGGTGGCCATCGCCAGCCA |
| | | CGATGGCGGCAAGCAGGCGCTGGAGACGGTCCAGCGGCTGTTGCCGGTGCTGTGCCAGGCC |
| | | CACGGCTTGACCCCCCAGCAGGTGGTGGCCATCGCCAGCAATAATGGTGGCAAGCAGGCGCT |
| | | GGAGACGGTCCAGCGGCTGTTGCCGGTGCTGTGCCAGGCCCACGGCTTGACCCCTCAGCAGG |
| | | TGGTGGCCATCGCCAGCAATGGCGGCGGCAGGCCGGCGCTGGAGAGCATTGTTGCCCAGTTA |
| | | TCTCGCCCTGATCCGGCGTTGGCCGCGTTGACCAACGACCACCTCGTCGCCTTGGCCTGCCTC |
| | | GGCGGGCGTCCTGCGCTGGATGCAGTGAAAAAGGGATTGGGGGATCCTATCAGCCGTTCCCA |
| | | GCTGGTGAAGTCCGAGCTGGAGGAGAAGAAATCCGAGTTGAGGCACAAGCTGAAGTACGTGCC |
| | | CCACGAGTACATCGAGCTGATCGAGATCGCCCGGAACAGCACCCAGGACCGTATCCTGGAGAT |
| | | GAAGGTGATGGAGTTCTTCATGAAGGTGTACGGCTACAGGGGCAAGCACCTGGGCGGCTCCA |
| | | GGAAGCCCGACGGCGCCATCTACACCGTGGGCTCCCCCATCGACTACGGCGTGATCGTGGAC |
| | | ACCAAGGCCTACTCCGGCGGCTACAACCTGCCCATCGGCCAGGCCGACGAAATGCAGAGGTA |
| | | CGTGGAGGAGAACCAGACCAGGAACAAGCACATCAACCCCAACGAGTGGTGGAAGGTGTACC |
| | | CCTCCAGCGTGACCGAGTTCAAGTTCCTGTTCGTGTCCGGCCACTTCAAGGGCAACTACAAGG |
| | | CCCAGCTGACCAGGCTGAACCACATCACCAACTGCAACGGCGCCGTGCTGTCCGTGGAGGAG |
| | | CTCCTGATCGGCGGCGAGATGATCAAGGCCGGCACCCTGACCCTGGAGGAGGTGAGGAGGAA |
| | | GTTCAACAACGGCGAGATCAACTTCGCGGCCGACTGATAA |
| 24 | TALEN CD25 | ATGGGCGATCCTAAAAAGAAACGTAAGGTCATCGATTACCCATACGATGTTCCAGATTACGCTAT |
| | pCLS30480 | CGATATCGCCGATCTACGCACGCTCGGCTACAGCCAGCAGCAACAGGAGAAGATCAAACCGAA |
| | | GGTTCGTTCGACAGTGGCGCAGCACCACGAGGCACTGGTCGGCCACGGGTTTACACACGCGC |
| | | ACATCGTTGCGTTAAGCCAACACCCGGCAGCGTTAGGGACCGTCGCTGTCAAGTATCAGGACA |
| | | TGATCGCAGCGTTGCCAGAGGCGACACACGAAGCGATCGTTGGCGTCGGCAAACAGTGGTCC |
| | | GGCGCACGCGCTCTGGAGGCCTTGCTCACGGTGGCGGGAGAGTTGAGAGGTCCACCGTTACA |
| | | GTTGGACACAGGCCAACTTCTCAAGATTGCAAAACGTGGCGGCGTGACCGCAGTGGAGGCAGT |
| | | GCATGCATGGCGCAATGCACTGACGGGTGCCCCGCTCAACTTGACCCCCCAGCAGGTGGTGG |
| | | CCATCGCCAGCAATAATGGTGGCAAGCAGGCGCTGGAGACGGTCCAGCGGCTGTTGCCGGTG |
| | | CTGTGCCAGGCCCACGGCTTGACCCCCCAGCAGGTGGTGGCCATCGCCAGCAATGGCGGTGG |
| | | CAAGCAGGCGCTGGAGACGGTCCAGCGGCTGTTGCCGGTGCTGTGCCAGGCCCACGGCTTGA |
| | | CCCCCCAGCAGGTGGTGGCCATCGCCAGCAATGGCGGTGGCAAGCAGGCGCTGGAGACGGT |
| | | CCAGCGGCTGTTGCCGGTGCTGTGCCAGGCCCACGGCTTGACCCCGGAGCAGGTGGTGGCCA |
| | | TCGCCAGCCACGATGGCGGCAAGCAGGCGCTGGAGACGGTCCAGCGGCTGTTGCCGGTGCTG |
| | | TGCCAGGCCCACGGCTTGACCCCCCAGCAGGTGGTGGCCATCGCCAGCAATGGCGGTGGCAA |
| | | GCAGGCGCTGGAGACGGTCCAGCGGCTGTTGCCGGTGCTGTGCCAGGCCCACGGCTTGACCC |
| | | CCCAGCAGGTGGTGGCCATCGCCAGCAATGGCGGTGGCAAGCAGGCGCTGGAGACGGTCCA |
| | | GCGGCTGTTGCCGGTGCTGTGCCAGGCCCACGGCTTGACCCCCCAGCAGGTGGTGGCCATCG |
| | | CCAGCAATGGCGGTGGCAAGCAGGCGCTGGAGACGGTCCAGCGGCTGTTGCCGGTGCTGTGC |
| | | CAGGCCCACGGCTTGACCCCCCAGCAGGTGGTGGCCATCGCCAGCAATGGCGGTGGCAAGCA |
| | | GGCGCTGGAGACGGTCCAGCGGCTGTTGCCGGTGCTGTGCCAGGCCCACGGCTTGACCCCCC |
| | | AGCAGGTGGTGGCCATCGCCAGCAATAATGGTGGCAAGCAGGCGCTGGAGACGGTCCAGCGG |
| | | CTGTTGCCGGTGCTGTGCCAGGCCCACGGCTTGACCCCCCAGCAGGTGGTGGCCATCGCCAG |
| | | CAATAATGGTGGCAAGCAGGCGCTGGAGACGGTCCAGCGGCTGTTGCCGGTGCTGTGCCAGG |
| | | CCCACGGCTTGACCCCCCAGCAGGTGGTGGCCATCGCCAGCAATGGCGGTGGCAAGCAGGCG |
| | | CTGGAGACGGTCCAGCGGCTGTTGCCGGTGCTGTGCCAGGCCCACGGCTTGACCCCCCAGCA |
| | | GGTGGTGGCCATCGCCAGCAATGGCGGTGGCAAGCAGGCGCTGGAGACGGTCCAGCGGCTG |
| | | TTGCCGGTGCTGTGCCAGGCCCACGGCTTGACCCCCCAGCAGGTGGTGGCCATCGCCAGCAA |
| | | TGGCGGTGGCAAGCAGGCGCTGGAGACGGTCCAGCGGCTGTTGCCGGTGCTGTGCCAGGCC |
| | | CACGGCTTGACCCCCCAGCAGGTGGTGGCCATCGCCAGCAATGGCGGTGGCAAGCAGGCGCT |
| | | GGAGACGGTCCAGCGGCTGTTGCCGGTGCTGTGCCAGGCCCACGGCTTGACCCCGGAGCAG |
| | | GTGGTGGCCATCGCCAGCCACGATGGCGGCAAGCAGGCGCTGGAGACGGTCCAGCGGCTGTT |
| | | GCCGGTGCTGTGCCAGGCCCACGGCTTGACCCCTCAGCAGGTGGTGGCCATCGCCAGCAATG |
| | | GCGGCGGCAGGCCGGCGCTGGAGAGCATTGTTGCCCAGTTATCTCGCCCTGATCCGAGTGGC |
| | | AGCGGAAGTGGCGGGGATCCTATCAGCCGTTCCCAGCTGGTGAAGTCCGAGCTGGAGGAGAA |
| | | GAAATCCGAGTTGAGGCACAAGCTGAAGTACGTGCCCCACGAGTACATCGAGCTGATCGAGAT |
| | | CGCCCGGAACAGCACCCAGGACCGTATCCTGGAGATGAAGGTGATGGAGTTCTTCATGAAGGT |
| | | GTACGGCTACAGGGGCAAGCACCTGGGCGGCTCCAGGAAGCCCGACGGCGCCATCTACACCG |
| | | TGGGCTCCCCCATCGACTACGGCGTGATCGTGGACACCAAGGCCTACTCCGGCGGCTACAACC |
| | | TGCCCATCGGCCAGGCCGACGAAATGCAGAGGTACGTGGAGGAGAACCAGACCAGGAACAAG |
| | | CACATCAACCCCAACGAGTGGTGGAAGGTGTACCCCTCCAGCGTGACCGAGTTCAAGTTCCTG |
| | | TTCGTGTCCGGCCACTTCAAGGGCAACTACAAGGCCCAGCTGACCAGGCTGAACCACATCACC |
| | | AACTGCAACGGCGCCGTGCTGTCCGTGGAGGAGCTCCTGATCGGCGGCGAGATGATCAAGGC |
| | | CGGCACCCTGACCCTGGAGGAGGTGAGGAGGAAGTTCAACAACGGCGAGATCAACTTCGCGG |
| | | CCGACTGATAA |
| 25 | TALEN CD25 | ATGGGCGATCCTAAAAAGAAACGTAAGGTCATCGATTACCCATACGATGTTCCAGATTACGCTAT |
| | pCLS30479 | CGATATCGCCGATCTACGCACGCTCGGCTACAGCCAGCAGCAACAGGAGAAGATCAAACCGAA |
| | | GGTTCGTTCGACAGTGGCGCAGCACCACGAGGCACTGGTCGGCCACGGGTTTACACACGCGC |
| | | ACATCGTTGCGTTAAGCCAACACCCGGCAGCGTTAGGGACCGTCGCTGTCAAGTATCAGGACA |
| | | TGATCGCAGCGTTGCCAGAGGCGACACACGAAGCGATCGTTGGCGTCGGCAAACAGTGGTCC |
| | | GGCGCACGCGCTCTGGAGGCCTTGCTCACGGTGGCGGGAGAGTTGAGAGGTCCACCGTTACA |
| | | GTTGGACACAGGCCAACTTCTCAAGATTGCAAAACGTGGCGGCGTGACCGCAGTGGAGGCAGT |
| | | GCATGCATGGCGCAATGCACTGACGGGTGCCCCGCTCAACTTGACCCCGGAGCAGGTGGTGG |
| | | CCATCGCCAGCAATATTGGTGGCAAGCAGGCGCTGGAGACGGTGCAGGCGCTGTTGCCGGTG |
| | | CTGTGCCAGGCCCACGGCTTGACCCCGGAGCAGGTGGTGGCCATCGCCAGCCACGATGGCGG |
| | | CAAGCAGGCGCTGGAGACGGTCCAGCGGCTGTTGCCGGTGCTGTGCCAGGCCCACGGCTTGA |
| | | CCCCGGAGCAGGTGGTGGCCATCGCCAGCAATATTGGTGGCAAGCAGGCGCTGGAGACGGTG |
| | | CAGGCGCTGTTGCCGGTGCTGTGCCAGGCCCACGGCTTGACCCCCCAGCAGGTGGTGGCCAT |
| | | CGCCAGCAATAATGGTGGCAAGCAGGCGCTGGAGACGGTCCAGCGGCTGTTGCCGGTGCTGT |
| | | GCCAGGCCCACGGCTTGACCCCCCAGCAGGTGGTGGCCATCGCCAGCAATAATGGTGGCAAG |
| | | CAGGCGCTGGAGACGGTCCAGCGGCTGTTGCCGGTGCTGTGCCAGGCCCACGGCTTGACCCC |
| | | GGAGCAGGTGGTGGCCATCGCCAGCAATATTGGTGGCAAGCAGGCGCTGGAGACGGTGCAGG |
| | | CGCTGTTGCCGGTGCTGTGCCAGGCCCACGGCTTGACCCCCCAGCAGGTGGTGGCCATCGCC |
| | | AGCAATAATGGTGGCAAGCAGGCGCTGGAGACGGTCCAGCGGCTGTTGCCGGTGCTGTGCCA |
| | | GGCCCACGGCTTGACCCCCCAGCAGGTGGTGGCCATCGCCAGCAATAATGGTGGCAAGCAGG |
| | | CGCTGGAGACGGTCCAGCGGCTGTTGCCGGTGCTGTGCCAGGCCCACGGCTTGACCCCGGAG |
| | | CAGGTGGTGGCCATCGCCAGCAATATTGGTGGCAAGCAGGCGCTGGAGACGGTGCAGGCGCT |
| | | GTTGCCGGTGCTGTGCCAGGCCCACGGCTTGACCCCGGAGCAGGTGGTGGCCATCGCCAGCA |
| | | ATATTGGTGGCAAGCAGGCGCTGGAGACGGTGCAGGCGCTGTTGCCGGTGCTGTGCCAGGCC |
| | | CACGGCTTGACCCCCCAGCAGGTGGTGGCCATCGCCAGCAATAATGGTGGCAAGCAGGCGCT |
| | | GGAGACGGTCCAGCGGCTGTTGCCGGTGCTGTGCCAGGCCCACGGCTTGACCCCGGAGCAG |
| | | GTGGTGGCCATCGCCAGCAATATTGGTGGCAAGCAGGCGCTGGAGACGGTGCAGGCGCTGTT |
| | | GCCGGTGCTGTGCCAGGCCCACGGCTTGACCCCCCAGCAGGTGGTGGCCATCGCCAGCAATA |
| | | ATGGTGGCAAGCAGGCGCTGGAGACGGTCCAGCGGCTGTTGCCGGTGCTGTGCCAGGCCCAC |
| | | GGCTTGACCCCCCAGCAGGTGGTGGCCATCGCCAGCAATGGCGGTGGCAAGCAGGCGCTGGA |
| | | GACGGTCCAGCGGCTGTTGCCGGTGCTGTGCCAGGCCCACGGCTTGACCCCGGAGCAGGTGG |
| | | TGGCCATCGCCAGCAATATTGGTGGCAAGCAGGCGCTGGAGACGGTGCAGGCGCTGTTGCCG |
| | | GTGCTGTGCCAGGCCCACGGCTTGACCCCTCAGCAGGTGGTGGCCATCGCCAGCAATGGCGG |
| | | CGGCAGGCCGGCGCTGGAGAGCATTGTTGCCCAGTTATCTCGCCCTGATCCGAGTGGCAGCG |
| | | GAAGTGGCGGGGATCCTATCAGCCGTTCCCAGCTGGTGAAGTCCGAGCTGGAGGAGAAGAAAT |
| | | CCGAGTTGAGGCACAAGCTGAAGTACGTGCCCCACGAGTACATCGAGCTGATCGAGATCGCCC |
| | | GGAACAGCACCCAGGACCGTATCCTGGAGATGAAGGTGATGGAGTTCTTCATGAAGGTGTACG |
| | | GCTACAGGGGCAAGCACCTGGGCGGCTCCAGGAAGCCCGACGGCGCCATCTACACCGTGGGC |
| | | TCCCCCATCGACTACGGCGTGATCGTGGACACCAAGGCCTACTCCGGCGGCTACAACCTGCCC |
| | | ATCGGCCAGGCCGACGAAATGCAGAGGTACGTGGAGGAGAACCAGACCAGGAACAAGCACAT |
| | | CAACCCCAACGAGTGGTGGAAGGTGTACCCCTCCAGCGTGACCGAGTTCAAGTTCCTGTTCGT |
| | | GTCCGGCCACTTCAAGGGCAACTACAAGGCCCAGCTGACCAGGCTGAACCACATCACCAACTG |
| | | CAACGGCGCCGTGCTGTCCGTGGAGGAGCTCCTGATCGGCGGCGAGATGATCAAGGCCGGCA |
| | | CCCTGACCCTGGAGGAGGTGAGGAGGAAGTTCAACAACGGCGAGATCAACTTCGCGGCCGAC |
| | | TGATAA |
| 26 | TALEN PD1 | ATGGGCGATCCTAAAAAGAAACGTAAGGTCATCGATTACCCATACGATGTTCCAGATTACGCTAT |
| | pCLS28959 | CGATATCGCCGATCTACGCACGCTCGGCTACAGCCAGCAGCAACAGGAGAAGATCAAACCGAA |
| | | GGTTCGTTCGACAGTGGCGCAGCACCACGAGGCACTGGTCGGCCACGGGTTTACACACGCGC |
| | | ACATCGTTGCGTTAAGCCAACACCCGGCAGCGTTAGGGACCGTCGCTGTCAAGTATCAGGACA |
| | | TGATCGCAGCGTTGCCAGAGGCGACACACGAAGCGATCGTTGGCGTCGGCAAACAGTGGTCC |
| | | GGCGCACGCGCTCTGGAGGCCTTGCTCACGGTGGCGGGAGAGTTGAGAGGTCCACCGTTACA |
| | | GTTGGACACAGGCCAACTTCTCAAGATTGCAAAACGTGGCGGCGTGACCGCAGTGGAGGCAGT |
| | | GCATGCATGGCGCAATGCACTGACGGGTGCCCCGCTCAACTTGACCCCCGAGCAAGTGGTGG |
| | | CTATCGCTTCCAAGCTGGGGGGAAAGCAGGCCCTGGAGACCGTCCAGGCCCTTCTCCCAGTG |
| | | CTTTGCCAGGCTCACGGACTGACCCCTGAACAGGTGGTGGCAATTGCCTCACACGACGGGGG |
| | | CAAGCAGGCACTGGAGACTGTCCAGCGGCTGCTGCCTGTCCTCTGCCAGGCCCACGGACTCA |
| | | CTCCTGAGCAGGTCGTGGCCATTGCCAGCCACGATGGGGGCAAACAGGCTCTGGAGACCGTG |
| | | CAGCGCCTCCTCCCAGTGCTGTGCCAGGCTCATGGGCTGACCCCACAGCAGGTCGTCGCCATT |
| | | GCCAGTAACGGCGGGGGGAAGCAGGCCCTCGAAACAGTGCAGAGGCTGCTGCCCGTCTTGTG |
| | | CCAAGCACACGGCCTGACACCCGAGCAGGTGGTGGCCATCGCCTCTCATGACGGCGGCAAGC |
| | | AGGCCCTTGAGACAGTGCAGAGACTGTTGCCCGTGTTGTGTCAGGCCCACGGGTTGACACCCC |
| | | AGCAGGTGGTCGCCATCGCCAGCAATGGCGGGGGAAAGCAGGCCCTTGAGACCGTGCAGCGG |
| | | TTGCTTCCAGTGTTGTGCCAGGCACACGGACTGACCCCTCAACAGGTGGTCGCAATCGCCAGC |
| | | TACAAGGGCGGAAAGCAGGCTCTGGAGACAGTGCAGCGCCTCCTGCCCGTGCTGTGTCAGGC |
| | | TCACGGACTGACACCACAGCAGGTGGTCGCCATCGCCAGTAACGGGGGCGGCAAGCAGGCTT |
| | | TGGAGACCGTCCAGAGACTCCTCCCCGTCCTTTGCCAGGCCCACGGGTTGACACCTCAGCAGG |
| | | TCGTCGCCATTGCCTCCAACAACGGGGGCAAGCAGGCCCTCGAAACTGTGCAGAGGCTGCTG |
| | | CCTGTGCTGTGCCAGGCTCATGGGCTGACACCCCAGCAGGTGGTGGCCATTGCCTCTAACAAC |
| | | GGCGGCAAACAGGCACTGGAGACCGTGCAAAGGCTGCTGCCCGTCCTCTGCCAAGCCCACGG |
| | | GCTCACTCCACAGCAGGTCGTGGCCATCGCCTCAAACAATGGCGGGAAGCAGGCCCTGGAGA |
| | | CTGTGCAAAGGCTGCTCCCTGTGCTCTGCCAGGCACACGGACTGACCCCTCAGCAGGTGGTG |
| | | GCAATCGCTTCCAACAACGGGGGAAAGCAGGCCCTCGAAACCGTGCAGCGCCTCCTCCCAGT |
| | | GCTGTGCCAGGCACATGGCCTCACACCCGAGCAAGTGGTGGCTATCGCCAGCCACGACGGAG |
| | | GGAAGCAGGCTCTGGAGACCGTGCAGAGGCTGCTGCCTGTCCTGTGCCAGGCCCACGGGCTT |
| | | ACTCCAGAGCAGGTCGTCGCCATCGCCAGTCATGATGGGGGGAAGCAGGCCCTTGAGACAGT |
| | | CCAGCGGCTGCTGCCAGTCCTTTGCCAGGCTCACGGCTTGACTCCCGAGCAGGTCGTGGCCAT |
| | | TGCCTCAAACATTGGGGGCAAACAGGCCCTGGAGACAGTGCAGGCCCTGCTGCCCGTGTTGTG |
| | | TCAGGCCCACGGCTTGACACCCCAGCAGGTGGTCGCCATTGCCTCTAATGGCGGCGGGAGAC |
| | | CCGCCTTGGAGAGCATTGTTGCCCAGTTATCTCGCCCTGATCCGGCGTTGGCCGCGTTGACCA |
| | | ACGACCACCTCGTCGCCTTGGCCTGCCTCGGCGGGCGTCCTGCGCTGGATGCAGTGAAAAAG |
| | | GGATTGGGGGATCCTATCAGCCGTTCCCAGCTGGTGAAGTCCGAGCTGGAGGAGAAGAAATCC |
| | | GAGTTGAGGCACAAGCTGAAGTACGTGCCCCACGAGTACATCGAGCTGATCGAGATCGCCCGG |
| | | AACAGCACCCAGGACCGTATCCTGGAGATGAAGGTGATGGAGTTCTTCATGAAGGTGTACGGC |
| | | TACAGGGGCAAGCACCTGGGCGGCTCCAGGAAGCCCGACGGCGCCATCTACACCGTGGGCTC |
| | | CCCCATCGACTACGGCGTGATCGTGGACACCAAGGCCTACTCCGGCGGCTACAACCTGCCCAT |
| | | CGGCCAGGCCGACGAAATGCAGAGGTACGTGGAGGAGAACCAGACCAGGAACAAGCACATCA |
| | | ACCCCAACGAGTGGTGGAAGGTGTACCCCTCCAGCGTGACCGAGTTCAAGTTCCTGTTCGTGT |
| | | CCGGCCACTTCAAGGGCAACTACAAGGCCCAGCTGACCAGGCTGAACCACATCACCAACTGCA |
| | | ACGGCGCCGTGCTGTCCGTGGAGGAGCTCCTGATCGGCGGCGAGATGATCAAGGCCGGCACC |
| | | CTGACCCTGGAGGAGGTGAGGAGGAAGTTCAACAACGGCGAGATCAACTTCGCGGCCGACTG |
| | | ATAA |
| 27 | TALEN PD1 | ATGGGCGATCCTAAAAAGAAACGTAAGGTCATCGATAAGGAGACCGCCGCTGCCAAGTTCGAG |
| | pCLS18792 | AGACAGCACATGGACAGCATCGATATCGCCGATCTACGCACGCTCGGCTACAGCCAGCAGCAA |
| | | CAGGAGAAGATCAAACCGAAGGTTCGTTCGACAGTGGCGCAGCACCACGAGGCACTGGTCGG |
| | | CCACGGGTTTACACACGCGCACATCGTTGCGTTAAGCCAACACCCGGCAGCGTTAGGGACCGT |
| | | CGCTGTCAAGTATCAGGACATGATCGCAGCGTTGCCAGAGGCGACACACGAAGCGATCGTTGG |
| | | CGTCGGCAAACAGTGGTCCGGCGCACGCGCTCTGGAGGCCTTGCTCACGGTGGCGGGAGAGT |
| | | TGAGAGGTCCACCGTTACAGTTGGACACAGGCCAACTTCTCAAGATTGCAAAACGTGGCGGCG |
| | | TGACCGCAGTGGAGGCAGTGCATGCATGGCGCAATGCACTGACGGGTGCCCCGCTCAACTTG |
| | | ACCCCCGAGCAAGTCGTCGCAATCGCCAGCCATGATGGAGGGAAGCAAGCCCTCGAAACCGT |
| | | GCAGCGGTTGCTTCCTGTGCTCTGCCAGGCCCACGGCCTTACCCCTCAGCAGGTGGTGGCCAT |
| | | CGCAAGTAACGGAGGAGGAAAGCAAGCCTTGGAGACAGTGCAGCGCCTGTTGCCCGTGCTGT |
| | | GCCAGGCACACGGCCTCACACCAGAGCAGGTCGTGGCCATTGCCTCCCATGACGGGGGGAAA |
| | | CAGGCTCTGGAGACCGTCCAGAGGCTGCTGCCCGTCCTCTGTCAAGCTCACGGCCTGACTCCC |
| | | CAACAAGTGGTCGCCATCGCCTCTAATGGCGGCGGGAAGCAGGCACTGGAAACAGTGCAGAG |
| | | ACTGCTCCCTGTGCTTTGCCAAGCTCATGGGTTGACCCCCCAACAGGTCGTCGCTATTGCCTCA |
| | | AACGGGGGGGGCAAGCAGGCCCTTGAGACTGTGCAGAGGCTGTTGCCAGTGCTGTGTCAGGC |
| | | TCACGGGCTCACTCCACAACAGGTGGTCGCAATTGCCAGCAACGGCGGCGGAAAGCAAGCTCT |
| | | TGAAACCGTGCAACGCCTCCTGCCCGTGCTCTGTCAGGCTCATGGCCTGACACCACAACAAGT |
| | | CGTGGCCATCGCCAGTAATAATGGCGGGAAACAGGCTCTTGAGACCGTCCAGAGGCTGCTCCC |
| | | AGTGCTCTGCCAGGCACACGGGCTGACCCCCGAGCAGGTGGTGGCTATCGCCAGCAATATTG |
| | | GGGGCAAGCAGGCCCTGGAAACAGTCCAGGCCCTGCTGCCAGTGCTTTGCCAGGCTCACGGG |
| | | CTCACTCCCCAGCAGGTCGTGGCAATCGCCTCCAACGGCGGAGGGAAGCAGGCTCTGGAGAC |
| | | CGTGCAGAGACTGCTGCCCGTCTTGTGCCAGGCCCACGGACTCACACCTGAACAGGTCGTCGC |
| | | CATTGCCTCTCACGATGGGGGCAAACAAGCCCTGGAGACAGTGCAGCGGCTGTTGCCTGTGTT |
| | | GTGCCAAGCCCACGGCTTGACTCCTCAACAAGTGGTCGCCATCGCCTCAAATGGCGGCGGAAA |
| | | ACAAGCTCTGGAGACAGTGCAGAGGTTGCTGCCCGTCCTCTGCCAAGCCCACGGCCTGACTCC |
| | | CCAACAGGTCGTCGCCATTGCCAGCAACAACGGAGGAAAGCAGGCTCTCGAAACTGTGCAGCG |
| | | GCTGCTTCCTGTGCTGTGTCAGGCTCATGGGCTGACCCCCGAGCAAGTGGTGGCTATTGCCTC |
| | | TAATGGAGGCAAGCAAGCCCTTGAGACAGTCCAGAGGCTGTTGCCAGTGCTGTGCCAGGCCCA |
| | | CGGGCTCACACCCCAGCAGGTGGTCGCCATCGCCAGTAACAACGGGGGCAAACAGGCATTGG |
| | | AAACCGTCCAGCGCCTGCTTCCAGTGCTCTGCCAGGCACACGGACTGACACCCGAACAGGTGG |
| | | TGGCCATTGCATCCCATGATGGGGGCAAGCAGGCCCTGGAGACCGTGCAGAGACTCCTGCCA |
| | | GTGTTGTGCCAAGCTCACGGCCTCACCCCTCAGCAAGTCGTGGCCATCGCCTCAAACGGGGG |
| | | GGGCCGGCCTGCACTGGAGAGCATTGTTGCCCAGTTATCTCGCCCTGATCCGGCGTTGGCCG |
| | | CGTTGACCAACGACCACCTCGTCGCCTTGGCCTGCCTCGGCGGGCGTCCTGCGCTGGATGCA |
| | | GTGAAAAAGGGATTGGGGGATCCTATCAGCCGTTCCCAGCTGGTGAAGTCCGAGCTGGAGGAG |
| | | AAGAAATCCGAGTTGAGGCACAAGCTGAAGTACGTGCCCCACGAGTACATCGAGCTGATCGAG |
| | | ATCGCCCGGAACAGCACCCAGGACCGTATCCTGGAGATGAAGGTGATGGAGTTCTTCATGAAG |
| | | GTGTACGGCTACAGGGGCAAGCACCTGGGCGGCTCCAGGAAGCCCGACGGCGCCATCTACAC |
| | | CGTGGGCTCCCCCATCGACTACGGCGTGATCGTGGACACCAAGGCCTACTCCGGCGGCTACA |
| | | ACCTGCCCATCGGCCAGGCCGACGAAATGCAGAGGTACGTGGAGGAGAACCAGACCAGGAAC |
| | | AAGCACATCAACCCCAACGAGTGGTGGAAGGTGTACCCCTCCAGCGTGACCGAGTTCAAGTTC |
| | | CTGTTCGTGTCCGGCCACTTCAAGGGCAACTACAAGGCCCAGCTGACCAGGCTGAACCACATC |
| | | ACCAACTGCAACGGCGCCGTGCTGTCCGTGGAGGAGCTCCTGATCGGCGGCGAGATGATCAA |
| | | GGCCGGCACCCTGACCCTGGAGGAGGTGAGGAGGAAGTTCAACAACGGCGAGATCAACTTCG |
| | | CGGCCGACTGATAA |
| 28 | TALEN | TTGTCCCACAGATATCCAGAACCCTGACCCTGCCGTGTACCAGCTGAGA |
| | target TRAC | |
| 29 | TALEN | TACAGGAGGAAGAGTAGAAGAACAATCTAGAAAACCAAAAGAACA |
| | target CD25 | |
| 30 | TALEN | TACCTCTGTGGGGCCATCTCCCTGGCCCCCAAGGCGCAGATCAAAGAGA |
| | target PD1 | |
| 31 | Matrice TRAC | TTGCTGGGCCTTTTTCCCATGCCTGCCTTTACTCTGCCAGAGTTATATTGCTGGGGTTTTGAAGA |
| | locus_CubiCAR | AGATCCTATTAAATAAAAGAATAAGCAGTATTATTAAGTAGCCCTGCATTTCAGGTTTCCTTGAGT |
| | CD22 | GGCAGGCCAGGCCTGGCCGTGAACGTTCACTGAAATCATGGCCTCTTGGCCAAGATTGATAGC |
| | pCLS30056 | TTGTGCCTGTCCCTGAGTCCCAGTCCATCACGAGCAGCTGGTTTCTAAGATGCTATTTCCCGTA |
| | | TAAAGCATGAGACCGTGACTTGCCAGCCCCACAGAGCCCCGCCCTTGTCCATCACTGGCATCT |
| | | GGACTCCAGCCTGGGTTGGGGCAAAGAGGGAAATGAGATCATGTCCTAACCCTGATCCTCTTG |
| | | TCCCACAGATATCCAGTACCCCTACGACGTGCCCGACTACGCCTCCGGTGAGGGCAGAGGAAG |
| | | TCTTCTAACATGCGGTGACGTGGAGGAGAATCCGGGCCCCGGATCCGCTCTGCCCGTCACCGC |
| | | TCTGCTGCTGCCACTGGCACTGCTGCTGCACGCTGCTAGGCCCGGAGGGGGAGGCAGCTGCC |
| | | CCTACAGCAACCCCAGCCTGTGCAGCGGAGGCGGCGGCAGCGGCGGAGGGGGTAGCCAGGT |
| | | GCAGCTGCAGCAGAGCGGCCCTGGCCTGGTGAAGCCAAGCCAGACACTGTCCCTGACCTGCG |
| | | CCATCAGCGGCGATTCCGTGAGCTCCAACTCCGCCGCCTGGAATTGGATCAGGCAGTCCCCTT |
| | | CTCGGGGCCTGGAGTGGCTGGGAAGGACATACTATCGGTCTAAGTGGTACAACGATTATGCCG |
| | | TGTCTGTGAAGAGCAGAATCACAATCAACCCTGACACCTCCAAGAATCAGTTCTCTCTGCAGCT |
| | | GAATAGCGTGACACCAGAGGACACCGCCGTGTACTATTGCGCCAGGGAGGTGACCGGCGACC |
| | | TGGAGGATGCCTTTGACATCTGGGGCCAGGGCACAATGGTGACCGTGAGCTCCGGAGGCGGC |
| | | GGATCTGGCGGAGGAGGAAGTGGGGGCGGCGGGAGTGATATCCAGATGACACAGTCCCCATC |
| | | CTCTCTGAGCGCCTCCGTGGGCGACAGAGTGACAATCACCTGTAGGGCCTCCCAGACCATCTG |
| | | GTCTTACCTGAACTGGTATCAGCAGAGGCCCGGCAAGGCCCCTAATCTGCTGATCTACGCAGC |
| | | AAGCTCCCTGCAGAGCGGAGTGCCATCCAGATTCTCTGGCAGGGGCTCCGGCACAGACTTCAC |
| | | CCTGACCATCTCTAGCCTGCAGGCCGAGGACTTCGCCACCTACTATTGCCAGCAGTCTTATAGC |
| | | ATCCCCCAGACATTTGGCCAGGGCACCAAGCTGGAGATCAAGTCGGATCCCGGAAGCGGAGG |
| | | GGGAGGCAGCTGCCCCTACAGCAACCCCAGCCTGTGCAGCGGAGGCGGCGGCAGCGAGCTG |
| | | CCCACCCAGGGCACCTTCTCCAACGTGTCCACCAACGTGAGCCCAGCCAAGCCCACCACCACC |
| | | GCCTGTCCTTATTCCAATCCTTCCCTGTGTGCTCCCACCACAACCCCCGCTCCAAGGCCCCCTA |
| | | CCCCCGCACCAACTATTGCCTCCCAGCCACTCTCACTGCGGCCTGAGGCCTGTCGGCCCGCTG |
| | | CTGGAGGCGCAGTGCATACAAGGGGCCTCGATTTCGCCTGCGATATTTACATCTGGGCACCCC |
| | | TCGCCGGCACCTGCGGGGTGCTTCTCCTCTCCCTGGTGATTACCCTGTATTGCAGACGGGGCC |
| | | GGAAGAAGCTCCTCTACATTTTTAAGCAGCCTTTCATGCGGCCAGTGCAGACAACCCAAGAGGA |
| | | GGATGGGTGTTCCTGCAGATTCCCTGAGGAAGAGGAAGGCGGGTGCGAGCTGAGAGTGAAGT |
| | | TCTCCAGGAGCGCAGATGCCCCCGCCTATCAACAGGGCCAGAACCAGCTCTACAACGAGCTTA |
| | | ACCTCGGGAGGCGCGAAGAATACGACGTGTTGGATAAGAGAAGGGGGCGGGACCCCGAGATG |
| | | GGAGGAAAGCCCCGGAGGAAGAACCCTCAGGAGGGCCTGTACAACGAGCTGCAGAAGGATAA |
| | | GATGGCCGAGGCCTACTCAGAGATCGGGATGAAGGGGGAGCGGCGCCGCGGGAAGGGGCAC |
| | | GATGGGCTCTACCAGGGGCTGAGCACAGCCACAAAGGACACATACGACGCCTTGCACATGCAG |
| | | GCCCTTCCACCCCGGGAATAGTCTAGAGGGCCCGTTTAAACCCGCTGATCAGCCTCGACTGTG |
| | | CCTTCTAGTTGCCAGCCATCTGTTGTTTGCCCCTCCCCCGTGCCTTCCTTGACCCTGGAAGGTG |
| | | CCACTCCCACTGTCCTTTCCTAATAAAATGAGGAAATTGCATCGCATTGTCTGAGTAGGTGTCAT |
| | | TCTATTCTGGGGGGTGGGGTGGGGCAGGACAGCAAGGGGGAGGATTGGGAAGACAATAGCAG |
| | | GCATGCTGGGGATGCGGTGGGCTCTATGACTAGTGGCGAATTCCCGTGTACCAGCTGAGAGAC |
| | | TCTAAATCCAGTGACAAGTCTGTCTGCCTATTCACCGATTTTGATTCTCAAACAAATGTGTCACA |
| | | AAGTAAGGATTCTGATGTGTATATCACAGACAAAACTGTGCTAGACATGAGGTCTATGGACTTCA |
| | | AGAGCAACAGTGCTGTGGCCTGGAGCAACAAATCTGACTTTGCATGTGCAAACGCCTTCAACAA |
| | | CAGCATTATTCCAGAAGACACCTTCTTCCCCAGCCCAGGTAAGGGCAGCTTTGGTGCCTTCGCA |
| | | GGCTGTTTCCTTGCTTCAGGAATGGCCAGGTTCTGCCCAGAGCTCTGGTCAATGATGTCTAAAA |
| | | CTCCTCTGATTGGTGGTCTCGGCCTTATCCATTGCCACCAAAACCCTCTTTTTACTAA |
| 32 | Matrice CD25 | GTTTATTATTCCTGTTCCACAGCTATTGTCTGCCATATAAAAACTTAGGCCAGGCACAGTGGCTC |
| | locus_IL15_ | ACACCTGTAATCCCAGCACTTTGGAAGGCCGAGGCAGGCAGATCACAAGGTCAGGAGTTCGAG |
| | 2A_sIL15Ra | ACCAGCCTGGCCAACATAGCAAAACCCCATCTCTACTAAAAATACAAAAATTAGCCAGGCATGG |
| | pCLS30519 | TGGCGTGTGCACTGGTTTAGAGTGAGGACCACATTTTTTTGGTGCCGTGTTACACATATGACCG |
| | | TGACTTTGTTACACCACTACAGGAGGAAGAGTAGAAGAACAATCGGTTCTGGCGTGAAACAGAC |
| | | TTTGAATTTTGACCTTCTCAAGTTGGCGGGAGACGTGGAGTCCAACCCAGGGCCCGGTACCGG |
| | | GTCCGCCACCATGGACTGGACCTGGATTCTGTTCCTCGTGGCTGCTGCTACAAGAGTGCACAG |
| | | CGGCATTCATGTCTTCATTTTGGGCTGTTTCAGTGCAGGGCTTCCTAAAACAGAAGCCAACTGG |
| | | GTGAATGTAATAAGTGATTTGAAAAAAATTGAAGATCTTATTCAATCTATGCATATTGATGCTACT |
| | | TTATATACGGAAAGTGATGTTCACCCCAGTTGCAAAGTAACAGCAATGAAGTGCTTTCTCTTGGA |
| | | GTTACAAGTTATTTCACTTGAGTCCGGAGATGCAAGTATTCATGATACAGTAGAAAATCTGATCA |
| | | TCCTAGCAAACAACAGTTTGTCTTCTAATGGGAATGTAACAGAATCTGGATGCAAAGAATGTGAG |
| | | GAACTGGAGGAAAAAAATATTAAAGAATTTTTGCAGAGTTTTGTACATATTGTCCAAATGTTCATC |
| | | AACACTTCTGGAAGCGGAGCTACTAACTTCAGCCTGCTGAAGCAGGCTGGAGACGTGGAGGAG |
| | | AACCCTGGACCTGGGACCGGCTCTGCAACCATGGATTGGACGTGGATCCTGTTTCTCGTGGCA |
| | | GCTGCCACAAGAGTTCACAGTATCACGTGCCCTCCCCCCATGTCCGTGGAACACGCAGACATC |
| | | TGGGTCAAGAGCTACAGCTTGTACTCCAGGGAGCGGTACATTTGTAACTCTGGTTTCAAGCGTA |
| | | AAGCCGGCACGTCCAGCCTGACGGAGTGCGTGTTGAACAAGGCCACGAATGTCGCCCACTGG |
| | | ACAACCCCCAGTCTCAAATGCATTAGAGACCCTGCCCTGGTTCACCAAAGGCCAGCGCCACCC |
| | | TCCACAGTAACGACGGCAGGGGTGACCCCACAGCCAGAGAGCCTCTCCCCTTCTGGAAAAGAG |
| | | CCCGCAGCTTCATCTCCCAGCTCAAACAACACAGCGGCCACAACAGCAGCTATTGTCCCGGGC |
| | | TCCCAGCTGATGCCTTCAAAATCACCTTCCACAGGAACCACAGAGATAAGCAGTCATGAGTCCT |
| | | CCCACGGCACCCCCTCTCAGACAACAGCCAAGAACTGGGAACTCACAGCATCCGCCTCCCACC |
| | | AGCCGCCAGGTGTGTATCCACAGGGCCACAGCGACACCACTGAGGGCAGAGGCAGCCTGCTG |
| | | ACCTGCGGCGACGTCGAGGAGAACCCCGGGCCCATGGGGGCAGGTGCCACCGGCCGCGCCA |
| | | TGGACGGGCCGCGCCTGCTGCTGTTGCTGCTTCTGGGGGTGTCCCTTGGAGGTGCCAAGGAG |
| | | GCATGCCCCACAGGCCTGTACACACACAGCGGTGAGTGCTGCAAAGCCTGCAACCTGGGCGA |
| | | GGGTGTGGCCCAGCCTTGTGGAGCCAACCAGACCGTGTGTGAGCCCTGCCTGGACAGCGTGA |
| | | CGTTCTCCGACGTGGTGAGCGCGACCGAGCCGTGCAAGCCGTGCACCGAGTGCGTGGGGCTC |
| | | CAGAGCATGTCGGCGCCGTGCGTGGAGGCCGATGACGCCGTGTGCCGCTGCGCCTACGGCTA |
| | | CTACCAGGATGAGACGACTGGGCGCTGCGAGGCGTGCCGCGTGTGCGAGGCGGGCTCGGGC |
| | | CTCGTGTTCTCCTGCCAGGACAAGCAGAACACCGTGTGCGAGGAGTGCCCCGACGGCACGTAT |
| | | TCCGACGAGGCCAACCACGTGGACCCGTGCCTGCCCTGCACCGTGTGCGAGGACACCGAGCG |
| | | CCAGCTCCGCGAGTGCACACGCTGGGCCGACGCCGAGTGCGAGGAGATCCCTGGCCGTTGGA |
| | | TTACACGGTCCACACCCCCAGAGGGCTCGGACAGCACAGCCCCCAGCACCCAGGAGCCTGAG |
| | | GCACCTCCAGAACAAGACCTCATAGCCAGCACGGTGGCAGGTGTGGTGACCACAGTGATGGG |
| | | CAGCTCCCAGCCCGTGGTGACCCGAGGCACCACCGACAACCTCATCCCTGTCTATTGCTCCAT |
| | | CCTGGCTGCTGTGGTTGTGGGTCTTGTGGCCTACATAGCCTTCAAGAGGTGAAAAACCAAAAGA |
| | | ACAAGAATTTCTTGGTAAGAAGCCGGGAACAGACAACAGAAGTCATGAAGCCCAAGTGAAATCA |
| | | AAGGTGCTAAATGGTCGCCCAGGAGACATCCGTTGTGCTTGCCTGCGTTTTGGAAGCTCTGAA |
| | | GTCACATCACAGGACACGGGGCAGTGGCAACCTTGTCTCTATGCCAGCTCAGTCCCATCAGAG |
| | | AGCGAGCGCTACCCACTTCTAAATAGCAATTTCGCCGTTGAAGAGGAAGGGCAAAACCACTAGA |
| | | ACTCTCCATCTTATTTTCATGTATATGTGTTCAT |
| 33 | Matrice PD1 | GACTCCCCAGACAGGCCCTGGAACCCCCCCACCTTCTCCCCAGCCCTGCTCGTGGTGACCGAA |
| | locus_IL15_ | GGGGACAACGCCACCTTCACCTGCAGCTTCTCCAACACATCGGAGAGCTTCGTGCTAAACTGG |
| | 2A_sIL15Ra | TACCGCATGAGCCCCAGCAACCAGACGGACAAGCTGGCCGCCTTCCCCGAGGACCGCAGCCA |
| | pCLS30513 | GCCCGGCCAGGACTGCCGCTTCCGTGTCACACAACTGCCCAACGGGCGTGACTTCCACATGAG |
| | | CGTGGTCAGGGCCCGGCGCAATGACAGCGGCACCTACCTCTGTGGGGCCGGTTCTGGCGTGA |
| | | AACAGACTTTGAATTTTGACCTTCTCAAGTTGGCGGGAGACGTGGAGTCCAACCCAGGGCCCG |
| | | GTACCGGGTCCGCCACCATGGACTGGACCTGGATTCTGTTCCTCGTGGCTGCTGCTACAAGAG |
| | | TGCACAGCGGCATTCATGTCTTCATTTTGGGCTGTTTCAGTGCAGGGCTTCCTAAAACAGAAGC |
| | | CAACTGGGTGAATGTAATAAGTGATTTGAAAAAAATTGAAGATCTTATTCAATCTATGCATATTGA |
| | | TGCTACTTTATATACGGAAAGTGATGTTCACCCCAGTTGCAAAGTAACAGCAATGAAGTGCTTTC |
| | | TCTTGGAGTTACAAGTTATTTCACTTGAGTCCGGAGATGCAAGTATTCATGATACAGTAGAAAAT |
| | | CTGATCATCCTAGCAAACAACAGTTTGTCTTCTAATGGGAATGTAACAGAATCTGGATGCAAAGA |
| | | ATGTGAGGAACTGGAGGAAAAAAATATTAAAGAATTTTTGCAGAGTTTTGTACATATTGTCCAAAT |
| | | GTTCATCAACACTTCTGGAAGCGGAGCTACTAACTTCAGCCTGCTGAAGCAGGCTGGAGACGT |
| | | GGAGGAGAACCCTGGACCTGGGACCGGCTCTGCAACCATGGATTGGACGTGGATCCTGTTTCT |
| | | CGTGGCAGCTGCCACAAGAGTTCACAGTATCACGTGCCCTCCCCCCATGTCCGTGGAACACGC |
| | | AGACATCTGGGTCAAGAGCTACAGCTTGTACTCCAGGGAGCGGTACATTTGTAACTCTGGTTTC |
| | | AAGCGTAAAGCCGGCACGTCCAGCCTGACGGAGTGCGTGTTGAACAAGGCCACGAATGTCGC |
| | | CCACTGGACAACCCCCAGTCTCAAATGCATTAGAGACCCTGCCCTGGTTCACCAAAGGCCAGC |
| | | GCCACCCTCCACAGTAACGACGGCAGGGGTGACCCCACAGCCAGAGAGCCTCTCCCCTTCTG |
| | | GAAAAGAGCCCGCAGCTTCATCTCCCAGCTCAAACAACACAGCGGCCACAACAGCAGCTATTG |
| | | TCCCGGGCTCCCAGCTGATGCCTTCAAAATCACCTTCCACAGGAACCACAGAGATAAGCAGTCA |
| | | TGAGTCCTCCCACGGCACCCCCTCTCAGACAACAGCCAAGAACTGGGAACTCACAGCATCCGC |
| | | CTCCCACCAGCCGCCAGGTGTGTATCCACAGGGCCACAGCGACACCACTGAGGGCAGAGGCA |
| | | GCCTGCTGACCTGCGGCGACGTCGAGGAGAACCCCGGGCCCATGGGGGCAGGTGCCACCGG |
| | | CCGCGCCATGGACGGGCCGCGCCTGCTGCTGTTGCTGCTTCTGGGGGTGTCCCTTGGAGGTG |
| | | CCAAGGAGGCATGCCCCACAGGCCTGTACACACACAGCGGTGAGTGCTGCAAAGCCTGCAAC |
| | | CTGGGCGAGGGTGTGGCCCAGCCTTGTGGAGCCAACCAGACCGTGTGTGAGCCCTGCCTGGA |
| | | CAGCGTGACGTTCTCCGACGTGGTGAGCGCGACCGAGCCGTGCAAGCCGTGCACCGAGTGCG |
| | | TGGGGCTCCAGAGCATGTCGGCGCCGTGCGTGGAGGCCGATGACGCCGTGTGCCGCTGCGC |
| | | CTACGGCTACTACCAGGATGAGACGACTGGGCGCTGCGAGGCGTGCCGCGTGTGCGAGGCGG |
| | | GCTCGGGCCTCGTGTTCTCCTGCCAGGACAAGCAGAACACCGTGTGCGAGGAGTGCCCCGAC |
| | | GGCACGTATTCCGACGAGGCCAACCACGTGGACCCGTGCCTGCCCTGCACCGTGTGCGAGGA |
| | | CACCGAGCGCCAGCTCCGCGAGTGCACACGCTGGGCCGACGCCGAGTGCGAGGAGATCCCT |
| | | GGCCGTTGGATTACACGGTCCACACCCCCAGAGGGCTCGGACAGCACAGCCCCCAGCACCCA |
| | | GGAGCCTGAGGCACCTCCAGAACAAGACCTCATAGCCAGCACGGTGGCAGGTGTGGTGACCA |
| | | CAGTGATGGGCAGCTCCCAGCCCGTGGTGACCCGAGGCACCACCGACAACCTCATCCCTGTCT |
| | | ATTGCTCCATCCTGGCTGCTGTGGTTGTGGGTCTTGTGGCCTACATAGCCTTCAAGAGGTGATC |
| | | TAGAGGGCCCGTTTAAACCCGCTGATCAGCCTCGACTGTGCCTTCTAGTTGCCAGCCATCTGTT |
| | | GTTTGCCCCTCCCCCGTGCCTTCCTTGACCCTGGAAGGTGCCACTCCCACTGTCCTTTCCTAAT |
| | | AAAATGAGGAAATTGCATCGCATTGTCTGAGTAGGTGTCATTCTATTCTGGGGGGTGGGGTGGG |
| | | GCAGGACAGCAAGGGGGAGGATTGGGAAGACAATAGCAGGCATGCTGGGGATGCGGTGGGCT |
| | | CTATGACTAGTGGCGAATTCGGCGCAGATCAAAGAGAGCCTGCGGGCAGAGCTCAGGGTGACA |
| | | GGTGCGGCCTCGGAGGCCCCGGGGCAGGGGTGAGCTGAGCCGGTCCTGGGGTGGGTGTCCC |
| | | CTCCTGCACAGGATCAGGAGCTCCAGGGTCGTAGGGCAGGGACCCCCCAGCTCCAGTCCAGG |
| | | GCTCTGTCCTGCACCTGGGGAATGGTGACCGGCATCTCTGTCCTCTAGCTCTGGAAGCACCCC |
| | | AGCCCCTCTAGTCTGCCCTCACCCCTGACCCTGACCCTCCACCCTGACCCCGTCCTAACCCCT |
| | | GACCTTTG |
| 34 | Matrice CD25 | GTTTATTATTCCTGTTCCACAGCTATTGTCTGCCATATAAAAACTTAGGCCAGGCACAGTGGCTC |
| | locus_IL12a_ | ACACCTGTAATCCCAGCACTTTGGAAGGCCGAGGCAGGCAGATCACAAGGTCAGGAGTTCGAG |
| | 2A_IL12b | ACCAGCCTGGCCAACATAGCAAAACCCCATCTCTACTAAAAATACAAAAATTAGCCAGGCATGG |
| | pCLS30520 | TGGCGTGTGCACTGGTTTAGAGTGAGGACCACATTTTTTTGGTGCCGTGTTACACATATGACCG |
| | | TGACTTTGTTACACCACTACAGGAGGAAGAGTAGAAGAACAATCGGTTCTGGCGTGAAACAGAC |
| | | TTTGAATTTTGACCTTCTCAAGTTGGCGGGAGACGTGGAGTCCAACCCAGGGCCCATGTGGCC |
| | | CCCTGGGTCAGCCTCCCAGCCACCGCCCTCACCTGCCGCGGCCACAGGTCTGCATCCAGCGG |
| | | CTCGCCCTGTGTCCCTGCAGTGCCGGCTCAGCATGTGTCCAGCGCGCAGCCTCCTCCTTGTGG |
| | | CTACCCTGGTCCTCCTGGACCACCTCAGTTTGGCCAGAAACCTCCCCGTGGCCACTCCAGACC |
| | | CAGGAATGTTCCCATGCCTTCACCACTCCCAAAACCTGCTGAGGGCCGTCAGCAACATGCTCCA |
| | | GAAGGCCAGACAAACTCTAGAATTTTACCCTTGCACTTCTGAAGAGATTGATCATGAAGATATCA |
| | | CAAAAGATAAAACCAGCACAGTGGAGGCCTGTTTACCATTGGAATTAACCAAGAATGAGAGTTG |
| | | CCTAAATTCCAGAGAGACCTCTTTCATAACTAATGGGAGTTGCCTGGCCTCCAGAAAGACCTCT |
| | | TTTATGATGGCCCTGTGCCTTAGTAGTATTTATGAAGACTTGAAGATGTACCAGGTGGAGTTCAA |
| | | GACCATGAATGCAAAGCTTCTGATGGATCCTAAGAGGCAGATCTTTCTAGATCAAAACATGCTG |
| | | GCAGTTATTGATGAGCTGATGCAGGCCCTGAATTTCAACAGTGAGACTGTGCCACAAAAATCCT |
| | | CCCTTGAAGAACCGGATTTTTATAAAACTAAAATCAAGCTCTGCATACTTCTTCATGCTTTCAGAA |
| | | TTCGGGCAGTGACTATTGATAGAGTGATGAGCTATCTGAATGCTTCCGGAAGCGGAGCTACTAA |
| | | CTTCAGCCTGCTGAAGCAGGCTGGAGACGTGGAGGAGAACCCTGGACCTATGTGTCACCAGCA |
| | | GTTGGTCATCTCTTGGTTTTCCCTGGTTTTTCTGGCATCTCCCCTCGTGGCCATATGGGAACTGA |
| | | AGAAAGATGTTTATGTCGTAGAATTGGATTGGTATCCGGATGCCCCTGGAGAAATGGTGGTCCT |
| | | CACCTGTGACACCCCTGAAGAAGATGGTATCACCTGGACCTTGGACCAGAGCAGTGAGGTCTT |
| | | AGGCTCTGGCAAAACCCTGACCATCCAAGTCAAAGAGTTTGGAGATGCTGGCCAGTACACCTGT |
| | | CACAAAGGAGGCGAGGTTCTAAGCCATTCGCTCCTGCTGCTTCACAAAAAGGAAGATGGAATTT |
| | | GGTCCACTGATATTTTAAAGGACCAGAAAGAACCCAAAAATAAGACCTTTCTAAGATGCGAGGC |
| | | CAAGAATTATTCTGGACGTTTCACCTGCTGGTGGCTGACGACAATCAGTACTGATTTGACATTCA |
| | | GTGTCAAAAGCAGCAGAGGCTCTTCTGACCCCCAAGGGGTGACGTGCGGAGCTGCTACACTCT |
| | | CTGCAGAGAGAGTCAGAGGGGACAACAAGGAGTATGAGTACTCAGTGGAGTGCCAGGAGGAC |
| | | AGTGCCTGCCCAGCTGCTGAGGAGAGTCTGCCCATTGAGGTCATGGTGGATGCCGTTCACAAG |
| | | CTCAAGTATGAAAACTACACCAGCAGCTTCTTCATCAGGGACATCATCAAACCTGACCCACCCA |
| | | AGAACTTGCAGCTGAAGCCATTAAAGAATTCTCGGCAGGTGGAGGTCAGCTGGGAGTACCCTG |
| | | ACACCTGGAGTACTCCACATTCCTACTTCTCCCTGACATTCTGCGTTCAGGTCCAGGGCAAGAG |
| | | CAAGAGAGAAAAGAAAGATAGAGTCTTCACGGACAAGACCTCAGCCACGGTCATCTGCCGCAA |
| | | AAATGCCAGCATTAGCGTGCGGGCCCAGGACCGCTACTATAGCTCATCTTGGAGCGAATGGGC |
| | | ATCTGTGCCCTGCAGTGAGGGCAGAGGCAGCCTGCTGACCTGCGGCGACGTCGAGGAGAACC |
| | | CCGGGCCCATGGGGGCAGGTGCCACCGGCCGCGCCATGGACGGGCCGCGCCTGCTGCTGTT |
| | | GCTGCTTCTGGGGGTGTCCCTTGGAGGTGCCAAGGAGGCATGCCCCACAGGCCTGTACACAC |
| | | ACAGCGGTGAGTGCTGCAAAGCCTGCAACCTGGGCGAGGGTGTGGCCCAGCCTTGTGGAGCC |
| | | AACCAGACCGTGTGTGAGCCCTGCCTGGACAGCGTGACGTTCTCCGACGTGGTGAGCGCGAC |
| | | CGAGCCGTGCAAGCCGTGCACCGAGTGCGTGGGGCTCCAGAGCATGTCGGCGCCGTGCGTG |
| | | GAGGCCGATGACGCCGTGTGCCGCTGCGCCTACGGCTACTACCAGGATGAGACGACTGGGCG |
| | | CTGCGAGGCGTGCCGCGTGTGCGAGGCGGGCTCGGGCCTCGTGTTCTCCTGCCAGGACAAGC |
| | | AGAACACCGTGTGCGAGGAGTGCCCCGACGGCACGTATTCCGACGAGGCCAACCACGTGGAC |
| | | CCGTGCCTGCCCTGCACCGTGTGCGAGGACACCGAGCGCCAGCTCCGCGAGTGCACACGCTG |
| | | GGCCGACGCCGAGTGCGAGGAGATCCCTGGCCGTTGGATTACACGGTCCACACCCCCAGAGG |
| | | GCTCGGACAGCACAGCCCCCAGCACCCAGGAGCCTGAGGCACCTCCAGAACAAGACCTCATA |
| | | GCCAGCACGGTGGCAGGTGTGGTGACCACAGTGATGGGCAGCTCCCAGCCCGTGGTGACCCG |
| | | AGGCACCACCGACAACCTCATCCCTGTCTATTGCTCCATCCTGGCTGCTGTGGTTGTGGGTCTT |
| | | GTGGCCTACATAGCCTTCAAGAGGTGAAAAACCAAAAGAACAAGAATTTCTTGGTAAGAAGCCG |
| | | GGAACAGACAACAGAAGTCATGAAGCCCAAGTGAAATCAAAGGTGCTAAATGGTCGCCCAGGA |
| | | GACATCCGTTGTGCTTGCCTGCGTTTTGGAAGCTCTGAAGTCACATCACAGGACACGGGGCAG |
| | | TGGCAACCTTGTCTCTATGCCAGCTCAGTCCCATCAGAGAGCGAGCGCTACCCACTTCTAAATA |
| | | GCAATTTCGCCGTTGAAGAGGAAGGGCAAAACCACTAGAACTCTCCATCTTATTTTCATGTATAT |
| | | GTGTTCAT |
| 35 | Matrice PD1 | GACTCCCCAGACAGGCCCTGGAACCCCCCCACCTTCTCCCCAGCCCTGCTCGTGGTGACCGAA |
| | locus_IL12a_ | GGGGACAACGCCACCTTCACCTGCAGCTTCTCCAACACATCGGAGAGCTTCGTGCTAAACTGG |
| | 2A_IL12b | TACCGCATGAGCCCCAGCAACCAGACGGACAAGCTGGCCGCCTTCCCCGAGGACCGCAGCCA |
| | pCLS30511 | GCCCGGCCAGGACTGCCGCTTCCGTGTCACACAACTGCCCAACGGGCGTGACTTCCACATGAG |
| | | CGTGGTCAGGGCCCGGCGCAATGACAGCGGCACCTACCTCTGTGGGGCCGGTTCTGGCGTGA |
| | | AACAGACTTTGAATTTTGACCTTCTCAAGTTGGCGGGAGACGTGGAGTCCAACCCAGGGCCCAT |
| | | GTGGCCCCCTGGGTCAGCCTCCCAGCCACCGCCCTCACCTGCCGCGGCCACAGGTCTGCATC |
| | | CAGCGGCTCGCCCTGTGTCCCTGCAGTGCCGGCTCAGCATGTGTCCAGCGCGCAGCCTCCTC |
| | | CTTGTGGCTACCCTGGTCCTCCTGGACCACCTCAGTTTGGCCAGAAACCTCCCCGTGGCCACT |
| | | CCAGACCCAGGAATGTTCCCATGCCTTCACCACTCCCAAAACCTGCTGAGGGCCGTCAGCAAC |
| | | ATGCTCCAGAAGGCCAGACAAACTCTAGAATTTTACCCTTGCACTTCTGAAGAGATTGATCATGA |
| | | AGATATCACAAAAGATAAAACCAGCACAGTGGAGGCCTGTTTACCATTGGAATTAACCAAGAAT |
| | | GAGAGTTGCCTAAATTCCAGAGAGACCTCTTTCATAACTAATGGGAGTTGCCTGGCCTCCAGAA |
| | | AGACCTCTTTTATGATGGCCCTGTGCCTTAGTAGTATTTATGAAGACTTGAAGATGTACCAGGTG |
| | | GAGTTCAAGACCATGAATGCAAAGCTTCTGATGGATCCTAAGAGGCAGATCTTTCTAGATCAAAA |
| | | CATGCTGGCAGTTATTGATGAGCTGATGCAGGCCCTGAATTTCAACAGTGAGACTGTGCCACAA |
| | | AAATCCTCCCTTGAAGAACCGGATTTTTATAAAACTAAAATCAAGCTCTGCATACTTCTTCATGCT |
| | | TTCAGAATTCGGGCAGTGACTATTGATAGAGTGATGAGCTATCTGAATGCTTCCGGAAGCGGAG |
| | | CTACTAACTTCAGCCTGCTGAAGCAGGCTGGAGACGTGGAGGAGAACCCTGGACCTATGTGTC |
| | | ACCAGCAGTTGGTCATCTCTTGGTTTTCCCTGGTTTTTCTGGCATCTCCCCTCGTGGCCATATGG |
| | | GAACTGAAGAAAGATGTTTATGTCGTAGAATTGGATTGGTATCCGGATGCCCCTGGAGAAATGG |
| | | TGGTCCTCACCTGTGACACCCCTGAAGAAGATGGTATCACCTGGACCTTGGACCAGAGCAGTG |
| | | AGGTCTTAGGCTCTGGCAAAACCCTGACCATCCAAGTCAAAGAGTTTGGAGATGCTGGCCAGTA |
| | | CACCTGTCACAAAGGAGGCGAGGTTCTAAGCCATTCGCTCCTGCTGCTTCACAAAAAGGAAGAT |
| | | GGAATTTGGTCCACTGATATTTTAAAGGACCAGAAAGAACCCAAAAATAAGACCTTTCTAAGATG |
| | | CGAGGCCAAGAATTATTCTGGACGTTTCACCTGCTGGTGGCTGACGACAATCAGTACTGATTTG |
| | | ACATTCAGTGTCAAAAGCAGCAGAGGCTCTTCTGACCCCCAAGGGGTGACGTGCGGAGCTGCT |
| | | ACACTCTCTGCAGAGAGAGTCAGAGGGGACAACAAGGAGTATGAGTACTCAGTGGAGTGCCAG |
| | | GAGGACAGTGCCTGCCCAGCTGCTGAGGAGAGTCTGCCCATTGAGGTCATGGTGGATGCCGTT |
| | | CACAAGCTCAAGTATGAAAACTACACCAGCAGCTTCTTCATCAGGGACATCATCAAACCTGACC |
| | | CACCCAAGAACTTGCAGCTGAAGCCATTAAAGAATTCTCGGCAGGTGGAGGTCAGCTGGGAGT |
| | | ACCCTGACACCTGGAGTACTCCACATTCCTACTTCTCCCTGACATTCTGCGTTCAGGTCCAGGG |
| | | CAAGAGCAAGAGAGAAAAGAAAGATAGAGTCTTCACGGACAAGACCTCAGCCACGGTCATCTG |
| | | CCGCAAAAATGCCAGCATTAGCGTGCGGGCCCAGGACCGCTACTATAGCTCATCTTGGAGCGA |
| | | ATGGGCATCTGTGCCCTGCAGTGAGGGCAGAGGCAGCCTGCTGACCTGCGGCGACGTCGAGG |
| | | AGAACCCCGGGCCCATGGGGGCAGGTGCCACCGGCCGCGCCATGGACGGGCCGCGCCTGCT |
| | | GCTGTTGCTGCTTCTGGGGGTGTCCCTTGGAGGTGCCAAGGAGGCATGCCCCACAGGCCTGTA |
| | | CACACACAGCGGTGAGTGCTGCAAAGCCTGCAACCTGGGCGAGGGTGTGGCCCAGCCTTGTG |
| | | GAGCCAACCAGACCGTGTGTGAGCCCTGCCTGGACAGCGTGACGTTCTCCGACGTGGTGAGC |
| | | GCGACCGAGCCGTGCAAGCCGTGCACCGAGTGCGTGGGGCTCCAGAGCATGTCGGCGCCGT |
| | | GCGTGGAGGCCGATGACGCCGTGTGCCGCTGCGCCTACGGCTACTACCAGGATGAGACGACT |
| | | GGGCGCTGCGAGGCGTGCCGCGTGTGCGAGGCGGGCTCGGGCCTCGTGTTCTCCTGCCAGG |
| | | ACAAGCAGAACACCGTGTGCGAGGAGTGCCCCGACGGCACGTATTCCGACGAGGCCAACCAC |
| | | GTGGACCCGTGCCTGCCCTGCACCGTGTGCGAGGACACCGAGCGCCAGCTCCGCGAGTGCAC |
| | | ACGCTGGGCCGACGCCGAGTGCGAGGAGATCCCTGGCCGTTGGATTACACGGTCCACACCCC |
| | | CAGAGGGCTCGGACAGCACAGCCCCCAGCACCCAGGAGCCTGAGGCACCTCCAGAACAAGAC |
| | | CTCATAGCCAGCACGGTGGCAGGTGTGGTGACCACAGTGATGGGCAGCTCCCAGCCCGTGGT |
| | | GACCCGAGGCACCACCGACAACCTCATCCCTGTCTATTGCTCCATCCTGGCTGCTGTGGTTGT |
| | | GGGTCTTGTGGCCTACATAGCCTTCAAGAGGTGATCTAGAGGGCCCGTTTAAACCCGCTGATCA |
| | | GCCTCGACTGTGCCTTCTAGTTGCCAGCCATCTGTTGTTTGCCCCTCCCCCGTGCCTTCCTTGA |
| | | CCCTGGAAGGTGCCACTCCCACTGTCCTTTCCTAATAAAATGAGGAAATTGCATCGCATTGTCT |
| | | GAGTAGGTGTCATTCTATTCTGGGGGGTGGGGTGGGGCAGGACAGCAAGGGGGAGGATTGGG |
| | | AAGACAATAGCAGGCATGCTGGGGATGCGGTGGGCTCTATGACTAGTGGCGAATTCGGCGCAG |
| | | ATCAAAGAGAGCCTGCGGGCAGAGCTCAGGGTGACAGGTGCGGCCTCGGAGGCCCCGGGGC |
| | | AGGGGTGAGCTGAGCCGGTCCTGGGGTGGGTGTCCCCTCCTGCACAGGATCAGGAGCTCCAG |
| | | GGTCGTAGGGCAGGGACCCCCCAGCTCCAGTCCAGGGCTCTGTCCTGCACCTGGGGAATGGT |
| | | GACCGGCATCTCTGTCCTCTAGCTCTGGAAGCACCCCAGCCCCTCTAGTCTGCCCTCACCCCT |
| | | GACCCTGACCCTCCACCCTGACCCCGTCCTAACCCCTGACCTTTG |
| 36 | Inserted | ATGAGATCATGTCCTAACCCTGATCCTCTTGTCCCACAGATATCCAGAACCCTGACCCTGTTGCT |
| | matrice TRAC | GGGCCTTTTTCCCATGCCTGCCTTTACTCTGCCAGAGTTATATTGCTGGGGTTTTGAAGAAGATC |
| | locus_CubiCAR | CTATTAAATAAAAGAATAAGCAGTATTATTAAGTAGCCCTGCATTTCAGGTTTCCTTGAGTGGCA |
| | CD22 (60 | GGCCAGGCCTGGCCGTGAACGTTCACTGAAATCATGGCCTCTTGGCCAAGATTGATAGCTTGT |
| | nucleotides | GCCTGTCCCTGAGTCCCAGTCCATCACGAGCAGCTGGTTTCTAAGATGCTATTTCCCGTATAAA |
| | upstream and | GCATGAGACCGTGACTTGCCAGCCCCACAGAGCCCCGCCCTTGTCCATCACTGGCATCTGGAC |
| | downstream) | TCCAGCCTGGGTTGGGGCAAAGAGGGAAATGAGATCATGTCCTAACCCTGATCCTCTTGTCCCA |
| | | CAGATATCCAGTACCCCTACGACGTGCCCGACTACGCCTCCGGTGAGGGCAGAGGAAGTCTTC |
| | | TAACATGCGGTGACGTGGAGGAGAATCCGGGCCCCGGATCCGCTCTGCCCGTCACCGCTCTG |
| | | CTGCTGCCACTGGCACTGCTGCTGCACGCTGCTAGGCCCGGAGGGGGAGGCAGCTGCCCCTA |
| | | CAGCAACCCCAGCCTGTGCAGCGGAGGCGGCGGCAGCGGCGGAGGGGGTAGCCAGGTGCAG |
| | | CTGCAGCAGAGCGGCCCTGGCCTGGTGAAGCCAAGCCAGACACTGTCCCTGACCTGCGCCAT |
| | | CAGCGGCGATTCCGTGAGCTCCAACTCCGCCGCCTGGAATTGGATCAGGCAGTCCCCTTCTCG |
| | | GGGCCTGGAGTGGCTGGGAAGGACATACTATCGGTCTAAGTGGTACAACGATTATGCCGTGTC |
| | | TGTGAAGAGCAGAATCACAATCAACCCTGACACCTCCAAGAATCAGTTCTCTCTGCAGCTGAAT |
| | | AGCGTGACACCAGAGGACACCGCCGTGTACTATTGCGCCAGGGAGGTGACCGGCGACCTGGA |
| | | GGATGCCTTTGACATCTGGGGCCAGGGCACAATGGTGACCGTGAGCTCCGGAGGCGGCGGAT |
| | | CTGGCGGAGGAGGAAGTGGGGGCGGCGGGAGTGATATCCAGATGACACAGTCCCCATCCTCT |
| | | CTGAGCGCCTCCGTGGGCGACAGAGTGACAATCACCTGTAGGGCCTCCCAGACCATCTGGTCT |
| | | TACCTGAACTGGTATCAGCAGAGGCCCGGCAAGGCCCCTAATCTGCTGATCTACGCAGCAAGC |
| | | TCCCTGCAGAGCGGAGTGCCATCCAGATTCTCTGGCAGGGGCTCCGGCACAGACTTCACCCTG |
| | | ACCATCTCTAGCCTGCAGGCCGAGGACTTCGCCACCTACTATTGCCAGCAGTCTTATAGCATCC |
| | | CCCAGACATTTGGCCAGGGCACCAAGCTGGAGATCAAGTCGGATCCCGGAAGCGGAGGGGGA |
| | | GGCAGCTGCCCCTACAGCAACCCCAGCCTGTGCAGCGGAGGCGGCGGCAGCGAGCTGCCCA |
| | | CCCAGGGCACCTTCTCCAACGTGTCCACCAACGTGAGCCCAGCCAAGCCCACCACCACCGCCT |
| | | GTCCTTATTCCAATCCTTCCCTGTGTGCTCCCACCACAACCCCCGCTCCAAGGCCCCCTACCCC |
| | | CGCACCAACTATTGCCTCCCAGCCACTCTCACTGCGGCCTGAGGCCTGTCGGCCCGCTGCTGG |
| | | AGGCGCAGTGCATACAAGGGGCCTCGATTTCGCCTGCGATATTTACATCTGGGCACCCCTCGC |
| | | CGGCACCTGCGGGGTGCTTCTCCTCTCCCTGGTGATTACCCTGTATTGCAGACGGGGCCGGAA |
| | | GAAGCTCCTCTACATTTTTAAGCAGCCTTTCATGCGGCCAGTGCAGACAACCCAAGAGGAGGAT |
| | | GGGTGTTCCTGCAGATTCCCTGAGGAAGAGGAAGGCGGGTGCGAGCTGAGAGTGAAGTTCTC |
| | | CAGGAGCGCAGATGCCCCCGCCTATCAACAGGGCCAGAACCAGCTCTACAACGAGCTTAACCT |
| | | CGGGAGGCGCGAAGAATACGACGTGTTGGATAAGAGAAGGGGGCGGGACCCCGAGATGGGA |
| | | GGAAAGCCCCGGAGGAAGAACCCTCAGGAGGGCCTGTACAACGAGCTGCAGAAGGATAAGAT |
| | | GGCCGAGGCCTACTCAGAGATCGGGATGAAGGGGGAGCGGCGCCGCGGGAAGGGGCACGAT |
| | | GGGCTCTACCAGGGGCTGAGCACAGCCACAAAGGACACATACGACGCCTTGCACATGCAGGC |
| | | CCTTCCACCCCGGGAATAGTCTAGAGGGCCCGTTTAAACCCGCTGATCAGCCTCGACTGTGCC |
| | | TTCTAGTTGCCAGCCATCTGTTGTTTGCCCCTCCCCCGTGCCTTCCTTGACCCTGGAAGGTGCC |
| | | ACTCCCACTGTCCTTTCCTAATAAAATGAGGAAATTGCATCGCATTGTCTGAGTAGGTGTCATTC |
| | | TATTCTGGGGGGTGGGGTGGGGCAGGACAGCAAGGGGGAGGATTGGGAAGACAATAGCAGGC |
| | | ATGCTGGGGATGCGGTGGGCTCTATGACTAGTGGCGAATTCCCGTGTACCAGCTGAGAGACTC |
| | | TAAATCCAGTGACAAGTCTGTCTGCCTATTCACCGATTTTGATTCTCAAACAAATGTGTCACAAA |
| | | GTAAGGATTCTGATGTGTATATCACAGACAAAACTGTGCTAGACATGAGGTCTATGGACTTCAAG |
| | | AGCAACAGTGCTGTGGCCTGGAGCAACAAATCTGACTTTGCATGTGCAAACGCCTTCAACAACA |
| | | GCATTATTCCAGAAGACACCTTCTTCCCCAGCCCAGGTAAGGGCAGCTTTGGTGCCTTCGCAG |
| | | GCTGTTTCCTTGCTTCAGGAATGGCCAGGTTCTGCCCAGAGCTCTGGTCAATGATGTCTAAAAC |
| | | TCCTCTGATTGGTGGTCTCGGCCTTATCCATTGCCACCAAAACCCTCTTTTTACTAAGAAACAGT |
| | | GAGCCTTGTTCTGGCAGTCCAGAGAATGACACGGGAAAAAAGCAGATGAAGA |
| 37 | Inserted | AGTGCTGGCTAGAAACCAAGTGCTTTACTGCATGCACATCATTTAGCACAGTTAGTTGCTGTTTA |
| | matrice CD25 | TTATTCCTGTTCCACAGCTATTGTCTGCCATATAAAAACTTAGGCCAGGCACAGTGGCTCACACC |
| | locus_IL15_ | TGTAATCCCAGCACTTTGGAAGGCCGAGGCAGGCAGATCACAAGGTCAGGAGTTCGAGACCAG |
| | 2A_sIL15Ra | CCTGGCCAACATAGCAAAACCCCATCTCTACTAAAAATACAAAAATTAGCCAGGCATGGTGGCG |
| | (60 | TGTGCACTGGTTTAGAGTGAGGACCACATTTTTTTGGTGCCGTGTTACACATATGACCGTGACTT |
| | nucleotides | TGTTACACCACTACAGGAGGAAGAGTAGAAGAACAATCGGTTCTGGCGTGAAACAGACTTTGAA |
| | upstream and | TTTTGACCTTCTCAAGTTGGCGGGAGACGTGGAGTCCAACCCAGGGCCCGGTACCGGGTCCGC |
| | downstream) | CACCATGGACTGGACCTGGATTCTGTTCCTCGTGGCTGCTGCTACAAGAGTGCACAGCGGCAT |
| | | TCATGTCTTCATTTTGGGCTGTTTCAGTGCAGGGCTTCCTAAAACAGAAGCCAACTGGGTGAAT |
| | | GTAATAAGTGATTTGAAAAAAATTGAAGATCTTATTCAATCTATGCATATTGATGCTACTTTATATA |
| | | CGGAAAGTGATGTTCACCCCAGTTGCAAAGTAACAGCAATGAAGTGCTTTCTCTTGGAGTTACA |
| | | AGTTATTTCACTTGAGTCCGGAGATGCAAGTATTCATGATACAGTAGAAAATCTGATCATCCTAG |
| | | CAAACAACAGTTTGTCTTCTAATGGGAATGTAACAGAATCTGGATGCAAAGAATGTGAGGAACT |
| | | GGAGGAAAAAAATATTAAAGAATTTTTGCAGAGTTTTGTACATATTGTCCAAATGTTCATCAACAC |
| | | TTCTGGAAGCGGAGCTACTAACTTCAGCCTGCTGAAGCAGGCTGGAGACGTGGAGGAGAACCC |
| | | TGGACCTGGGACCGGCTCTGCAACCATGGATTGGACGTGGATCCTGTTTCTCGTGGCAGCTGC |
| | | CACAAGAGTTCACAGTATCACGTGCCCTCCCCCCATGTCCGTGGAACACGCAGACATCTGGGT |
| | | CAAGAGCTACAGCTTGTACTCCAGGGAGCGGTACATTTGTAACTCTGGTTTCAAGCGTAAAGCC |
| | | GGCACGTCCAGCCTGACGGAGTGCGTGTTGAACAAGGCCACGAATGTCGCCCACTGGACAAC |
| | | CCCCAGTCTCAAATGCATTAGAGACCCTGCCCTGGTTCACCAAAGGCCAGCGCCACCCTCCAC |
| | | AGTAACGACGGCAGGGGTGACCCCACAGCCAGAGAGCCTCTCCCCTTCTGGAAAAGAGCCCG |
| | | CAGCTTCATCTCCCAGCTCAAACAACACAGCGGCCACAACAGCAGCTATTGTCCCGGGCTCCC |
| | | AGCTGATGCCTTCAAAATCACCTTCCACAGGAACCACAGAGATAAGCAGTCATGAGTCCTCCCA |
| | | CGGCACCCCCTCTCAGACAACAGCCAAGAACTGGGAACTCACAGCATCCGCCTCCCACCAGCC |
| | | GCCAGGTGTGTATCCACAGGGCCACAGCGACACCACTGAGGGCAGAGGCAGCCTGCTGACCT |
| | | GCGGCGACGTCGAGGAGAACCCCGGGCCCATGGGGGCAGGTGCCACCGGCCGCGCCATGGA |
| | | CGGGCCGCGCCTGCTGCTGTTGCTGCTTCTGGGGGTGTCCCTTGGAGGTGCCAAGGAGGCAT |
| | | GCCCCACAGGCCTGTACACACACAGCGGTGAGTGCTGCAAAGCCTGCAACCTGGGCGAGGGT |
| | | GTGGCCCAGCCTTGTGGAGCCAACCAGACCGTGTGTGAGCCCTGCCTGGACAGCGTGACGTT |
| | | CTCCGACGTGGTGAGCGCGACCGAGCCGTGCAAGCCGTGCACCGAGTGCGTGGGGCTCCAGA |
| | | GCATGTCGGCGCCGTGCGTGGAGGCCGATGACGCCGTGTGCCGCTGCGCCTACGGCTACTAC |
| | | CAGGATGAGACGACTGGGCGCTGCGAGGCGTGCCGCGTGTGCGAGGCGGGCTCGGGCCTCG |
| | | TGTTCTCCTGCCAGGACAAGCAGAACACCGTGTGCGAGGAGTGCCCCGACGGCACGTATTCCG |
| | | ACGAGGCCAACCACGTGGACCCGTGCCTGCCCTGCACCGTGTGCGAGGACACCGAGCGCCAG |
| | | CTCCGCGAGTGCACACGCTGGGCCGACGCCGAGTGCGAGGAGATCCCTGGCCGTTGGATTAC |
| | | ACGGTCCACACCCCCAGAGGGCTCGGACAGCACAGCCCCCAGCACCCAGGAGCCTGAGGCAC |
| | | CTCCAGAACAAGACCTCATAGCCAGCACGGTGGCAGGTGTGGTGACCACAGTGATGGGCAGCT |
| | | CCCAGCCCGTGGTGACCCGAGGCACCACCGACAACCTCATCCCTGTCTATTGCTCCATCCTGG |
| | | CTGCTGTGGTTGTGGGTCTTGTGGCCTACATAGCCTTCAAGAGGTGAAAAACCAAAAGAACAAG |
| | | AATTTCTTGGTAAGAAGCCGGGAACAGACAACAGAAGTCATGAAGCCCAAGTGAAATCAAAGGT |
| | | GCTAAATGGTCGCCCAGGAGACATCCGTTGTGCTTGCCTGCGTTTTGGAAGCTCTGAAGTCACA |
| | | TCACAGGACACGGGGCAGTGGCAACCTTGTCTCTATGCCAGCTCAGTCCCATCAGAGAGCGAG |
| | | CGCTACCCACTTCTAAATAGCAATTTCGCCGTTGAAGAGGAAGGGCAAAACCACTAGAACTCTC |
| | | CATCTTATTTTCATGTATATGTGTTCATTAAAGCATGAATGGTATGGAACTCTCTCCACCCTATAT |
| | | GTAGTATAAAGAAAAGTAGGTT |
| 38 | Inserted | GGTGGCCGGGGAGGCTTTGTGGGGCCACCCAGCCCCTTCCTCACCTCTCTCCATCTCTCAGAC |
| | matrice PD1 | TCCCCAGACAGGCCCTGGAACCCCCCCACCTTCTCCCCAGCCCTGCTCGTGGTGACCGAAGG |
| | locus_IL15_ | GGACAACGCCACCTTCACCTGCAGCTTCTCCAACACATCGGAGAGCTTCGTGCTAAACTGGTAC |
| | 2A_sIL15Ra | CGCATGAGCCCCAGCAACCAGACGGACAAGCTGGCCGCCTTCCCCGAGGACCGCAGCCAGCC |
| | (60 | CGGCCAGGACTGCCGCTTCCGTGTCACACAACTGCCCAACGGGCGTGACTTCCACATGAGCGT |
| | nucleotides | GGTCAGGGCCCGGCGCAATGACAGCGGCACCTACCTCTGTGGGGCCGGTTCTGGCGTGAAAC |
| | upstream and | AGACTTTGAATTTTGACCTTCTCAAGTTGGCGGGAGACGTGGAGTCCAACCCAGGGCCCGGTA |
| | downstream) | CCGGGTCCGCCACCATGGACTGGACCTGGATTCTGTTCCTCGTGGCTGCTGCTACAAGAGTGC |
| | | ACAGCGGCATTCATGTCTTCATTTTGGGCTGTTTCAGTGCAGGGCTTCCTAAAACAGAAGCCAA |
| | | CTGGGTGAATGTAATAAGTGATTTGAAAAAAATTGAAGATCTTATTCAATCTATGCATATTGATGC |
| | | TACTTTATATACGGAAAGTGATGTTCACCCCAGTTGCAAAGTAACAGCAATGAAGTGCTTTCTCT |
| | | TGGAGTTACAAGTTATTTCACTTGAGTCCGGAGATGCAAGTATTCATGATACAGTAGAAAATCTG |
| | | ATCATCCTAGCAAACAACAGTTTGTCTTCTAATGGGAATGTAACAGAATCTGGATGCAAAGAATG |
| | | TGAGGAACTGGAGGAAAAAAATATTAAAGAATTTTTGCAGAGTTTTGTACATATTGTCCAAATGTT |
| | | CATCAACACTTCTGGAAGCGGAGCTACTAACTTCAGCCTGCTGAAGCAGGCTGGAGACGTGGA |
| | | GGAGAACCCTGGACCTGGGACCGGCTCTGCAACCATGGATTGGACGTGGATCCTGTTTCTCGT |
| | | GGCAGCTGCCACAAGAGTTCACAGTATCACGTGCCCTCCCCCCATGTCCGTGGAACACGCAGA |
| | | CATCTGGGTCAAGAGCTACAGCTTGTACTCCAGGGAGCGGTACATTTGTAACTCTGGTTTCAAG |
| | | CGTAAAGCCGGCACGTCCAGCCTGACGGAGTGCGTGTTGAACAAGGCCACGAATGTCGCCCA |
| | | CTGGACAACCCCCAGTCTCAAATGCATTAGAGACCCTGCCCTGGTTCACCAAAGGCCAGCGCC |
| | | ACCCTCCACAGTAACGACGGCAGGGGTGACCCCACAGCCAGAGAGCCTCTCCCCTTCTGGAAA |
| | | AGAGCCCGCAGCTTCATCTCCCAGCTCAAACAACACAGCGGCCACAACAGCAGCTATTGTCCC |
| | | GGGCTCCCAGCTGATGCCTTCAAAATCACCTTCCACAGGAACCACAGAGATAAGCAGTCATGAG |
| | | TCCTCCCACGGCACCCCCTCTCAGACAACAGCCAAGAACTGGGAACTCACAGCATCCGCCTCC |
| | | CACCAGCCGCCAGGTGTGTATCCACAGGGCCACAGCGACACCACTGAGGGCAGAGGCAGCCT |
| | | GCTGACCTGCGGCGACGTCGAGGAGAACCCCGGGCCCATGGGGGCAGGTGCCACCGGCCGC |
| | | GCCATGGACGGGCCGCGCCTGCTGCTGTTGCTGCTTCTGGGGGTGTCCCTTGGAGGTGCCAA |
| | | GGAGGCATGCCCCACAGGCCTGTACACACACAGCGGTGAGTGCTGCAAAGCCTGCAACCTGG |
| | | GCGAGGGTGTGGCCCAGCCTTGTGGAGCCAACCAGACCGTGTGTGAGCCCTGCCTGGACAGC |
| | | GTGACGTTCTCCGACGTGGTGAGCGCGACCGAGCCGTGCAAGCCGTGCACCGAGTGCGTGGG |
| | | GCTCCAGAGCATGTCGGCGCCGTGCGTGGAGGCCGATGACGCCGTGTGCCGCTGCGCCTACG |
| | | GCTACTACCAGGATGAGACGACTGGGCGCTGCGAGGCGTGCCGCGTGTGCGAGGCGGGCTC |
| | | GGGCCTCGTGTTCTCCTGCCAGGACAAGCAGAACACCGTGTGCGAGGAGTGCCCCGACGGCA |
| | | CGTATTCCGACGAGGCCAACCACGTGGACCCGTGCCTGCCCTGCACCGTGTGCGAGGACACC |
| | | GAGCGCCAGCTCCGCGAGTGCACACGCTGGGCCGACGCCGAGTGCGAGGAGATCCCTGGCC |
| | | GTTGGATTACACGGTCCACACCCCCAGAGGGCTCGGACAGCACAGCCCCCAGCACCCAGGAG |
| | | CCTGAGGCACCTCCAGAACAAGACCTCATAGCCAGCACGGTGGCAGGTGTGGTGACCACAGTG |
| | | ATGGGCAGCTCCCAGCCCGTGGTGACCCGAGGCACCACCGACAACCTCATCCCTGTCTATTGC |
| | | TCCATCCTGGCTGCTGTGGTTGTGGGTCTTGTGGCCTACATAGCCTTCAAGAGGTGATCTAGAG |
| | | GGCCCGTTTAAACCCGCTGATCAGCCTCGACTGTGCCTTCTAGTTGCCAGCCATCTGTTGTTTG |
| | | CCCCTCCCCCGTGCCTTCCTTGACCCTGGAAGGTGCCACTCCCACTGTCCTTTCCTAATAAAAT |
| | | GAGGAAATTGCATCGCATTGTCTGAGTAGGTGTCATTCTATTCTGGGGGGTGGGGTGGGGCAG |
| | | GACAGCAAGGGGGAGGATTGGGAAGACAATAGCAGGCATGCTGGGGATGCGGTGGGCTCTAT |
| | | GACTAGTGGCGAATTCGGCGCAGATCAAAGAGAGCCTGCGGGCAGAGCTCAGGGTGACAGGT |
| | | GCGGCCTCGGAGGCCCCGGGGCAGGGGTGAGCTGAGCCGGTCCTGGGGTGGGTGTCCCCTC |
| | | CTGCACAGGATCAGGAGCTCCAGGGTCGTAGGGCAGGGACCCCCCAGCTCCAGTCCAGGGCT |
| | | CTGTCCTGCACCTGGGGAATGGTGACCGGCATCTCTGTCCTCTAGCTCTGGAAGCACCCCAGC |
| | | CCCTCTAGTCTGCCCTCACCCCTGACCCTGACCCTCCACCCTGACCCCGTCCTAACCCCTGAC |
| | | CTTTGTGCCCTTCCAGAGAGAAGGGCAGAAGTGCCCACAGCCCACCCCAGCCCCTCACCCAGG |
| | | CC |
| 39 | Inserted | AGTGCTGGCTAGAAACCAAGTGCTTTACTGCATGCACATCATTTAGCACAGTTAGTTGCTGTTTA |
| | matrice CD25 | TTATTCCTGTTCCACAGCTATTGTCTGCCATATAAAAACTTAGGCCAGGCACAGTGGCTCACACC |
| | locus_IL12a_ | TGTAATCCCAGCACTTTGGAAGGCCGAGGCAGGCAGATCACAAGGTCAGGAGTTCGAGACCAG |
| | 2A_IL12b (60 | CCTGGCCAACATAGCAAAACCCCATCTCTACTAAAAATACAAAAATTAGCCAGGCATGGTGGCG |
| | nucleotides | TGTGCACTGGTTTAGAGTGAGGACCACATTTTTTTGGTGCCGTGTTACACATATGACCGTGACTT |
| | upstream and | TGTTACACCACTACAGGAGGAAGAGTAGAAGAACAATCGGTTCTGGCGTGAAACAGACTTTGAA |
| | downstream) | TTTTGACCTTCTCAAGTTGGCGGGAGACGTGGAGTCCAACCCAGGGCCCATGTGGCCCCCTGG |
| | | GTCAGCCTCCCAGCCACCGCCCTCACCTGCCGCGGCCACAGGTCTGCATCCAGCGGCTCGCC |
| | | CTGTGTCCCTGCAGTGCCGGCTCAGCATGTGTCCAGCGCGCAGCCTCCTCCTTGTGGCTACCC |
| | | TGGTCCTCCTGGACCACCTCAGTTTGGCCAGAAACCTCCCCGTGGCCACTCCAGACCCAGGAA |
| | | TGTTCCCATGCCTTCACCACTCCCAAAACCTGCTGAGGGCCGTCAGCAACATGCTCCAGAAGG |
| | | CCAGACAAACTCTAGAATTTTACCCTTGCACTTCTGAAGAGATTGATCATGAAGATATCACAAAA |
| | | GATAAAACCAGCACAGTGGAGGCCTGTTTACCATTGGAATTAACCAAGAATGAGAGTTGCCTAA |
| | | ATTCCAGAGAGACCTCTTTCATAACTAATGGGAGTTGCCTGGCCTCCAGAAAGACCTCTTTTATG |
| | | ATGGCCCTGTGCCTTAGTAGTATTTATGAAGACTTGAAGATGTACCAGGTGGAGTTCAAGACCA |
| | | TGAATGCAAAGCTTCTGATGGATCCTAAGAGGCAGATCTTTCTAGATCAAAACATGCTGGCAGTT |
| | | ATTGATGAGCTGATGCAGGCCCTGAATTTCAACAGTGAGACTGTGCCACAAAAATCCTCCCTTG |
| | | AAGAACCGGATTTTTATAAAACTAAAATCAAGCTCTGCATACTTCTTCATGCTTTCAGAATTCGGG |
| | | CAGTGACTATTGATAGAGTGATGAGCTATCTGAATGCTTCCGGAAGCGGAGCTACTAACTTCAG |
| | | CCTGCTGAAGCAGGCTGGAGACGTGGAGGAGAACCCTGGACCTATGTGTCACCAGCAGTTGGT |
| | | CATCTCTTGGTTTTCCCTGGTTTTTCTGGCATCTCCCCTCGTGGCCATATGGGAACTGAAGAAA |
| | | GATGTTTATGTCGTAGAATTGGATTGGTATCCGGATGCCCCTGGAGAAATGGTGGTCCTCACCT |
| | | GTGACACCCCTGAAGAAGATGGTATCACCTGGACCTTGGACCAGAGCAGTGAGGTCTTAGGCT |
| | | CTGGCAAAACCCTGACCATCCAAGTCAAAGAGTTTGGAGATGCTGGCCAGTACACCTGTCACAA |
| | | AGGAGGCGAGGTTCTAAGCCATTCGCTCCTGCTGCTTCACAAAAAGGAAGATGGAATTTGGTCC |
| | | ACTGATATTTTAAAGGACCAGAAAGAACCCAAAAATAAGACCTTTCTAAGATGCGAGGCCAAGAA |
| | | TTATTCTGGACGTTTCACCTGCTGGTGGCTGACGACAATCAGTACTGATTTGACATTCAGTGTCA |
| | | AAAGCAGCAGAGGCTCTTCTGACCCCCAAGGGGTGACGTGCGGAGCTGCTACACTCTCTGCAG |
| | | AGAGAGTCAGAGGGGACAACAAGGAGTATGAGTACTCAGTGGAGTGCCAGGAGGACAGTGCC |
| | | TGCCCAGCTGCTGAGGAGAGTCTGCCCATTGAGGTCATGGTGGATGCCGTTCACAAGCTCAAG |
| | | TATGAAAACTACACCAGCAGCTTCTTCATCAGGGACATCATCAAACCTGACCCACCCAAGAACTT |
| | | GCAGCTGAAGCCATTAAAGAATTCTCGGCAGGTGGAGGTCAGCTGGGAGTACCCTGACACCTG |
| | | GAGTACTCCACATTCCTACTTCTCCCTGACATTCTGCGTTCAGGTCCAGGGCAAGAGCAAGAGA |
| | | GAAAAGAAAGATAGAGTCTTCACGGACAAGACCTCAGCCACGGTCATCTGCCGCAAAAATGCCA |
| | | GCATTAGCGTGCGGGCCCAGGACCGCTACTATAGCTCATCTTGGAGCGAATGGGCATCTGTGC |
| | | CCTGCAGTGAGGGCAGAGGCAGCCTGCTGACCTGCGGCGACGTCGAGGAGAACCCCGGGCC |
| | | CATGGGGGCAGGTGCCACCGGCCGCGCCATGGACGGGCCGCGCCTGCTGCTGTTGCTGCTTC |
| | | TGGGGGTGTCCCTTGGAGGTGCCAAGGAGGCATGCCCCACAGGCCTGTACACACACAGCGGT |
| | | GAGTGCTGCAAAGCCTGCAACCTGGGCGAGGGTGTGGCCCAGCCTTGTGGAGCCAACCAGAC |
| | | CGTGTGTGAGCCCTGCCTGGACAGCGTGACGTTCTCCGACGTGGTGAGCGCGACCGAGCCGT |
| | | GCAAGCCGTGCACCGAGTGCGTGGGGCTCCAGAGCATGTCGGCGCCGTGCGTGGAGGCCGA |
| | | TGACGCCGTGTGCCGCTGCGCCTACGGCTACTACCAGGATGAGACGACTGGGCGCTGCGAGG |
| | | CGTGCCGCGTGTGCGAGGCGGGCTCGGGCCTCGTGTTCTCCTGCCAGGACAAGCAGAACACC |
| | | GTGTGCGAGGAGTGCCCCGACGGCACGTATTCCGACGAGGCCAACCACGTGGACCCGTGCCT |
| | | GCCCTGCACCGTGTGCGAGGACACCGAGCGCCAGCTCCGCGAGTGCACACGCTGGGCCGAC |
| | | GCCGAGTGCGAGGAGATCCCTGGCCGTTGGATTACACGGTCCACACCCCCAGAGGGCTCGGA |
| | | CAGCACAGCCCCCAGCACCCAGGAGCCTGAGGCACCTCCAGAACAAGACCTCATAGCCAGCA |
| | | CGGTGGCAGGTGTGGTGACCACAGTGATGGGCAGCTCCCAGCCCGTGGTGACCCGAGGCACC |
| | | ACCGACAACCTCATCCCTGTCTATTGCTCCATCCTGGCTGCTGTGGTTGTGGGTCTTGTGGCCT |
| | | ACATAGCCTTCAAGAGGTGAAAAACCAAAAGAACAAGAATTTCTTGGTAAGAAGCCGGGAACAG |
| | | ACAACAGAAGTCATGAAGCCCAAGTGAAATCAAAGGTGCTAAATGGTCGCCCAGGAGACATCC |
| | | GTTGTGCTTGCCTGCGTTTTGGAAGCTCTGAAGTCACATCACAGGACACGGGGCAGTGGCAAC |
| | | CTTGTCTCTATGCCAGCTCAGTCCCATCAGAGAGCGAGCGCTACCCACTTCTAAATAGCAATTT |
| | | CGCCGTTGAAGAGGAAGGGCAAAACCACTAGAACTCTCCATCTTATTTTCATGTATATGTGTTCA |
| | | TGAATGGTATGGAACTCTCTCCACCCTATATGTAGTATAAAGAAAAGTAGGTT |
| 40 | Inserted | GGTGGCCGGGGAGGCTTTGTGGGGCCACCCAGCCCCTTCCTCACCTCTCTCCATCTCTCAGAC |
| | matrice PD1 | TCCCCAGACAGGCCCTGGAACCCCCCCACCTTCTCCCCAGCCCTGCTCGTGGTGACCGAAGG |
| | locus_IL12a_ | GGACAACGCCACCTTCACCTGCAGCTTCTCCAACACATCGGAGAGCTTCGTGCTAAACTGGTAC |
| | 2A_IL12b (60 | CGCATGAGCCCCAGCAACCAGACGGACAAGCTGGCCGCCTTCCCCGAGGACCGCAGCCAGCC |
| | nucleotides | CGGCCAGGACTGCCGCTTCCGTGTCACACAACTGCCCAACGGGCGTGACTTCCACATGAGCGT |
| | upstream and | GGTCAGGGCCCGGCGCAATGACAGCGGCACCTACCTCTGTGGGGCCGGTTCTGGCGTGAAAC |
| | downstream) | AGACTTTGAATTTTGACCTTCTCAAGTTGGCGGGAGACGTGGAGTCCAACCCAGGGCCCATGT |
| | | GGCCCCCTGGGTCAGCCTCCCAGCCACCGCCCTCACCTGCCGCGGCCACAGGTCTGCATCCA |
| | | GCGGCTCGCCCTGTGTCCCTGCAGTGCCGGCTCAGCATGTGTCCAGCGCGCAGCCTCCTCCTT |
| | | GTGGCTACCCTGGTCCTCCTGGACCACCTCAGTTTGGCCAGAAACCTCCCCGTGGCCACTCCA |
| | | GACCCAGGAATGTTCCCATGCCTTCACCACTCCCAAAACCTGCTGAGGGCCGTCAGCAACATG |
| | | CTCCAGAAGGCCAGACAAACTCTAGAATTTTACCCTTGCACTTCTGAAGAGATTGATCATGAAGA |
| | | TATCACAAAAGATAAAACCAGCACAGTGGAGGCCTGTTTACCATTGGAATTAACCAAGAATGAG |
| | | AGTTGCCTAAATTCCAGAGAGACCTCTTTCATAACTAATGGGAGTTGCCTGGCCTCCAGAAAGA |
| | | CCTCTTTTATGATGGCCCTGTGCCTTAGTAGTATTTATGAAGACTTGAAGATGTACCAGGTGGAG |
| | | TTCAAGACCATGAATGCAAAGCTTCTGATGGATCCTAAGAGGCAGATCTTTCTAGATCAAAACAT |
| | | GCTGGCAGTTATTGATGAGCTGATGCAGGCCCTGAATTTCAACAGTGAGACTGTGCCACAAAAA |
| | | TCCTCCCTTGAAGAACCGGATTTTTATAAAACTAAAATCAAGCTCTGCATACTTCTTCATGCTTTC |
| | | AGAATTCGGGCAGTGACTATTGATAGAGTGATGAGCTATCTGAATGCTTCCGGAAGCGGAGCTA |
| | | CTAACTTCAGCCTGCTGAAGCAGGCTGGAGACGTGGAGGAGAACCCTGGACCTATGTGTCACC |
| | | AGCAGTTGGTCATCTCTTGGTTTTCCCTGGTTTTTCTGGCATCTCCCCTCGTGGCCATATGGGAA |
| | | CTGAAGAAAGATGTTTATGTCGTAGAATTGGATTGGTATCCGGATGCCCCTGGAGAAATGGTGG |
| | | TCCTCACCTGTGACACCCCTGAAGAAGATGGTATCACCTGGACCTTGGACCAGAGCAGTGAGG |
| | | TCTTAGGCTCTGGCAAAACCCTGACCATCCAAGTCAAAGAGTTTGGAGATGCTGGCCAGTACAC |
| | | CTGTCACAAAGGAGGCGAGGTTCTAAGCCATTCGCTCCTGCTGCTTCACAAAAAGGAAGATGGA |
| | | ATTTGGTCCACTGATATTTTAAAGGACCAGAAAGAACCCAAAAATAAGACCTTTCTAAGATGCGA |
| | | GGCCAAGAATTATTCTGGACGTTTCACCTGCTGGTGGCTGACGACAATCAGTACTGATTTGACA |
| | | TTCAGTGTCAAAAGCAGCAGAGGCTCTTCTGACCCCCAAGGGGTGACGTGCGGAGCTGCTACA |
| | | CTCTCTGCAGAGAGAGTCAGAGGGGACAACAAGGAGTATGAGTACTCAGTGGAGTGCCAGGAG |
| | | GACAGTGCCTGCCCAGCTGCTGAGGAGAGTCTGCCCATTGAGGTCATGGTGGATGCCGTTCAC |
| | | AAGCTCAAGTATGAAAACTACACCAGCAGCTTCTTCATCAGGGACATCATCAAACCTGACCCAC |
| | | CCAAGAACTTGCAGCTGAAGCCATTAAAGAATTCTCGGCAGGTGGAGGTCAGCTGGGAGTACC |
| | | CTGACACCTGGAGTACTCCACATTCCTACTTCTCCCTGACATTCTGCGTTCAGGTCCAGGGCAA |
| | | GAGCAAGAGAGAAAAGAAAGATAGAGTCTTCACGGACAAGACCTCAGCCACGGTCATCTGCCG |
| | | CAAAAATGCCAGCATTAGCGTGCGGGCCCAGGACCGCTACTATAGCTCATCTTGGAGCGAATG |
| | | GGCATCTGTGCCCTGCAGTGAGGGCAGAGGCAGCCTGCTGACCTGCGGCGACGTCGAGGAGA |
| | | ACCCCGGGCCCATGGGGGCAGGTGCCACCGGCCGCGCCATGGACGGGCCGCGCCTGCTGCT |
| | | GTTGCTGCTTCTGGGGGTGTCCCTTGGAGGTGCCAAGGAGGCATGCCCCACAGGCCTGTACAC |
| | | ACACAGCGGTGAGTGCTGCAAAGCCTGCAACCTGGGCGAGGGTGTGGCCCAGCCTTGTGGAG |
| | | CCAACCAGACCGTGTGTGAGCCCTGCCTGGACAGCGTGACGTTCTCCGACGTGGTGAGCGCG |
| | | ACCGAGCCGTGCAAGCCGTGCACCGAGTGCGTGGGGCTCCAGAGCATGTCGGCGCCGTGCGT |
| | | GGAGGCCGATGACGCCGTGTGCCGCTGCGCCTACGGCTACTACCAGGATGAGACGACTGGGC |
| | | GCTGCGAGGCGTGCCGCGTGTGCGAGGCGGGCTCGGGCCTCGTGTTCTCCTGCCAGGACAAG |
| | | CAGAACACCGTGTGCGAGGAGTGCCCCGACGGCACGTATTCCGACGAGGCCAACCACGTGGA |
| | | CCCGTGCCTGCCCTGCACCGTGTGCGAGGACACCGAGCGCCAGCTCCGCGAGTGCACACGCT |
| | | GGGCCGACGCCGAGTGCGAGGAGATCCCTGGCCGTTGGATTACACGGTCCACACCCCCAGAG |
| | | GGCTCGGACAGCACAGCCCCCAGCACCCAGGAGCCTGAGGCACCTCCAGAACAAGACCTCAT |
| | | AGCCAGCACGGTGGCAGGTGTGGTGACCACAGTGATGGGCAGCTCCCAGCCCGTGGTGACCC |
| | | GAGGCACCACCGACAACCTCATCCCTGTCTATTGCTCCATCCTGGCTGCTGTGGTTGTGGGTCT |
| | | TGTGGCCTACATAGCCTTCAAGAGGTGATCTAGAGGGCCCGTTTAAACCCGCTGATCAGCCTC |
| | | GACTGTGCCTTCTAGTTGCCAGCCATCTGTTGTTTGCCCCTCCCCCGTGCCTTCCTTGACCCTG |
| | | GAAGGTGCCACTCCCACTGTCCTTTCCTAATAAAATGAGGAAATTGCATCGCATTGTCTGAGTA |
| | | GGTGTCATTCTATTCTGGGGGGTGGGGTGGGGCAGGACAGCAAGGGGGAGGATTGGGAAGAC |
| | | AATAGCAGGCATGCTGGGGATGCGGTGGGCTCTATGACTAGTGGCGAATTCGGCGCAGATCAA |
| | | AGAGAGCCTGCGGGCAGAGCTCAGGGTGACAGGTGCGGCCTCGGAGGCCCCGGGGCAGGGG |
| | | TGAGCTGAGCCGGTCCTGGGGTGGGTGTCCCCTCCTGCACAGGATCAGGAGCTCCAGGGTCG |
| | | TAGGGCAGGGACCCCCCAGCTCCAGTCCAGGGCTCTGTCCTGCACCTGGGGAATGGTGACCG |
| | | GCATCTCTGTCCTCTAGCTCTGGAAGCACCCCAGCCCCTCTAGTCTGCCCTCACCCCTGACCCT |
| | | GACCCTCCACCCTGACCCCGTCCTAACCCCTGACCTTTGTGCCCTTCCAGAGAGAAGGGCAGA |
| | | AGTGCCCACAGCCCACCCCAGCCCCTCACCCAGGCC |
| 41 | upstream | ATGAGATCATGTCCTAACCCTGATCCTCTTGTCCCACAGATATCCAGAACCCTGACC |
| | TRAC locus | CTG |
| | polynucleotide | |
| | sequence | |
| 42 | downstream | GAAACAGTGAGCCTTGTTCTGGCAGTCCAGAGAATGACACGGGAAAAAAGCAGATG |
| | TRAC locus | AAGA |
| | polynucleotide | |
| | sequence | |
| 43 | upstream | AGTGCTGGCTAGAAACCAAGTGCTTTACTGCATGCACATCATTTAGCACAGTTAGTT |
| | CD25 locus | GCT |
| | polynucleotide | |
| | sequence | |
| 44 | downstream | GAATGGTATGGAACTCTCTCCACCCTATATGTAGTATAAAGAAAAGTAGGTT |
| | CD25 locus | |
| | polynucleotide | |
| | sequence | |
| 45 | upstream PD1 | GGTGGCCGGGGAGGCTTTGTGGGGCCACCCAGCCCCTTCCTCACCTCTCTCCATCT |
| | locus | CTCA |
| | polynucleotide | |
| | sequence | |
| 46 | downstream | TGCCCTTCCAGAGAGAAGGGCAGAAGTGCCCACAGCCCACCCCAGCCCCTCACCC |
| | PD1 locus | AGGCC |
| | polynucleotide | |
| | sequence | |
| 47 | IL-12a | ATGTGGCCCCCTGGGTCAGCCTCCCAGCCACCGCCCTCACCTGCCGCGGCCACAG |
| | polynucleotide | GTCTGCATCCAGCGGCTCGCCCTGTGTCCCTGCAGTGCCGGCTCAGCATGTGTCCA |
| | | GCGCGCAGCCTCCTCCTTGTGGCTACCCTGGTCCTCCTGGACCACCTCAGTTTGGC |
| | | CAGAAACCTCCCCGTGGCCACTCCAGACCCAGGAATGTTCCCATGCCTTCACCACT |
| | | CCCAAAACCTGCTGAGGGCCGTCAGCAACATGCTCCAGAAGGCCAGACAAACTCTA |
| | | GAATTTTACCCTTGCACTTCTGAAGAGATTGATCATGAAGATATCACAAAAGATAAAA |
| | | CCAGCACAGTGGAGGCCTGTTTACCATTGGAATTAACCAAGAATGAGAGTTGCCTAA |
| | | ATTCCAGAGAGACCTCTTTCATAACTAATGGGAGTTGCCTGGCCTCCAGAAAGACCT |
| | | CTTTTATGATGGCCCTGTGCCTTAGTAGTATTTATGAAGACTTGAAGATGTACCAGGT |
| | | GGAGTTCAAGACCATGAATGCAAAGCTTCTGATGGATCCTAAGAGGCAGATCTTTCT |
| | | AGATCAAAACATGCTGGCAGTTATTGATGAGCTGATGCAGGCCCTGAATTTCAACAG |
| | | TGAGACTGTGCCACAAAAATCCTCCCTTGAAGAACCGGATTTTTATAAAACTAAAATC |
| | | AAGCTCTGCATACTTCTTCATGCTTTCAGAATTCGGGCAGTGACTATTGATAGAGTGA |
| | | TGAGCTATCTGAATGCTTCC |
| 48 | IL12b | ATGTGTCACCAGCAGTTGGTCATCTCTTGGTTTTCCCTGGTTTTTCTGGCATCTCCCC |
| | polynucleotide | TCGTGGCCATATGGGAACTGAAGAAAGATGTTTATGTCGTAGAATTGGATTGGTATC |
| | | CGGATGCCCCTGGAGAAATGGTGGTCCTCACCTGTGACACCCCTGAAGAAGATGGT |
| | | ATCACCTGGACCTTGGACCAGAGCAGTGAGGTCTTAGGCTCTGGCAAAACCCTGAC |
| | | CATCCAAGTCAAAGAGTTTGGAGATGCTGGCCAGTACACCTGTCACAAAGGAGGCG |
| | | AGGTTCTAAGCCATTCGCTCCTGCTGCTTCACAAAAAGGAAGATGGAATTTGGTCCA |
| | | CTGATATTTTAAAGGACCAGAAAGAACCCAAAAATAAGACCTTTCTAAGATGCGAGG |
| | | CCAAGAATTATTCTGGACGTTTCACCTGCTGGTGGCTGACGACAATCAGTACTGATT |
| | | TGACATTCAGTGTCAAAAGCAGCAGAGGCTCTTCTGACCCCCAAGGGGTGACGTGC |
| | | GGAGCTGCTACACTCTCTGCAGAGAGAGTCAGAGGGGACAACAAGGAGTATGAGTA |
| | | CTCAGTGGAGTGCCAGGAGGACAGTGCCTGCCCAGCTGCTGAGGAGAGTCTGCCC |
| | | ATTGAGGTCATGGTGGATGCCGTTCACAAGCTCAAGTATGAAAACTACACCAGCAGC |
| | | TTCTTCATCAGGGACATCATCAAACCTGACCCACCCAAGAACTTGCAGCTGAAGCCA |
| | | TTAAAGAATTCTCGGCAGGTGGAGGTCAGCTGGGAGTACCCTGACACCTGGAGTAC |
| | | TCCACATTCCTACTTCTCCCTGACATTCTGCGTTCAGGTCCAGGGCAAGAGCAAGAG |
| | | AGAAAAGAAAGATAGAGTCTTCACGGACAAGACCTCAGCCACGGTCATCTGCCGCA |
| | | AAAATGCCAGCATTAGCGTGCGGGCCCAGGACCGCTACTATAGCTCATCTTGGAGC |
| | | GAATGGGCATCTGTGCCCTGCAGT |
| 49 | IL15 | GGCATTCATGTCTTCATTTTGGGCTGTTTCAGTGCAGGGCTTCCTAAAACAGAAGCC |
| | polynucleotide | AACTGGGTGAATGTAATAAGTGATTTGAAAAAAATTGAAGATCTTATTCAATCTATGC |
| | | ATATTGATGCTACTTTATATACGGAAAGTGATGTTCACCCCAGTTGCAAAGTAACAGC |
| | | AATGAAGTGCTTTCTCTTGGAGTTACAAGTTATTTCACTTGAGTCCGGAGATGCAAGT |
| | | ATTCATGATACAGTAGAAAATCTGATCATCCTAGCAAACAACAGTTTGTCTTCTAATG |
| | | GGAATGTAACAGAATCTGGATGCAAAGAATGTGAGGAACTGGAGGAAAAAAATATTA |
| | | AAGAATTTTTGCAGAGTTTTGTACATATTGTCCAAATGTTCATCAACACTTCT |
| 50 | sIL15ra | ATCACGTGCCCTCCCCCCATGTCCGTGGAACACGCAGACATCTGGGTCAAGAGCTA |
| | polynucleotide | CAGCTTGTACTCCAGGGAGCGGTACATTTGTAACTCTGGTTTCAAGCGTAAAGCCGG |
| | | CACGTCCAGCCTGACGGAGTGCGTGTTGAACAAGGCCACGAATGTCGCCCACTGGA |
| | | CAACCCCCAGTCTCAAATGCATTAGAGACCCTGCCCTGGTTCACCAAAGGCCAGCG |
| | | CCACCCTCCACAGTAACGACGGCAGGGGTGACCCCACAGCCAGAGAGCCTCTCCC |
| | | CTTCTGGAAAAGAGCCCGCAGCTTCATCTCCCAGCTCAAACAACACAGCGGCCACA |
| | | ACAGCAGCTATTGTCCCGGGCTCCCAGCTGATGCCTTCAAAATCACCTTCCACAGGA |
| | | ACCACAGAGATAAGCAGTCATGAGTCCTCCCACGGCACCCCCTCTCAGACAACAGC |
| | | CAAGAACTGGGAACTCACAGCATCCGCCTCCCACCAGCCGCCAGGTGTGTATCCAC |
| | | AGGGCCACAGCGACACCACT |
| 51 | soluble | ATGCTGACACTGCAGACTTGGCTGGTGCAGGCACTGTTTATTTTTCTGACTACTGAA |
| | GP130 | TCAACTGGCGAACTGCTGGACCCTTGTGGCTACATCAGCCCTGAGTCCCCAGTGGT |
| | polynucleotide | GCAGCTGCACAGCAACTTCACCGCCGTGTGCGTGCTGAAGGAGAAGTGTATGGACT |
| | | ACTTTCACGTGAACGCCAATTATATCGTGTGGAAAACCAACCACTTCACAATCCCCAA |
| | | GGAGCAGTACACCATCATCAATAGGACAGCCAGCTCCGTGACCTTTACAGACATCG |
| | | CCTCCCTGAACATCCAGCTGACCTGCAATATCCTGACATTCGGCCAGCTGGAGCAG |
| | | AACGTGTATGGCATCACCATCATCTCTGGCCTGCCCCCTGAGAAGCCTAAGAACCTG |
| | | AGCTGCATCGTGAATGAGGGCAAGAAGATGCGGTGTGAGTGGGACGGCGGCAGAG |
| | | AGACACACCTGGAGACAAACTTCACCCTGAAGTCCGAGTGGGCCACACACAAGTTT |
| | | GCCGACTGCAAGGCCAAGCGCGATACCCCAACATCCTGTACCGTGGATTACTCTAC |
| | | AGTGTATTTTGTGAACATCGAAGTGTGGGTGGAGGCCGAGAATGCCCTGGGCAAGG |
| | | TGACCTCCGACCACATCAACTTCGATCCCGTGTACAAGGTGAAGCCTAACCCACCCC |
| | | ACAATCTGAGCGTGATCAATTCCGAGGAGCTGTCTAGCATCCTGAAGCTGACCTGGA |
| | | CAAACCCATCTATCAAGAGCGTGATCATCCTGAAGTACAATATCCAGTATCGGACCA |
| | | AGGACGCCTCCACATGGAGCCAGATCCCTCCAGAGGATACCGCCAGCACAAGATCC |
| | | TCTTTCACCGTGCAGGACCTGAAGCCCTTCACAGAGTACGTGTTTCGGATCAGATGT |
| | | ATGAAGGAGGACGGCAAGGGCTACTGGAGCGATTGGTCCGAGGAGGCCAGCGGCA |
| | | TCACCTATGAGGACAGGCCTTCTAAGGCCCCCAGCTTCTGGTACAAGATCGATCCAT |
| | | CCCACACCCAGGGCTATCGCACAGTGCAGCTGGTGTGGAAAACCCTGCCCCCTTTC |
| | | GAGGCCAACGGCAAGATCCTGGACTACGAGGTGACCCTGACACGGTGGAAGTCCC |
| | | ACCTGCAGAACTATACCGTGAATGCCACCAAGCTGACAGTGAACCTGACAAATGATC |
| | | GGTACCTGGCCACCCTGACAGTGAGAAACCTGGTGGGCAAGTCTGACGCCGCCGT |
| | | GCTGACCATCCCTGCCTGCGATTTCCAGGCCACACACCCAGTGATGGACCTGAAGG |
| | | CCTTTCCCAAGGATAATATGCTGTGGGTGGAGTGGACCACACCTAGAGAGTCCGTG |
| | | AAGAAGTACATCCTGGAGTGGTGCGTGCTGTCTGACAAGGCCCCATGTATCACCGA |
| | | CTGGCAGCAGGAGGATGGCACCGTGCACAGGACATATCTGCGCGGCAACCTGGCC |
| | | GAGTCTAAGTGTTACCTGATCACCGTGACACCCGTGTATGCAGACGGACCAGGCTC |
| | | TCCTGAGAGCATCAAGGCCTACCTGAAGCAGGCACCACCAAGCAAGGGACCAACCG |
| | | TGCGGACAAAGAAGGTCGGCAAGAATGAGGCCGTGCTGGAGTGGGACCAGCTGCC |
| | | TGTGGATGTGCAGAACGGCTTCATCAGGAATTACACCATCTTTTATCGCACAATCATC |
| | | GGCAACGAGACAGCCGTGAATGTGGACAGCTCCCACACCGAGTATACACTGTCTAG |
| | | CCTGACCTCCGATACACTGTACATGGTGAGGATGGCCGCCTATACAGACGAGGGCG |
| | | GCAAGGATGGCCCCGAGTTT |
| 52 | IgE signal | GGTACCGGGTCCGCCACCATGGACTGGACCTGGATTCTGTTCCTCGTGGCTGCTGC |
| | sequence | TACAAGAGTGCACAGC |
| 53 | F2A | GGTTCTGGCGTGAAACAGACTTTGAATTTTGACCTTCTCAAGTTGGCGGGAGACGTG |
| | | GAGTCCAACCCAGGGCCC |
| 54 | P2A | GGAAGCGGAGCTACTAACTTCAGCCTGCTGAAGCAGGCTGGAGACGTGGAGGAGA |
| | | ACCCTGGACCT |
| 55 | T2A | GAGGGCAGAGGCAGCCTGCTGACCTGCGGCGACGTCGAGGAGAACCCCGGGCCC |
| 56 | LNGFR | ATGGGGGCAGGTGCCACCGGCCGCGCCATGGACGGGCCGCGCCTGCTGCTGTTG |
| | | CTGCTTCTGGGGGTGTCCCTTGGAGGTGCCAAGGAGGCATGCCCCACAGGCCTGT |
| | | ACACACACAGCGGTGAGTGCTGCAAAGCCTGCAACCTGGGCGAGGGTGTGGCCCA |
| | | GCCTTGTGGAGCCAACCAGACCGTGTGTGAGCCCTGCCTGGACAGCGTGACGTTCT |
| | | CCGACGTGGTGAGCGCGACCGAGCCGTGCAAGCCGTGCACCGAGTGCGTGGGGC |
| | | TCCAGAGCATGTCGGCGCCGTGCGTGGAGGCCGATGACGCCGTGTGCCGCTGCGC |
| | | CTACGGCTACTACCAGGATGAGACGACTGGGCGCTGCGAGGCGTGCCGCGTGTGC |
| | | GAGGCGGGCTCGGGCCTCGTGTTCTCCTGCCAGGACAAGCAGAACACCGTGTGCG |
| | | AGGAGTGCCCCGACGGCACGTATTCCGACGAGGCCAACCACGTGGACCCGTGCCT |
| | | GCCCTGCACCGTGTGCGAGGACACCGAGCGCCAGCTCCGCGAGTGCACACGCTGG |
| | | GCCGACGCCGAGTGCGAGGAGATCCCTGGCCGTTGGATTACACGGTCCACACCCC |
| | | CAGAGGGCTCGGACAGCACAGCCCCCAGCACCCAGGAGCCTGAGGCACCTCCAGA |
| | | ACAAGACCTCATAGCCAGCACGGTGGCAGGTGTGGTGACCACAGTGATGGGCAGCT |
| | | CCCAGCCCGTGGTGACCCGAGGCACCACCGACAACCTCATCCCTGTCTATTGCTCC |
| | | ATCCTGGCTGCTGTGGTTGTGGGTCTTGTGGCCTACATAGCCTTCAAGAGGTGA |
| SEQ | | |
| ID | Sequence | |
| NO# | Name | Polypeptide sequence |
| 57 | IL-12a | MWPPGSASQPPPSPAAATGLHPAARPVSLQCRLSMCPARSLLLVATLVLLDHLSLARNL |
| | polypeptide | PVATPDPGMFPCLHHSQNLLRAVSNMLQKARQTLEFYPCTSEEIDHEDITKDKTSTVEA |
| | | CLPLELTKNESCLNSRETSFITNGSCLASRKTSFMMALCLSSIYEDLKMYQVEFKTMNAK |
| | | LLMDPKRQIFLDQNMLAVIDELMQALNFNSETVPQKSSLEEPDFYKTKIKLCILLHAFRIRA |
| | | VTIDRVMSYLNAS |
| 58 | IL12b | MCHQQLVISWFSLVFLASPLVAIWELKKDVYVVELDWYPDAPGEMVVLTCDTPEEDGIT |
| | polypeptide | WTLDQSSEVLGSGKTLTIQVKEFGDAGQYTCHKGGEVLSHSLLLLHKKEDGIWSTDILKD |
| | | QKEPKNKTFLRCEAKNYSGRFTCWWLTTISTDLTFSVKSSRGSSDPQGVTCGAATLSAE |
| | | RVRGDNKEYEYSVECQEDSACPAAEESLPIEVMVDAVHKLKYENYTSSFFIRDIIKPDPP |
| | | KNLQLKPLKNSRQVEVSWEYPDTWSTPHSYFSLTFCVQVQGKSKREKKDRVFTDKTSA |
| | | TVICRKNASISVRAQDRYYSSSWSEWASVPCS |
| 59 | IL15 | GIHVFILGCFSAGLPKTEANWVNVISDLKKIEDLIQSMHIDATLYTESDVHPSCKVTAMKC |
| | polypeptide | FLLELQVISLESGDASIHDTVENLIILANNSLSSNGNVTESGCKECEELEEKNIKEFLQSFV |
| | | HIVQMFINTS |
| 60 | sIL15ra | ITCPPPMSVEHADIWVKSYSLYSRERYICNSGFKRKAGTSSLTECVLNKATNVAHWTTPS |
| | polypeptide | LKCIRDPALVHQRPAPPSTVTTAGVTPQPESLSPSGKEPAASSPSSNNTAATTAAIVPGS |
| | | QLMPSKSPSTGTTEISSHESSHGTPSQTTAKNWELTASASHQPPGVYPQGHSDTT |
| 61 | soluble gp130 | MLTLQTWLVQALFIFLTTESTGELLDPCGYISPESPVVQLHSNFTAVCVLKEKCMDYFHV |
| | | NANYIVWKTNHFTIPKEQYTIINRTASSVTFTDIASLNIQLTCNILTFGQLEQNVYGITIISGL |
| | | PPEKPKNLSCIVNEGKKMRCEWDGGRETHLETNFTLKSEWATHKFADCKAKRDTPTSC |
| | | TVDYSTVYFVNIEVWVEAENALGKVTSDHINFDPVYKVKPNPPHNLSVINSEELSSILKLT |
| | | WTNPSIKSVIILKYNIQYRTKDASTWSQIPPEDTASTRSSFTVQDLKPFTEYVFRIRCMKE |
| | | DGKGYWSDWSEEASGITYEDRPSKAPSFWYKIDPSHTQGYRTVQLVWKTLPPFEANGK |
| | | ILDYEVTLTRWKSHLQNYTVNATKLTVNLTNDRYLATLTVRNLVGKSDAAVLTIPACDFQA |
| | | THPVMDLKAFPKDNMLWVEWTTPRESVKKYILEWCVLSDKAPCITDWQQEDGTVHRTY |
| | | LRGNLAESKCYLITVTPVYADGPGSPESIKAYLKQAPPSKGPTVRTKKVGKNEAVLEWD |
| | | QLPVDVQNGFIRNYTIFYRTIIGNETAVNVDSSHTEYTLSSLTSDTLYMVRMAAYTDEGG |
| | | KDGPEF |
| 62 | soluble gp130 | MLTLQTWLVQALFIFLTTESTGELLDPCGYISPESPVVQLHSNFTAVCVLKEKCMDYFHV |
| | fused to a Fc | NANYIVWKTNHFTIPKEQYTIINRTASSVTFTDIASLNIQLTCNILTFGQLEQNVYGITIISGL |
| | | PPEKPKNLSCIVNEGKKMRCEWDGGRETHLETNFTLKSEWATHKFADCKAKRDTPTSC |
| | | TVDYSTVYFVNIEVWVEAENALGKVTSDHINFDPVYKVKPNPPHNLSVINSEELSSILKLT |
| | | WTNPSIKSVIILKYNIQYRTKDASTWSQIPPEDTASTRSSFTVQDLKPFTEYVFRIRCMKE |
| | | DGKGYWSDWSEEASGITYEDRPSKAPSFWYKIDPSHTQGYRTVQLVWKTLPPFEANGK |
| | | ILDYEVTLTRWKSHLQNYTVNATKLTVNLTNDRYLATLTVRNLVGKSDAAVLTIPACDFQA |
| | | THPVMDLKAFPKDNMLWVEWTTPRESVKKYILEWCVLSDKAPCITDWQQEDGTVHRTY |
| | | LRGNLAESKCYLITVTPVYADGPGSPESIKAYLKQAPPSKGPTVRTKKVGKNEAVLEWD |
| | | QLPVDVQNGFIRNYTIFYRTIIGNETAVNVDSSHTEYTLSSLTSDTLYMVRMAAYTDEGG |
| | | KDGPEFRSCDKTHTCPPCPAPEAEGGPSVFLFPPKPKDTLMISRTPEVTCVVVDVSHED |
| | | PEVKFNWYVDGVEVHNAKTKPREEQYNSTYRVVSVLTVLHQDWLNGKEYKCKVSNKAL |
| | | PAPIEKTISKAKGQPREPQVYTLPPSREEMTKNQVSLTCLVKGFYPSDIAVEWESNGQP |
| | | ENNYKTTPPVLDSDGSFFLYSKLTVDKSRWQQGNVFSCSVMHEALHNHYTQKSLSLSP |
| | | GK |
| SEQ | | |
| ID | Sequence | |
| NO# | Name | Polynucleotide sequence |
| 63 | Matrice TRAC | GTGGCACTTTTCGGGGAAATGTGCGCGGAACCCCTATTTGTTTATTTTTCTAAATACA |
| | locus_CubiCAR | TTCAAATATGTATCCGCTCATGAGACAATAACCCTGATAAATGCTTCAATAATATTGAA |
| | CD22 | AAAGGAAGAGTATGAGTATTCAACATTTCCGTGTCGCCCTTATTCCCTTTTTTGCGGC |
| | pCLS30056 | ATTTTGCCTTCCTGTTTTTGCTCACCCAGAAACGCTGGTGAAAGTAAAAGATGCTGAA |
| | full sequence | GATCAGTTGGGTGCACGAGTGGGTTACATCGAACTGGATCTCAACAGCGGTAAGAT |
| | | CCTTGAGAGTTTTCGCCCCGAAGAACGTTTTCCAATGATGAGCACTTTTAAAGTTCTG |
| | | CTATGTGGCGCGGTATTATCCCGTATTGACGCCGGGCAAGAGCAACTCGGTCGCCG |
| | | CATACACTATTCTCAGAATGACTTGGTTGAGTACTCACCAGTCACAGAAAAGCATCTT |
| | | ACGGATGGCATGACAGTAAGAGAATTATGCAGTGCTGCCATAACCATGAGTGATAAC |
| | | ACTGCGGCCAACTTACTTCTGACAACGATCGGAGGACCGAAGGAGCTAACCGCTTT |
| | | TTTGCACAACATGGGGGATCATGTAACTCGCCTTGATCGTTGGGAACCGGAGCTGA |
| | | ATGAAGCCATACCAAACGACGAGCGTGACACCACGATGCCTGTAGCAATGGCAACA |
| | | ACGTTGCGCAAACTATTAACTGGCGAACTACTTACTCTAGCTTCCCGGCAACAATTAA |
| | | TAGACTGGATGGAGGCGGATAAAGTTGCAGGACCACTTCTGCGCTCGGCCCTTCCG |
| | | GCTGGCTGGTTTATTGCTGATAAATCTGGAGCCGGTGAGCGTGGTTCTCGCGGTAT |
| | | CATTGCAGCACTGGGGCCAGATGGTAAGCCCTCCCGTATCGTAGTTATCTACACGA |
| | | CGGGGAGTCAGGCAACTATGGATGAACGAAATAGACAGATCGCTGAGATAGGTGCC |
| | | TCACTGATTAAGCATTGGTAACTGTCAGACCAAGTTTACTCATATATACTTTAGATTGA |
| | | TTTAAAACTTCATTTTTAATTTAAAAGGATCTAGGTGAAGATCCTTTTTGATAATCTCAT |
| | | GACCAAAATCCCTTAACGTGAGTTTTCGTTCCACTGAGCGTCAGACCCCGTAGAAAA |
| | | GATCAAAGGATCTTCTTGAGATCCTTTTTTTCTGCGCGTAATCTGCTGCTTGCAAACA |
| | | AAAAAACCACCGCTACCAGCGGTGGTTTGTTTGCCGGATCAAGAGCTACCAACTCTT |
| | | TTTCCGAAGGTAACTGGCTTCAGCAGAGCGCAGATACCAAATACTGTTCTTCTAGTG |
| | | TAGCCGTAGTTAGGCCACCACTTCAAGAACTCTGTAGCACCGCCTACATACCTCGCT |
| | | CTGCTAATCCTGTTACCAGTGGCTGCTGCCAGTGGCGATAAGTCGTGTCTTACCGG |
| | | GTTGGACTCAAGACGATAGTTACCGGATAAGGCGCAGCGGTCGGGCTGAACGGGG |
| | | GGTTCGTGCACACAGCCCAGCTTGGAGCGAACGACCTACACCGAACTGAGATACCT |
| | | ACAGCGTGAGCTATGAGAAAGCGCCACGCTTCCCGAAGGGAGAAAGGCGGACAGG |
| | | TATCCGGTAAGCGGCAGGGTCGGAACAGGAGAGCGCACGAGGGAGCTTCCAGGGG |
| | | GAAACGCCTGGTATCTTTATAGTCCTGTCGGGTTTCGCCACCTCTGACTTGAGCGTC |
| | | GATTTTTGTGATGCTCGTCAGGGGGGCGGAGCCTATGGAAAAACGCCAGCAACGCG |
| | | GCCTTTTTACGGTTCCTGGCCTTTTGCTGGCCTTTTGCTCACATGGTCTTTCCTGCGT |
| | | TATCCCCTGATTCTGTGGATAACCGTATTACCGCCTTTGAGTGAGCTGATACCGCTC |
| | | GCCGCAGCCGAACGACCGAGCGCAGCGAGTCAGTGAGCGAGGAAGCGGAGAGCG |
| | | CCCAATACGCAAACCGCCTCTCCCCGCGCGTTGGCCGATTCATTAATGCAGCTGGC |
| | | ACGACAGGTTTCCCGACTGGAAAGCGGGCAGTGAGCGCAACGCAATTAATGTGAGT |
| | | TAGCTCACTCATTAGGCACCCCAGGCTTTACACTTTATGCTTCCGGCTCGTATGTTGT |
| | | GTGGAATTGTGAGCGGATAACAATTTCACACAGGAAACAGCTATGACCATGATTACG |
| | | CCAAGCGCGTCAATTAACCCTCACTAAAGGGAACAAAAGCTGTTAATTAATTGCTGG |
| | | GCCTTTTTCCCATGCCTGCCTTTACTCTGCCAGAGTTATATTGCTGGGGTTTTGAAGA |
| | | AGATCCTATTAAATAAAAGAATAAGCAGTATTATTAAGTAGCCCTGCATTTCAGGTTT |
| | | CCTTGAGTGGCAGGCCAGGCCTGGCCGTGAACGTTCACTGAAATCATGGCCTCTTG |
| | | GCCAAGATTGATAGCTTGTGCCTGTCCCTGAGTCCCAGTCCATCACGAGCAGCTGG |
| | | TTTCTAAGATGCTATTTCCCGTATAAAGCATGAGACCGTGACTTGCCAGCCCCACAG |
| | | AGCCCCGCCCTTGTCCATCACTGGCATCTGGACTCCAGCCTGGGTTGGGGCAAAGA |
| | | GGGAAATGAGATCATGTCCTAACCCTGATCCTCTTGTCCCACAGATATCCAGTACCC |
| | | CTACGACGTGCCCGACTACGCCTCCGGTGAGGGCAGAGGAAGTCTTCTAACATGCG |
| | | GTGACGTGGAGGAGAATCCGGGCCCCGGATCCGCTCTGCCCGTCACCGCTCTGCT |
| | | GCTGCCACTGGCACTGCTGCTGCACGCTGCTAGGCCCGGAGGGGGAGGCAGCTGC |
| | | CCCTACAGCAACCCCAGCCTGTGCAGCGGAGGCGGCGGCAGCGGCGGAGGGGGT |
| | | AGCCAGGTGCAGCTGCAGCAGAGCGGCCCTGGCCTGGTGAAGCCAAGCCAGACAC |
| | | TGTCCCTGACCTGCGCCATCAGCGGCGATTCCGTGAGCTCCAACTCCGCCGCCTGG |
| | | AATTGGATCAGGCAGTCCCCTTCTCGGGGCCTGGAGTGGCTGGGAAGGACATACTA |
| | | TCGGTCTAAGTGGTACAACGATTATGCCGTGTCTGTGAAGAGCAGAATCACAATCAA |
| | | CCCTGACACCTCCAAGAATCAGTTCTCTCTGCAGCTGAATAGCGTGACACCAGAGGA |
| | | CACCGCCGTGTACTATTGCGCCAGGGAGGTGACCGGCGACCTGGAGGATGCCTTT |
| | | GACATCTGGGGCCAGGGCACAATGGTGACCGTGAGCTCCGGAGGCGGCGGATCTG |
| | | GCGGAGGAGGAAGTGGGGGCGGCGGGAGTGATATCCAGATGACACAGTCCCCATC |
| | | CTCTCTGAGCGCCTCCGTGGGCGACAGAGTGACAATCACCTGTAGGGCCTCCCAGA |
| | | CCATCTGGTCTTACCTGAACTGGTATCAGCAGAGGCCCGGCAAGGCCCCTAATCTG |
| | | CTGATCTACGCAGCAAGCTCCCTGCAGAGCGGAGTGCCATCCAGATTCTCTGGCAG |
| | | GGGCTCCGGCACAGACTTCACCCTGACCATCTCTAGCCTGCAGGCCGAGGACTTCG |
| | | CCACCTACTATTGCCAGCAGTCTTATAGCATCCCCCAGACATTTGGCCAGGGCACCA |
| | | AGCTGGAGATCAAGTCGGATCCCGGAAGCGGAGGGGGAGGCAGCTGCCCCTACAG |
| | | CAACCCCAGCCTGTGCAGCGGAGGCGGCGGCAGCGAGCTGCCCACCCAGGGCAC |
| | | CTTCTCCAACGTGTCCACCAACGTGAGCCCAGCCAAGCCCACCACCACCGCCTGTC |
| | | CTTATTCCAATCCTTCCCTGTGTGCTCCCACCACAACCCCCGCTCCAAGGCCCCCTA |
| | | CCCCCGCACCAACTATTGCCTCCCAGCCACTCTCACTGCGGCCTGAGGCCTGTCGG |
| | | CCCGCTGCTGGAGGCGCAGTGCATACAAGGGGCCTCGATTTCGCCTGCGATATTTA |
| | | CATCTGGGCACCCCTCGCCGGCACCTGCGGGGTGCTTCTCCTCTCCCTGGTGATTA |
| | | CCCTGTATTGCAGACGGGGCCGGAAGAAGCTCCTCTACATTTTTAAGCAGCCTTTCA |
| | | TGCGGCCAGTGCAGACAACCCAAGAGGAGGATGGGTGTTCCTGCAGATTCCCTGAG |
| | | GAAGAGGAAGGCGGGTGCGAGCTGAGAGTGAAGTTCTCCAGGAGCGCAGATGCCC |
| | | CCGCCTATCAACAGGGCCAGAACCAGCTCTACAACGAGCTTAACCTCGGGAGGCGC |
| | | GAAGAATACGACGTGTTGGATAAGAGAAGGGGGCGGGACCCCGAGATGGGAGGAA |
| | | AGCCCCGGAGGAAGAACCCTCAGGAGGGCCTGTACAACGAGCTGCAGAAGGATAA |
| | | GATGGCCGAGGCCTACTCAGAGATCGGGATGAAGGGGGAGCGGCGCCGCGGGAA |
| | | GGGGCACGATGGGCTCTACCAGGGGCTGAGCACAGCCACAAAGGACACATACGAC |
| | | GCCTTGCACATGCAGGCCCTTCCACCCCGGGAATAGTCTAGAGGGCCCGTTTAAAC |
| | | CCGCTGATCAGCCTCGACTGTGCCTTCTAGTTGCCAGCCATCTGTTGTTTGCCCCTC |
| | | CCCCGTGCCTTCCTTGACCCTGGAAGGTGCCACTCCCACTGTCCTTTCCTAATAAAA |
| | | TGAGGAAATTGCATCGCATTGTCTGAGTAGGTGTCATTCTATTCTGGGGGGTGGGGT |
| | | GGGGCAGGACAGCAAGGGGGAGGATTGGGAAGACAATAGCAGGCATGCTGGGGAT |
| | | GCGGTGGGCTCTATGACTAGTGGCGAATTCCCGTGTACCAGCTGAGAGACTCTAAA |
| | | TCCAGTGACAAGTCTGTCTGCCTATTCACCGATTTTGATTCTCAAACAAATGTGTCAC |
| | | AAAGTAAGGATTCTGATGTGTATATCACAGACAAAACTGTGCTAGACATGAGGTCTAT |
| | | GGACTTCAAGAGCAACAGTGCTGTGGCCTGGAGCAACAAATCTGACTTTGCATGTG |
| | | CAAACGCCTTCAACAACAGCATTATTCCAGAAGACACCTTCTTCCCCAGCCCAGGTA |
| | | AGGGCAGCTTTGGTGCCTTCGCAGGCTGTTTCCTTGCTTCAGGAATGGCCAGGTTC |
| | | TGCCCAGAGCTCTGGTCAATGATGTCTAAAACTCCTCTGATTGGTGGTCTCGGCCTT |
| | | ATCCATTGCCACCAAAACCCTCTTTTTACTAAGCGATCGCTCCGGTGCCCGTCAGTG |
| | | GGCAGAGCGCACATCGCCCACAGTCCCCGAGAAGTTGGGGGGAGGGGTCGGCAAT |
| | | TGAACGGGTGCCTAGAGAAGGTGGCGCGGGGTAAACTGGGAAAGTGATGTCGTGT |
| | | ACTGGCTCCGCCTTTTTCCCGAGGGTGGGGGAGAACCGTATATAAGTGCAGTAGTC |
| | | GCCGTGAACGTTCTTTTTCGCAACGGGTTTGCCGCCAGAACACAGCTGAAGCTTCG |
| | | AGGGGCTCGCATCTCTCCTTCACGCGCCCGCCGCCCTACCTGAGGCCGCCATCCA |
| | | CGCCGGTTGAGTCGCGTTCTGCCGCCTCCCGCCTGTGGTGCCTCCTGAACTGCGTC |
| | | CGCCGTCTAGGTAAGTTTAAAGCTCAGGTCGAGACCGGGCCTTTGTCCGGCGCTCC |
| | | CTTGGAGCCTACCTAGACTCAGCCGGCTCTCCACGCTTTGCCTGACCCTGCTTGCT |
| | | CAACTCTACGTCTTTGTTTCGTTTTCTGTTCTGCGCCGTTACAGATCCAAGCTGTGAC |
| | | CGGCGCCTACCTGAGATCACCGGCGCCACCATGGCTTCTTACCCTGGACACCAGCA |
| | | TGCTTCTGCCTTTGACCAGGCTGCCAGATCCAGGGGCCACTCCAACAGGAGAACTG |
| | | CCCTAAGACCCAGAAGACAGCAGGAAGCCACTGAGGTGAGGCCTGAGCAGAAGAT |
| | | GCCAACCCTGCTGAGGGTGTACATTGATGGACCTCATGGCATGGGCAAGACCACCA |
| | | CCACTCAACTGCTGGTGGCACTGGGCTCCAGGGATGACATTGTGTATGTGCCTGAG |
| | | CCAATGACCTACTGGAGAGTGCTAGGAGCCTCTGAGACCATTGCCAACATCTACACC |
| | | ACCCAGCACAGGCTGGACCAGGGAGAAATCTCTGCTGGAGATGCTGCTGTGGTGAT |
| | | GACCTCTGCCCAGATCACAATGGGAATGCCCTATGCTGTGACTGATGCTGTTCTGGC |
| | | TCCTCACATTGGAGGAGAGGCTGGCTCTTCTCATGCCCCTCCACCTGCCCTGACCC |
| | | TGATCTTTGACAGACACCCCATTGCAGCCCTGCTGTGCTACCCAGCAGCAAGGTAC |
| | | CTCATGGGCTCCATGACCCCACAGGCTGTGCTGGCTTTTGTGGCCCTGATCCCTCC |
| | | AACCCTCCCTGGCACCAACATTGTTCTGGGAGCACTGCCTGAAGACAGACACATTGA |
| | | CAGGCTGGCAAAGAGGCAGAGACCTGGAGAGAGACTGGACCTGGCCATGCTGGCT |
| | | GCAATCAGAAGGGTGTATGGACTGCTGGCAAACACTGTGAGATACCTCCAGTGTGG |
| | | AGGCTCTTGGAGAGAGGACTGGGGACAGCTCTCTGGAACAGCAGTGCCCCCTCAA |
| | | GGAGCTGAGCCCCAGTCCAATGCTGGTCCAAGACCCCACATTGGGGACACCCTGTT |
| | | CACCCTGTTCAGAGCCCCTGAGCTGCTGGCTCCCAATGGAGACCTGTACAATGTGT |
| | | TTGCCTGGGCTCTGGATGTTCTAGCCAAGAGGCTGAGGTCCATGCATGTGTTCATCC |
| | | TGGACTATGACCAGTCCCCTGCTGGATGCAGAGATGCTCTGCTGCAACTAACCTCTG |
| | | GCATGGTGCAGACCCATGTGACCACCCCTGGCAGCATCCCCACCATCTGTGACCTA |
| | | GCCAGAACCTTTGCCAGGGAGATGGGAGAGGCCAACTAAGGCGCGCCACTCGAGC |
| | | GCTAGCTGGCCAGACATGATAAGATACATTGATGAGTTTGGACAAACCACAACTAGA |
| | | ATGCAGTGAAAAAAATGCTTTATTTGTGAAATTTGTGATGCTATTGCTTTATTTGTAAC |
| | | CATTATAAGCTGCAATAAACAAGTTAACAACAACAATTGCATTCATTTTATGTTTCAGG |
| | | TTCAGGGGGAGGTGTGGGAGGTTTTTTAAAGCAAGTAAAACCTCTACAAATGTGGTA |
| | | TGGAAGGCGCGCCCAATTCGCCCTATAGTGAGTCGTATTACGTCGCGCTCACTGGC |
| | | CGTCGTTTTACAACGTCGTGACTGGGAAAACCCTGGCGTTACCCAACTTAATCGCCT |
| | | TGCAGCACATCCCCCTTTCGCCAGCTGGCGTAATAGCGAAGAGGCCCGCACCGAAA |
| | | CGCCCTTCCCAACAGTTGCGCAGCCTGAATGGCGAATGGGAGCGCCCTGTAGCGG |
| | | CGCATTAAGCGCGGCGGGTGTGGTGGTTACGCGCAGCGTGACCGCTACACTTGCC |
| | | AGCGCCCTAGCGCCCGCTCCTTTCGCTTTCTTCCCTTCCTTTCTCGCCACGTTCGCC |
| | | GGCTTTCCCCGTCAAGCTCTAAATCGGGGGCTCCCTTTAGGGTTCCGATTTAGTGCT |
| | | TTACGGCACCTCGACCCCAAAAAACTTGATTAGGGTGATGGTTGGCCTGTAGTGGG |
| | | CCATAGCCCTGATAGACGGTTTTTCGCCCTTTGACGTTGGAGTCCACGTTCTTTAATA |
| | | GTGGACTCTTGTTCCAAACTGGAACAACACTCAACCCTATCTCGGTCTATTCTTTTGA |
| | | TTTATAAGGGATTTTGCCGATTTCGGCCTATTGGTTAAAAAATGAGCTGATTTAACAA |
| | | AAATTTAACGCGAATTTTAACAAAATATTAACGCTTACAATTTAG |
| 64 | Matrice CD25 | GTTTATTATTCCTGTTCCACAGCTATTGTCTGCCATATAAAAACTTAGGCCAGGCACA |
| | locus_IL15_ | GTGGCTCACACCTGTAATCCCAGCACTTTGGAAGGCCGAGGCAGGCAGATCACAAG |
| | 2A_sIL15Ra | GTCAGGAGTTCGAGACCAGCCTGGCCAACATAGCAAAACCCCATCTCTACTAAAAAT |
| | pCLS30519 | ACAAAAATTAGCCAGGCATGGTGGCGTGTGCACTGGTTTAGAGTGAGGACCACATTT |
| | full sequence | TTTTGGTGCCGTGTTACACATATGACCGTGACTTTGTTACACCACTACAGGAGGAAG |
| | | AGTAGAAGAACAATCGGTTCTGGCGTGAAACAGACTTTGAATTTTGACCTTCTCAAGT |
| | | TGGCGGGAGACGTGGAGTCCAACCCAGGGCCCGGTACCGGGTCCGCCACCATGGA |
| | | CTGGACCTGGATTCTGTTCCTCGTGGCTGCTGCTACAAGAGTGCACAGCGGCATTC |
| | | ATGTCTTCATTTTGGGCTGTTTCAGTGCAGGGCTTCCTAAAACAGAAGCCAACTGGG |
| | | TGAATGTAATAAGTGATTTGAAAAAAATTGAAGATCTTATTCAATCTATGCATATTGAT |
| | | GCTACTTTATATACGGAAAGTGATGTTCACCCCAGTTGCAAAGTAACAGCAATGAAG |
| | | TGCTTTCTCTTGGAGTTACAAGTTATTTCACTTGAGTCCGGAGATGCAAGTATTCATG |
| | | ATACAGTAGAAAATCTGATCATCCTAGCAAACAACAGTTTGTCTTCTAATGGGAATGT |
| | | AACAGAATCTGGATGCAAAGAATGTGAGGAACTGGAGGAAAAAAATATTAAAGAATT |
| | | TTTGCAGAGTTTTGTACATATTGTCCAAATGTTCATCAACACTTCTGGAAGCGGAGCT |
| | | ACTAACTTCAGCCTGCTGAAGCAGGCTGGAGACGTGGAGGAGAACCCTGGACCTGG |
| | | GACCGGCTCTGCAACCATGGATTGGACGTGGATCCTGTTTCTCGTGGCAGCTGCCA |
| | | CAAGAGTTCACAGTATCACGTGCCCTCCCCCCATGTCCGTGGAACACGCAGACATC |
| | | TGGGTCAAGAGCTACAGCTTGTACTCCAGGGAGCGGTACATTTGTAACTCTGGTTTC |
| | | AAGCGTAAAGCCGGCACGTCCAGCCTGACGGAGTGCGTGTTGAACAAGGCCACGA |
| | | ATGTCGCCCACTGGACAACCCCCAGTCTCAAATGCATTAGAGACCCTGCCCTGGTTC |
| | | ACCAAAGGCCAGCGCCACCCTCCACAGTAACGACGGCAGGGGTGACCCCACAGCC |
| | | AGAGAGCCTCTCCCCTTCTGGAAAAGAGCCCGCAGCTTCATCTCCCAGCTCAAACAA |
| | | CACAGCGGCCACAACAGCAGCTATTGTCCCGGGCTCCCAGCTGATGCCTTCAAAAT |
| | | CACCTTCCACAGGAACCACAGAGATAAGCAGTCATGAGTCCTCCCACGGCACCCCC |
| | | TCTCAGACAACAGCCAAGAACTGGGAACTCACAGCATCCGCCTCCCACCAGCCGCC |
| | | AGGTGTGTATCCACAGGGCCACAGCGACACCACTGAGGGCAGAGGCAGCCTGCTG |
| | | ACCTGCGGCGACGTCGAGGAGAACCCCGGGCCCATGGGGGCAGGTGCCACCGGC |
| | | CGCGCCATGGACGGGCCGCGCCTGCTGCTGTTGCTGCTTCTGGGGGTGTCCCTTG |
| | | GAGGTGCCAAGGAGGCATGCCCCACAGGCCTGTACACACACAGCGGTGAGTGCTG |
| | | CAAAGCCTGCAACCTGGGCGAGGGTGTGGCCCAGCCTTGTGGAGCCAACCAGACC |
| | | GTGTGTGAGCCCTGCCTGGACAGCGTGACGTTCTCCGACGTGGTGAGCGCGACCG |
| | | AGCCGTGCAAGCCGTGCACCGAGTGCGTGGGGCTCCAGAGCATGTCGGCGCCGTG |
| | | CGTGGAGGCCGATGACGCCGTGTGCCGCTGCGCCTACGGCTACTACCAGGATGAG |
| | | ACGACTGGGCGCTGCGAGGCGTGCCGCGTGTGCGAGGCGGGCTCGGGCCTCGTG |
| | | TTCTCCTGCCAGGACAAGCAGAACACCGTGTGCGAGGAGTGCCCCGACGGCACGT |
| | | ATTCCGACGAGGCCAACCACGTGGACCCGTGCCTGCCCTGCACCGTGTGCGAGGA |
| | | CACCGAGCGCCAGCTCCGCGAGTGCACACGCTGGGCCGACGCCGAGTGCGAGGA |
| | | GATCCCTGGCCGTTGGATTACACGGTCCACACCCCCAGAGGGCTCGGACAGCACA |
| | | GCCCCCAGCACCCAGGAGCCTGAGGCACCTCCAGAACAAGACCTCATAGCCAGCA |
| | | CGGTGGCAGGTGTGGTGACCACAGTGATGGGCAGCTCCCAGCCCGTGGTGACCCG |
| | | AGGCACCACCGACAACCTCATCCCTGTCTATTGCTCCATCCTGGCTGCTGTGGTTGT |
| | | GGGTCTTGTGGCCTACATAGCCTTCAAGAGGTGAAAAACCAAAAGAACAAGAATTTC |
| | | TTGGTAAGAAGCCGGGAACAGACAACAGAAGTCATGAAGCCCAAGTGAAATCAAAG |
| | | GTGCTAAATGGTCGCCCAGGAGACATCCGTTGTGCTTGCCTGCGTTTTGGAAGCTCT |
| | | GAAGTCACATCACAGGACACGGGGCAGTGGCAACCTTGTCTCTATGCCAGCTCAGT |
| | | CCCATCAGAGAGCGAGCGCTACCCACTTCTAAATAGCAATTTCGCCGTTGAAGAGGA |
| | | AGGGCAAAACCACTAGAACTCTCCATCTTATTTTCATGTATATGTGTTCATGCGATCG |
| | | CTCCGGTGCCCGTCAGTGGGCAGAGCGCACATCGCCCACAGTCCCCGAGAAGTTG |
| | | GGGGGAGGGGTCGGCAATTGAACGGGTGCCTAGAGAAGGTGGCGCGGGGTAAACT |
| | | GGGAAAGTGATGTCGTGTACTGGCTCCGCCTTTTTCCCGAGGGTGGGGGAGAACCG |
| | | TATATAAGTGCAGTAGTCGCCGTGAACGTTCTTTTTCGCAACGGGTTTGCCGCCAGA |
| | | ACACAGCTGAAGCTTCGAGGGGCTCGCATCTCTCCTTCACGCGCCCGCCGCCCTAC |
| | | CTGAGGCCGCCATCCACGCCGGTTGAGTCGCGTTCTGCCGCCTCCCGCCTGTGGT |
| | | GCCTCCTGAACTGCGTCCGCCGTCTAGGTAAGTTTAAAGCTCAGGTCGAGACCGGG |
| | | CCTTTGTCCGGCGCTCCCTTGGAGCCTACCTAGACTCAGCCGGCTCTCCACGCTTT |
| | | GCCTGACCCTGCTTGCTCAACTCTACGTCTTTGTTTCGTTTTCTGTTCTGCGCCGTTA |
| | | CAGATCCAAGCTGTGACCGGCGCCTACCTGAGATCACCGGCGCCACCATGGCTTCT |
| | | TACCCTGGACACCAGCATGCTTCTGCCTTTGACCAGGCTGCCAGATCCAGGGGCCA |
| | | CTCCAACAGGAGAACTGCCCTAAGACCCAGAAGACAGCAGGAAGCCACTGAGGTGA |
| | | GGCCTGAGCAGAAGATGCCAACCCTGCTGAGGGTGTACATTGATGGACCTCATGGC |
| | | ATGGGCAAGACCACCACCACTCAACTGCTGGTGGCACTGGGCTCCAGGGATGACAT |
| | | TGTGTATGTGCCTGAGCCAATGACCTACTGGAGAGTGCTAGGAGCCTCTGAGACCA |
| | | TTGCCAACATCTACACCACCCAGCACAGGCTGGACCAGGGAGAAATCTCTGCTGGA |
| | | GATGCTGCTGTGGTGATGACCTCTGCCCAGATCACAATGGGAATGCCCTATGCTGT |
| | | GACTGATGCTGTTCTGGCTCCTCACATTGGAGGAGAGGCTGGCTCTTCTCATGCCC |
| | | CTCCACCTGCCCTGACCCTGATCTTTGACAGACACCCCATTGCAGCCCTGCTGTGCT |
| | | ACCCAGCAGCAAGGTACCTCATGGGCTCCATGACCCCACAGGCTGTGCTGGCTTTT |
| | | GTGGCCCTGATCCCTCCAACCCTCCCTGGCACCAACATTGTTCTGGGAGCACTGCC |
| | | TGAAGACAGACACATTGACAGGCTGGCAAAGAGGCAGAGACCTGGAGAGAGACTG |
| | | GACCTGGCCATGCTGGCTGCAATCAGAAGGGTGTATGGACTGCTGGCAAACACTGT |
| | | GAGATACCTCCAGTGTGGAGGCTCTTGGAGAGAGGACTGGGGACAGCTCTCTGGAA |
| | | CAGCAGTGCCCCCTCAAGGAGCTGAGCCCCAGTCCAATGCTGGTCCAAGACCCCAC |
| | | ATTGGGGACACCCTGTTCACCCTGTTCAGAGCCCCTGAGCTGCTGGCTCCCAATGG |
| | | AGACCTGTACAATGTGTTTGCCTGGGCTCTGGATGTTCTAGCCAAGAGGCTGAGGT |
| | | CCATGCATGTGTTCATCCTGGACTATGACCAGTCCCCTGCTGGATGCAGAGATGCTC |
| | | TGCTGCAACTAACCTCTGGCATGGTGCAGACCCATGTGACCACCCCTGGCAGCATC |
| | | CCCACCATCTGTGACCTAGCCAGAACCTTTGCCAGGGAGATGGGAGAGGCCAACTA |
| | | AGGCGCGCCACTCGAGCGCTAGCTGGCCAGACATGATAAGATACATTGATGAGTTT |
| | | GGACAAACCACAACTAGAATGCAGTGAAAAAAATGCTTTATTTGTGAAATTTGTGATG |
| | | CTATTGCTTTATTTGTAACCATTATAAGCTGCAATAAACAAGTTAACAACAACAATTGC |
| | | ATTCATTTTATGTTTCAGGTTCAGGGGGAGGTGTGGGAGGTTTTTTAAAGCAAGTAAA |
| | | ACCTCTACAAATGTGGTATGGAAGGCGCGCCCAATTCGCCCTATAGTGAGTCGTATT |
| | | ACGTCGCGCTCACTGGCCGTCGTTTTACAACGTCGTGACTGGGAAAACCCTGGCGT |
| | | TACCCAACTTAATCGCCTTGCAGCACATCCCCCTTTCGCCAGCTGGCGTAATAGCGA |
| | | AGAGGCCCGCACCGAAACGCCCTTCCCAACAGTTGCGCAGCCTGAATGGCGAATG |
| | | GGAGCGCCCTGTAGCGGCGCATTAAGCGCGGCGGGTGTGGTGGTTACGCGCAGCG |
| | | TGACCGCTACACTTGCCAGCGCCCTAGCGCCCGCTCCTTTCGCTTTCTTCCCTTCCT |
| | | TTCTCGCCACGTTCGCCGGCTTTCCCCGTCAAGCTCTAAATCGGGGGCTCCCTTTAG |
| | | GGTTCCGATTTAGTGCTTTACGGCACCTCGACCCCAAAAAACTTGATTAGGGTGATG |
| | | GTTGGCCTGTAGTGGGCCATAGCCCTGATAGACGGTTTTTCGCCCTTTGACGTTGGA |
| | | GTCCACGTTCTTTAATAGTGGACTCTTGTTCCAAACTGGAACAACACTCAACCCTATC |
| | | TCGGTCTATTCTTTTGATTTATAAGGGATTTTGCCGATTTCGGCCTATTGGTTAAAAAA |
| | | TGAGCTGATTTAACAAAAATTTAACGCGAATTTTAACAAAATATTAACGCTTACAATTT |
| | | AGGTGGCACTTTTCGGGGAAATGTGCGCGGAACCCCTATTTGTTTATTTTTCTAAATA |
| | | CATTCAAATATGTATCCGCTCATGAGACAATAACCCTGATAAATGCTTCAATAATATT |
| | | GAAAAAGGAAGAGTATGAGTATTCAACATTTCCGTGTCGCCCTTATTCCCTTTTTTGC |
| | | GGCATTTTGCCTTCCTGTTTTTGCTCACCCAGAAACGCTGGTGAAAGTAAAAGATGC |
| | | TGAAGATCAGTTGGGTGCACGAGTGGGTTACATCGAACTGGATCTCAACAGCGGTA |
| | | AGATCCTTGAGAGTTTTCGCCCCGAAGAACGTTTTCCAATGATGAGCACTTTTAAAGT |
| | | TCTGCTATGTGGCGCGGTATTATCCCGTATTGACGCCGGGCAAGAGCAACTCGGTC |
| | | GCCGCATACACTATTCTCAGAATGACTTGGTTGAGTACTCACCAGTCACAGAAAAGC |
| | | ATCTTACGGATGGCATGACAGTAAGAGAATTATGCAGTGCTGCCATAACCATGAGTG |
| | | ATAACACTGCGGCCAACTTACTTCTGACAACGATCGGAGGACCGAAGGAGCTAACC |
| | | GCTTTTTTGCACAACATGGGGGATCATGTAACTCGCCTTGATCGTTGGGAACCGGAG |
| | | CTGAATGAAGCCATACCAAACGACGAGCGTGACACCACGATGCCTGTAGCAATGGC |
| | | AACAACGTTGCGCAAACTATTAACTGGCGAACTACTTACTCTAGCTTCCCGGCAACA |
| | | ATTAATAGACTGGATGGAGGCGGATAAAGTTGCAGGACCACTTCTGCGCTCGGCCC |
| | | TTCCGGCTGGCTGGTTTATTGCTGATAAATCTGGAGCCGGTGAGCGTGGTTCTCGC |
| | | GGTATCATTGCAGCACTGGGGCCAGATGGTAAGCCCTCCCGTATCGTAGTTATCTAC |
| | | ACGACGGGGAGTCAGGCAACTATGGATGAACGAAATAGACAGATCGCTGAGATAGG |
| | | TGCCTCACTGATTAAGCATTGGTAACTGTCAGACCAAGTTTACTCATATATACTTTAG |
| | | ATTGATTTAAAACTTCATTTTTAATTTAAAAGGATCTAGGTGAAGATCCTTTTTGATAAT |
| | | CTCATGACCAAAATCCCTTAACGTGAGTTTTCGTTCCACTGAGCGTCAGACCCCGTA |
| | | GAAAAGATCAAAGGATCTTCTTGAGATCCTTTTTTTCTGCGCGTAATCTGCTGCTTGC |
| | | AAACAAAAAAACCACCGCTACCAGCGGTGGTTTGTTTGCCGGATCAAGAGCTACCAA |
| | | CTCTTTTTCCGAAGGTAACTGGCTTCAGCAGAGCGCAGATACCAAATACTGTTCTTCT |
| | | AGTGTAGCCGTAGTTAGGCCACCACTTCAAGAACTCTGTAGCACCGCCTACATACCT |
| | | CGCTCTGCTAATCCTGTTACCAGTGGCTGCTGCCAGTGGCGATAAGTCGTGTCTTAC |
| | | CGGGTTGGACTCAAGACGATAGTTACCGGATAAGGCGCAGCGGTCGGGCTGAACG |
| | | GGGGGTTCGTGCACACAGCCCAGCTTGGAGCGAACGACCTACACCGAACTGAGATA |
| | | CCTACAGCGTGAGCTATGAGAAAGCGCCACGCTTCCCGAAGGGAGAAAGGCGGAC |
| | | AGGTATCCGGTAAGCGGCAGGGTCGGAACAGGAGAGCGCACGAGGGAGCTTCCAG |
| | | GGGGAAACGCCTGGTATCTTTATAGTCCTGTCGGGTTTCGCCACCTCTGACTTGAGC |
| | | GTCGATTTTTGTGATGCTCGTCAGGGGGGCGGAGCCTATGGAAAAACGCCAGCAAC |
| | | GCGGCCTTTTTACGGTTCCTGGCCTTTTGCTGGCCTTTTGCTCACATGGTCTTTCCT |
| | | GCGTTATCCCCTGATTCTGTGGATAACCGTATTACCGCCTTTGAGTGAGCTGATACC |
| | | GCTCGCCGCAGCCGAACGACCGAGCGCAGCGAGTCAGTGAGCGAGGAAGCGGAG |
| | | AGCGCCCAATACGCAAACCGCCTCTCCCCGCGCGTTGGCCGATTCATTAATGCAGC |
| | | TGGCACGACAGGTTTCCCGACTGGAAAGCGGGCAGTGAGCGCAACGCAATTAATGT |
| | | GAGTTAGCTCACTCATTAGGCACCCCAGGCTTTACACTTTATGCTTCCGGCTCGTAT |
| | | GTTGTGTGGAATTGTGAGCGGATAACAATTTCACACAGGAAACAGCTATGACCATGA |
| | | TTACGCCAAGCGCGTCAATTAACCCTCACTAAAGGGAACAAAAGCTGTTAATTAA |
| 65 | Matrice PD1 | GACTCCCCAGACAGGCCCTGGAACCCCCCCACCTTCTCCCCAGCCCTGCTCGTGGT |
| | locus_IL15_ | GACCGAAGGGGACAACGCCACCTTCACCTGCAGCTTCTCCAACACATCGGAGAGCT |
| | 2A_sIL15Ra | TCGTGCTAAACTGGTACCGCATGAGCCCCAGCAACCAGACGGACAAGCTGGCCGCC |
| | pCLS30513 | TTCCCCGAGGACCGCAGCCAGCCCGGCCAGGACTGCCGCTTCCGTGTCACACAAC |
| | full sequence | TGCCCAACGGGCGTGACTTCCACATGAGCGTGGTCAGGGCCCGGCGCAATGACAG |
| | | CGGCACCTACCTCTGTGGGGCCGGTTCTGGCGTGAAACAGACTTTGAATTTTGACCT |
| | | TCTCAAGTTGGCGGGAGACGTGGAGTCCAACCCAGGGCCCGGTACCGGGTCCGCC |
| | | ACCATGGACTGGACCTGGATTCTGTTCCTCGTGGCTGCTGCTACAAGAGTGCACAG |
| | | CGGCATTCATGTCTTCATTTTGGGCTGTTTCAGTGCAGGGCTTCCTAAAACAGAAGC |
| | | CAACTGGGTGAATGTAATAAGTGATTTGAAAAAAATTGAAGATCTTATTCAATCTATG |
| | | CATATTGATGCTACTTTATATACGGAAAGTGATGTTCACCCCAGTTGCAAAGTAACAG |
| | | CAATGAAGTGCTTTCTCTTGGAGTTACAAGTTATTTCACTTGAGTCCGGAGATGCAAG |
| | | TATTCATGATACAGTAGAAAATCTGATCATCCTAGCAAACAACAGTTTGTCTTCTAAT |
| | | GGGAATGTAACAGAATCTGGATGCAAAGAATGTGAGGAACTGGAGGAAAAAAATATT |
| | | AAAGAATTTTTGCAGAGTTTTGTACATATTGTCCAAATGTTCATCAACACTTCTGGAA |
| | | GCGGAGCTACTAACTTCAGCCTGCTGAAGCAGGCTGGAGACGTGGAGGAGAACCCT |
| | | GGACCTGGGACCGGCTCTGCAACCATGGATTGGACGTGGATCCTGTTTCTCGTGGC |
| | | AGCTGCCACAAGAGTTCACAGTATCACGTGCCCTCCCCCCATGTCCGTGGAACACG |
| | | CAGACATCTGGGTCAAGAGCTACAGCTTGTACTCCAGGGAGCGGTACATTTGTAACT |
| | | CTGGTTTCAAGCGTAAAGCCGGCACGTCCAGCCTGACGGAGTGCGTGTTGAACAAG |
| | | GCCACGAATGTCGCCCACTGGACAACCCCCAGTCTCAAATGCATTAGAGACCCTGC |
| | | CCTGGTTCACCAAAGGCCAGCGCCACCCTCCACAGTAACGACGGCAGGGGTGACC |
| | | CCACAGCCAGAGAGCCTCTCCCCTTCTGGAAAAGAGCCCGCAGCTTCATCTCCCAG |
| | | CTCAAACAACACAGCGGCCACAACAGCAGCTATTGTCCCGGGCTCCCAGCTGATGC |
| | | CTTCAAAATCACCTTCCACAGGAACCACAGAGATAAGCAGTCATGAGTCCTCCCACG |
| | | GCACCCCCTCTCAGACAACAGCCAAGAACTGGGAACTCACAGCATCCGCCTCCCAC |
| | | CAGCCGCCAGGTGTGTATCCACAGGGCCACAGCGACACCACTGAGGGCAGAGGCA |
| | | GCCTGCTGACCTGCGGCGACGTCGAGGAGAACCCCGGGCCCATGGGGGCAGGTG |
| | | CCACCGGCCGCGCCATGGACGGGCCGCGCCTGCTGCTGTTGCTGCTTCTGGGGGT |
| | | GTCCCTTGGAGGTGCCAAGGAGGCATGCCCCACAGGCCTGTACACACACAGCGGT |
| | | GAGTGCTGCAAAGCCTGCAACCTGGGCGAGGGTGTGGCCCAGCCTTGTGGAGCCA |
| | | ACCAGACCGTGTGTGAGCCCTGCCTGGACAGCGTGACGTTCTCCGACGTGGTGAG |
| | | CGCGACCGAGCCGTGCAAGCCGTGCACCGAGTGCGTGGGGCTCCAGAGCATGTCG |
| | | GCGCCGTGCGTGGAGGCCGATGACGCCGTGTGCCGCTGCGCCTACGGCTACTACC |
| | | AGGATGAGACGACTGGGCGCTGCGAGGCGTGCCGCGTGTGCGAGGCGGGCTCGG |
| | | GCCTCGTGTTCTCCTGCCAGGACAAGCAGAACACCGTGTGCGAGGAGTGCCCCGA |
| | | CGGCACGTATTCCGACGAGGCCAACCACGTGGACCCGTGCCTGCCCTGCACCGTG |
| | | TGCGAGGACACCGAGCGCCAGCTCCGCGAGTGCACACGCTGGGCCGACGCCGAGT |
| | | GCGAGGAGATCCCTGGCCGTTGGATTACACGGTCCACACCCCCAGAGGGCTCGGA |
| | | CAGCACAGCCCCCAGCACCCAGGAGCCTGAGGCACCTCCAGAACAAGACCTCATAG |
| | | CCAGCACGGTGGCAGGTGTGGTGACCACAGTGATGGGCAGCTCCCAGCCCGTGGT |
| | | GACCCGAGGCACCACCGACAACCTCATCCCTGTCTATTGCTCCATCCTGGCTGCTG |
| | | TGGTTGTGGGTCTTGTGGCCTACATAGCCTTCAAGAGGTGATCTAGAGGGCCCGTTT |
| | | AAACCCGCTGATCAGCCTCGACTGTGCCTTCTAGTTGCCAGCCATCTGTTGTTTGCC |
| | | CCTCCCCCGTGCCTTCCTTGACCCTGGAAGGTGCCACTCCCACTGTCCTTTCCTAAT |
| | | AAAATGAGGAAATTGCATCGCATTGTCTGAGTAGGTGTCATTCTATTCTGGGGGGTG |
| | | GGGTGGGGCAGGACAGCAAGGGGGAGGATTGGGAAGACAATAGCAGGCATGCTGG |
| | | GGATGCGGTGGGCTCTATGACTAGTGGCGAATTCGGCGCAGATCAAAGAGAGCCTG |
| | | CGGGCAGAGCTCAGGGTGACAGGTGCGGCCTCGGAGGCCCCGGGGCAGGGGTGA |
| | | GCTGAGCCGGTCCTGGGGTGGGTGTCCCCTCCTGCACAGGATCAGGAGCTCCAGG |
| | | GTCGTAGGGCAGGGACCCCCCAGCTCCAGTCCAGGGCTCTGTCCTGCACCTGGGG |
| | | AATGGTGACCGGCATCTCTGTCCTCTAGCTCTGGAAGCACCCCAGCCCCTCTAGTCT |
| | | GCCCTCACCCCTGACCCTGACCCTCCACCCTGACCCCGTCCTAACCCCTGACCTTT |
| | | GGCGATCGCTCCGGTGCCCGTCAGTGGGCAGAGCGCACATCGCCCACAGTCCCCG |
| | | AGAAGTTGGGGGGAGGGGTCGGCAATTGAACGGGTGCCTAGAGAAGGTGGCGCGG |
| | | GGTAAACTGGGAAAGTGATGTCGTGTACTGGCTCCGCCTTTTTCCCGAGGGTGGGG |
| | | GAGAACCGTATATAAGTGCAGTAGTCGCCGTGAACGTTCTTTTTCGCAACGGGTTTG |
| | | CCGCCAGAACACAGCTGAAGCTTCGAGGGGCTCGCATCTCTCCTTCACGCGCCCGC |
| | | CGCCCTACCTGAGGCCGCCATCCACGCCGGTTGAGTCGCGTTCTGCCGCCTCCCG |
| | | CCTGTGGTGCCTCCTGAACTGCGTCCGCCGTCTAGGTAAGTTTAAAGCTCAGGTCG |
| | | AGACCGGGCCTTTGTCCGGCGCTCCCTTGGAGCCTACCTAGACTCAGCCGGCTCTC |
| | | CACGCTTTGCCTGACCCTGCTTGCTCAACTCTACGTCTTTGTTTCGTTTTCTGTTCTG |
| | | CGCCGTTACAGATCCAAGCTGTGACCGGCGCCTACCTGAGATCACCGGCGCCACCA |
| | | TGGCTTCTTACCCTGGACACCAGCATGCTTCTGCCTTTGACCAGGCTGCCAGATCCA |
| | | GGGGCCACTCCAACAGGAGAACTGCCCTAAGACCCAGAAGACAGCAGGAAGCCAC |
| | | TGAGGTGAGGCCTGAGCAGAAGATGCCAACCCTGCTGAGGGTGTACATTGATGGAC |
| | | CTCATGGCATGGGCAAGACCACCACCACTCAACTGCTGGTGGCACTGGGCTCCAGG |
| | | GATGACATTGTGTATGTGCCTGAGCCAATGACCTACTGGAGAGTGCTAGGAGCCTCT |
| | | GAGACCATTGCCAACATCTACACCACCCAGCACAGGCTGGACCAGGGAGAAATCTC |
| | | TGCTGGAGATGCTGCTGTGGTGATGACCTCTGCCCAGATCACAATGGGAATGCCCT |
| | | ATGCTGTGACTGATGCTGTTCTGGCTCCTCACATTGGAGGAGAGGCTGGCTCTTCTC |
| | | ATGCCCCTCCACCTGCCCTGACCCTGATCTTTGACAGACACCCCATTGCAGCCCTG |
| | | CTGTGCTACCCAGCAGCAAGGTACCTCATGGGCTCCATGACCCCACAGGCTGTGCT |
| | | GGCTTTTGTGGCCCTGATCCCTCCAACCCTCCCTGGCACCAACATTGTTCTGGGAG |
| | | CACTGCCTGAAGACAGACACATTGACAGGCTGGCAAAGAGGCAGAGACCTGGAGAG |
| | | AGACTGGACCTGGCCATGCTGGCTGCAATCAGAAGGGTGTATGGACTGCTGGCAAA |
| | | CACTGTGAGATACCTCCAGTGTGGAGGCTCTTGGAGAGAGGACTGGGGACAGCTCT |
| | | CTGGAACAGCAGTGCCCCCTCAAGGAGCTGAGCCCCAGTCCAATGCTGGTCCAAGA |
| | | CCCCACATTGGGGACACCCTGTTCACCCTGTTCAGAGCCCCTGAGCTGCTGGCTCC |
| | | CAATGGAGACCTGTACAATGTGTTTGCCTGGGCTCTGGATGTTCTAGCCAAGAGGCT |
| | | GAGGTCCATGCATGTGTTCATCCTGGACTATGACCAGTCCCCTGCTGGATGCAGAG |
| | | ATGCTCTGCTGCAACTAACCTCTGGCATGGTGCAGACCCATGTGACCACCCCTGGC |
| | | AGCATCCCCACCATCTGTGACCTAGCCAGAACCTTTGCCAGGGAGATGGGAGAGGC |
| | | CAACTAAGGCGCGCCACTCGAGCGCTAGCTGGCCAGACATGATAAGATACATTGAT |
| | | GAGTTTGGACAAACCACAACTAGAATGCAGTGAAAAAAATGCTTTATTTGTGAAATTT |
| | | GTGATGCTATTGCTTTATTTGTAACCATTATAAGCTGCAATAAACAAGTTAACAACAAC |
| | | AATTGCATTCATTTTATGTTTCAGGTTCAGGGGGAGGTGTGGGAGGTTTTTTAAAGCA |
| | | AGTAAAACCTCTACAAATGTGGTATGGAAGGCGCGCCCAATTCGCCCTATAGTGAGT |
| | | CGTATTACGTCGCGCTCACTGGCCGTCGTTTTACAACGTCGTGACTGGGAAAACCCT |
| | | GGCGTTACCCAACTTAATCGCCTTGCAGCACATCCCCCTTTCGCCAGCTGGCGTAAT |
| | | AGCGAAGAGGCCCGCACCGAAACGCCCTTCCCAACAGTTGCGCAGCCTGAATGGC |
| | | GAATGGGAGCGCCCTGTAGCGGCGCATTAAGCGCGGCGGGTGTGGTGGTTACGCG |
| | | CAGCGTGACCGCTACACTTGCCAGCGCCCTAGCGCCCGCTCCTTTCGCTTTCTTCC |
| | | CTTCCTTTCTCGCCACGTTCGCCGGCTTTCCCCGTCAAGCTCTAAATCGGGGGCTCC |
| | | CTTTAGGGTTCCGATTTAGTGCTTTACGGCACCTCGACCCCAAAAAACTTGATTAGG |
| | | GTGATGGTTGGCCTGTAGTGGGCCATAGCCCTGATAGACGGTTTTTCGCCCTTTGAC |
| | | GTTGGAGTCCACGTTCTTTAATAGTGGACTCTTGTTCCAAACTGGAACAACACTCAAC |
| | | CCTATCTCGGTCTATTCTTTTGATTTATAAGGGATTTTGCCGATTTCGGCCTATTGGT |
| | | TAAAAAATGAGCTGATTTAACAAAAATTTAACGCGAATTTTAACAAAATATTAACGCTT |
| | | ACAATTTAGGTGGCACTTTTCGGGGAAATGTGCGCGGAACCCCTATTTGTTTATTTTT |
| | | CTAAATACATTCAAATATGTATCCGCTCATGAGACAATAACCCTGATAAATGCTTCAA |
| | | TAATATTGAAAAAGGAAGAGTATGAGTATTCAACATTTCCGTGTCGCCCTTATTCCCT |
| | | TTTTTGCGGCATTTTGCCTTCCTGTTTTTGCTCACCCAGAAACGCTGGTGAAAGTAAA |
| | | AGATGCTGAAGATCAGTTGGGTGCACGAGTGGGTTACATCGAACTGGATCTCAACA |
| | | GCGGTAAGATCCTTGAGAGTTTTCGCCCCGAAGAACGTTTTCCAATGATGAGCACTT |
| | | TTAAAGTTCTGCTATGTGGCGCGGTATTATCCCGTATTGACGCCGGGCAAGAGCAAC |
| | | TCGGTCGCCGCATACACTATTCTCAGAATGACTTGGTTGAGTACTCACCAGTCACAG |
| | | AAAAGCATCTTACGGATGGCATGACAGTAAGAGAATTATGCAGTGCTGCCATAACCA |
| | | TGAGTGATAACACTGCGGCCAACTTACTTCTGACAACGATCGGAGGACCGAAGGAG |
| | | CTAACCGCTTTTTTGCACAACATGGGGGATCATGTAACTCGCCTTGATCGTTGGGAA |
| | | CCGGAGCTGAATGAAGCCATACCAAACGACGAGCGTGACACCACGATGCCTGTAGC |
| | | AATGGCAACAACGTTGCGCAAACTATTAACTGGCGAACTACTTACTCTAGCTTCCCG |
| | | GCAACAATTAATAGACTGGATGGAGGCGGATAAAGTTGCAGGACCACTTCTGCGCT |
| | | CGGCCCTTCCGGCTGGCTGGTTTATTGCTGATAAATCTGGAGCCGGTGAGCGTGGT |
| | | TCTCGCGGTATCATTGCAGCACTGGGGCCAGATGGTAAGCCCTCCCGTATCGTAGT |
| | | TATCTACACGACGGGGAGTCAGGCAACTATGGATGAACGAAATAGACAGATCGCTG |
| | | AGATAGGTGCCTCACTGATTAAGCATTGGTAACTGTCAGACCAAGTTTACTCATATAT |
| | | ACTTTAGATTGATTTAAAACTTCATTTTTAATTTAAAAGGATCTAGGTGAAGATCCTTT |
| | | TTGATAATCTCATGACCAAAATCCCTTAACGTGAGTTTTCGTTCCACTGAGCGTCAGA |
| | | CCCCGTAGAAAAGATCAAAGGATCTTCTTGAGATCCTTTTTTTCTGCGCGTAATCTGC |
| | | TGCTTGCAAACAAAAAAACCACCGCTACCAGCGGTGGTTTGTTTGCCGGATCAAGAG |
| | | CTACCAACTCTTTTTCCGAAGGTAACTGGCTTCAGCAGAGCGCAGATACCAAATACT |
| | | GTTCTTCTAGTGTAGCCGTAGTTAGGCCACCACTTCAAGAACTCTGTAGCACCGCCT |
| | | ACATACCTCGCTCTGCTAATCCTGTTACCAGTGGCTGCTGCCAGTGGCGATAAGTCG |
| | | TGTCTTACCGGGTTGGACTCAAGACGATAGTTACCGGATAAGGCGCAGCGGTCGGG |
| | | CTGAACGGGGGGTTCGTGCACACAGCCCAGCTTGGAGCGAACGACCTACACCGAA |
| | | CTGAGATACCTACAGCGTGAGCTATGAGAAAGCGCCACGCTTCCCGAAGGGAGAAA |
| | | GGCGGACAGGTATCCGGTAAGCGGCAGGGTCGGAACAGGAGAGCGCACGAGGGA |
| | | GCTTCCAGGGGGAAACGCCTGGTATCTTTATAGTCCTGTCGGGTTTCGCCACCTCTG |
| | | ACTTGAGCGTCGATTTTTGTGATGCTCGTCAGGGGGGCGGAGCCTATGGAAAAACG |
| | | CCAGCAACGCGGCCTTTTTACGGTTCCTGGCCTTTTGCTGGCCTTTTGCTCACATGG |
| | | TCTTTCCTGCGTTATCCCCTGATTCTGTGGATAACCGTATTACCGCCTTTGAGTGAGC |
| | | TGATACCGCTCGCCGCAGCCGAACGACCGAGCGCAGCGAGTCAGTGAGCGAGGAA |
| | | GCGGAGAGCGCCCAATACGCAAACCGCCTCTCCCCGCGCGTTGGCCGATTCATTAA |
| | | TGCAGCTGGCACGACAGGTTTCCCGACTGGAAAGCGGGCAGTGAGCGCAACGCAA |
| | | TTAATGTGAGTTAGCTCACTCATTAGGCACCCCAGGCTTTACACTTTATGCTTCCGGC |
| | | TCGTATGTTGTGTGGAATTGTGAGCGGATAACAATTTCACACAGGAAACAGCTATGA |
| | | CCATGATTACGCCAAGCGCGTCAATTAACCCTCACTAAAGGGAACAAAAGCTGTTAA |
| | | TTAA |
| 66 | Matrice CD25 | GTTTATTATTCCTGTTCCACAGCTATTGTCTGCCATATAAAAACTTAGGCCAGGCACA |
| | locus_IL12a_ | GTGGCTCACACCTGTAATCCCAGCACTTTGGAAGGCCGAGGCAGGCAGATCACAAG |
| | 2A_IL12b | GTCAGGAGTTCGAGACCAGCCTGGCCAACATAGCAAAACCCCATCTCTACTAAAAAT |
| | pCLS30520 | ACAAAAATTAGCCAGGCATGGTGGCGTGTGCACTGGTTTAGAGTGAGGACCACATTT |
| | full sequence | TTTTGGTGCCGTGTTACACATATGACCGTGACTTTGTTACACCACTACAGGAGGAAG |
| | | AGTAGAAGAACAATCGGTTCTGGCGTGAAACAGACTTTGAATTTTGACCTTCTCAAGT |
| | | TGGCGGGAGACGTGGAGTCCAACCCAGGGCCCATGTGGCCCCCTGGGTCAGCCTC |
| | | CCAGCCACCGCCCTCACCTGCCGCGGCCACAGGTCTGCATCCAGCGGCTCGCCCT |
| | | GTGTCCCTGCAGTGCCGGCTCAGCATGTGTCCAGCGCGCAGCCTCCTCCTTGTGGC |
| | | TACCCTGGTCCTCCTGGACCACCTCAGTTTGGCCAGAAACCTCCCCGTGGCCACTC |
| | | CAGACCCAGGAATGTTCCCATGCCTTCACCACTCCCAAAACCTGCTGAGGGCCGTC |
| | | AGCAACATGCTCCAGAAGGCCAGACAAACTCTAGAATTTTACCCTTGCACTTCTGAA |
| | | GAGATTGATCATGAAGATATCACAAAAGATAAAACCAGCACAGTGGAGGCCTGTTTA |
| | | CCATTGGAATTAACCAAGAATGAGAGTTGCCTAAATTCCAGAGAGACCTCTTTCATAA |
| | | CTAATGGGAGTTGCCTGGCCTCCAGAAAGACCTCTTTTATGATGGCCCTGTGCCTTA |
| | | GTAGTATTTATGAAGACTTGAAGATGTACCAGGTGGAGTTCAAGACCATGAATGCAA |
| | | AGCTTCTGATGGATCCTAAGAGGCAGATCTTTCTAGATCAAAACATGCTGGCAGTTA |
| | | TTGATGAGCTGATGCAGGCCCTGAATTTCAACAGTGAGACTGTGCCACAAAAATCCT |
| | | CCCTTGAAGAACCGGATTTTTATAAAACTAAAATCAAGCTCTGCATACTTCTTCATGC |
| | | TTTCAGAATTCGGGCAGTGACTATTGATAGAGTGATGAGCTATCTGAATGCTTCCGG |
| | | AAGCGGAGCTACTAACTTCAGCCTGCTGAAGCAGGCTGGAGACGTGGAGGAGAACC |
| | | CTGGACCTATGTGTCACCAGCAGTTGGTCATCTCTTGGTTTTCCCTGGTTTTTCTGG |
| | | CATCTCCCCTCGTGGCCATATGGGAACTGAAGAAAGATGTTTATGTCGTAGAATTGG |
| | | ATTGGTATCCGGATGCCCCTGGAGAAATGGTGGTCCTCACCTGTGACACCCCTGAA |
| | | GAAGATGGTATCACCTGGACCTTGGACCAGAGCAGTGAGGTCTTAGGCTCTGGCAA |
| | | AACCCTGACCATCCAAGTCAAAGAGTTTGGAGATGCTGGCCAGTACACCTGTCACAA |
| | | AGGAGGCGAGGTTCTAAGCCATTCGCTCCTGCTGCTTCACAAAAAGGAAGATGGAA |
| | | TTTGGTCCACTGATATTTTAAAGGACCAGAAAGAACCCAAAAATAAGACCTTTCTAAG |
| | | ATGCGAGGCCAAGAATTATTCTGGACGTTTCACCTGCTGGTGGCTGACGACAATCAG |
| | | TACTGATTTGACATTCAGTGTCAAAAGCAGCAGAGGCTCTTCTGACCCCCAAGGGGT |
| | | GACGTGCGGAGCTGCTACACTCTCTGCAGAGAGAGTCAGAGGGGACAACAAGGAG |
| | | TATGAGTACTCAGTGGAGTGCCAGGAGGACAGTGCCTGCCCAGCTGCTGAGGAGA |
| | | GTCTGCCCATTGAGGTCATGGTGGATGCCGTTCACAAGCTCAAGTATGAAAACTACA |
| | | CCAGCAGCTTCTTCATCAGGGACATCATCAAACCTGACCCACCCAAGAACTTGCAGC |
| | | TGAAGCCATTAAAGAATTCTCGGCAGGTGGAGGTCAGCTGGGAGTACCCTGACACC |
| | | TGGAGTACTCCACATTCCTACTTCTCCCTGACATTCTGCGTTCAGGTCCAGGGCAAG |
| | | AGCAAGAGAGAAAAGAAAGATAGAGTCTTCACGGACAAGACCTCAGCCACGGTCAT |
| | | CTGCCGCAAAAATGCCAGCATTAGCGTGCGGGCCCAGGACCGCTACTATAGCTCAT |
| | | CTTGGAGCGAATGGGCATCTGTGCCCTGCAGTGAGGGCAGAGGCAGCCTGCTGAC |
| | | CTGCGGCGACGTCGAGGAGAACCCCGGGCCCATGGGGGCAGGTGCCACCGGCCG |
| | | CGCCATGGACGGGCCGCGCCTGCTGCTGTTGCTGCTTCTGGGGGTGTCCCTTGGA |
| | | GGTGCCAAGGAGGCATGCCCCACAGGCCTGTACACACACAGCGGTGAGTGCTGCA |
| | | AAGCCTGCAACCTGGGCGAGGGTGTGGCCCAGCCTTGTGGAGCCAACCAGACCGT |
| | | GTGTGAGCCCTGCCTGGACAGCGTGACGTTCTCCGACGTGGTGAGCGCGACCGAG |
| | | CCGTGCAAGCCGTGCACCGAGTGCGTGGGGCTCCAGAGCATGTCGGCGCCGTGCG |
| | | TGGAGGCCGATGACGCCGTGTGCCGCTGCGCCTACGGCTACTACCAGGATGAGAC |
| | | GACTGGGCGCTGCGAGGCGTGCCGCGTGTGCGAGGCGGGCTCGGGCCTCGTGTT |
| | | CTCCTGCCAGGACAAGCAGAACACCGTGTGCGAGGAGTGCCCCGACGGCACGTAT |
| | | TCCGACGAGGCCAACCACGTGGACCCGTGCCTGCCCTGCACCGTGTGCGAGGACA |
| | | CCGAGCGCCAGCTCCGCGAGTGCACACGCTGGGCCGACGCCGAGTGCGAGGAGA |
| | | TCCCTGGCCGTTGGATTACACGGTCCACACCCCCAGAGGGCTCGGACAGCACAGC |
| | | CCCCAGCACCCAGGAGCCTGAGGCACCTCCAGAACAAGACCTCATAGCCAGCACG |
| | | GTGGCAGGTGTGGTGACCACAGTGATGGGCAGCTCCCAGCCCGTGGTGACCCGAG |
| | | GCACCACCGACAACCTCATCCCTGTCTATTGCTCCATCCTGGCTGCTGTGGTTGTGG |
| | | GTCTTGTGGCCTACATAGCCTTCAAGAGGTGAAAAACCAAAAGAACAAGAATTTCTT |
| | | GGTAAGAAGCCGGGAACAGACAACAGAAGTCATGAAGCCCAAGTGAAATCAAAGGT |
| | | GCTAAATGGTCGCCCAGGAGACATCCGTTGTGCTTGCCTGCGTTTTGGAAGCTCTG |
| | | AAGTCACATCACAGGACACGGGGCAGTGGCAACCTTGTCTCTATGCCAGCTCAGTC |
| | | CCATCAGAGAGCGAGCGCTACCCACTTCTAAATAGCAATTTCGCCGTTGAAGAGGAA |
| | | GGGCAAAACCACTAGAACTCTCCATCTTATTTTCATGTATATGTGTTCATGCGATCGC |
| | | TCCGGTGCCCGTCAGTGGGCAGAGCGCACATCGCCCACAGTCCCCGAGAAGTTGG |
| | | GGGGAGGGGTCGGCAATTGAACGGGTGCCTAGAGAAGGTGGCGCGGGGTAAACTG |
| | | GGAAAGTGATGTCGTGTACTGGCTCCGCCTTTTTCCCGAGGGTGGGGGAGAACCGT |
| | | ATATAAGTGCAGTAGTCGCCGTGAACGTTCTTTTTCGCAACGGGTTTGCCGCCAGAA |
| | | CACAGCTGAAGCTTCGAGGGGCTCGCATCTCTCCTTCACGCGCCCGCCGCCCTACC |
| | | TGAGGCCGCCATCCACGCCGGTTGAGTCGCGTTCTGCCGCCTCCCGCCTGTGGTG |
| | | CCTCCTGAACTGCGTCCGCCGTCTAGGTAAGTTTAAAGCTCAGGTCGAGACCGGGC |
| | | CTTTGTCCGGCGCTCCCTTGGAGCCTACCTAGACTCAGCCGGCTCTCCACGCTTTG |
| | | CCTGACCCTGCTTGCTCAACTCTACGTCTTTGTTTCGTTTTCTGTTCTGCGCCGTTAC |
| | | AGATCCAAGCTGTGACCGGCGCCTACCTGAGATCACCGGCGCCACCATGGCTTCTT |
| | | ACCCTGGACACCAGCATGCTTCTGCCTTTGACCAGGCTGCCAGATCCAGGGGCCAC |
| | | TCCAACAGGAGAACTGCCCTAAGACCCAGAAGACAGCAGGAAGCCACTGAGGTGAG |
| | | GCCTGAGCAGAAGATGCCAACCCTGCTGAGGGTGTACATTGATGGACCTCATGGCA |
| | | TGGGCAAGACCACCACCACTCAACTGCTGGTGGCACTGGGCTCCAGGGATGACATT |
| | | GTGTATGTGCCTGAGCCAATGACCTACTGGAGAGTGCTAGGAGCCTCTGAGACCAT |
| | | TGCCAACATCTACACCACCCAGCACAGGCTGGACCAGGGAGAAATCTCTGCTGGAG |
| | | ATGCTGCTGTGGTGATGACCTCTGCCCAGATCACAATGGGAATGCCCTATGCTGTGA |
| | | CTGATGCTGTTCTGGCTCCTCACATTGGAGGAGAGGCTGGCTCTTCTCATGCCCCTC |
| | | CACCTGCCCTGACCCTGATCTTTGACAGACACCCCATTGCAGCCCTGCTGTGCTACC |
| | | CAGCAGCAAGGTACCTCATGGGCTCCATGACCCCACAGGCTGTGCTGGCTTTTGTG |
| | | GCCCTGATCCCTCCAACCCTCCCTGGCACCAACATTGTTCTGGGAGCACTGCCTGA |
| | | AGACAGACACATTGACAGGCTGGCAAAGAGGCAGAGACCTGGAGAGAGACTGGAC |
| | | CTGGCCATGCTGGCTGCAATCAGAAGGGTGTATGGACTGCTGGCAAACACTGTGAG |
| | | ATACCTCCAGTGTGGAGGCTCTTGGAGAGAGGACTGGGGACAGCTCTCTGGAACAG |
| | | CAGTGCCCCCTCAAGGAGCTGAGCCCCAGTCCAATGCTGGTCCAAGACCCCACATT |
| | | GGGGACACCCTGTTCACCCTGTTCAGAGCCCCTGAGCTGCTGGCTCCCAATGGAGA |
| | | CCTGTACAATGTGTTTGCCTGGGCTCTGGATGTTCTAGCCAAGAGGCTGAGGTCCAT |
| | | GCATGTGTTCATCCTGGACTATGACCAGTCCCCTGCTGGATGCAGAGATGCTCTGCT |
| | | GCAACTAACCTCTGGCATGGTGCAGACCCATGTGACCACCCCTGGCAGCATCCCCA |
| | | CCATCTGTGACCTAGCCAGAACCTTTGCCAGGGAGATGGGAGAGGCCAACTAAGGC |
| | | GCGCCACTCGAGCGCTAGCTGGCCAGACATGATAAGATACATTGATGAGTTTGGAC |
| | | AAACCACAACTAGAATGCAGTGAAAAAAATGCTTTATTTGTGAAATTTGTGATGCTAT |
| | | TGCTTTATTTGTAACCATTATAAGCTGCAATAAACAAGTTAACAACAACAATTGCATTC |
| | | ATTTTATGTTTCAGGTTCAGGGGGAGGTGTGGGAGGTTTTTTAAAGCAAGTAAAACC |
| | | TCTACAAATGTGGTATGGAAGGCGCGCCCAATTCGCCCTATAGTGAGTCGTATTACG |
| | | TCGCGCTCACTGGCCGTCGTTTTACAACGTCGTGACTGGGAAAACCCTGGCGTTAC |
| | | CCAACTTAATCGCCTTGCAGCACATCCCCCTTTCGCCAGCTGGCGTAATAGCGAAGA |
| | | GGCCCGCACCGAAACGCCCTTCCCAACAGTTGCGCAGCCTGAATGGCGAATGGGA |
| | | GCGCCCTGTAGCGGCGCATTAAGCGCGGCGGGTGTGGTGGTTACGCGCAGCGTGA |
| | | CCGCTACACTTGCCAGCGCCCTAGCGCCCGCTCCTTTCGCTTTCTTCCCTTCCTTTC |
| | | TCGCCACGTTCGCCGGCTTTCCCCGTCAAGCTCTAAATCGGGGGCTCCCTTTAGGG |
| | | TTCCGATTTAGTGCTTTACGGCACCTCGACCCCAAAAAACTTGATTAGGGTGATGGT |
| | | TGGCCTGTAGTGGGCCATAGCCCTGATAGACGGTTTTTCGCCCTTTGACGTTGGAGT |
| | | CCACGTTCTTTAATAGTGGACTCTTGTTCCAAACTGGAACAACACTCAACCCTATCTC |
| | | GGTCTATTCTTTTGATTTATAAGGGATTTTGCCGATTTCGGCCTATTGGTTAAAAAAT |
| | | GAGCTGATTTAACAAAAATTTAACGCGAATTTTAACAAAATATTAACGCTTACAATTTA |
| | | GGTGGCACTTTTCGGGGAAATGTGCGCGGAACCCCTATTTGTTTATTTTTCTAAATAC |
| | | ATTCAAATATGTATCCGCTCATGAGACAATAACCCTGATAAATGCTTCAATAATATTGA |
| | | AAAAGGAAGAGTATGAGTATTCAACATTTCCGTGTCGCCCTTATTCCCTTTTTTGCGG |
| | | CATTTTGCCTTCCTGTTTTTGCTCACCCAGAAACGCTGGTGAAAGTAAAAGATGCTGA |
| | | AGATCAGTTGGGTGCACGAGTGGGTTACATCGAACTGGATCTCAACAGCGGTAAGA |
| | | TCCTTGAGAGTTTTCGCCCCGAAGAACGTTTTCCAATGATGAGCACTTTTAAAGTTCT |
| | | GCTATGTGGCGCGGTATTATCCCGTATTGACGCCGGGCAAGAGCAACTCGGTCGCC |
| | | GCATACACTATTCTCAGAATGACTTGGTTGAGTACTCACCAGTCACAGAAAAGCATCT |
| | | TACGGATGGCATGACAGTAAGAGAATTATGCAGTGCTGCCATAACCATGAGTGATAA |
| | | CACTGCGGCCAACTTACTTCTGACAACGATCGGAGGACCGAAGGAGCTAACCGCTT |
| | | TTTTGCACAACATGGGGGATCATGTAACTCGCCTTGATCGTTGGGAACCGGAGCTGA |
| | | ATGAAGCCATACCAAACGACGAGCGTGACACCACGATGCCTGTAGCAATGGCAACA |
| | | ACGTTGCGCAAACTATTAACTGGCGAACTACTTACTCTAGCTTCCCGGCAACAATTAA |
| | | TAGACTGGATGGAGGCGGATAAAGTTGCAGGACCACTTCTGCGCTCGGCCCTTCCG |
| | | GCTGGCTGGTTTATTGCTGATAAATCTGGAGCCGGTGAGCGTGGTTCTCGCGGTAT |
| | | CATTGCAGCACTGGGGCCAGATGGTAAGCCCTCCCGTATCGTAGTTATCTACACGA |
| | | CGGGGAGTCAGGCAACTATGGATGAACGAAATAGACAGATCGCTGAGATAGGTGCC |
| | | TCACTGATTAAGCATTGGTAACTGTCAGACCAAGTTTACTCATATATACTTTAGATTGA |
| | | TTTAAAACTTCATTTTTAATTTAAAAGGATCTAGGTGAAGATCCTTTTTGATAATCTCAT |
| | | GACCAAAATCCCTTAACGTGAGTTTTCGTTCCACTGAGCGTCAGACCCCGTAGAAAA |
| | | GATCAAAGGATCTTCTTGAGATCCTTTTTTTCTGCGCGTAATCTGCTGCTTGCAAACA |
| | | AAAAAACCACCGCTACCAGCGGTGGTTTGTTTGCCGGATCAAGAGCTACCAACTCTT |
| | | TTTCCGAAGGTAACTGGCTTCAGCAGAGCGCAGATACCAAATACTGTTCTTCTAGTG |
| | | TAGCCGTAGTTAGGCCACCACTTCAAGAACTCTGTAGCACCGCCTACATACCTCGCT |
| | | CTGCTAATCCTGTTACCAGTGGCTGCTGCCAGTGGCGATAAGTCGTGTCTTACCGG |
| | | GTTGGACTCAAGACGATAGTTACCGGATAAGGCGCAGCGGTCGGGCTGAACGGGG |
| | | GGTTCGTGCACACAGCCCAGCTTGGAGCGAACGACCTACACCGAACTGAGATACCT |
| | | ACAGCGTGAGCTATGAGAAAGCGCCACGCTTCCCGAAGGGAGAAAGGCGGACAGG |
| | | TATCCGGTAAGCGGCAGGGTCGGAACAGGAGAGCGCACGAGGGAGCTTCCAGGGG |
| | | GAAACGCCTGGTATCTTTATAGTCCTGTCGGGTTTCGCCACCTCTGACTTGAGCGTC |
| | | GATTTTTGTGATGCTCGTCAGGGGGGCGGAGCCTATGGAAAAACGCCAGCAACGCG |
| | | GCCTTTTTACGGTTCCTGGCCTTTTGCTGGCCTTTTGCTCACATGGTCTTTCCTGCGT |
| | | TATCCCCTGATTCTGTGGATAACCGTATTACCGCCTTTGAGTGAGCTGATACCGCTC |
| | | GCCGCAGCCGAACGACCGAGCGCAGCGAGTCAGTGAGCGAGGAAGCGGAGAGCG |
| | | CCCAATACGCAAACCGCCTCTCCCCGCGCGTTGGCCGATTCATTAATGCAGCTGGC |
| | | ACGACAGGTTTCCCGACTGGAAAGCGGGCAGTGAGCGCAACGCAATTAATGTGAGT |
| | | TAGCTCACTCATTAGGCACCCCAGGCTTTACACTTTATGCTTCCGGCTCGTATGTTGT |
| | | GTGGAATTGTGAGCGGATAACAATTTCACACAGGAAACAGCTATGACCATGATTACG |
| | | CCAAGCGCGTCAATTAACCCTCACTAAAGGGAACAAAAGCTGTTAATTAA |
| 67 | Matrice PD1 | TCGCGCGTTTCGGTGATGACGGTGAAAACCTCTGACACATGCAGCTCCCGGAGACG |
| | locus_IL12a_ | GTCACAGCTTGTCTGTAAGCGGATGCCGGGAGCAGACAAGCCCGTCAGGGCGCGT |
| | 2A_IL12b | CAGCGGGTGTTGGCGGGTGTCGGGGCTGGCTTAACTATGCGGCATCAGAGCAGAT |
| | pCLS30511 | TGTACTGAGAGTGCACCATATGCGGTGTGAAATACCGCACAGATGCGTAAGGAGAA |
| | full sequence | AATACCGCATCAGGCGCCATTCGCCATTCAGGCTGCGCAACTGTTGGGAAGGGCGA |
| | | TCGGTGCGGGCCTCTTCGCTATTACGCCAGCTGGCGAAAGGGGGATGTGCTGCAA |
| | | GGCGATTAAGTTGGGTAACGCCAGGGTTTTCCCAGTCACGACGTTGTAAAACGACG |
| | | GCCAGTGAATTCGAGCTCGGTACCTCGCGAATGCATCTAGATGACTCCCCAGACAG |
| | | GCCCTGGAACCCCCCCACCTTCTCCCCAGCCCTGCTCGTGGTGACCGAAGGGGAC |
| | | AACGCCACCTTCACCTGCAGCTTCTCCAACACATCGGAGAGCTTCGTGCTAAACTGG |
| | | TACCGCATGAGCCCCAGCAACCAGACGGACAAGCTGGCCGCCTTCCCCGAGGACC |
| | | GCAGCCAGCCCGGCCAGGACTGCCGCTTCCGTGTCACACAACTGCCCAACGGGCG |
| | | TGACTTCCACATGAGCGTGGTCAGGGCCCGGCGCAATGACAGCGGCACCTACCTCT |
| | | GTGGGGCCGGTTCTGGCGTGAAACAGACTTTGAATTTTGACCTTCTCAAGTTGGCG |
| | | GGAGACGTGGAGTCCAACCCAGGGCCCATGTGGCCCCCTGGGTCAGCCTCCCAGC |
| | | CACCGCCCTCACCTGCCGCGGCCACAGGTCTGCATCCAGCGGCTCGCCCTGTGTC |
| | | CCTGCAGTGCCGGCTCAGCATGTGTCCAGCGCGCAGCCTCCTCCTTGTGGCTACCC |
| | | TGGTCCTCCTGGACCACCTCAGTTTGGCCAGAAACCTCCCCGTGGCCACTCCAGAC |
| | | CCAGGAATGTTCCCATGCCTTCACCACTCCCAAAACCTGCTGAGGGCCGTCAGCAA |
| | | CATGCTCCAGAAGGCCAGACAAACTCTAGAATTTTACCCTTGCACTTCTGAAGAGAT |
| | | TGATCATGAAGATATCACAAAAGATAAAACCAGCACAGTGGAGGCCTGTTTACCATT |
| | | GGAATTAACCAAGAATGAGAGTTGCCTAAATTCCAGAGAGACCTCTTTCATAACTAAT |
| | | GGGAGTTGCCTGGCCTCCAGAAAGACCTCTTTTATGATGGCCCTGTGCCTTAGTAGT |
| | | ATTTATGAAGACTTGAAGATGTACCAGGTGGAGTTCAAGACCATGAATGCAAAGCTT |
| | | CTGATGGATCCTAAGAGGCAGATCTTTCTAGATCAAAACATGCTGGCAGTTATTGAT |
| | | GAGCTGATGCAGGCCCTGAATTTCAACAGTGAGACTGTGCCACAAAAATCCTCCCTT |
| | | GAAGAACCGGATTTTTATAAAACTAAAATCAAGCTCTGCATACTTCTTCATGCTTTCA |
| | | GAATTCGGGCAGTGACTATTGATAGAGTGATGAGCTATCTGAATGCTTCCGGAAGCG |
| | | GAGCTACTAACTTCAGCCTGCTGAAGCAGGCTGGAGACGTGGAGGAGAACCCTGGA |
| | | CCTATGTGTCACCAGCAGTTGGTCATCTCTTGGTTTTCCCTGGTTTTTCTGGCATCTC |
| | | CCCTCGTGGCCATATGGGAACTGAAGAAAGATGTTTATGTCGTAGAATTGGATTGGT |
| | | ATCCGGATGCCCCTGGAGAAATGGTGGTCCTCACCTGTGACACCCCTGAAGAAGAT |
| | | GGTATCACCTGGACCTTGGACCAGAGCAGTGAGGTCTTAGGCTCTGGCAAAACCCT |
| | | GACCATCCAAGTCAAAGAGTTTGGAGATGCTGGCCAGTACACCTGTCACAAAGGAG |
| | | GCGAGGTTCTAAGCCATTCGCTCCTGCTGCTTCACAAAAAGGAAGATGGAATTTGGT |
| | | CCACTGATATTTTAAAGGACCAGAAAGAACCCAAAAATAAGACCTTTCTAAGATGCGA |
| | | GGCCAAGAATTATTCTGGACGTTTCACCTGCTGGTGGCTGACGACAATCAGTACTGA |
| | | TTTGACATTCAGTGTCAAAAGCAGCAGAGGCTCTTCTGACCCCCAAGGGGTGACGT |
| | | GCGGAGCTGCTACACTCTCTGCAGAGAGAGTCAGAGGGGACAACAAGGAGTATGAG |
| | | TACTCAGTGGAGTGCCAGGAGGACAGTGCCTGCCCAGCTGCTGAGGAGAGTCTGC |
| | | CCATTGAGGTCATGGTGGATGCCGTTCACAAGCTCAAGTATGAAAACTACACCAGCA |
| | | GCTTCTTCATCAGGGACATCATCAAACCTGACCCACCCAAGAACTTGCAGCTGAAGC |
| | | CATTAAAGAATTCTCGGCAGGTGGAGGTCAGCTGGGAGTACCCTGACACCTGGAGT |
| | | ACTCCACATTCCTACTTCTCCCTGACATTCTGCGTTCAGGTCCAGGGCAAGAGCAAG |
| | | AGAGAAAAGAAAGATAGAGTCTTCACGGACAAGACCTCAGCCACGGTCATCTGCCG |
| | | CAAAAATGCCAGCATTAGCGTGCGGGCCCAGGACCGCTACTATAGCTCATCTTGGA |
| | | GCGAATGGGCATCTGTGCCCTGCAGTGAGGGCAGAGGCAGCCTGCTGACCTGCGG |
| | | CGACGTCGAGGAGAACCCCGGGCCCATGGGGGCAGGTGCCACCGGCCGCGCCAT |
| | | GGACGGGCCGCGCCTGCTGCTGTTGCTGCTTCTGGGGGTGTCCCTTGGAGGTGCC |
| | | AAGGAGGCATGCCCCACAGGCCTGTACACACACAGCGGTGAGTGCTGCAAAGCCT |
| | | GCAACCTGGGCGAGGGTGTGGCCCAGCCTTGTGGAGCCAACCAGACCGTGTGTGA |
| | | GCCCTGCCTGGACAGCGTGACGTTCTCCGACGTGGTGAGCGCGACCGAGCCGTGC |
| | | AAGCCGTGCACCGAGTGCGTGGGGCTCCAGAGCATGTCGGCGCCGTGCGTGGAGG |
| | | CCGATGACGCCGTGTGCCGCTGCGCCTACGGCTACTACCAGGATGAGACGACTGG |
| | | GCGCTGCGAGGCGTGCCGCGTGTGCGAGGCGGGCTCGGGCCTCGTGTTCTCCTGC |
| | | CAGGACAAGCAGAACACCGTGTGCGAGGAGTGCCCCGACGGCACGTATTCCGACG |
| | | AGGCCAACCACGTGGACCCGTGCCTGCCCTGCACCGTGTGCGAGGACACCGAGCG |
| | | CCAGCTCCGCGAGTGCACACGCTGGGCCGACGCCGAGTGCGAGGAGATCCCTGGC |
| | | CGTTGGATTACACGGTCCACACCCCCAGAGGGCTCGGACAGCACAGCCCCCAGCA |
| | | CCCAGGAGCCTGAGGCACCTCCAGAACAAGACCTCATAGCCAGCACGGTGGCAGG |
| | | TGTGGTGACCACAGTGATGGGCAGCTCCCAGCCCGTGGTGACCCGAGGCACCACC |
| | | GACAACCTCATCCCTGTCTATTGCTCCATCCTGGCTGCTGTGGTTGTGGGTCTTGTG |
| | | GCCTACATAGCCTTCAAGAGGTGATCTAGAGGGCCCGTTTAAACCCGCTGATCAGC |
| | | CTCGACTGTGCCTTCTAGTTGCCAGCCATCTGTTGTTTGCCCCTCCCCCGTGCCTTC |
| | | CTTGACCCTGGAAGGTGCCACTCCCACTGTCCTTTCCTAATAAAATGAGGAAATTGC |
| | | ATCGCATTGTCTGAGTAGGTGTCATTCTATTCTGGGGGGTGGGGTGGGGCAGGACA |
| | | GCAAGGGGGAGGATTGGGAAGACAATAGCAGGCATGCTGGGGATGCGGTGGGCTC |
| | | TATGACTAGTGGCGAATTCGGCGCAGATCAAAGAGAGCCTGCGGGCAGAGCTCAGG |
| | | GTGACAGGTGCGGCCTCGGAGGCCCCGGGGCAGGGGTGAGCTGAGCCGGTCCTG |
| | | GGGTGGGTGTCCCCTCCTGCACAGGATCAGGAGCTCCAGGGTCGTAGGGCAGGGA |
| | | CCCCCCAGCTCCAGTCCAGGGCTCTGTCCTGCACCTGGGGAATGGTGACCGGCAT |
| | | CTCTGTCCTCTAGCTCTGGAAGCACCCCAGCCCCTCTAGTCTGCCCTCACCCCTGA |
| | | CCCTGACCCTCCACCCTGACCCCGTCCTAACCCCTGACCTTTGATCGGATCCCGGG |
| | | CCCGTCGACTGCAGAGGCCTGCATGCAAGCTTGGCGTAATCATGGTCATAGCTGTT |
| | | TCCTGTGTGAAATTGTTATCCGCTCACAATTCCACACAACATACGAGCCGGAAGCAT |
| | | AAAGTGTAAAGCCTGGGGTGCCTAATGAGTGAGCTAACTCACATTAATTGCGTTGCG |
| | | CTCACTGCCCGCTTTCCAGTCGGGAAACCTGTCGTGCCAGCTGCATTAATGAATCG |
| | | GCCAACGCGCGGGGAGAGGCGGTTTGCGTATTGGGCGCTCTTCCGCTTCCTCGCT |
| | | CACTGACTCGCTGCGCTCGGTCGTTCGGCTGCGGCGAGCGGTATCAGCTCACTCAA |
| | | AGGCGGTAATACGGTTATCCACAGAATCAGGGGATAACGCAGGAAAGAACATGTGA |
| | | GCAAAAGGCCAGCAAAAGGCCAGGAACCGTAAAAAGGCCGCGTTGCTGGCGTTTTT |
| | | CCATAGGCTCCGCCCCCCTGACGAGCATCACAAAAATCGACGCTCAAGTCAGAGGT |
| | | GGCGAAACCCGACAGGACTATAAAGATACCAGGCGTTTCCCCCTGGAAGCTCCCTC |
| | | GTGCGCTCTCCTGTTCCGACCCTGCCGCTTACCGGATACCTGTCCGCCTTTCTCCCT |
| | | TCGGGAAGCGTGGCGCTTTCTCATAGCTCACGCTGTAGGTATCTCAGTTCGGTGTA |
| | | GGTCGTTCGCTCCAAGCTGGGCTGTGTGCACGAACCCCCCGTTCAGCCCGACCGCT |
| | | GCGCCTTATCCGGTAACTATCGTCTTGAGTCCAACCCGGTAAGACACGACTTATCGC |
| | | CACTGGCAGCAGCCACTGGTAACAGGATTAGCAGAGCGAGGTATGTAGGCGGTGCT |
| | | ACAGAGTTCTTGAAGTGGTGGCCTAACTACGGCTACACTAGAAGAACAGTATTTGGT |
| | | ATCTGCGCTCTGCTGAAGCCAGTTACCTTCGGAAAAAGAGTTGGTAGCTCTTGATCC |
| | | GGCAAACAAACCACCGCTGGTAGCGGTGGTTTTTTTGTTTGCAAGCAGCAGATTACG |
| | | CGCAGAAAAAAAGGATCTCAAGAAGATCCTTTGATCTTTTCTACGGGGTCTGACGCT |
| | | CAGTGGAACGAAAACTCACGTTAAGGGATTTTGGTCATGAGATTATCAAAAAGGATC |
| | | TTCACCTAGATCCTTTTAAATTAAAAATGAAGTTTTAAATCAATCTAAAGTATATATGA |
| | | GTAAACTTGGTCTGACAGTTACCAATGCTTAATCAGTGAGGCACCTATCTCAGCGAT |
| | | CTGTCTATTTCGTTCATCCATAGTTGCCTGACTCCCCGTCGTGTAGATAACTACGATA |
| | | CGGGAGGGCTTACCATCTGGCCCCAGTGCTGCAATGATACCGCGAGACCCACGCTC |
| | | ACCGGCTCCAGATTTATCAGCAATAAACCAGCCAGCCGGAAGGGCCGAGCGCAGAA |
| | | GTGGTCCTGCAACTTTATCCGCCTCCATCCAGTCTATTAATTGTTGCCGGGAAGCTA |
| | | GAGTAAGTAGTTCGCCAGTTAATAGTTTGCGCAACGTTGTTGCCATTGCTACAGGCA |
| | | TCGTGGTGTCACGCTCGTCGTTTGGTATGGCTTCATTCAGCTCCGGTTCCCAACGAT |
| | | CAAGGCGAGTTACATGATCCCCCATGTTGTGCAAAAAAGCGGTTAGCTCCTTCGGTC |
| | | CTCCGATCGTTGTCAGAAGTAAGTTGGCCGCAGTGTTATCACTCATGGTTATGGCAG |
| | | CACTGCATAATTCTCTTACTGTCATGCCATCCGTAAGATGCTTTTCTGTGACTGGTGA |
| | | GTACTCAACCAAGTCATTCTGAGAATAGTGTATGCGGCGACCGAGTTGCTCTTGCCC |
| | | GGCGTCAATACGGGATAATACCGCGCCACATAGCAGAACTTTAAAAGTGCTCATCAT |
| | | TGGAAAACGTTCTTCGGGGCGAAAACTCTCAAGGATCTTACCGCTGTTGAGATCCAG |
| | | TTCGATGTAACCCACTCGTGCACCCAACTGATCTTCAGCATCTTTTACTTTCACCAGC |
| | | GTTTCTGGGTGAGCAAAAACAGGAAGGCAAAATGCCGCAAAAAAGGGAATAAGGGC |
| | | GACACGGAAATGTTGAATACTCATACTCTTCCTTTTTCAATATTATTGAAGCATTTATC |
| | | AGGGTTATTGTCTCATGAGCGGATACATATTTGAATGTATTTAGAAAAATAAACAAAT |
| | | AGGGGTTCCGCGCACATTTCCCCGAAAAGTGCCACCTGACGTCTAAGAAACCATTAT |
| | | TATCATGACATTAACCTATAAAAATAGGCGTATCACGAGGCCCTTTCGTC |

TABLE 6
Preferred human endogenous gene loci responsive to T-cell activation

| | | inductionRatio12 | | T.8Eff.Sp.OT1. | T.8Eff.Sp.OT1. | T.8Eff.Sp.OT1. |
|---|---|---|---|---|---|---|
| symbol | description | hr | T.8Nve.Sp.OT1 | 12 hr.LisOva | 48 hr.LisOva | d6.LisOva |
| Il3 | interleukin 21 | 16.4 | 12.8 | 208.9 | 18.4 | 13.6 |
| Il2 | interleukin 3 | 97.0 | 16.0 | 1554.4 | 17.7 | 18.1 |
| CcI4 | isopentenyl-diphosphate delta isomerase 2 | 2.1 | 16.8 | 35.6 | 17.6 | 19.7 |
| Il21 | granzyme C | 9.2 | 17.4 | 160.5 | 20.4 | 24.9 |
| Gp49a | chemokine (C-C motif) receptor 8 | 5.9 | 18.5 | 108.4 | 31.5 | 20.9 |
| Cxcl10 | interleukin 2 | 58.4 | 21.1 | 1229.6 | 32.7 | 17.9 |
| Nr4a3 | interleukin 1 receptor, type I | 2.6 | 21.2 | 54.6 | 35.5 | 21.7 |
| Lilrb4 | tumor necrosis factor (ligand) superfamily, | 4.1 | 21.8 | 88.8 | 29.3 | 20.0 |
| | member 4 | | | | | |
| Cd200 | neuronal calcium sensor 1 | 4.5 | 24.1 | 109.6 | 46.3 | 23.2 |
| Cdkn1a | CDK5 and Abl enzyme substrate 1 | 3.1 | 26.2 | 80.9 | 49.1 | 32.8 |
| Gzmc | transmembrane and tetratricopeptide repeat | 2.0 | 26.8 | 53.9 | 26.2 | 29.4 |
| | containing 2 | | | | | |
| Nr4a2 | LON peptidase N-terminal domain and ring | 3.2 | 28.4 | 90.4 | 50.4 | 28.3 |
| | finger 1 | | | | | |
| Cish | glycoprotein 49 A | 15.0 | 31.6 | 472.4 | 30.6 | 212.5 |
| Nr4a1 | polo-like kinase 2 | 3.6 | 31.7 | 114.3 | 39.0 | 32.5 |
| Tnf | lipase, endothelial | 2.1 | 32.4 | 66.7 | 35.9 | 33.3 |
| Ccr8 | cyclin-dependent kinase inhibitor 1A (P21) | 9.7 | 34.6 | 335.4 | 54.4 | 71.0 |
| Lad1 | grainyhead-like 1 (Drosophila) | 2.1 | 35.1 | 73.4 | 52.0 | 44.1 |
| Slamf1 | cellular retinoic acid binding protein II | 5.3 | 35.4 | 187.2 | 43.3 | 36.3 |
| Crabp2 | adenylate kinase 4 | 2.2 | 35.9 | 80.4 | 58.5 | 39.8 |
| Furin | microtubule-associated protein 1B | 2.1 | 36.2 | 77.7 | 36.4 | 38.4 |
| Gadd45g | acyl-CoA synthetase long-chain family | 2.0 | 37.2 | 76.0 | 45.2 | 41.3 |
| | member 6 | | | | | |
| Bcl2l1 | zinc finger E-box binding homeobox 2 | 2.1 | 38.6 | 80.7 | 44.9 | 455.4 |
| Ncs1 | CD200 antigen | 9.8 | 41.2 | 404.3 | 70.4 | 36.8 |
| Ciart | carboxypeptidase D | 3.1 | 41.6 | 127.7 | 71.4 | 71.6 |
| Ahr | thioredoxin reductase 3 | 3.6 | 43.4 | 157.8 | 61.7 | 28.8 |
| Spry1 | myosin IE | 2.3 | 43.6 | 100.2 | 61.3 | 77.0 |
| Tnfsf4 | RNA binding protein with multiple splicing 2 | 2.1 | 43.6 | 91.5 | 49.8 | 36.5 |
| Myo10 | mitogen-activated protein kinase kinase 3, | 2.9 | 44.8 | 127.9 | 66.4 | 43.1 |
| | opposite strand | | | | | |
| Dusp5 | PERP, TP53 apoptosis effector | 2.8 | 44.9 | 127.2 | 78.4 | 72.4 |
| Myc | myosin X | 4.1 | 45.5 | 184.9 | 81.6 | 57.5 |
| Psrc1 | immediate early response 3 | 2.7 | 45.6 | 121.6 | 63.9 | 66.2 |
| St6galnac4 | folliculin interacting protein 2 | 2.6 | 47.5 | 124.2 | 87.4 | 96.6 |
| Nfkbid | leukocyte immunoglobulin-like receptor, | 9.9 | 48.9 | 483.3 | 64.5 | 179.1 |
| | subfamily B, member 4 | | | | | |
| Bst2 | circadian associated repressor of | 4.5 | 50.6 | 225.5 | 100.3 | 33.8 |
| | transcription | | | | | |
| Txnrd3 | RAR-related orphan receptor gamma | 2.1 | 51.7 | 106.7 | 47.5 | 52.8 |
| Plk2 | proline/serine-rich coiled-coil 1 | 3.9 | 52.9 | 205.9 | 92.3 | 79.6 |
| Gfi1 | cysteine rich protein 2 | 2.4 | 54.2 | 127.7 | 90.3 | 182.9 |
| Pim1 | cAMP responsive element modulator | 2.0 | 55.7 | 112.6 | 54.4 | 57.3 |
| Pvt1 | chemokine (C-C motif) ligand 4 | 20.2 | 55.8 | 1125.8 | 103.1 | 89.0 |
| Nfkbib | nuclear receptor subfamily 4, group A, | 7.8 | 58.5 | 457.6 | 78.7 | 72.0 |
| | member 2 | | | | | |
| Gnl2 | transglutaminase 2, C polypeptide | 2.3 | 58.7 | 132.1 | 69.8 | 64.7 |
| Cd69 | synapse defective 1, Rho GTPase, homolog 2 | | | | | |
| | (C, elegans) | 2.1 | 62.5 | 132.7 | 111.3 | 31.0 |
| Dgat2 | sprouty homolog 1 (Drosophila) | 4.2 | 63.8 | 268.5 | 76.8 | 61.4 |
| Atf3 | activating transcription factor 3 | 3.2 | 65.8 | 210.3 | 88.3 | 75.8 |
| Tnfrsf21 | pogo transposable element with KRAB | | | | | |
| | domain | 2.9 | 68.6 | 196.9 | 91.1 | 293.2 |
| Lonrfl | tumor necrosis factor receptor superfamily, | 3.2 | 70.6 | 224.5 | 126.5 | 72.9 |
| | member 21 | | | | | |
| Cables1 | cytokine inducible SH2-containing protein | 7.5 | 74.3 | 558.7 | 82.5 | 133.9 |
| Cpd | lymphotoxin A | 2.6 | 74.6 | 197.2 | 93.4 | 58.6 |
| Qtrtdl | FBJ osteosarcoma oncogene | 3.0 | 74.9 | 224.1 | 89.0 | 61.1 |
| Polr3d | signaling lymphocytic activation molecule | 5.4 | 75.6 | 412.0 | 108.4 | 190.4 |
| | family member 1 | | | | | |
| Kcnq5 | syndecan 3 | 2.4 | 76.0 | 180.0 | 77.2 | 85.3 |
| Fos | mitochondrial ribosomal protein L47 | 2.1 | 77.2 | 161.7 | 152.0 | 72.3 |
| Slc19a2 | ladinin | 5.5 | 77.3 | 423.2 | 152.5 | 70.4 |
| Hif1a | E2F transcription factor 5 | 2.5 | 77.7 | 198.0 | 92.0 | 65.2 |
| Il15ra | ISG15 ubiquitin-like modifier | 2.8 | 77.9 | 221.0 | 88.9 | 45.1 |
| Nfkb1 | aryl-hydrocarbon receptor | 4.2 | 78.7 | 333.2 | 145.7 | 91.4 |
| PhIda3 | diacylglycerol O-acyltransferase 2 | 3.2 | 81.0 | 259.2 | 150.0 | 84.4 |
| Mtrr | FBJ osteosarcoma oncogene B | 2.0 | 81.3 | 163.7 | 139.3 | 98.5 |
| Pogk | pleckstrin homology-like domain, family A, | 2.9 | 84.8 | 244.5 | 126.9 | 83.8 |
| | member 3 | | | | | |
| Map2k3os | potassium voltage-gated channel, subfamily | 3.0 | 86.3 | 261.0 | 118.1 | 63.4 |
| | Q, member 5 | | | | | |
| Egr2 | tumor necrosis factor receptor superfamily, | 2.5 | 88.6 | 219.0 | 106.1 | 51.0 |
| | member 10b | | | | | |
| Isg15 | Mir17 host gene 1 (non-protein coding) | 2.1 | 90.4 | 190.1 | 120.0 | 51.2 |
| Perp | glucose-fructose oxidoreductase domain | | | | | |
| | containing 1 | 2.2 | 92.9 | 208.5 | 168.7 | 237.4 |
| Ipo4 | plexin Al | 2.1 | 94.8 | 200.7 | 118.0 | 90.3 |
| Mphosph10 | heat shock factor 2 | 2.4 | 96.8 | 233.2 | 191.0 | 104.8 |
| Plk3 | carbohydrate sulfotransferase 11 | 2.4 | 96.8 | 235.1 | 180.8 | 385.7 |
| Ifitm3 | growth arrest and DNA-damage-inducible 45 | | | | | |
| | gamma | 4.8 | 104.6 | 504.8 | 109.3 | 95.0 |
| Polr1b | solute carrier family 5 (sodium-dependent | 2.1 | 107.0 | 227.3 | 192.8 | 75.8 |
| | vitamin transporter), member 6 | | | | | |
| Usp18 | interferon induced transmembrane protein 3 | 2.8 | 109.2 | 302.6 | 43.9 | 106.4 |
| Top1mt | DENN/MADD domain containing 5A | 2.6 | 109.5 | 279.9 | 102.0 | 517.4 |
| Dkc1 | plasminogen activator, urokinase receptor | 2.1 | 112.4 | 234.8 | 55.7 | 57.3 |
| Polr1c | solute carrier family 19 (thiamine | 3.0 | 115.4 | 343.1 | 221.7 | 138.4 |
| | transporter), member 2 | | | | | |
| Cdk6 | ubiquitin domain containing 2 | 2.2 | 117.4 | 255.7 | 198.9 | 122.2 |
| Ier3 | nuclear receptor subfamily 4, group A, | | | | | |
| | member 3 | 11.8 | 118.0 | 1394.1 | 114.2 | 69.6 |
| Lta | zinc finger protein 52 | 2.5 | 118.8 | 295.6 | 160.9 | 167.4 |
| Ptprs | SH3 domain containing ring finger 1 | 2.4 | 119.3 | 280.9 | 116.5 | 156.5 |
| Fnip2 | dihydrouridine synthase 2 | 2.1 | 122.7 | 260.3 | 237.7 | 202.8 |
| Asna1 | cyclin-dependent kinase 5, regulatory subunit | | | | | |
| | 1 (p35) | 2.1 | 122.7 | 259.3 | 168.4 | 124.0 |
| Mybbp1a | processing of precursor 7, ribonuclease P | | | | | |
| | family, (S, cerevisiae) | 2.1 | 125.9 | 264.9 | 235.7 | 150.6 |
| Il1r1 | growth factor independent 1 | 3.5 | 126.8 | 437.7 | 212.0 | 156.6 |
| Dennd5a | interleukin 15 receptor, alpha chain | 2.9 | 130.9 | 380.1 | 144.3 | 167.8 |
| E2f5 | BCL2-like 1 | 4.7 | 133.7 | 627.4 | 257.4 | 231.2 |
| Rcl1 | protein tyrosine phosphatase, receptor type, | 2.6 | 136.6 | 358.8 | 157.5 | 125.0 |
| | S | | | | | |
| FosI2 | plasmacytoma variant translocation 1 | 3.4 | 136.7 | 465.5 | 179.8 | 140.7 |
| Atad3a | fos-like antigen 2 | 2.5 | 137.0 | 347.5 | 107.2 | 177.8 |
| Bax | BCL2-associated X protein | 2.5 | 138.0 | 347.3 | 260.1 | 150.2 |
| Phf6 | solute carrier family 4, sodium bicarbonate | | | | | |
| | cotransporter, member 7 | 2.3 | 140.3 | 328.2 | 258.7 | 397.5 |
| Zfp52 | tumor necrosis factor receptor superfamily, | | | | | |
| | member 4 | 2.2 | 141.7 | 311.1 | 161.7 | 111.6 |
| Crtam | chemokine (C—X—C motif) ligand 10 | 12.7 | 141.7 | 1798.3 | 242.1 | 59.4 |
| Nop14 | polo-like kinase 3 | 2.8 | 144.8 | 406.3 | 200.1 | 119.9 |
| Rel | CD3E antigen, epsilon polypeptide associated | 2.2 | 158.7 | 350.2 | 260.9 | 111.4 |
| | protein | | | | | |
| Gramd1b | tumor necrosis factor (ligand) superfamily, | 2.1 | 162.4 | 342.1 | 242.1 | 169.7 |
| | member 11 | | | | | |
| Ifi27l2a | polymerase (RNA) III (DNA directed) | 3.0 | 166.3 | 503.7 | 296.1 | 121.6 |
| | polypeptide D | | | | | |
| Tnfrsf10b | early growth response 2 | 2.8 | 173.5 | 494.0 | 136.3 | 68.2 |
| Rpl7l1 | DnaJ (Hsp40) homolog, subfamily C, member | 2.1 | 173.6 | 369.4 | 346.2 | 254.3 |
| | 2 | | | | | |
| Eif1a | DNA topoisomerase 1, mitochondrial | 2.7 | 182.2 | 498.2 | 338.6 | 114.4 |
| Nfkb2 | tripartite motif-containing 30D | 2.3 | 182.6 | 423.4 | 65.8 | 90.6 |
| Heatr1 | DnaJ (Hsp40) homolog, subfamily C, member | | | | | |
| | 21 | 2.0 | 190.1 | 389.4 | 285.5 | 228.2 |
| | SAM domain, SH3 domain and nuclear | | | | | |
| Utp20 | localization signals, 1 | 2.2 | 191.5 | 422.1 | 222.8 | 304.1 |
| Chst11 | solute carrier family 5 (inositol transporters), | 2.1 | 191.6 | 400.2 | 210.0 | 123.4 |
| | member 3 | | | | | |
| Ddx21 | mitochondrial ribosomal protein L15 | 2.1 | 191.6 | 396.3 | 329.8 | 137.7 |
| Hsf2 | dual specificity phosphatase 5 | 4.0 | 203.5 | 818.1 | 307.5 | 560.7 |
| Bccip | apoptosis enhancing nuclease | 2.3 | 211.1 | 478.5 | 288.2 | 137.9 |
| Tagap | ets variant 6 | 2.3 | 218.3 | 508.1 | 220.5 | 297.3 |
| Sdc3 | DIM1 dimethyladenosine transferase 1-like | | | | | |
| | (S, cerevisiae) | 2.2 | 218.4 | 486.0 | 356.0 | 129.7 |
| SytI3 | 2'-5′ oligoadenylate synthetase-like 1 | 2.1 | 229.0 | 473.3 | 130.7 | 124.3 |
| Gtpbp4 | UTP18, small subunit (SSU) processome | 2.1 | 232.0 | 494.3 | 384.9 | 189.5 |
| | component, homolog (yeast) | | | | | |
| Crip2 | BRCA2 and CDKN1A interacting protein | 2.4 | 234.6 | 563.3 | 437.5 | 269.8 |
| Sh3rf1 | synaptotagmin-like 3 | 2.4 | 242.4 | 572.9 | 316.7 | 700.7 |
| Nsfl1c | 5-methyltetrahydrofolate-homocysteine | 2.9 | 245.7 | 706.5 | 334.6 | 150.6 |
| | methyltransferase reductase | | | | | |
| Gtf2f1 | URB2 ribosome biogenesis 2 homolog | 2.0 | 245.7 | 500.2 | 489.8 | 184.6 |
| | (S, cerevisiae) | | | | | |
| Slc4a7 | ubiquitin-conjugating enzyme E2C binding | 2.1 | 251.2 | 530.5 | 288.2 | 85.2 |
| | protein | | | | | |
| Etv6 | lysine (K)-specific demethylase 2B | 2.2 | 251.8 | 547.1 | 332.7 | 262.1 |
| Trim30d | queuine tRNA-ribosyltransferase domain | 3.0 | 260.3 | 788.7 | 358.0 | 75.5 |
| | containing 1 | | | | | |
| Ddx27 | ubiquitin specific peptidase 31 | 2.0 | 265.2 | 533.2 | 277.1 | 176.2 |
| Pwp2 | eukaryotic translation initiation factor 2- | 2.0 | 267.7 | 540.5 | 260.8 | 244.8 |
| | alpha kinase 2 | | | | | |
| Chchd2 | ATPase family, AAA domain containing 3A | 2.5 | 268.8 | 679.7 | 523.1 | 147.1 |
| Myo1e | adhesion molecule, interacts with CXADR | | | | | |
| | antigen 1 | 2.3 | 269.5 | 610.9 | 272.9 | 182.8 |
| Eif5b | SUMO/sentrin specific peptidase 3 | 2.0 | 272.5 | 548.7 | 544.5 | 298.4 |
| Stat5a | ESF1, nucleolar pre-rRNA processing protein, | 2.2 | 276.3 | 610.4 | 482.2 | 266.5 |
| | homolog (S, cerevisiae) | | | | | |
| Cops6 | deoxynucleotidyltransferase, terminal, | 2.1 | 282.9 | 600.4 | 359.9 | 326.1 |
| | interacting protein 2 | | | | | |
| D19Bwg1357e | TGFB-induced factor homeobox 1 | 2.1 | 300.5 | 618.9 | 217.5 | 210.6 |
| Aatf | eukaryotic translation initiation factor 1A | 2.5 | 300.8 | 738.7 | 597.7 | 262.8 |
| Aen | interferon-stimulated protein | 2.1 | 305.7 | 651.2 | 144.3 | 138.4 |
| Amica1 | pleiomorphic adenoma gene-like 2 | 2.1 | 311.5 | 651.9 | 376.2 | 405.9 |
| Wdr43 | PWP2 periodic tryptophan protein homolog | 2.3 | 321.8 | 743.3 | 586.5 | 189.3 |
| | (yeast) | | | | | |
| Cct4 | furin (paired basic amino acid cleaving | 5.2 | 329.7 | 1728.3 | 271.7 | 421.5 |
| | enzyme) | | | | | |
| Nifk | tumor necrosis factor | 6.6 | 330.7 | 2188.4 | 489.9 | 213.3 |
| Tgm2 | apoptosis antagonizing transcription factor | 2.3 | 331.4 | 754.8 | 523.1 | 221.5 |
| Ero1l | interferon, alpha-inducible protein 27 like 2A | 2.5 | 334.0 | 828.1 | 296.0 | 221.4 |
| Gfod1 | ST6 (alpha-N-acetyl-neuraminyl-2,3-beta- | 3.9 | 338.4 | 1311.3 | 636.0 | 298.2 |
| | galactosyl-1,3)-N-acetylgalactosaminide | | | | | |
| | alpha-2,6-sialyltransferase 4 | | | | | |
| Ak4 | methyltransferase like 1 | 2.2 | 339.4 | 744.7 | 662.8 | 94.5 |
| Sdad1 | notchless homolog 1 (Drosophila) | 2.0 | 339.4 | 690.3 | 610.3 | 158.1 |
| Dimt1 | mitochondrial ribosomal protein L3 | 2.1 | 340.0 | 725.5 | 651.4 | 359.8 |
| Esf1 | UBX domain protein 2A | 2.1 | 343.8 | 732.9 | 532.1 | 428.5 |
| Cd3eap | guanine nucleotide binding protein-like 2 | 3.2 | 347.6 | 1124.7 | 647.4 | 227.5 |
| | (nucleolar) | | | | | |
| Samsn1 | programmed cell death 11 | 2.0 | 353.9 | 711.8 | 435.9 | 287.4 |
| Tnfrsf4 | cyclin-dependent kinase 8 | 2.0 | 364.0 | 731.1 | 702.5 | 346.2 |
| Mettl1 | eukaryotic translation initiation factor 5B | 2.3 | 365.1 | 838.2 | 544.5 | 355.5 |
| Cd274 | RNA terminal phosphate cyclase-like 1 | 2.5 | 373.3 | 948.8 | 746.4 | 155.8 |
| Ubtd2 | NSFL1 (p97) cofactor (p47) | 2.3 | 374.1 | 876.1 | 725.9 | 369.7 |
| Icos | nuclear factor of kappa light polypeptide | | | | | |
| | gene enhancer in B cells inhibitor, delta | 3.9 | 378.5 | 1465.1 | 389.9 | 224.0 |
| Kdm2b | M-phase phosphoprotein 10 (U3 small | | | | | |
| | nucleolar ribonucleoprotein) | 2.8 | 379.8 | 1069.3 | 738.4 | 290.8 |
| Larp4 | GRAM domain containing 1B | 2.5 | 382.7 | 949.6 | 363.4 | 659.2 |
| Eif3d | ERO1-like (S, cerevisiae) | 2.2 | 387.7 | 872.3 | 773.0 | 520.9 |
| Tnfaip3 | nuclear receptor subfamily 4, group A, | 6.8 | 387.8 | 2639.0 | 343.7 | 220.7 |
| | member 1 | | | | | |
| Map1b | surfeit gene 2 | 2.1 | 399.8 | 852.2 | 696.3 | 204.0 |
| Cdv3 | N(alpha)-acetyltransferase 25, NatB auxiliary | 2.1 | 405.7 | 847.3 | 669.5 | 194.1 |
| | subunit | | | | | |
| Plac8 | yrdC domain containing (E, coli) | 2.0 | 406.7 | 830.8 | 635.3 | 267.0 |
| Mrpl3 | La ribonucleoprotein domain family, member | 2.2 | 408.8 | 887.9 | 586.6 | 358.3 |
| | 4 | | | | | |
| Surf2 | SDA1 domain containing 1 | 2.2 | 419.8 | 939.9 | 631.4 | 284.7 |
| Ubxn2a | importin 4 | 2.8 | 420.3 | 1183.6 | 777.8 | 173.5 |
| Utp18 | inducible T cell co-stimulator | 2.2 | 423.9 | 920.9 | 818.8 | 796.9 |
| Isg20 | solute carrier family 7 (cationic amino acid | 2.1 | 439.4 | 934.4 | 842.6 | 344.6 |
| | transporter, y+ system), member 1 | | | | | |
| Dnajc2 | arsA arsenite transporter, ATP-binding, | 2.6 | 446.6 | 1165.0 | 717.9 | 963.9 |
| | homolog 1 (bacterial) | | | | | |
| Jak2 | polymerase (RNA) I polypeptide C | 2.7 | 447.8 | 1208.4 | 854.0 | 295.9 |
| Slc7a1 | spermatogenesis associated 5 | 2.0 | 450.8 | 920.2 | 516.0 | 361.6 |
| Syde2 | ubiquitin specific peptidase 18 | 2.7 | 451.8 | 1240.5 | 296.0 | 250.7 |
| Slc5a6 | placenta-specific 8 | 2.1 | 452.4 | 967.3 | 888.6 | 590.8 |
| Dnttip2 | general transcription factor IIF, polypeptide 1 | 2.3 | 454.8 | 1063.9 | 890.0 | 680.8 |
| Idi2 | nuclear factor of kappa light polypeptide | 3.4 | 456.4 | 1535.5 | 679.1 | 502.7 |
| | gene enhancer in B cells inhibitor, beta | | | | | |
| Dus2 | PHD finger protein 6 | 2.5 | 462.0 | 1159.5 | 775.8 | 510.4 |
| Pitrm1 | RRN3 RNA polymerase I transcription factor | 2.1 | 462.2 | 948.4 | 913.2 | 388.9 |
| | homolog (yeast) | | | | | |
| Plxna1 | cytotoxic and regulatory T cell molecule | 2.5 | 473.7 | 1177.8 | 586.8 | 431.8 |
| Cdk5r1 | COP9 (constitutive photomorphogenic) | | | | | |
| | homolog, subunit 6 (Arabidopsis thaliana) | 2.3 | 483.6 | 1101.9 | 947.8 | 560.3 |
| Ube2cbp | asparagine-linked glycosylation 3 (alpha-1,3- | | | | | |
| | mannosyltransferase) | 2.1 | 485.9 | 1006.3 | 758.7 | 339.4 |
| Tnfsf11 | tryptophanyl-tRNA synthetase | 2.0 | 486.1 | 987.1 | 897.1 | 504.7 |
| Pop7 | hypoxia up-regulated 1 | 2.0 | 494.3 | 996.6 | 802.4 | 690.3 |
| Psme3 | family with sequence similarity 60, member A | 2.0 | 500.8 | 1002.1 | 834.7 | 417.6 |
| Mir17hg | bone marrow stromal cell antigen 2 | 3.8 | 502.5 | 1922.9 | 925.5 | 246.0 |
| Tsr1 | nuclear factor of kappa light polypeptide | 2.4 | 503.2 | 1231.8 | 494.0 | 341.8 |
| | gene enhancer in B cells 2, p49/p100 | | | | | |
| Rbpms2 | UTP20, small subunit (SSU) processome | 2.4 | 510.5 | 1240.2 | 696.4 | 245.8 |
| | component, homolog (yeast) | | | | | |
| Mrpl47 | CD274 antigen | 2.2 | 516.6 | 1128.7 | 246.9 | 220.2 |
| Rab8b | proviral integration site 1 | 3.4 | 518.4 | 1766.4 | 676.9 | 970.0 |
| Plagl2 | signal transducer and activator of | | | | | |
| | transcription 5A | 2.3 | 530.0 | 1210.4 | 496.6 | 507.8 |
| GrhI1 | CD69 antigen | 3.2 | 535.7 | 1725.8 | 289.5 | 153.9 |
| Zeb2 | pitrilysin metallepetidase 1 | 2.1 | 544.9 | 1153.8 | 968.4 | 349.3 |
| sept-02 | cyclin-dependent kinase 6 | 2.7 | 550.3 | 1476.5 | 1064.0 | 642.1 |
| Slc5a3 | DEAD (Asp-Glu-Ala-Asp) box polypeptide 27 | 2.3 | 556.2 | 1286.9 | 987.2 | 480.4 |
| Naa25 | polymerase (RNA) I polypeptide B | 2.8 | 556.2 | 1536.0 | 1070.4 | 201.3 |
| Plaur | tumor necrosis factor, alpha-induced protein | 2.2 | 560.6 | 1212.2 | 255.5 | 446.0 |
| | 3 | | | | | |
| Metap1 | nodal modulator 1 | 2.1 | 563.0 | 1161.0 | 988.9 | 439.8 |
| Alg3 | NOP14 nucleolar protein | 2.5 | 570.9 | 1418.9 | 925.3 | 398.0 |
| Mrpl15 | ribosomal protein L7-like 1 | 2.5 | 586.7 | 1448.7 | 1030.2 | 687.2 |
| Oasl1 | methionyl aminopeptidase 1 | 2.1 | 597.5 | 1244.1 | 1139.3 | 433.4 |
| Rorc | hypoxia inducible factor 1, alpha subunit | 3.0 | 624.2 | 1854.6 | 809.4 | 838.4 |
| Nomo1 | Janus kinase 2 | 2.1 | 624.5 | 1328.7 | 390.6 | 917.8 |
| Tgif1 | nuclear factor of kappa light polypeptide | 2.9 | 661.5 | 1913.3 | 713.9 | 720.5 |
| | gene enhancer in B cells 1, p105 | | | | | |
| Lipg | reticuloendotheliosis oncogene | 2.5 | 678.9 | 1686.4 | 409.8 | 580.5 |
| Rrn3 | septin 2 | 2.1 | 687.3 | 1436.0 | 1354.1 | 1181.3 |
| Dnajc21 | nucleolar protein interacting with the FHA | 2.3 | 733.4 | 1658.2 | 1280.0 | 407.2 |
| | domain of MKI67 | | | | | |
| Yrdc | elongation factor Tu GTP binding domain | 2.0 | 739.3 | 1483.5 | 1439.0 | 904.3 |
| | containing 2 | | | | | |
| Acsl6 | myelocytomatosis oncogene | 4.0 | 761.0 | 3022.8 | 1064.0 | 211.5 |
| Spata5 | dyskeratosis congenita 1, dyskerin | 2.7 | 778.2 | 2112.0 | 1549.5 | 484.2 |
| Urb2 | carnitine deficiency-associated gene | 2.1 | 801.6 | 1718.2 | 1274.7 | 1010.3 |
| | expressed in ventricle 3 | | | | | |
| Nlel | GTP binding protein 4 | 2.4 | 824.2 | 1942.6 | 1578.7 | 567.3 |
| Wars | HEAT repeat containing 1 | 2.4 | 830.3 | 2020.6 | 1235.5 | 495.4 |
| Crem | proteaseome (prosome, macropain) activator | 2.1 | 838.4 | 1763.5 | 1471.1 | 936.1 |
| | subunit 3 (PA28 gamma, Ki) | | | | | |
| Larp1 | La ribonucleoprotein domain family, member | 2.0 | 861.7 | 1742.1 | 1250.9 | 854.3 |
| | 1 | | | | | |
| Eif2ak2 | DNA segment, Chr 19, Brigham & Women's | 2.3 | 868.6 | 1978.4 | 1218.0 | 653.4 |
| | Genetics 1357 expressed | | | | | |
| Hyou1 | eukaryotic translation initiation factor 3, | 2.2 | 909.1 | 1971.6 | 1641.9 | 920.6 |
| | subunit D | | | | | |
| Senp3 | TSR1 20S rRNA accumulation | 2.1 | 913.9 | 1915.9 | 1474.6 | 477.2 |
| Tmtc2 | MYB binding protein (P160) 1a | 2.6 | 1140.0 | 2962.9 | 2200.7 | 459.8 |
| Fosb | T cell activation Rho GTPase activating | 2.4 | 1176.7 | 2794.4 | 489.3 | 704.2 |
| | protein | | | | | |
| Pdcd11 | RAB8B, member RAS oncogene family | 2.1 | 1189.5 | 2492.2 | 1671.3 | 2512.5 |
| Usp31 | DEAD (Asp-Glu-Ala-Asp) box polypeptide 21 | 2.4 | 1210.2 | 2928.0 | 2221.1 | 1098.2 |
| Cdk8 | chaperonin containing Tcp1, subunit 4 (delta) | 2.3 | 1321.4 | 2989.7 | 2462.5 | 1294.8 |
| Eftud2 | coiled-coil-helix-coiled-coil-helix domain | 2.3 | 1374.2 | 3171.2 | 2636.9 | 1008.9 |
| | containing 2 | | | | | |
| Fam60a | WD repeat domain 43 | 2.3 | 1727.6 | 3912.6 | 2927.5 | 1014.9 |

TABLE 7
Selection of preferred endogenous genes that are constantly active during
immune cell activation (dependent or independent from T-cell activation).

| Symbol | Gene description |
|---|---|
| CD3G | CD3 gamma |
| Rn28s1 | 28S ribosomal RNA |
| Rn18s | 18S ribosomal RNA |
| Rn7sk | RNA, 7SK, nuclear |
| Actg1 | actin, gamma, cytoplasmic 1 |
| B2m | beta-2 microglobulin |
| Rpl18a | ribosomal protein L18A |
| Pabpc1 | poly(A) binding protein, cytoplasmic 1 |
| Gapdh | glyceraldehyde-3-phosphate dehydrogenase |
| Rpl19 | ribosomal protein L19 |
| Rpl17 | ribosomal protein L17 |
| Rplp0 | ribosomal protein, large, P0 |
| Cfl1 | cofilin 1, non-muscle |
| Pfn1 | profilin 1 |

TABLE 8
Selection of genes that are transiently
upregulated upon T-cell activation.

| Symbol | Gene description |
|---|---|
| II3 | interleukin 3 |
| II2 | interleukin 2 |
| Ccl4 | chemokine (C-C motif) ligand 4 |
| II21 | interleukin 21 |
| Gp49a | glycoprotein 49 A |
| Nr4a3 | nuclear receptor subfamily 4, group A, member 3 |
| Lilrb4 | leukocyte immunoglobulin-like receptor, subfamily B, |
| | member 4 |
| Cd200 | CD200 antigen |
| Cdkn1a | cyclin-dependent kinase inhibitor 1A (P21) |
| Gzmc | granzyme C |
| Nr4a2 | nuclear receptor subfamily 4, group A, member 2 |
| Cish | cytokine inducible SH2-containing protein |
| Ccr8 | chemokine (C-C motif) receptor 8 |
| Lad1 | ladinin |
| Crabp2 | cellular retinoic acid binding protein II |

TABLE 9
Selection of genes that are upregulated
over more than 24 hours upon T-cell activation.

| Symbol | Description |
|---|---|
| Gzmb | granzyme B |
| Tbx21 | T-box 21 |
| Pdcd1 | programmed cell death 1 |
| Plek | pleckstrin |
| Chek1 | checkpoint kinase 1 |
| Slamf7 | SLAM family member 7 |
| Zbtb32 | zinc finger and BTB domain containing 32 |
| Tigit | T cell immunoreceptor with Ig and ITIM domains |
| Lag3 | lymphocyte-activation gene 3 |
| Gzma | granzyme A |
| Wee1 | WEE 1 homolog 1 (S. pombe) |
| II12rb2 | interleukin 12 receptor, beta 2 |
| Ccr5 | chemokine (C-C motif) receptor 5 |
| Eea1 | early endosome antigen 1 |
| Dtl | denticleless homolog (Drosophila) |

TABLE 10
Selection of genes that are down-regulated
upon immune cell activation.

| Symbol | Gene description |
|---|---|
| Spata6 | spermatogenesis associated 6 |
| Itga6 | integrin alpha 6 |
| Rcbtb2 | regulator of chromosome condensation (RCC1) and |
| | BTB (POZ) domain containing protein 2 |
| Cd1d1 | CD1d1 antigen |
| St8sia4 | ST8 alpha-N-acetyl-neuraminide alpha-2,8- |
| | sialyltransferase 4 |
| Itgae | integrin alpha E, epithelial-associated |
| Fam214a | family with sequence similarity 214, member A |
| Slc6a19 | solute carrier family 6 (neurotransmitter transporter), |
| | member 19 |
| Cd55 | CD55 antigen |
| Xkrx | X Kell blood group precursor related X linked |
| Mturn | maturin, neural progenitor differentiation regulator |
| | homolog (Xenopus) |
| H2-Ob | histocompatibility 2, O region beta locus |
| Cnr2 | cannabinoid receptor 2 (macrophage) |
| Itgae | integrin alpha E, epithelial-associated |
| Raver2 | ribonucleoprotein, PTB-binding 2 |
| Zbtb20 | zinc finger and BTB domain containing 20 |
| Arrb1 | arrestin, beta 1 |
| Abca1 | ATP-binding cassette, sub-family A (ABC1), member 1 |
| Tet1 | tet methylcytosine dioxygenase 1 |
| Slc16a5 | solute carrier family 16 (monocarboxylic acid |
| | transporters), member 5 |
| Trav14-1 | T cell receptor alpha variable 14-1 |
| Ampd3 | adenosine monophosphate deaminase 3 |

TABLE 11
Selection of human genes that are silent upon T-cell activation
(safe harbor gene targeted integration loci).

| Symbol | Gene description |
|---|---|
| Zfp640 | zinc finger protein 640 |
| LOC100038422 | uncharacterized LOC100038422 |
| Zfp600 | zinc finger protein 600 |
| Serpinb3a | serine (or cysteine) peptidase inhibitor, |
| | clade B (ovalbumin), member 3A |
| Tas2r106 | taste receptor, type 2, member 106 |
| Magea3 | melanoma antigen, family A, 3 |
| Omt2a | oocyte maturation, alpha |
| Cpxcr1 | CPX chromosome region, candidate 1 |
| Hsf3 | heat shock transcription factor 3 |
| Pbsn | Probasin |
| Sbp | spermine binding protein |
| Wfdc6b | WAP four-disulfide core domain 6B |
| Meiob | meiosis specific with OB domains |
| Dnm3os | dynamin 3, opposite strand |
| Skint11 | selection and upkeep of intraepithelial T cells 11 |

TABLE 12
List of gene loci upregulated in tumor exhausted infiltrating
lymphocytes (compiled from multiple tumors) useful for gene integration
of exogenous coding sequences as per the present invention

| Gene names | Uniprot ID (human) |
|---|---|
| CXCL13 | O43927 |
| TNFRSF1B | P20333 |
| RGS2 | P41220 |
| TIGIT | Q495A1 |
| CD27 | P26842 |
| TNFRSF9 | Q12933 |
| SLA | Q13239 |
| INPP5F | Q01968 |
| XCL2 | Q9UBD3 |
| HLA-DMA | P28067 |
| FAM3C | Q92520 |
| WARS | P23381 |
| EIF3L | Q9Y262 |
| KCNK5 | O95279 |
| TMBIM6 | P55061 |
| CD200 | P41217 |
| C3H7A | O60880 |
| SH2D1A | O60880 |
| ATP1B3 | P54709 |
| THADA | Q6YHU6 |
| PARK7 | Q99497 |
| EGR2 | P11161 |
| FDFT1 | P37268 |
| CRTAM | O95727 |
| IFl16 | Q16666 |

TABLE 13
List of gene loci upregulated in hypoxic tumor conditions useful for gene
integration of exogenous coding sequences as per the present invention

| Gene names | Strategy | |
|---|---|---|
| CTLA-4 | KO/KI | Target shown to be upregulated |
| LAG-3 (CD223) | KO/KI | in T-cells upon hypoxia exposure |
| PD1 | KO/KI | and T cell exhaustion |
| 4-1BB (CD137) | KI | |
| GITR | KI | |
| OX40 | KI | |
| IL10 | KO/KI | |
| ABCB1 | KI | HIF target |
| ABCG2 | KI | |
| ADM | KI | |
| ADRA1B | KI | |
| AK3 | KI | |
| ALDOA | KI | |
| BHLHB2 | KI | |
| BHLHB3 | KI | |
| BNIP3 | KI | |
| BNIP3L | KI | |
| CA9 | KI | |
| CCNG2 | KI | |
| CD99 | KI | |
| CDKN1A | KI | |
| CITED2 | KI | |
| COL5A1 | KI | |
| CP | KI | |
| CTGF | KI | |
| CTSD | KI | |
| CXCL12 | KI | |
| CXCR4 | KI | |
| CYP2S1 | KI | |
| DDIT4 | KI | |
| DEC1 | KI | |
| EDN1 | KI | |
| EGLN1 | KI | |
| EGLN3 | KI | |
| ENG | KI | |
| ENO1 | KI | |
| EPO | KI | |
| ETS1 | KI | |
| FECH | KI | |
| FN1 | KI | |
| FURIN | KI | |
| GAPDH | KI | |
| GPI | KI | |
| GPX3 | KI | |
| HK1 | KI | |
| HK2 | KI | |
| HMOX1 | KI | |
| HSP90B1 | KI | |
| ID2 | KI | |
| IGF2 | KI | |
| IGFBP1 | KI | |
| IGFBP2 | KI | |
| IGFBP3 | KI | |
| ITGB2 | KI | |
| KRT14 | KI | |
| KRT18 | KI | |
| KRT19 | KI | |
| LDHA | KI | |
| LEP | KI | |
| LOX | KI | |
| LRP1 | KI | |
| MCL1 | KI | |
| MET | KI | |
| MMP14 | KI | |
| MMP2 | KI | |
| MXI1 | KI | |
| NOS2A | KI | |
| NOS3 | KI | |
| NPM1 | KI | |
| NR4A1 | KI | |
| NT5E | KI | |
| PDGFA | KI | |
| PDK1 | KI | |
| PFKFB3 | KI | |
| PFKL | KI | |
| PGK1 | KI | |
| PH-4 | KI | |
| PKM2 | KI | |
| PLAUR | KI | |
| PMAIP1 | KI | |
| PPP5C | KI | |
| PROK1 | KI | |
| SERPINE1 | KI | |
| SLC2A1 | KI | |
| TERT | KI | |
| TF | KI | |
| TFF3 | KI | |
| TFRC | KI | |
| TGFA | KI | |
| TGFB3 | KI | |
| TGM2 | KI | |
| TPI1 | KI | |
| VEGFA | KI | |
| VIM | KI | |
| TMEM45A | KI | |
| AKAP12 | KI | |
| SEC24A | KI | |
| ANKRD37 | KI | |
| RSBN1 | KI | |
| GOPC | KI | |
| SAMD12 | KI | |
| CRKL | KI | |
| EDEM3 | KI | |
| TRIM9 | KI | |
| GOSR2 | KI | |
| MIF | KI | |
| ASPH | KI | |
| WDR33 | KI | |
| DHX40 | KI | |
| KLF10 | KI | |
| R3HDM1 | KI | |
| RARA | KI | |
| LOC162073 | KI | |
| PGRMC2 | KI | |
| ZWILCH | KI | |
| TPCN1 | KI | |
| WSB1 | KI | |
| SPAG4 | KI | |
| GYS1 | KI | |
| RRP9 | KI | |
| SLC25A28 | KI | |
| NTRK2 | KI | |
| NARF | KI | |
| ASCC1 | KI | |
| UFM1 | KI | |
| TXNIP | KI | |
| MGAT2 | KI | |
| VDAC1 | KI | |
| SEC61G | KI | |
| SRP19 | KI | |
| JMJD2C | KI | |
| SNRPD1 | KI | |
| RASSF4 | KI | |

1-71. (canceled)

72. A method for preparing a population of engineered primary human NK or T cells for immunotherapy comprising:
providing primary human NK or T cells;
introducing an exogenous coding sequence encoding an interleukin selected from IL-15, IL-12, or IL-2 with a sequence-specific endonuclease targeting an endogenous gene into the primary human NK or T cells;
cleaving the endogenous gene and inserting the exogenous coding sequence into the endogenous gene such that said interleukin is under transcriptional control of the promoter of the endogenous gene, while disrupting the coding sequence of the endogenous gene,
wherein the endogenous gene is selected from one encoding PD1, LAG3, TIGIT, CRTAM, CASP3, SMAD2, SKIL, TGIF1, BATF, IL-10, or PRDM1; and
introducing an exogenous coding sequence encoding a chimeric antigen receptor (CAR) or a recombinant TCR into the primary human NK or T cells;
wherein said engineered primary human NK or T cells secrete a level of the interleukin sufficient to enhance the antitumor activity of the cells.

73. The method of claim 72, wherein said interleukin is IL-2.

74. The method of claim 72, wherein said interleukin is IL-12.

75. The method of claim 72, wherein said interleukin is IL-15.

76. The method of claim 72, wherein said exogenous sequence is introduced into the PD1 gene.

77. The method of claim 72, wherein more than 50% of said engineered primary human NK or T cells are TCR negative T-cells and/or more than 50% of said engineered primary human NK or T cells are CAR positive cells.

78. The method of claim 72, wherein the CAR is an antiCD22 CAR.

79. The method of claim 73, wherein the CAR is an antiCD22 CAR.

80. The method of claim 74, wherein the CAR is an antiCD22 CAR.

81. The method of claim 75, wherein the CAR is an antiCD22 CAR.

82. The method of claim 76, wherein the CAR is an antiCD22 CAR.

83. The method of claim 77, wherein the CAR is an antiCD22 CAR.

84. The method of claim 72, wherein the exogenous coding sequence encoding an interleukin is inserted into the middle of the PD1 open reading frame using TALENS having the sequence of SEQ ID NO:20 and 21.

85. The method of claim 73, wherein the exogenous coding sequence encoding an interleukin is inserted into the middle of the PD1 open reading frame using TALENS having the sequence of SEQ ID NO:20 and 21.

86. The method of claim 74, wherein the exogenous coding sequence encoding an interleukin is inserted into the middle of the PD1 open reading frame using TALENS having the sequence of SEQ ID NO:20 and 21.

87. The method of claim 75, wherein the exogenous coding sequence encoding an interleukin is inserted into the middle of the PD1 open reading frame using TALENS having the sequence of SEQ ID NO:20 and 21.

88. The method of claim 76, wherein the exogenous coding sequence encoding an interleukin is inserted into the middle of the PD1 open reading frame using TALENS having the sequence of SEQ ID NO:20 and 21.

89. The method of claim 77, wherein the exogenous coding sequence encoding an interleukin is inserted into the middle of the PD1 open reading frame using TALENS having the sequence of SEQ ID NO:20 and 21.

90. The method of claim 72, wherein said exogenous sequence is introduced into the LAG3 gene.

91. The method of claim 72, wherein said exogenous sequence is introduced into the TIGIT gene.
